(19) United States
(12) Patent Application Publication
(10) Pub. No.: US 2026/0008830 A1
(43) Pub. Date: Jan. 8, 2026

(54) THERAPEUTIC CYTOKINES AND METHODS

(71) Applicants: FUNDACIÓ CENTRE DE REGULACIÓ GENÒMICA, Barcelona (ES); INSTITUCIÓ CATALANA DE RECERCA I ESTUDIS AVANÇATS, BARCELONA (ES)

(72) Inventors: Francisco Javier DELGADO BLANCO, BARCELONA (ES); Luís SERRANO PUBUL, Barcelone (ES); Ariadna MONTERO BLAY, BARCELONE (ES)

(21) Appl. No.: 18/832,680

(22) PCT Filed: Jan. 30, 2023
(86) PCT No.: PCT/EP2023/052202

§ 371 Date: Jul. 24, 2024

(30) Foreign Application Priority Data
Jan. 28, 2022 (EP) 22382073.9

(51) Int. Cl. C07K 14/55; A61K 38/00; C07K 14/535; C07K 14/54; C07K 14/56; C07K 14/565; C07K 14/57

(52) U.S. Cl. CPC C07K 14/55; C07K 14/535; C07K 14/5403; C07K 14/5406; C07K 14/5412; C07K 14/5418; C07K 14/5425; C07K 14/5431; C07K 14/5434; C07K 14/5443; C07K 14/56; C07K 14/565; C07K 14/57; A61K 38/00; C07K 2319/74

(57) ABSTRACT

Disclosed herein are single chain dimeric cytokines having improved physiological activity over wild-type cytokine monomers. The single chain dimeric cytokines of the disclosure may be useful in the treatment of various diseases or disorders relating to inflammatory conditions or proliferative conditions. Also disclosed are delivery vectors or agents for delivering the single chain dimeric cytokines to a subject. Such delivery vectors or agents include cells, bacteria and bacteriophages.

This application is the U.S. national phase of International Application No. PCT/EP2023/052202 filed Jan. 30, 2023, which designated the U.S. and claims priority to EP patent application Ser. No. 22/382,073.9 filed Jan. 28, 2022, the entire contents of each of which are hereby incorporated by reference.

The content of the electronically submitted sequence listing (Name: FLDK_BET24P3003_Sequence_listing.xml, Size: 342 KB, and Date of Creation: Feb. 24, 2025) is herein incorporated by reference in its entirety.

### FIELD OF THE INVENTION

The invention relates to novel therapeutic cytokines and methods of their use in therapy. In particular, the invention relates to novel dimeric cytokines and more particularly to forced conformation/structurally constrained dimeric cytokines and their use in therapy.

### BACKGROUND OF THE INVENTION

Cytokines are biomolecules of great potential interest for human therapy. They modulate the immune system and play an important role in cancer, inflammation, immune response, tissue regeneration, among others. Despite their great potential, there are only a handful of cytokines approved for therapeutic purposes. This is because many of them can have adverse side effects as they usually target different cell types which adds to their low serum half-life, high production costs, pleiotropic effects, high toxicity, etc. (see FIG. 2).

Different methods have been proposed to improve the pharmacodynamics and pharmacokinetics of selected cytokines (e.g. fusion to the IgG-Fc region to IL-72 or the use of adjuvant polyethylene glycol for many of them³). Other properties that need improvement include their affinity and resistance to proteolysis, and/or their toxicity which could be partly solved by designing surface mutations targeting the interaction with one or more receptors to administrate lower doses but maximizing the therapeutic effect⁴).

One way to potentially decrease cytokine toxicity is to deliver them locally by using a therapeutic bacterium. The use of recombinant bacteria producing and delivering in situ the therapeutic molecule of interest has been extensively described⁵. Many such efforts have also shown high efficacy in animal models but have yet to yield clinical successes, in part because of difficulties in achieving and maintaining sufficiently effective concentrations of the therapeutic molecule at the site of disease. Nevertheless, the promise of effective treatments that can be produced cheaply and delivered orally or topically (e.g. Cutibacterium acnes for acne; https://pubmed.ncbi.nlm.nih.gov/31981578/), and with minimal systemic side effects has continued to fuel interest in the use of microbes as therapeutics. Several companies are developing bacteria for human therapy (e.g. Pulmobiotics, Eligo Bioscience, Synlogic, Azitra, Intrexon, Rebiotix), or using bacteriophages containing the genes of interest to be expressed in the target bacteria, (e.g. Armata Pharmaceuticals, Eligo Bioscience).

Another way to decrease toxicity and improve efficacy may be to fuse the cytokine to a molecule that will target it to the desired action site (e.g. an antibody), introduce mutations that will change relatively the affinity towards one of the different components of the receptor assembly taking into account eventually the different expression level of receptor components in different cells (e.g. IL2 and its receptor alpha, (IL-2RA)); or fusing it to other protein domains that prevent receptor recognition and that are released from the cytokine at the right location.

Against the above background, it would be desirable to have new and/or improved therapeutic cytokines, therapies and/or methods for improving the half-life, safety, efficacy, specificity and/or local targeting of cytokines when administered either by conventional methods or using a biological delivery vector.

The present invention seeks to overcome or at least alleviate one or more of the deficiencies in the prior art.

### SUMMARY OF THE INVENTION

The invention relates to a single chain dimeric cytokine polypeptide comprising: (a) a first cytokine monomer domain or a functional portion thereof; and (b) a second cytokine monomer domain or a functional portion thereof, wherein the sequence of one of the cytokine monomer domains or functional portion is not continuous and is interrupted by the sequence of the other cytokine monomer domain or a functional portion thereof.

In an embodiment, the sequence of the first cytokine monomer domains or functional portion is not continuous and is interrupted by the sequence of the second cytokine monomer domain or a functional portion thereof.

In some aspects of this embodiment, the first sequence portion of the first cytokine monomer domain corresponds to a N-terminal portion of a natural cytokine monomer domain, and the second sequence portion of the first cytokine monomer domain corresponds to an C-terminal portion of the natural cytokine monomer domain.

In other aspects of this embodiment, the first sequence portion of the first cytokine monomer domain corresponds to a C-terminal portion of a natural cytokine monomer domain, and the second sequence portion of the first cytokine monomer domain corresponds to an N-terminal portion of a natural cytokine monomer domain.

In another embodiment, the sequence of the second cytokine monomer domains or functional portion is not continuous and is interrupted by the sequence of the first cytokine monomer domain or a functional portion thereof.

In some aspects of this embodiment, the sequence of the first class I cytokine monomer domain or functional portion thereof is continuous in sequence, while the sequence of the second cytokine monomer domain is such that the first sequence portion of the second class I cytokine monomer which is arranged at the N-terminus of the first class I cytokine monomer or functional portion thereof corresponds to a N-terminal portion of a natural class I cytokine monomer, and the second sequence portion of the second class I cytokine monomer which is arranged at the C-terminus of the second class I cytokine monomer or functional portion thereof corresponds to a C-terminal portion of the natural class I cytokine monomer.

In other aspects of this embodiment, the sequence of the second class II cytokine monomer domain or functional portion thereof is continuous in sequence and in reverse order compared to its natural sequence order, while the sequence of the first class II cytokine monomer domain is such that the first sequence portion of the first class II cytokine monomer domain which is arranged at the N-terminus of the second class II cytokine monomer domain or functional portion thereof corresponds to a N-terminal portion of a natural class II cytokine monomer domain, and the second sequence portion of the first class II cytokine monomer domain which is arranged at the C-terminus of the second class II cytokine monomer domain or functional portion thereof corresponds to a C-terminal portion of the natural class II cytokine monomer domain.

In another embodiment, the invention relates to a single chain dimeric cytokine polypeptide that comprises a first cytokine monomer and a second cytokine monomer that form swapped domain dimers, wherein a linker peptide bridges the N-terminus of the first cytokine monomer with the C-terminus of a second cytokine monomer.

The invention further relates to a polypeptide comprising a single chain dimeric polypeptide of the invention.

The invention further relates to a nucleic acid encoding a single chain dimeric polypeptide of the invention, or a polypeptide comprising it.

The invention also relates to an expression vector comprising a nucleic acid of the invention. Also provided is a host cell that comprises a nucleic acid or expression vector of the invention.

The invention also relates to a pharmaceutical composition comprising a single chain dimeric polypeptide of the invention, or a polypeptide comprising it.

Also provided is a method of treating a disease, that comprises administering a single chain dimeric polypeptide of the invention, or a polypeptide comprising it, to a subject in need thereof, optionally in a subject that would benefit from a reduction or increase in an inflammatory response or in rate of cell proliferation.

The invention further relates to a single chain dimeric polypeptide of the invention, or a polypeptide comprising it, for use for treating a disease, optionally a disease that would benefit from a reduction or increase in an inflammatory response or in rate of cell proliferation.

The invention further relates to a single chain dimeric polypeptide of the invention, or a polypeptide comprising it, for the manufacture of a medicament for treating a disease, optionally a disease that would benefit from a reduction or increase in an inflammatory response or in rate of cell proliferation.

Broadly, this disclosure relates to the structural design of new cytokine polypeptide variants that may provide beneficial therapeutic effects. Such therapeutic polypeptides may be delivered to a target subject by any method available to the skilled person, for example, orally, by spray, injection or using biological vectors like viruses, bacteriophages and/or bacteria.

The new cytokine polypeptide variants of this disclosure comprise pairs of cytokine monomer sequences that are covalently linked to form single chain polypeptides. The cytokine monomer sequences may be derived from the same or different wild type cytokine protein sequences. Beneficially, the single chain polypeptides of the disclosure fold to form a tertiary structure that comprises two structural domains (e.g. left and right 3D domains), wherein each 3D domain may have the function and/or activity of a natural cytokine monomer on which it is based/derived; or may have a different function or activity.

In accordance with one aspect, it is disclosed a single chain dimeric cytokine polypeptide comprising: (a) a first cytokine monomer domain or a functional portion thereof; and (b) a second cytokine monomer domain or a functional portion thereof, wherein the first cytokine monomer domain or a functional portion thereof is fused to the second cytokine monomer domain or a functional portion thereof by a peptide linker, wherein the peptide linker sequence comprises 5 or less (preferably 3 or less) adjacent Gly and/or Ser residues. Suitably, the engineered peptide linker has a sequence of from about 3 to about 20 amino acids. In embodiments, the engineered peptide linker has about 3 to about 16 amino acids; about 3 to about 12 amino acids; about 3 to about 8 amino acids; about 4 to about 8 amino acids; or about 4 to about 6 amino acids.

In accordance with a second aspect, it is disclosed a single chain dimeric cytokine polypeptide comprising: (a) a first cytokine monomer domain or a functional portion thereof; and (b) a second cytokine monomer domain or a functional portion thereof, wherein the sequence of the second cytokine monomer domain of functional portion thereof is inserted within the sequence of the first cytokine monomer domain such that a first sequence portion of the first cytokine monomer is arranged at the N-terminus of the second cytokine monomer or functional portion thereof, and a second sequence portion of the first cytokine monomer is arranged at the C-terminus of the second cytokine monomer or functional portion thereof. Beneficially, the single chain dimeric cytokine polypeptide has a tertiary structure comprising a left 3D cytokine domain and a right 3D cytokine domain, and wherein the left 3D cytokine domain is connected to the right 3D cytokine domain by two bridging linker peptides. In embodiments, the left 3D cytokine domain is suitably a split domain cytokine formed from first and second sequence portions of the first cytokine monomer domain or a functional portion thereof and the right 3D cytokine domain is suitably a continuous domain cytokine formed from the second cytokine monomer domain or a functional portion thereof.

In accordance with a third aspect, it is disclosed a single chain dimeric cytokine polypeptide comprising: (a) a first cytokine monomer domain or a functional portion thereof; and (b) a second cytokine monomer domain or a functional portion thereof, wherein the sequence of the second cytokine monomer domain of functional portion thereof is inserted within the sequence of the first cytokine monomer domain such that a first sequence portion of the first cytokine monomer is arranged at the N-terminus of the second cytokine monomer or functional portion thereof, and a second sequence portion of the first cytokine monomer is arranged at the C-terminus of the second cytokine monomer or functional portion thereof. In various embodiments, the first sequence portion of the first cytokine monomer domain corresponds to a C-terminal portion of a natural cytokine monomer, and the second sequence portion of the first cytokine monomer domain corresponds to an N-terminal portion of a natural cytokine monomer.

In accordance with a fourth aspect, it is disclosed a single chain dimeric cytokine polypeptide comprising: (a) a first cytokine monomer domain or a functional portion thereof; and (b) a second cytokine monomer domain or a functional portion thereof, wherein the first cytokine monomer domain sequence is derived from a different type of cytokine to the second cytokine monomer domain sequence; and wherein the left cytokine domain is a split domain cytokine formed from first and second sequence portions of the first cytokine monomer domain or a functional portion thereof and the right 3D cytokine domain is a continuous domain cytokine formed from the second cytokine monomer domain or a functional portion thereof. Suitably, the single chain dimeric cytokine polypeptide has a tertiary structure comprising a left 3D cytokine domain formed of the first cytokine monomer domain or a functional portion thereof, and a right 3D cytokine domain formed of the second cytokine monomer domain or a functional portion thereof.

Beneficially, the first cytokine monomer domain is connected to the second cytokine monomer domain by two bridging linker peptides. In embodiments, the first cytokine monomer domain is derived from a class II cytokine and the second cytokine monomer is derived from a class I cytokine; or the first cytokine monomer domain is derived from a class I cytokine and the second cytokine monomer is derived from a class II cytokine.

In accordance with a fifth aspect, it is disclosed a single chain dimeric cytokine polypeptide comprising: (a) a first cytokine monomer domain or a functional portion thereof; and (b) a second cytokine monomer domain or a functional portion thereof, wherein the first cytokine monomer domain sequence is derived from a different type of cytokine to the second cytokine monomer domain sequence; and wherein the single chain dimeric cytokine polypeptide has a tertiary structure comprising a left 3D cytokine domain formed of the first cytokine monomer domain or a functional portion thereof, and a right 3D cytokine domain formed of the second cytokine monomer domain or a functional portion thereof. In various embodiments: (i) the first cytokine monomer domain sequence is derived from an IL-10 or IFNγ cytokine sequence and the second cytokine monomer domain sequence is derived from a class I or a class II cytokine sequence; or (ii) the first cytokine monomer domain sequence is derived from a class I or a class II cytokine sequence and the second cytokine monomer domain sequence is derived from an IL-10 or IFNγ cytokine sequence.

This disclosure also encompasses isolated polynucleotides that encompass any of the polypeptide of the disclosure. Vectors comprising such polynucleotides are also encompassed, such as bacteriophage or viral vectors.

In addition, the disclosure encompasses cells comprising the polynucleotides of the disclosure and/or cells expressing the polypeptides of the disclosure.

The disclosure also relates to pharmaceutical compositions comprising the polypeptides, polynucleotides, vectors and/or cells of this disclosure.

Furthermore, the disclosure relates to therapeutic uses and methods of medical treatment involving the polypeptides, polynucleotides, vectors, cells and/or (pharmaceutical/therapeutic) compositions of this disclosure.

Various aspects and embodiments of the disclosure are defined in the Clauses and/or in the appended claims. Further aspects and embodiments of the inventive concepts are described elsewhere herein.

It will be appreciated that any features of one aspect or embodiment of the invention may be combined with any combination of features in any other aspect or embodiment of the invention, unless otherwise stated, and such combinations fall within the scope of the claimed invention. Thus, the invention encompasses all of the clauses and claims of this disclosure in any and all combinations, unless such combination is incompatible.

### BRIEF DESCRIPTION OF THE DRAWINGS

The invention is further illustrated by the accompanying drawings in which:

FIG. 1: Schematic illustration of the general concept of foldikine. The basic foldikine concept is a single-chain molecule composed of a folded cytokine domain continuous in sequence and 3D space (clear ribbon; right hand side) linked via its N- and C-terminus (Xn linker in grey, which could be the natural N- and C-termini or which could result from opening a loop in the natural sequence to form new N- and C-termini in conjunction with closing the natural N- and C-termini with a NtCt linker—also illustrated in grey) to another domain based on a cytokine, which is split into two parts of equal or different size (black and medium grey, left hand side) where the N- and C-termini of the resulting foldikine molecule may be the natural N- and C-termini of the second (left hand side) cytokine domain or the new N- and C-termini resulting from the split of the second cytokine domain. In this particular example a foldkine is illustrated that is created based on a type II cytokine in which the natural N- and C-termini of the cytokine monomer that forms the continuous 3D domain have been linked with an engineered linker sequence (NtCt). To create the fused dimer structure a loop that naturally bridges α-helices 3 and 4 in the two monomers has been opened, and the newly created N- and C-termini of both domains have been linked via two Xn designed linkers (grey). In this way, the continuous 3D domain (helices 1B to 6B labelled in light grey) is connected with the two halves (helices 1A to 3A labelled in black, and helices 4A to 6A labelled in grey), respectively, of the discontinuous 3D domain. Helices are numerated in the order they appear in the sequence for each domain of the natural monomeric foldikine (helices 1A to 6A). In the continuous 3D domain of the foldikine, according to this embodiment, the helix order in the N- to C-terminus direction is 4B, 5B, 6B, 1B, 2B, 3B). The number of helices vary depending on the cytokines on which the foldikine is derived. For example, a type I cytokine typically includes 4 conserved α-helices, whereas a type II cytokine typically includes 6 conserved α-helices. The designed linkers are shown as ribbon with the protein backbone (N, CA, CO atoms) shown.

FIG. 2: Flow diagram illustrating the cytokine problem and solution using foldikines.

FIG. 3: Schematic illustration of the process for forming a foldikine based on a dimeric swapped domain dimeric cytokine type II. N-ter A and C-ter A indicate the N- and C-termini of the first cytokine monomer, and N-ter B and C-ter B indicate the N- and C-termini of the second cytokine monomer of the swapped domain dimer. A swapped domain foldikine like IL-10 is composed of two chains (dark and grey helices) forming two cytokine domains composed of the N- and C-regions of each molecule. The loop in black represents the new linker (N—C linker) designed to join the N-terminus of one of the cytokine monomers (in this example the B monomer) to the C-terminus of the other of the cytokine monomers (in this example the A monomer) to make a ‘continuous foldikine domain’ (see also FIG. 25), connected by two linkers to a split domain (left 3D domain).

FIG. 4: Schematic illustration of the process for designing a foldikine according to Way B based on two monomeric class I short or long chain cytokines. N-ter A and C-ter A indicate the N- and C-termini of one of the original or wild-type cytokine monomers (A), and N-ter B and C-ter B indicate the N- and C-termini of the other of the original cytokine monomers (B) that form a dimeric cytokine. The loop in black in the A cytokine domain is exemplary, and represents the loop that in this example is opened to create the foldikine in combination with joining the original N-ter A and C-ter B and the original N-ter B and C-ter A of monomers A and B (illustrated with black linkers; centre) to create a single chain foldikine. In this illustration a peptide loop in the A domain is opened (see also FIGS. 6 and 27) to form the N- and C-termini of the foldikine. In other embodiments, as different loop between helices may be opened in the A domain to form the new N- and C-termini. In other embodiments, a loop in the B cytokine may be opened to form the new N- and C-termini.

FIG. 5: Schematic illustration of the process for designing a foldikine according to Way A based on two monomeric class II cytokines. N-ter A and C-ter A indicate the N- and C-termini of one of the original or wild-type cytokine monomer (A), and N-ter B and C-ter B indicate the N- and C-termini of the other of the original cytokine monomer (B). The loops in black (top left panel) are exemplary and represent two loops that are opened to create new N- and C-terminal ends (N-N-ter A and B and N—C-ter A and B; top right panel). These new N- and C-termini can be connected with new peptide linkers (indicated by small black arrows; bottom right panel). The original N-ter and C-ter of one of the two original/wild-type cytokine monomers (in this example cytokine B) are then joined with a new peptide linker (small black arrow; bottom left panel) to form a single polypeptide with two foldikine domains: a continuous domain (right) sandwiched between two parts of a split domain (left-see also FIG. 27).

FIG. 6: Schematic illustration of the process for designing a chimeric foldikine based on a monomeric class I and a monomeric class II cytokine. According to Way B, N-ter A and C-ter A indicate the N- and C-termini of an original/wild-type class II cytokine monomer (i), and, according to Way C, N-ter B and C-ter B indicate the N- and C-termini of an original/wild-type class I cytokine monomer (ii) (see top left in panels A and B). Two possible strategies to create a foldikine composed of a type I and a type II monomer are illustrated. (i) The N-terminus of one monomer is covalently linked to the C-terminus of the other monomer and vice versa to create a single polypeptide (see black lines, top right panel). In addition, a linker region between adjacent alpha helices in one of the original monomer domains (see black line; bottom right panel) is opened to create new N- and C-termini (N-N-ter and N—C-ter; bottom left panel) in the foldikine. In the depicted embodiment the new termini are created in the original cytokine type I sequence of domain B (see also FIGS. 4 and 27). (ii) A linker region/loop is opened in the type II cytokine (cytokine A) to create new N- and C-termini (N-N-ter and N—C-ter; panel top right). Then the new N-terminus (N-N-ter) of monomer A is covalently linked to the natural C-terminus (C-ter B) of monomer B and the new C-terminus (N—C-ter) of monomer A is covalently linked to the natural N-terminus (N-ter B) of monomer B to join the two molecules (see black Xn labelled lines; bottom right panel) to create Foldikine II-I. Alternatively, the natural N- and C-terminal ends of monomer A are connected (NtCt) to form a circular permutant continuous domain of monomer A, and a linker region/loop is opened in the type I cytokine (monomer B) to create new N- and C-termini (bottom left panel) creating a Foldikine I-II.

FIG. 7: Schematic illustration showing how the structure of a swapped domain type II cytokine (SC-IL10) can be used as a reference for superimposition of two type II cytokine monomers, A and B. In the first step, the monomers A and B are superimposed over the respective folded 3D domains of the swapped domain template to create the desired relative positioning and orientation of the A and B monomers. In the second step, an opposing loop between helices of each monomer domain are opened to create new N- and C-terminal ends on each monomer that are joined with the compatible new ends of the opposing monomer to form a pair of bridging linkers joining the monomers (as illustrated in FIGS. 3 to 6). Bridging linker sequence lengths and composition may be) determined by modelling optimal linker sequences taking into account sequence and structure conformation of each monomer in order to form a two-domain foldikine that maintains an optimal or desired structural conformation and geometric configuration between the two foldikine domains. In another step, the natural N- and C-termini of one of the monomers (A or B) are linked to close one of the monomers and to create a single-chain foldikine polypeptide. The monomer, A or B, which is closed by linking its natural N- and C-termini becomes a ‘continuous’ domain in sequence and 3D space.

FIG. 8: Schematic representation of the structural confirmations of three different wild-type swapped domain cytokine dimers when bound to their respective receptors, showing the angle between the two halves of the dimeric cytokine and their approximate orientation with respect to the cell membrane: (A) The IFNγ cytokine with receptors R1 and R2 indicated, and exhibiting a V-angle between left and right hand side domains; (B) IL-10 cytokine with receptors R1 and R2 indicated, and exhibiting a V-angle between left and right hand side domains; and (C) IL-5 cytokine with one receptor bound, and exhibiting a planar 180-degree angle between the two structural 3D domains. Foldikines according to this disclosure are suitably designed and configured with covalent peptide linkers to constrain/encourage a tertiary structure that mimics the quaternary structure of a swapped wild-type cytokine dimer.

FIG. 9: Effects of recombinant IL-10 or IL-10 secreted by bacteria on monocyte primary cells. (A), (B) Mean fluorescence intensity (MFI) of flow cytometry data of cells from human donors (n=4) for anti-inflammatory markers (CD163, CD16, MerTK, CCR4 and PDL1) and pro-inflammatory markers (MHC2 and CD86) after adding monocytes media (control) or hIL-10 recombinant (hIL-10r) (A) or adding supernatant from wild-type (WT) M. pneumoniae (control) or M. pneumoniae expressing IL-10 ORF (WT_IL10) (B). Light grey bars represent the mean fluorescence intensity (MFI) for the control conditions; dark grey bars represent the MFI of the test conditions. * p<0.01 (2-way ANOVA and post-hoc Bonferroni multiple comparison test). (C), Western blot results of monocytes isolated from two independent donors, showing unstimulated cells (control) or cells stimulated with recombinant human IL-10 (hIL-10r), supernatant of WT M. pneumoniae (WT Control) or supernatant of M. pneumoniae expressing IL-10 (WT-IL-10).

FIG. 10: Schematic illustrations of IL-10 mutations generated in this work to increase IL-10 R1 and R2 receptor binding affinity. Details for each of the positions mutated to improve interactions with R1 or R2 are shown in the individual panels.

FIG. 11: Expression levels and apparent dissociation constant of selected IL-10 variants expressed in or by M. pneumoniae. (A) The apparent KD (molar) for IL-10 ORF, MutM, hIL-10r, Mut1, Mut2, Mut3, MutSC1 and MutSC2. The average of the apparent KD (picomolar) is shown in grey on top of each mutant. n>3 biological replicates. *, p<0.05 (one-way ANOVA and post-hoc Bonferroni multiple comparison test, selecting as reference IL-10 ORF condition). (B) Expression levels in femtograms (fg) of IL-10 secreted to the medium normalised by the number of bacterial colony-forming units (CFUs) at 48 h post-inoculum (hpi). *, p<0.05 (one-way ANOVA+Tukey post-hoc test). (C) Impact of linker composition on protein functionality for MutSC1 and MutSC1_control Gly. The x-axis shows the range of IL-10 concentration analysed, and the y-axis, the absorbance at 630 nm measured in HEK-Blue™ cells (N=3, n=2). (D) Western blot of p-STAT3 activation after induction with a fixed IL-10 concentration (20 ng/ml) in two different cell lines: THP-1 and HAFTL. The ratio p-STAT3/STAT3 was measured after 20 min of induction and is shown at the bottom of the graph. (E and F) Average fold change in apparent Kd determined by flow cytometry between IL-10 ORF and each of the single chain mutants MutSC1 and MutSC2 determined in BlaER1 (E) and HAFTL cells (F)

FIG. 12: Schematic illustrations depicting the design of single-chain (SC) IL-10 foldikines. (A) Steps to generate two different fusion patterns: (1) fringe residues deletion; (2) rewiring scheme; (3) structural rearrangement after rewiring the corresponding regions; (4) final numbering in the SC, including numerical gaps long enough to host peptides bridges with different lengths (up to 20) during linker search or bridging. Monomer 2 residue numbers are marked by ′ in left hand panel. (B) MutSC1 and MutSC2 built from the IL-10 fusion pattern 1. Linker sequences are shown as grey labels; post-rewiring mutations are included for MutSC2 using monomeric IL-10 numbering.

FIG. 13: Characterisation of Mycoplasma pneumoniae CV8 chassis in vivo. C57Bl/6 mice were infected with 107 CFUs of WT or CV8 strains and sacrificed at 2- or 4-days post-infection (dpi) (n≥3/group). (A) Similar bacterial loads (Log 10 CFU/lung homogenate) were found for the WT (circle) and CV8 (square) strains at the analysed time points. (B) Inflammatory profile of lungs inoculated with WT (middle bar), CV8 (right bar) or PBS (left bar). Gene expression was analysed by RT-qPCR using gapdh as an endogenous control (Methods). Data are shown as mean±SD of fold-change (FC) in mRNA expression (one-way ANOVA+Tukey post-hoc test) (*, p<0.05). (C) Histological findings of lung samples. Left, plots represent the quantitative evaluation (score: 0-5) of alveolar (top panel) and peribronchial/peribronchiolar infiltrate (bottom panel). Parameters were normalised using the average of the PBS group (FC=sample value/average PBS; FC control group, ˜1). Data are shown as mean±SEM of FC (one-way ANOVA+Tukey post-hoc test) (*, p<0.05). Right, representative images of lungs stained with hematoxylin-eosin are shown (line represents 250 μm). (D) Characterisation of WT and CV8 expressing cells. The IL-10 ORF production by the WT and CV8 strains analysed was measured by ELISA and normalised by protein content (biomass). The functionality of the IL-10 variants generated by the WT or CV8 strain was verified in HEK-Blue™ cells, using supernatants of the WT or CV8 strain as negative controls (one-way ANOVA+Tukey post-hoc test) (*, p<0.05).

FIG. 14: Immunomodulatory capacities of IL-10 variants in vivo. (A) Schematic representation of the mice model. (B) CFUs of PAO1 (left) or Mycoplasma strains (right) recovered from lung samples. Data are shown as mean±SEM of Log 10 CFU/lung homogenate. (C) Fold-change (FC) of mRNA gene expression of inflammatory markers. The 2-ΔΔCt method was used to normalise the values using gapdh as endogenous control. Data are shown as mean±SEM of FC. Statistical analysis was performed using unpaired t-test. (D) P values of statistical comparison of PAO1+ PBS group vs uninfected animals or PAO1+treatment groups with CV8, CV8 encoding IL-10 WT (CV8-IL-10 ORF), MutSC1 (CV8_MutSC1), MutSC2 (CV8_MutSC2) or human IL-10 recombinant protein (hIL-10r). Dark grey=significant increase; light grey=significant reduction of the gene expression. (E) Quantitative analysis of neutrophil elastase (NE), as shown by immunochemistry (IHC) of lung samples. Data are represented as the mean±SEM of percentage of positive cells, calculated as follows: %=100×(positive cells/positive cells+negative cells). (F) Representative images of IHC against NE are shown. Rows indicate NE-positive cells.

FIG. 15: Multiple sequence alignment of IL-10 polypeptides from different vertebrate species performed with the ClustalX algorithm. IL-10 human (SEQ ID NO: 349); IL-10 mouse (SEQ ID NO: 350); IL-10 macaque (SEQ ID NO: 351); IL-10 chicken (SEQ ID NO: 352); IL-10 guineapig (SEQ ID NO: 353) and IL-10 Danio rerio (SEQ ID NO: 354).

FIG. 16: Superimposition of the crystal structures of IL-10 bound to receptor R1 (1yI6k), and of IL-10 bound to both receptors R1 and R2 (6×93). The relatively unstructured region that adopts a different conformational structure when interacting with R2 is shown at the bottom right.

FIG. 17: Analysis of P. aeruginosa PAO1 infection of mice lungs. (A) Bacterial load (CFUs) obtained from mice lung homogenates at 24- or 48 hours post-infection (hpi). (B) Fold-change in mRNA expression of different inflammatory markers at 24 hpi with PAO1 at doses of 105 and 104 CFU/mouse.

FIG. 18: Generation of controls to validate the protein design loop in the foldikine-10. (A) Activation of HEK-blue cells-10 using the supernatant of Mycoplasma pneumoniae bacteria cell culture. (B) Measuring the Kdapp for the foldikine-10 (MutSC1) and the two controls to demonstrate the importance of the designed linkers.

FIG. 19: Graph showing the impact of linker composition over foldikine-22. Foldikine22_3 is foldikine-22_3 (SCIL22_3) as used herein, Foldikine22_controlcentre is foldikine-22_3_centrallinkers as used herein, and Foldikine22_N—C loop polyG is foldikine-22_3_linkerNCpolygly as used herein. Demonstrating that replacement of designed loops between N- and C-termini of one foldikine domain, or in bridges between foldikine domains with polyG causes a significant loss of activity.

FIG. 20: Analysis of the impact of the IL-22 hydrophobic mutations compared to IL22 wt.

FIG. 21: Analysis of the impact of individual mutations in the hydrophobic core of IL-22. IL-22 is IL-22 WT, C>A in termini is the mutation of C at position 7 to A; 56 is T56M point mutation in monomeric IL22; 66 is A66M point mutation in monomeric IL22; 95 is V95I point mutation in monomeric IL22; 99 is T99F point mutation in monomeric IL22; 173 is S173L point mutation in monomeric IL22. Bottom: apparent average Kds determined for WT and mutant constructs.

FIG. 22: Graphs showing response of HEK-Blue™ IL-22 cells to IL-22 engineered versions. The HEK-Blue™ IL-22 cells were incubated with supernatant of bacteria expressing either IL-22, foldikine-22_1, foldikine-22_3 or one of the controls with polyGly linkers in N- to C-termini (foldikine-22_3_linkerNCpolygly). After 24 h incubation, internal signalling activation was assessed by measuring SEAP levels in the supernatant using Quanti-Blue™. (A) non-diluted supernatants (concentration >30 ng/ml). (B) eight serial dilutions of IL-22 concentrations starting in 30 ng/L (diluting 0.5× each). In the x-axis the concentration is displayed in Molar.

FIG. 23: Response of HEK-Blue™ IL-10 and IL-22 cells to foldikine-10/22 (SCIL10IL22). The HEK-Blue™ IL-10 cells were incubated with supernatant of bacteria expressing two different chimeras (Chimera3 and Chimera5). After 24 h incubation, internal signalling activation was assessed by measuring SEAP levels in the supernatant using Quanti-Blue™. The non-diluted supernatants (concentration of quantified chimera >30 ng/mL) were assessed for IL-10 and IL-22 activity, respectively.

FIG. 24: Response of HEK-Blue™ IL-10 and IL-22 cells to heterodimers fused by short poly gly-ser linkers. The HEK-Blue™ IL-10 cells were incubated with supernatant from bacteria expressing IL-10, IL-22, heterodimer 10-22 (hetero1022) or heterodimer 2210 (hetero2210). After 24 h incubation, internal signalling activation was assessed by measuring SEAP levels in the supernatant using Quanti-Blue™. For (A) and (B) panels, non-diluted supernatants (concentration of quantified chimera >30 ng/mL) were assessed in IL-10 and IL-22 respectively.

FIG. 25: Schematic illustration of a mutation scheme for forming a foldikine based on a swapped domain dimeric class I cytokine: (i) wild-type domain swapped dimer showing «-helix D of each monomer unit aligning with the opposing monomer α-helices A, B and C, to form adjacent 3D domains comprising α-helices A, B and C of a first monomer sequence and α-helix D of a second monomer sequence; (ii) single-chain foldikine based on fused wild-type monomers in which monomer sequences are joined to make a single chain polypeptide by linking the C-terminus of one monomer sequence to the N-terminus of a second monomer sequence using a peptide linker (dashed line). Wild-type linkers shown in solid lines. Boxes indicate the α-helices that form each of the left and right 3D domains. As depicted, there are two peptide linkers spanning the first (left) and second (right) 3D tertiary-structural domains to provide structural/conformational stability.

FIG. 26: Schematic illustration of a mutation scheme for forming a foldikine based on a dimeric class I cytokine: (i) wild-type dimer showing left and right domains of the cytokine dimer, each comprising α-helices A, B, C and D of the first and second monomer sequences, respectively; (ii) single-chain foldikine based on fused wild-type monomers in which monomer sequences are joined to make a single chain polypeptide by linking the C-terminus of each monomer to a respective N-terminus of each monomer (dashed line), and then removing an internal wild-type linker from one of the original monomer sequences to create new N- and C-termini. Wild-type linkers shown in solid lines. Boxes indicate the α-helices that form each of the left and right 3D domains. As depicted, there are two peptide linkers spanning the first (left) and second (right) 3D tertiary-structural domains to provide structural/conformational stability.

FIG. 27: Schematic illustration of a mutation scheme for forming a foldikine based on a dimeric class II cytokine: (i) wild-type dimer showing left and right domains of the cytokine dimer, each comprising α-helices A, B, C, D, E, F and G of the first and second monomer sequences, respectively; (ii) single-chain foldikine based on fused wild-type monomers in which monomer sequences are joined to make a single chain polypeptide by linking the C-terminus of one monomer to the N-terminus of the same monomer, and removing an internal wild-type linker from each of the original monomer sequences to create to create new N- and C-termini, which are then joined by two new interdomain peptide linkers (dashed lines) to form a single chain polypeptide. Wild-type linkers shown in solid lines. Boxes indicate the α-helices that form each of the left and right 3D domains. As depicted, there are two peptide linkers spanning the first (left) and second (right) 3D tertiary-structural domains to provide structural/conformational stability.

FIG. 28: Schematic illustration of a mutation scheme for forming a foldikine based on a swapped domain dimeric class II cytokine: (i) wild-type domain swapped dimer showing «-helices D, E and F of each monomer unit aligning with the opposing monomer α-helices A, B and C, to form adjacent 3D domains comprising α-helices A, B and C of a first monomer sequence and α-helices D, E and F of a second monomer sequence; (ii) single-chain foldikine based on fused wild-type monomers in which monomer sequences are joined to make a single chain polypeptide by linking the C-terminus of one monomer sequence to the N-terminus of a second monomer sequence using a peptide linker (dashed line). Wild-type linkers shown in solid lines. Boxes indicate the α-helices that form each of the left and right 3D domains. As depicted, there are two peptide linkers spanning the first (left) and second (right) 3D tertiary-structural domains to provide structural/conformational stability.

FIG. 29: Graph demonstrating expression of MutSC1 foldikine in E. coli cells. IL-10 polypeptide sequence in wild type IL-10 and in MutSC1 foldikine was quantified by ELISA from both pelleted cells (pell) and the supernatant (sup). Colony-forming units (CFU) counts were normalised to fg of IL-10 estimated in each culture. MutSC1 expression was detected in both pellet and supernatant. The functionality of MutSC1 expressed by E. coli was assessed in HEK-blue cells. Both MutSC1 in the pellet and supernatant activated p-STAT3 cascade in this reporter cell line. MutSC1_His shows results for His-tagged foldikine.

FIG. 30: Graph demonstrating expression of MutSC1 foldikine in L. lactis cells. IL-10 polypeptide sequence in wild type IL-10 and MutSC1 was quantified by ELISA from both pelleted cells (pell) and the supernatant (sp). The functionality of the proteins expressed by L. lactis was verified using HEK-Blue10 reporter cells. For the y-axis, fg was quantified by ELISA and normalised to CFU counted in the two different cultures, IL-10 and MutSC1.

FIG. 31: Schematic illustration of a mutation scheme for forming a foldikine according to Way A, based on class I cytokine(s). (Upper part) wild-type cytokine type I monomers, each comprising α-helices A, B, C and D; (Bottom part) single-chain foldikine based on fused wild-type monomers in which monomer sequences are joined by opening a loop between α-helices B and C of both cytokine monomers, linking the new N-terminus of one cytokine monomer with the new C-terminus of the other cytokine monomer, and conversely, and linking the natural N-terminus and C-terminus of one of the cytokine monomer. Wild-type linkers shown in solid lines. Boxes indicate the α-helices that form each of the left and right 3D domains. As depicted, there are two peptide linkers spanning the first (left) and second (right) 3D tertiary-structural domains to provide structural/conformational stability.

FIG. 32: 3D representation of a Foldikine foldikine according to Way A, based on a dimeric class I cytokine with in black the newly introduced Interlinkers (linkers between two cytokine monomers) as well as the new intralinker (connecting the natural N-terminus and C-terminus of one of the cytokine monomer). Nt and Ct indicate the N- and C-termini of an original cytokine monomer.

FIG. 33: Schematic illustration of a mutation scheme for forming a foldikine according to Way B, based on two class II cytokines: (Uppert part) wild-type cytokine type II monomers, each comprising α-helices A, B, C, D, E, F and G of the first and second monomer sequences, respectively; (Bottom part) single-chain foldikine based on fused wild-type monomers in which monomer sequences are joined to make a single chain polypeptide by linking the C-terminus of one monomer to the N-terminus of the other monomer, and conversely, and removing an internal wild-type linker between α-helices C and D from one original monomer sequences to create to create new N- and C-termini (Nt and Ct), to form a single chain polypeptide. Wild-type linkers shown in solid lines. Boxes indicate the α-helices that form each of the left and right 3D domains. As depicted, there are two peptide linkers spanning the first (left) and second (right) 3D tertiary-structural domains to provide structural/conformational stability.

FIG. 34: 3D representation of a foldikine foldikine according to Way A based on a dimeric class II cytokine: in discontinuous black lines are the newly introduced interlinkers and new Nter and new Cter indicate the newly created N- and C-termini after opening a loop in one monomer.

FIG. 35: Titration plot of HEK Blue™ cells responding to IL-2 with the supernatant of CHO cells transiently expressing IL-2 WT and IL-2 foldikines (SCIL2 having different InterLinkers composed of Proline (ORK2_6 to 8) or Glycine/Serine (ORK_9 to 11). Proteins have been expressed using CHO as expression system. Vmax has been set to 0.9.

FIG. 36: Titration plot of HEK Blue™ cells responding to IL-22 with the supernatant of M. pneumoniae cells transiently expressing IL-22 WT or IL-22 foldikines (ORK22 having different InterLinkers composed of Proline (ORK17 to 19) or Glycine/Serine (ORK_24 to 26)).

FIG. 37: Titration plot of HEK Blue™ cells responding to IL22 with the supernatant of CHO cells transiently expressing IL-22 WT (9,76E-11 M) and IL-22 foldikines (ORIK22-12 (4,32E-11M) with polypro InterLinkers and ORIK22_14 (2,41E-09M) with polyGly InterLinkers). Proteins have been expressed using CHO as expression system.

FIG. 38: Titration plot of HEK Blue™ cells responding to IL-2 with the supernatant of CHO cells transiently expressing IL-2 WT and a chimeric foldikine IL2/IL-4 (IL2-IL4 PolyPro linker).

FIG. 39: Titration plot of HEK Blue™ cells responding to IL-4 with the supernatant of CHO cells transiently expressing IL-2 WT and a chimeric foldikine IL2/IL-4 (IL2-IL4 PolyPro linker). For comparative purpose we show the activity of a commercially purchased IL-4 (6507-IL-010/CF, Biotechne).

FIG. 40: Titration plot of HEK Blue™ cells responding to IL-2 with the supernatant of CHO cells transiently expressing IL-2 WT and the Foldikine ORK2_013.

FIG. 41: Titration plot of HEK Blue™ cells responding to IL-22. In the Y-axis we show the calculated EC-50 in nM. The X axis indicate the different IL variants expressed in CHO and tested. In all cases, we show for comparison the values for IL-22 WT. The different variants represent different circular permutant versions of the WT cytokines where we have joined the Nt and Ct by a protein designed (ModelX and FoldX) engineered linker and opening at different loops. LF means the protein is expressed but has lost the activity in HEK reporter. Proteins have been expressed using CHO as expression system. On top of each bar is displayed the mean EC-50 in nM for each variant.

FIG. 42: Titration plot of HEK Blue™ cells responding to IFNβ. The X axis indicate the different IL variants expressed in CHO and tested. In all cases, we show for comparison the values for the WT IFNβ. The different variants represent different circular permutant versions of the WT cytokines where we have joined the Nt and Ct by a protein designed (ModelX and FoldX) engineered linker and opening at different loops. Proteins have been expressed using CHO as expression system. On top of each bar is displayed the mean EC-50 in nM for each variant.

FIG. 43: Titration plot of HEK Blue™ cells responding to IL-2. The X axis indicate the different IL variants expressed in CHO and tested. In all cases, we show for comparison the WT IL-2 values. The different variants represent different circular permutant versions of the WT cytokines where we have joined the Nt and Ct by a protein designed (ModelX and FoldX) engineered linker and opening at different loops. Proteins have been expressed using CHO as expression system. On top of each bar is displayed the mean EC-50 in nM for each variant.

FIG. 44: Titration plot of HEK Dual cells responding to IFNγ with the supernatant of CHO cells transiently expressing IFNγ WT and the Foldikine ORKIFNg-002.

FIG. 45: Schematic illustration of a mutation scheme for forming a foldikine according to Way C, based on a dimeric class I cytokine(s). (Upper part) wild-type monomers type I (cytokine 1 and cytokine 2) r, each comprising α-helices A, B, C and D; (Lower part) single-chain foldikine based on fused wild-type monomers in which monomer sequences are joined by opening a loop between α-helices B and C of both cytokine monomers. The loop opened between helices B and C of monomer 2 is open and helix B of monomer 2 is linked to the natural N-terminal of monomer 1 and helix C is linked to the natural C-terminal of monomer 1 . . . . Boxes indicate the α-helices that form each of the left and right 3D domains. As depicted, there are two peptide linkers spanning the first (left) and second (right) 3D tertiary-structural domains to provide structural/conformational stability.

FIG. 46: 3D representation of a Foldikine foldikine according to Way C, based on a dimeric class I cytokine with in doted lines the newly introduced Interlinkers (linkers between two cytokine monomers). N-t and C-t indicate the N- and C-termini of an original cytokine monomer.

FIG. 47: Schematic illustration of a mutation scheme for forming a foldikine according to Way C, based on class II cytokine(s). (upper part) wild-type type II monomers (cytokines 1 and 2), each comprising α-helices A, B, C, D, E, F and G; (lower part) single-chain foldikine based on fused wild-type type II cytokine monomers in which monomer sequences are joined by opening a loop between α-helices B and C of one cytokine monomer (cytokine 1), linking the new C-terminus (Cytokine 1, Helix B) of the opened cytokine monomer with the natural N-terminus of the other cytokine monomer (Cytokine 2, Helix A), and linking the new N-terminus (Cytokine 1, Helix C) of the opened cytokine monomer with the natural C-terminus of the other cytokine monomer (Cytokine 2; helix F). Boxes indicate the α-helices that form each of the left and right 3D domains. As depicted, there are two peptide linkers spanning the first (left) and second (right) 3D tertiary-structural domains to provide structural/conformational stability.

FIG. 48: 3D representation of a Foldikine foldikine according to Way C, based on a dimeric class II cytokine with in bold lines the newly introduced Interlinkers (linkers between two cytokine monomers. N-t and C-t indicate the N- and C-termini of an original cytokine monomer.

### DETAILED DESCRIPTION OF THE INVENTION

All references cited herein are incorporated by reference in their entirety. Unless otherwise defined, all technical and scientific terms used herein have the same meaning as commonly understood by one of ordinary skill in the art to which this invention belongs (e.g. in cell culture, molecular genetics, nucleic acid chemistry and biochemistry).

Unless otherwise indicated, the practice of the present invention employs conventional techniques of chemistry, molecular biology, microbiology, recombinant DNA technology, chemical methods, pharmaceutical formulations and delivery and treatment of animals, which are within the capabilities of a person of ordinary skill in the art. Such techniques are also explained in the literature, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N. Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

In order to assist with the understanding of the invention several terms are defined herein.

The term ‘amino acid’ in the context of the present invention is used in its broadest sense and is meant to include naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term ‘amino acid’ further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as ‘functional equivalents’ of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The term ‘peptide’ as used herein (e.g. in the context of a foldikine protein as disclosed herein) refers to a plurality of amino acids joined together in a linear or circular chain. The term oligopeptide is typically used to describe peptides having between 2 and about 50 or more amino acids. Peptides larger than about 50 amino acids are often referred to as polypeptides or proteins. For purposes of the present invention, however, the term ‘peptide’ is not limited to any particular number of amino acids, and is used interchangeably with the terms ‘polypeptide’ and ‘protein’ unless otherwise indicated. Taking into account that minor modifications to the primary sequence of the peptides/proteins of this disclosure may be made without substantially altering the activity of the polypeptides according to the invention, this disclosure and the various aspects and embodiments of the invention should be considered to encompass, in addition, any polypeptide sequences that are substantially the same as the specific amino acid sequences disclosed herein. For example, the claimed invention encompasses polypeptide sequences that have at least 80% identity to the polypeptides disclosed herein (such as may be defined by the SEQ ID NOs of the polypeptide sequences disclosed herein), at least 85% identity, at least 90% identity, at least 95% identity, at least 98% identity, at least 99% identity or approx. 100% identity to the polypeptide sequences of the SEQ ID NOs explicitly disclosed herein.

As used herein, the term ‘cytokine’ refers to any one from a broad and loose category of small proteins important in cell signaling. Cytokines are peptides that cannot cross the lipid bilayer of cells to enter the cytoplasm. Cytokines have been shown to be involved in autocrine, paracrine and endocrine signalling as immunomodulating agents. The term ‘foldikine’, as used herein, refers to a single chain cytokine which is derived from two wild-type cytokine monomers that have been covalently linked and folds to form a dimeric 3D domain structure. In general, the peptide sequence of each 3D domain of the covalent dimer also does not exist in nature, as it includes one or more amino acid mutation, substitution and/or deletion in order to (i) dimerise the cytokine monomers; and/or (ii) structurally constrain the configuration of the novel 3D dimeric unit.

The terms ‘nucleic acid’, ‘polynucleotide’, and ‘oligonucleotide’ are used interchangeably and refer to a deoxyribonucleotide (DNA) or ribonucleotide (RNA) polymer, in linear or circular conformation, and in either single- or double-stranded form, or mixed polymers. For the purposes of the present invention such DNA or RNA polymers may include natural nucleotides, non-natural or synthetic nucleotides, and mixtures thereof. Non-natural nucleotides may include analogues of natural nucleotides, as well as nucleotides that are modified in the base, sugar and/or phosphate moieties (e.g. phosphorothioate backbones). Examples of modified nucleic acids are PNAs and morpholino nucleic acids. Generally, an analogue of a particular nucleotide has the same base-pairing specificity, i.e. an analogue of G will base-pair with C. For the purposes of the invention, these terms are not to be considered limiting with respect to the length of a polymer. In certain embodiments, the 5′ and/or 3′ end of the polynucleotides of this disclosure may be modified to improve the stability of the sequence in order to actively avoid degradation. Suitable modifications in this context include but are not limited to biotinylated nucleotides and phosphorothioate nucleotides.

A ‘gene’, as used herein, is the segment of nucleic acid (typically DNA) that is involved in producing a polypeptide or ribonucleic acid gene product. It includes regions preceding and following the coding region (leader and trailer) as well as intervening sequences (introns) between individual coding segments (exons). Conveniently, this term also includes the necessary control sequences for gene expression (e.g. enhancers, silencers, promoters, terminators etc.), which may be adjacent to or distant to the relevant coding sequence, as well as the coding and/or transcribed regions encoding the gene product. Preferred genes in accordance with the present invention are those that encode cytokines, and particularly those that encode dimeric cytokines or ‘foldikines’ according to this disclosure. Taking into account codon redundancy, this disclosure and the claimed invention encompasses polynucleotide sequences that have at least 70% identity to the polynucleotides disclosed herein-such as the SEQ ID NOs disclosed herein; at least 80% identity, at least 85% identity, at least 90% identity, at least 95% identity, at least 98% identity, at least 99% identity or approx. 100% identity to the polynucleotide sequences encoding the SEQ ID NOs explicitly disclosed herein.

In the context of the present disclosure, the terms ‘individual’, ‘subject’, or ‘patient’ are used interchangeably to indicate an animal that may be suffering from a medical (pathological) condition and may be responsive to a therapeutic molecule or medical treatment of the invention. The animal to be treated is suitably a mammal, such as a human, non-human primate, cow, sheep, pig, dog, cat, rabbit, mouse, or rat. Preferably the subject is a human.

The terms ‘treat’, ‘treating’, ‘treatment’ or ‘therapy’ as used herein in the context of a disease state or condition, refer to a reduction in severity and/or frequency of one or more pathogenic symptom, the elimination of symptoms and/or the prevention of symptoms and/or the underlying cause or causes of such disease or condition. In some cases, such treatment may relate to the reduction or improvement of a physiological damage caused by the disease or condition.

### The Foldikine Concept

Improving cytokine efficiency thus decreasing the concentration needed for the therapeutic effect could be achieved by improving their foldability or decreasing their degradation rate and clearance from the serum. This requires engineering mutations that will improve binding affinity, decrease aggregation or fuse to other them protein domains like Fcs (https://pubmed.ncbi.nlm.nih.gov/32994996/), or subject them to chemical modification (https://www.nature.com/articles/s41551-021-00797-8; https://www.mdpi.com/2079-4991/8/8/1074; https://link.springer.com/article/10.1007/s11912-019-0760-z). Regarding decreasing toxicity, in many cases, this is because a particular cytokine binds to different cells in the organism, some of them being the desired target and others having unwanted effects. The majority of cytokines are bound by two or three different receptors, some of them being promiscuous and recognising more than one cytokine. The receptors for a particular cytokine are not expressed proportionally at the same level in all cells and therefore it should be possible to alter the affinity for one receptor to favour binding to a particular group of cells, therefore, decreasing toxicity (https://doi.org/10.1111/all.15132; https://pubmed.ncbi.nlm.nih.gov/33737461/).

In some cases, a combination of two different cytokines could be useful for particular therapies with both of them being antagonists or agonists or having a mixture of both (https://academic.oup.com/abt/article/4/2/123/6309385). Linking two cytokines by inserting a linker sequence between the N-terminal of one molecule and the C-terminal of the other could be one way of solving part or all of the above issues. However, it is not always possible to link two cytokines via their N or C-terminus-even when such cytokines act by forming non-covalent dimers-because of incompatibility with the binding mode to their receptors. In addition, a flexible linker will be susceptible to proteolysis and may not increase binding affinity due to the conformational flexibility between the two monomers.

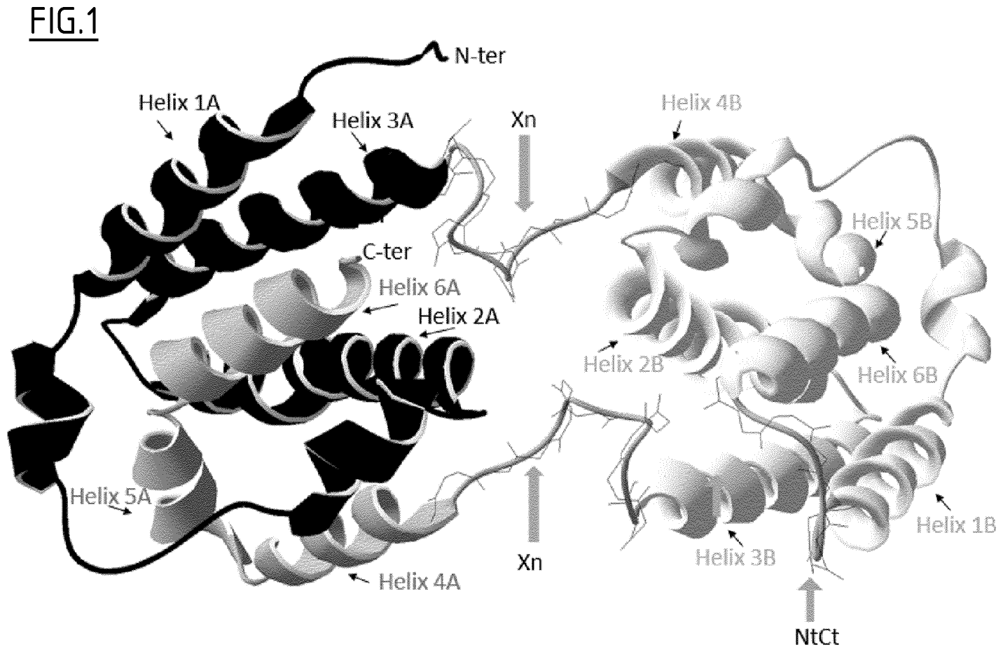

Here is where the concept of foldikine may provide significant advantages over both wild type cytokines and the prior art. As noted above, a foldikine is a molecule composed of two modified cytokine domains, each of which is termed a ‘foldikine domain’ herein; and it is noted in this respect that at least one of the two ‘foldikine domains’ is not structurally or sequentially the same as a cytokine monomer domain on which the foldikine domain is based, as will be described herein. In aspects and embodiments of the foldikines of the invention, it may be considered that one of the two foldikine domains is continuous in sequence. This foldikine domain could have a non-natural sequence order in comparison to a corresponding cytokine monomer/domain while the other foldikine domain is split into two pieces of equal or different size connected to the natural or newly created N- and C-termini of the continuous foldikine domain (see FIG. 1). The continuous and split domains of the foldikine may also be referred to herein as 3D domains.

It is therefore provided a single chain dimeric cytokine polypeptide comprising: (a) a first cytokine monomer domain or a functional portion thereof; and (b) a second cytokine monomer domain or a functional portion thereof, wherein the sequence of one of the cytokine monomer domains or functional portion thereof is not continuous and is interrupted by the sequence of the other cytokine monomer domain or a functional portion thereof.

The cytokine monomer domain of the single chain dimeric cytokine polypeptide which sequence is not continuous is the cytokine monomer domain that comprises the free N-terminus of the engineered single chain dimeric cytokine polypeptide. This cytokine monomer domain consists or makes part of the so-called “split domain” throughout the instant application.

The sequence of the other cytokine monomer domain or a functional portion thereof may have its natural sequence order, or a different one. The second cytokine monomer domain is also called “continuous domain” throughout the instant application; it is connected to the split domain by two peptide linkers (“interlinkers”).

### Advantages of the Foldikines

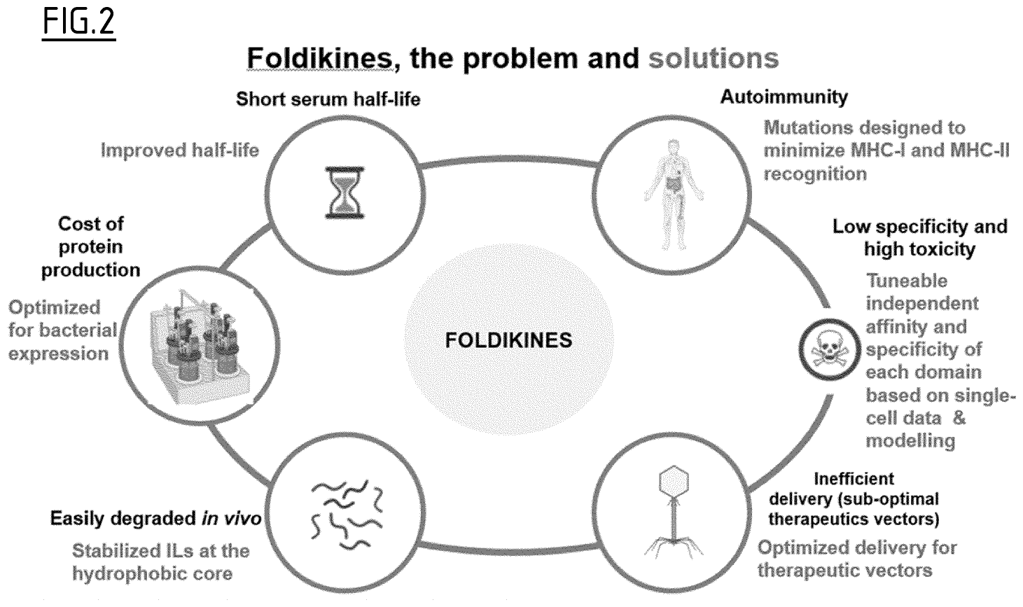

Having two functional independent domains with a fixed orientation within the same molecule offers several interesting possibilities that answer to some of the problems mentioned above, which are independent of the production and administration mode (see FIG. 2).

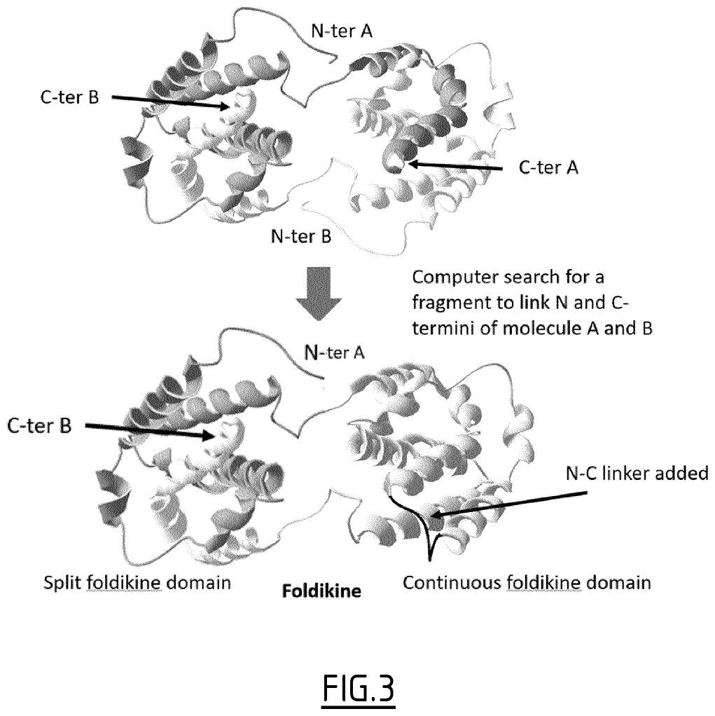

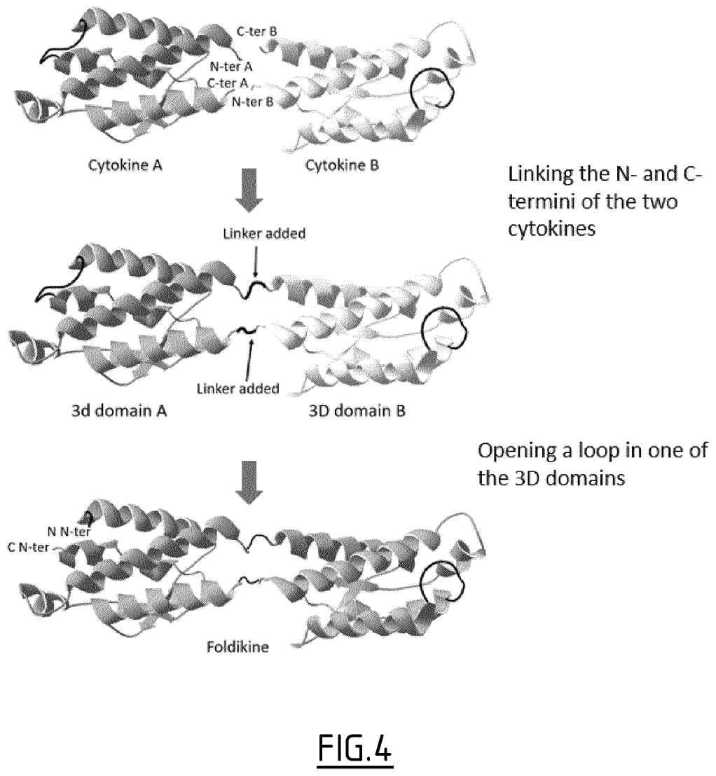

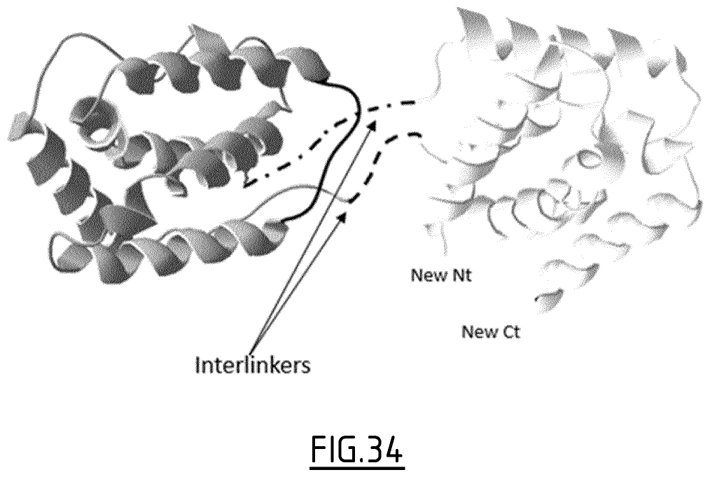

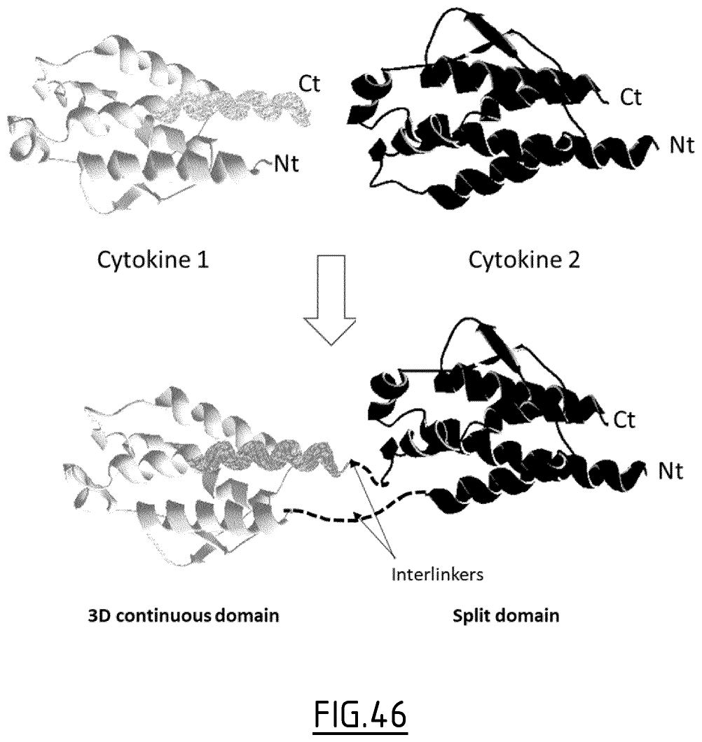

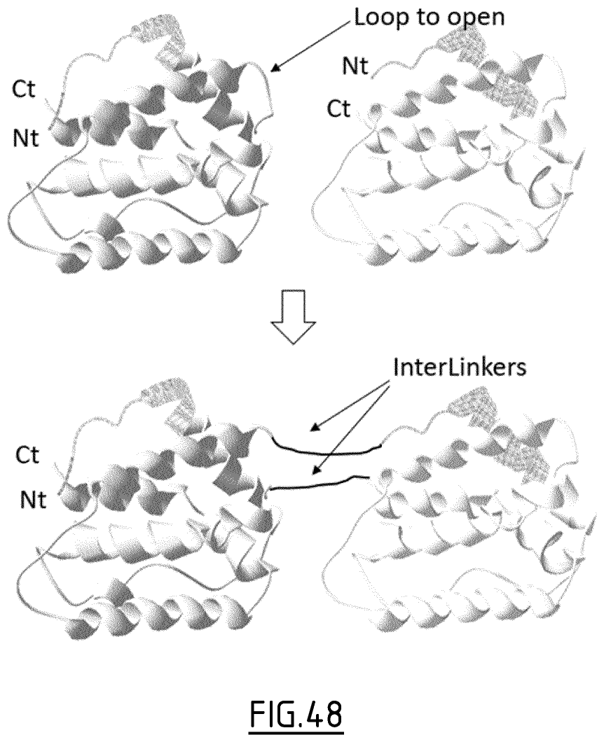

In embodiments, as depicted in e.g. FIGS. 3 to 6, 25 to 28, 31 to 34, and 45 to 48, foldikines have the advantage of having two linkers between the adjacent (left and right) 3D domains (which may themselves be considered to be ‘continuous’ and ‘split’), which may enhance the structural/conformation stability of the folded dimer and result in improved binding to its target receptors, since the entropic cost of binding will be mainly paid by the first 3D domain, and since the existence of two linkers restricts the mobility of the second 3D domain. Also, by providing two independent 3D domains connected by two linkers it allows to make homo or heterodimeric cytokines with new properties and gives more freedom to the mutations that can be introduced at the surface of the 3D domains, e.g. which interact with the receptors which could allow greater selectivity to a particular cell type depending of the level of expression of the target receptors.

In some advantageous embodiments, a heterodimeric foldikine according to the invention may exhibit improved binding of one domain to its target receptor but decreased binding of the other domain to its target receptor. In such embodiments, the first domain could advantageously be used to target specific cells; while since the second domain will have reduced affinity, it may beneficially only/primarily bind to only those cells also expressing receptors for the first domain. In embodiments, foldikines may also increase the binding affinity of both domains thus decreasing significantly the effective physiological concentration that may be necessary to achieve a particular physiological effect. For example, it may be desired to construct an antagonist molecule having two domains with high affinity towards one receptor and none to a second receptor (of a corresponding wild type cytokine), such that the foldikine can effectively compete against the corresponding monomeric physiological cytokines. Having two 3D domains allows many possible combinations of homing, affinity and specificity, as well as combining agonist and antagonist functions in one molecule.

### Foldikine Design Based on Cytokine Families

We have developed the foldikine concept for helix-bundle cytokines that comprise the majority of the Interleukins, but it is envisaged that it would be possible to do a similar approach for other proteins composed of helix bundle structure or for cytokines with other folds. Helix-bundle interleukins are classified as:
(A) Class I helical cytokines that can be sub-classified in long-chain helical (IL6, IL11, IL12A, IL23A, IL27A, IL31, CLCF1, CNTF, CTF1, LIF, OSM, and CSF3), or short-chain helical (IL2, IL3, IL4, IL5, IL7, IL9, IL13, IL15, IL21, TSLP, GM-CSF, CSF1, and CSF2); or
(B) Class II cytokines are IL10-like proteins including (IL10, IL19, IL20, IL22, IL24, IL26, IL28A, IL28B, and IL29), and type I interferons such as IFNα (IFNA1, IFNA2, IFNA4, IFNA5, IFNA6, IFNA7, IFNA8, IFNA10, IFNA13, IFNA14, IFNA16, IFNA17 and IFNA21), IFNω (IFNW1), IFNε (IFNE), IFNκ (IFNK) and IFNβ (IFNB1).

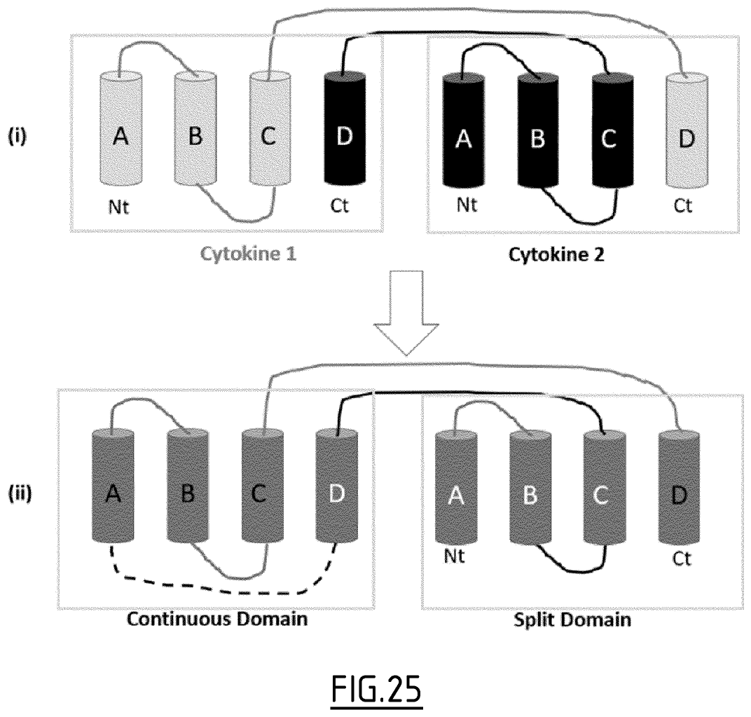

While the majority of helix-bundle cytokines share similar monomeric structures in each class, with type I cytokines typically including 4 conserved α-helices, while type II cytokines typically include 6 conserved α-helices, IFNγ, IL-5, and IL-10 are ‘swapped-domain’ dimeric proteins. In a swapped-domain dimeric protein like IL-10 the monomeric structure of the standard helix bundle cytokines opens and embraces in an antiparallel fashion another IL-10 opened monomer generating two functional split cytokine domains together defining two adjacent 3D domains (see e.g. FIGS. 3 and 25).

Based on the interleukin class and whether the interleukin is monomeric or a swapped dimer, different strategies for generating foldikines could be employed as described herein.

As used herein, α-helices of a cytokine monomer are numbered in the alphabetical order A, B, C, and D (for class I cytokines), or A, B, C, D, E, and F (for class II cytokines), or in the ordinal number (first to fourth, or sixth, depending on the cytokine class), from the N-terminus to the C-terminus.

### Foldikines Comprising Swapped Domain Cytokines

According to this embodiment, the single chain dimeric cytokine polypeptide according to the invention, comprises a first and/or a second cytokine monomer(s) form(s) swapped domain dimers.

According to an embodiment, the single chain dimeric cytokine polypeptide according to the invention comprises a first and a second cytokine monomers that form swapped domain dimers. In particular the first and second cytokine monomers that form swapped domain dimers may be from a same cytokine, or from two different cytokines (two different Class I cytokines, or two different Class II cytokines).

In the case of swapped domain cytokines (e.g. IFNγ, IL-5, and IL-10), the scheme for designing a single-chain dimeric cytokine of the disclosure (i.e. a foldikine) may depend on whether the swapped domain cytokine is to be fused to a second swapped domain cytokine or to a cytokine that does not form a swapped domain.

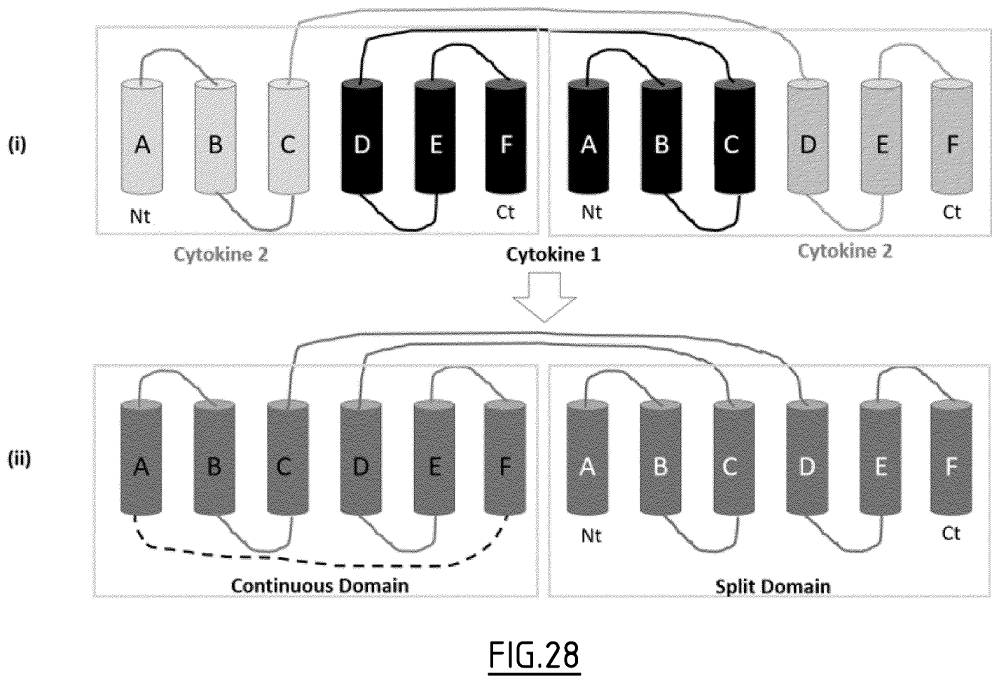

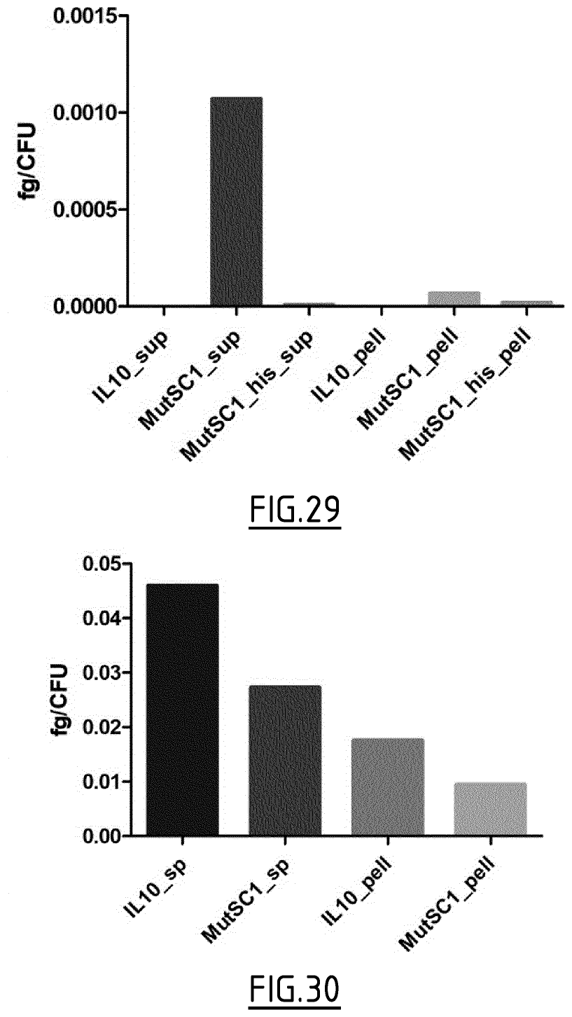

As described with reference to e.g. FIGS. 3, 25 and 28, in embodiments wherein both monomers from which a foldikine is created from natural swapped domain dimers, the process may involve linking the N-terminus of a first cytokine monomer to the C-terminus of a second cytokine monomer, such that in the swapped domain foldikine dimer a ‘continuous 3D (foldikine) domain’ is formed (right-hand domain in FIG. 3), and a split 3D (foldikine) domain is formed (left-hand domain in FIG. 3). In this case, however, it is noted that the ‘continuous’ 3D domain has a helix/sequence that is inverted in comparison to a wild type cytokine, because the C-terminal helix of the most N-terminal monomer is linked to the N-terminal helix of the most C-terminal monomer (FIGS. 25 and 28). Furthermore, it should be noted that each domain in 3D space is comprised of parts of two separate polypeptide monomers.

In any such aspects and embodiments, the linker sequence bridging between the C-terminus of one monomer and the N-terminus of the other monomer may have any suitable sequence; and preferentially comprises a sequence that defines a conformationally restrained structure, i.e. a ‘structured’ linker. In this way, the engineered linkers help to conformationally restrain the 3D domain structures in an appropriate/desired structural orientation. Such linkers may have from about 3 to about 20 amino acid residues; from about 3 to about 16 amino acid residues; from about 4 to about 12 amino acid residues, from about 4 to about 8 amino acid residues, from about 3 to 8 amino acid residues or from about 3 to 6 amino acid residues. Beneficially, such linkers do not contain a plurality of Gly and/or Ser residues adjacent each other; for example, beneficially the linker peptide contains 5 or less adjacent Gly and/or Ser residues; suitably 3 or less adjacent Gly and/or Ser residues; 2 or less Gly and/or Ser residues; contains only isolated Gly and/or Ser residues; or in some embodiments contains no Gly and/or Ser residues.

Accordingly, a single chain dimeric cytokine polypeptide is provided that comprises a first cytokine monomer and a second cytokine monomer that form swapped domain dimers, wherein a linker peptide bridges the N-terminus of the first cytokine monomer with the C-terminus of a second cytokine monomer.

In some embodiments, the cytokine monomers forming swapped domain dimers are Class I cytokines, and said single chain dimeric cytokine polypeptide comprises, from the N-terminus to the C-terminus, a first cytokine monomer domain that comprises helices A to C of the second cytokine monomer, and helix D of the first cytokine monomer (see e.g. “split domain” on FIG. 25), and a second cytokine monomer domain that comprises helix D of the second cytokine monomer and helices A to C of the first cytokine monomer (see e.g. “continuous domain” on FIG. 25), wherein a linker peptide bridges helix D of the second cytokine monomer with helix A of the first cytokine monomer.

Exemplary homomeric Foldikine based on a swapped domain Class I cytokine include dimeric IL-5 which comprises or consists of:

```
TEIPTSALVKETLALLSTHRTLLIANETLRIPVPVHKNHQLCTEEIFQGIGTLESQTVQGGIVERLFK
        hhhhhhhhhhhhh hhhh hhhhhhhhhh hhhhhh
                          HA HB
NLSLIKKYIDGQKKKCGEERRRVNQFLDYLQEFLGVMNTEWIIES(NtCt)
hhhhhhhhhhhh hhhhhhhhhhhhhhhhh
          HC HD
TEIPTSALVKETLALLSTHRTLLIANETLRIPVPVHKNHQLCTEEIFQGIGTLESQTVQGGTVERLFKNL
        hhhhhhhhhhhhh hhhh hhh hhhhhhhhhh hhhhhhhh
                  HA HB HC
SLIKKYIDGQKKKCGEERRRVNQFLDYLQEFLGVMNTEWIIES
hhhhhhhhhh hhhhhhhhhhhhhhhhh
                                                      HD
```

wherein (NtCt) is a peptide linker (sequence SEQ ID NO: 101-(NtCt)-SEQ ID NO:102), or a sequence at least 80%, at least 85%, at least 90%, at least 95%, at least 98%, at least 99% or 100% identical thereto and that retain at least the same stability, and/or at least the same level of interaction with IL-5 receptor.

Positions of the helices are represented by strings of ‘h’ in the above sequence

In some embodiments, the cytokine monomers forming swapped domain dimers are Class II cytokines, and said single chain dimeric cytokine polypeptide comprises, from the N-terminus to the C-terminus, a first cytokine monomer domain that comprises helices A to C of the second cytokine monomer, and helices D to F of the first cytokine monomer (see e.g. “split domain” on FIG. 28), and a second cytokine monomer domain that comprises helices D to F of the second cytokine monomer and helices A to C of the first cytokine monomer (see e.g. “continuous domain” on FIG. 28), wherein a linker peptide bridges helix F of the second cytokine monomer with helix A of the first cytokine monomer.

Exemplary homomeric Foldikine based on a swapped domain Class II cytokine include dimeric IL-10.

According to an embodiment, dimeric IL-10 comprises or consists of sequence

```
CTHFPGNLPNMLRDLRDAFSRVKTFFQMKDQLDNLLLKESLLEDFKGYLGCQALSEMIQFYLEEVMPQ
            hhh hhhhhhhhhhh hhhhhh hhhhhhhhhhhhhhhhhhh
                                          HA HB
AENQDPDIKAHVNSLGENLKTLRLRLRRCHRFLPCENKSKAVEQVKNAFNKLQEKGIYKAMSEFDIFINY
hhh hhhhhhhhhhhhhhhhhhh hhhhhhhhhhhh hhhhhhh hhhhhh
                            HC HD HE
IEAYMTMKIRN(NtCt)PNMLRDLRDAFSRVKTFFQMKDQLDNLLLKESLLEDFKGYLGCQALSEMIQFYLEEV
hhhhhhhh hhhhhhhhhhh hhhhhh hhhhhhhhhhhhhhhh
      HF HA HB
MPQAENQDPDIKAHVNSLGENLKTLRLRLRRCHRFLPCENKSKAVEQVKNAFNKLQEKGIYKAMSEFDIF
hhhhhh hhhhhhhhhhhhhhhhhhh hhhhhhhhhhhh hhhhhhh hhh
                                HC HD HE
INYIEAYMTM_YY
hhhhhhhhhh
HF
```

wherein (NtCt) is a peptide linker (sequence SEQ ID NO: 11-(NtCt)-SEQ ID NO: 12), or a sequence at least 80%, at least 85%, at least 90%, at least 95%, at least 98%, at least 99% or 100% identical thereto and that retain at least the same stability, and/or at least the same level of interaction with IL-10 receptor. Positions of the helices are represented by strings of ‘h’ in the above sequence.

According to an embodiment, the NtCt linker is a peptide linker and comprises or consists of sequence NGGLDY (SEQ ID NO: 13).

In particular, a single chain dimeric IL-10 may comprise or consist of SEQ ID NO: 9 (so-called MutSC1 polypeptide in example 1), or SEQ ID NO: 10 (so-called MutSC2 polypeptide in example 1), or a sequence at least 80%, at least 85%, at least 90%, at least 95%, at least 98%, at least 99% or 100% identical thereto and that retain at least the same stability, and/or at least the same level of interaction with IL-10 receptor.

Another exemplary homomeric Foldikine based on a swapped domain Class II cytokine include dimeric IFNγ.

According to an embodiment, dimeric IFNγ comprises or consists of sequence

```
QDPYVKEAENLKKYFNAGHSDVADNGTLFLGILKNWKEESDRKIMQSQIVSFYFKLFKNFKDDQSIQK
      hhhhhhhhhhhh hh hhhhh hhhhhhhhhhhhhhhhhhhh hh
                      HA HB
SVETIKEDMNVKFFNSNKKKRDDFEKLTNYSVTDLNVQRKAIHELIQVMAELSPMLF(NtCt)
hhhhhhhhhhhhhh hhhhhhhhhhh hhhhhhhh hhhhhh
          HC HD HE HF
MQDPYVKEAENLKKYFNAGHSDVADNGTLFLGILKNWKEESDRKIMQSQIVSFYFKLFKNFKDD
          hhhhhhhhhhhhh hhhhh hhhhhhhhhhhhhhhhhhhh
                    HA HB
QSIQKSVETIKEDMNVKFFNSNKKKRDDFEKLTNYSVTDLNVQRKAIHELIQVMAELSPAAKTGKRKRSQ
      hhhhhhhhhhhhhhhh hhhhhhhhhh hhhhhhh---hhhhhh
                      HC HD HE HF
MLFRGRRASQ_YY
```

wherein (NtCt) is a peptide linker (sequence SEQ ID NO: 14-(NtCt)-SEQ ID NO:15), or a sequence at least 80%, at least 85%, at least 90%, at least 95%, at least 98%, at least 99% or 100% identical thereto and that retain at least the same stability, and/or at least the same level of interaction with IFNγ receptor. Positions of the helices are represented by strings of ‘h’ in the above sequence.

According to an embodiment, the NtCt linker comprises or consists of sequence EGPG (SEQ ID NO: 16), or ASKPHPGQLWY (SEQ ID NO: 17).

In particular, a single chain dimeric IFNγ may comprise or consist of SEQ ID NO:18 or a sequence at least 80%, at least 85%, at least 90%, at least 95%, at least 98%, at least 99% or 100% identical thereto and that retain at least the same stability, and/or at least the same level of interaction with IFNγ receptor.

Foldikines Including Cytokine(s) that do not Form Swapped Domain Cytokines

According to this aspect of the invention, the single chain dimeric cytokine polypeptide comprises: (a) a first cytokine monomer domain or a functional portion thereof; and (b) a second cytokine monomer domain or a functional portion thereof, wherein the sequence of the second cytokine monomer domain or functional portion thereof is continuous in sequence while the sequence of the first cytokine monomer domain is such that a first sequence portion of the first cytokine monomer is arranged at the N-terminus of the second cytokine monomer or functional portion thereof, and a second sequence portion of the first cytokine monomer is arranged at the C-terminus of the second cytokine monomer or functional portion thereof.

There are three different ways of making a Foldikine that can involve two same or different Class I cytokines, two same or different Class II cytokines, or one Class I cytokine and one Class II cytokine.

According to an embodiment, the first and second cytokine monomers are class I cytokine monomers that do not form swapped domain dimers, wherein first and second cytokine monomers are identical or different.

According to another embodiment, the first and second cytokine monomers are class II cytokine monomers that do not form swapped domain dimers, wherein first and second cytokine monomers are identical or different.

According to another embodiment, the first cytokine monomer is a class I cytokine monomer and the second cytokine monomer is a class II cytokine monomer, or the first cytokine monomer is a class II cytokine monomer and the second cytokine monomer is a class I cytokine monomer.

### Way a of Forming Foldikines

According to this way of forming a foldikine, a loop is opened in each cytokine monomer, and the new N-terminus of the opened loop of cytokine monomer A is joined with the new C-terminus of the opened loop of cytokine monomer B, and the new C-terminus of the opened loop of monomer A is joined with the new N-terminus of the opened loop of cytokine monomer B. The natural (or original) N-terminus and natural (or original) C-terminus of cytokine monomer A are further joined (or, alternatively, the natural/original N-terminus and natural/original C-terminus of cytokine monomer B are joined).

According to this way A of making a foldikine, the single chain dimeric cytokine polypeptide comprises: (a) a first cytokine monomer domain or a functional portion thereof; and (b) a second cytokine monomer domain or a functional portion thereof, wherein the sequence of the second cytokine monomer domain or functional portion thereof is is continuous in sequence (in particular it is inserted within the sequence of the first cytokine monomer domain) while the sequence of the first cytokine monomer domain is such that the first sequence portion of the first cytokine monomer domain which is arranged at the N-terminus of the second cytokine monomer domain or functional portion thereof corresponds to a N-terminal portion of a natural cytokine monomer domain, and the second sequence portion of the first cytokine monomer domain which is arranged at the C-terminus of the second cytokine monomer domain or functional portion thereof corresponds to a C-terminal portion of the natural cytokine monomer domain.

Structurally, the single chain dimeric cytokine polypeptide comprises:
i) a split domain that comprises the free N- and C-termini of the single chain dimeric cytokine polypeptide, and the first and second sequence portions of the first cytokine monomer domain in their natural sequence order (i.e. the first portion of the first cytokine monomer domain is a N-terminal portion of the first cytokine monomer domain and is at the N-terminal end of the single chain dimeric cytokine polypeptide, while the second portion of the first cytokine monomer domain is a C-terminal portion of the first cytokine monomer domain and is at the C-terminal end of the single chain dimeric cytokine polypeptide); and
ii) a continuous domain that comprises the second cytokine monomer domain or a functional portion thereof organized into a first sequence portion and a second sequence portion of the second cytokine monomer domain, the first and second sequence portions being in an order inverted compared to their natural sequence order (i.e. the second portion is a C-terminal portion of the second cytokine monomer domain and is at the N-side of the first portion of the second cytokine monomer domain, which is a N-terminal portion of the second cytokine monomer domain);
   wherein the sequences of the first and second sequence portions of the first cytokine monomer domain, in the split domain, are separated by the sequence of the continuous domain.

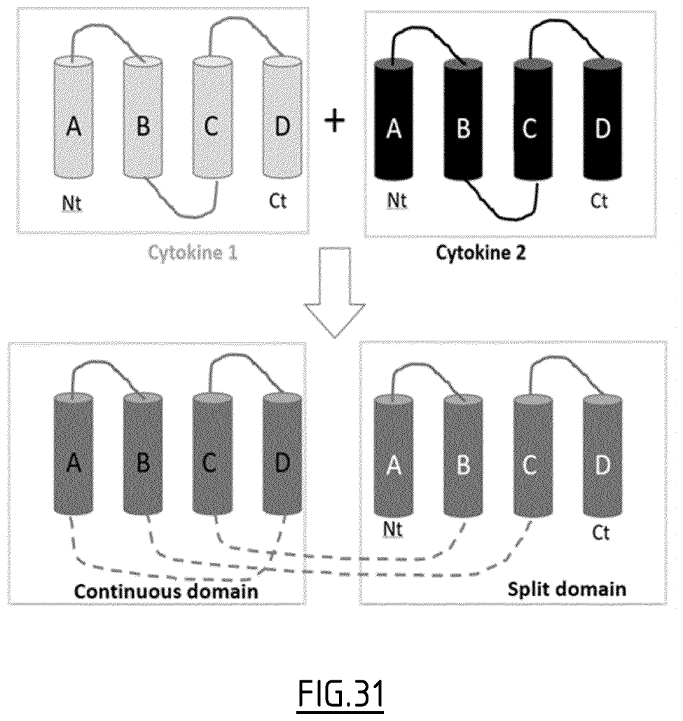

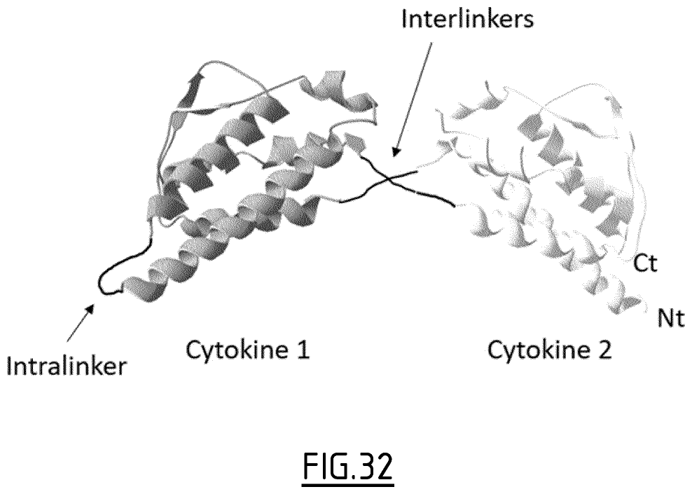

In the case of class I cytokines which do not form swapped domain dimers, forming a foldikine may involve linking two cytokines via two linkers after opening a loop in each monomer (in particular between α-helices B and C) and closing the N- and C-termini of one of the monomers via a linker (see e.g. FIGS. 31-32).

According to an embodiment, the loop between α-helices C and D is opened in each cytokine monomer, and a single chain dimeric class I polypeptide is provided that comprises two class I cytokine monomers, wherein said single chain dimeric cytokine polypeptide comprises:
a) a linker peptide bridges the second α-helix (α-helix B) of a first class I cytokine monomer (‘Cytokine 2’ on FIG. 31) with the third α-helix (α-helix C) of the second class I cytokine monomer (‘Cytokine 1’ on FIG. 31);
b) a linker peptide bridges the fourth α-helix (C-terminal α-helix, or α-helix D) of the second class I cytokine monomer with the first α-helix (N-terminal α-helix, or α-helix A) of the second class I cytokine monomer; and
c) a linker peptide bridges the second α-helix (α-helix B) of the second class I cytokine monomer with the third α-helix (α-helix C) of the first class I cytokine monomer.

In said single chain dimeric cytokine, the natural N-terminus and C-terminus of the first class I cytokine monomer are free.

Altogether, the single chain dimeric class I polypeptide comprises, from the N-terminus to the C-terminus, i) a N-terminal portion of a first class I cytokine monomer comprising α-helices A and B of said first class I cytokine monomer, ii) a linker peptide bridging α-helix B of said first class I cytokine monomer with α-helix C of a second class I cytokine monomer, iii) a C-terminal portion of said second class I cytokine monomer comprising α-helices C and D of said second class I cytokine monomer, iv) a linker peptide bridging α-helix D of said second class I cytokine monomer with α-helix A of said second class I cytokine monomer, v) a N-terminal portion of said second class I cytokine monomer comprising α-helices A and B of said second class I cytokine monomer, vi) a linker peptide bridging α-helix B of the second class I cytokine monomer with α-helix C of the first class I cytokine monomer, and vii) a C-terminal portion of said first class I cytokine monomer comprising α-helices C and D of said first class I cytokine monomer (FIGS. 31 and 32).

In some embodiments, once a class I Foldikine has been made following way A, a circular permutant variant is created where the natural Nt and Ct of the second-class I cytokine monomer are joined, and a loop is opened in the first second class I cytokine monomer.

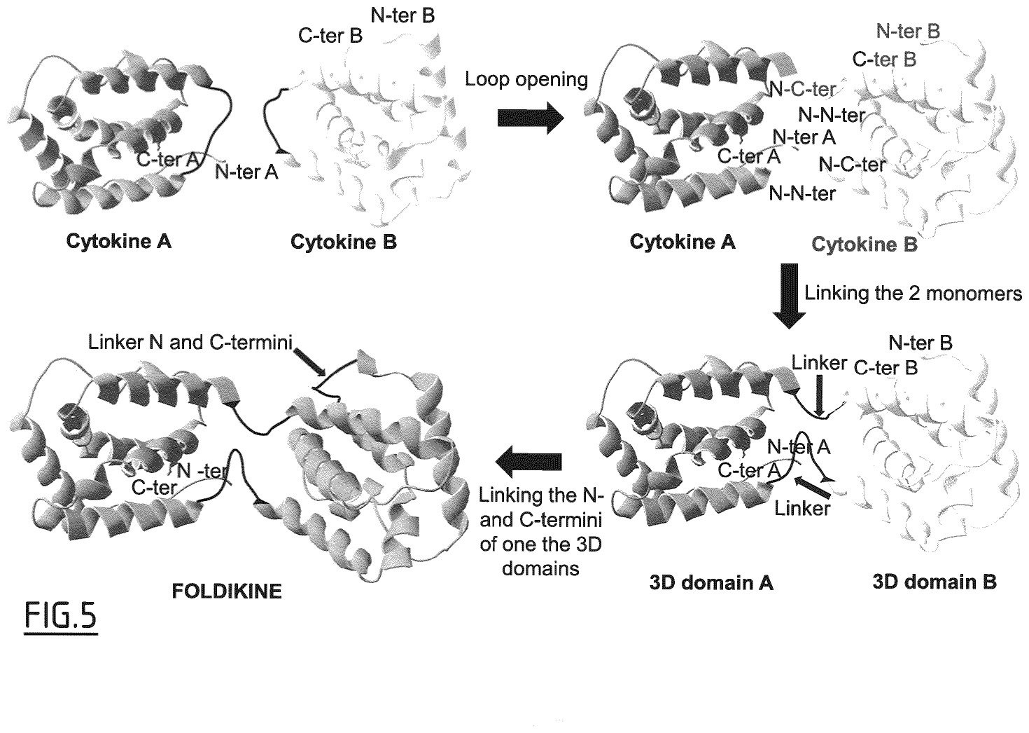

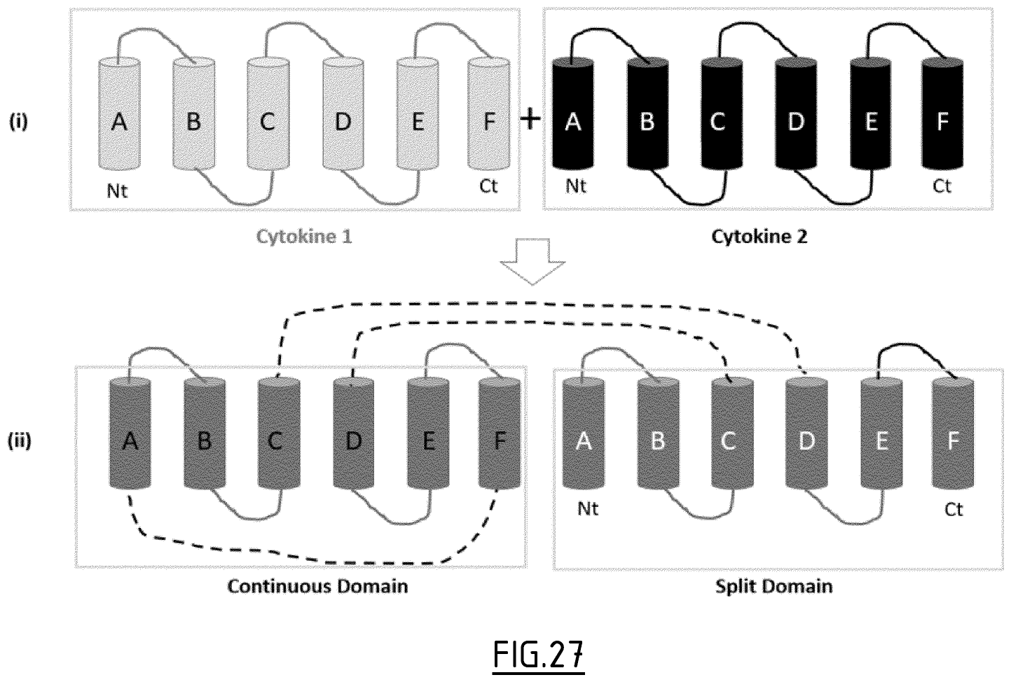

In the case of class II cytokines which do not form swapped domain dimers, forming a foldikine may involve linking two cytokines via two linkers after opening a loop in each monomer (in particular between α-helices C and D) and closing the N- and C-termini of one of the monomers via a linker (see e.g. FIGS. 5 and 27).

According to an embodiment, the loop between α-helices C and D is opened in each cytokine monomer, and a single chain dimeric type II cytokine polypeptide is provided that comprises two cytokine monomers from class II cytokine(s) that do(es) not form swapped domain dimers, wherein in said single chain dimeric type II cytokine polypeptide:
a) a linker sequence bridges the third α-helix (α-helix C) of a first class II cytokine monomer (‘Cytokine 1’ on FIG. 27) with the fourth α-helix (α-helix D) of a second class II cytokine monomer (‘Cytokine 2’ on FIG. 27);
b) a linker sequence bridges the fourth α-helix (α-helix D) of said first class II cytokine monomer with the third α-helix (α-helix C) of said second class II cytokine monomer;
c) a linker sequence bridges the first α-helix (N-terminal α-helix, or α-helix A) of said first class II cytokine monomer with the sixth α-helix (C-terminal α-helix, or α-helix F) of said first class II cytokine monomer; and
d) the third and fourth α-helices (α-helices C and D) in each of said first and second class II cytokine monomers are not bridged.

Altogether, the single chain dimeric class II polypeptide comprises, from the N-terminus to the C-terminus, i) a N-terminal portion of a first class II cytokine monomer comprising α-helices A to C of said first class II cytokine monomer, ii) a linker peptide bridging α-helix C of said first class II cytokine monomer with α-helix D of a second class II cytokine monomer, iii) a C-terminal portion of said second class II cytokine monomer comprising α-helices D to F of said second class II cytokine monomer, iv) a linker peptide bridging α-helix F of said second class I cytokine monomer with α-helix A of said second class II cytokine monomer, v) a N-terminal portion of said second class II cytokine monomer comprising α-helices A to C of said second class II cytokine monomer, vi) a linker peptide bridging α-helix C of the second class II cytokine monomer with α-helix D of the first class II cytokine monomer, and vii) a C-terminal portion of said first class II cytokine monomer comprising α-helices D to F of said first class II cytokine monomer.

In any such embodiments, the two class II cytokine monomers are from one class II cytokine that does not form swapped domain dimers, or from two different class II cytokines that do not form swapped domain dimers.

Exemplary homomeric Foldikine based on Class II cytokine include:
i) dimeric IL-22 which may comprise or consist of sequence

```
RLDKSNFQQPYITNRTFMLAKEASLADNNTDVRLIGEKLFHGVSMSERCYLMKQVLNFT
                  hhhhhhhhhhhhhhhhh hhh hhhhhhhhhhhh
                                  HA HB
LEEVLFPQSDRFQPYMQEVVPFLARLSNRLS(Xn1)HIQRNVQKLKDTVKKLGESGE
h---hhhh hhhhhhhhhhhhhhhhh hhhhhhhhhhhhhhh hhh
                                        HC HD
IKAIGELDLLFMSLRNACI(NtCt)LDKSNFQQPYITNRTFMLAKEASLADNNTDVRLIGEKL
hhhh hhhhhhhhhhh hhhhhhhhhhhhhhhhh hh
HE HF HA
FHGVSMSERCYLMKQVLNFTLEEVLFPQSDRFQPYMQEVVPFLARLSNRLS(Xn2)HIQR
h hhhhhhhhhhhhh hhh hhhhhhhhhhhhhhhhh hhhh
                          HB HC
NVQKLKDTVKKLGESGEIKAIGELDLLFMSLRNACI
hhhhhhhhhhh hhhhhhh hhhhhhhhhhh
    HD HE HF
```

(SEQ ID NO: 325-(Xn1)-SEQ ID NO: 326-(NtCt)-SEQ ID NO: 327-(Xn2)-SEQ ID NO: 328) or a sequence at least 80%, 85%, 90%, 95%, 98% or 99% identical thereto that retains at least the same stability, and/or at least the same level of interaction with the respective receptor(s) of said monomeric class II cytokine (IL-22);
ii) dimeric IL-19 (which may comprise or consist of sequence SEQ ID NO: 195-(Xn1)-SEQ ID NO: 196-(NtCt)-SEQ ID NO: 197-(Xn2)-SEQ ID NO: 198 or a sequence at least 80%, 85%, 90%, 95%, 98% or 99% identical thereto that retains at least the same stability, and/or at least the same level of interaction with the respective receptor(s) of said monomeric class II cytokine),
iii) dimeric IL-20 (which may comprise or consist of sequence SEQ ID NO: 200-(Xn1)-SEQ ID NO: 201-(NtCt)-SEQ ID NO: 202-(Xn2)-SEQ ID NO: 203 or a sequence at least 80%, 85%, 90%, 95%, 98% or 99% identical thereto that retains at least the same stability, and/or at least the same level of interaction with the respective receptor(s) of said monomeric class II cytokine),
iv) dimeric IL-26 (which may comprise or consist of sequence SEQ ID NO: 210-(Xn1)-SEQ ID NO: 211-(NtCt)-SEQ ID NO: 212-(Xn2)-SEQ ID NO: 213 or a sequence at least 80%, 85%, 90%, 95%, 98% or 99% identical thereto that retains at least the same stability, and/or at least the same level of interaction with the respective receptor(s) of said monomeric class II cytokine),
v) dimeric IFNA2 (which may comprise or consist of sequence SEQ ID NO: 215-(Xn1)-SEQ ID NO: 216-(NtCt)-SEQ ID NO: 217-(Xn2)-SEQ ID NO: 218 or a sequence at least 80%, 85%, 90%, 95%, 98% or 99% identical thereto that retains at least the same stability, and/or at least the same level of interaction with the respective receptor(s) of said monomeric class II cytokine),
vi) dimeric IFNA3 (which may comprise or consist of sequence SEQ ID NO: 220-(Xn1)-SEQ ID NO: 221-(NtCt)-SEQ ID NO: 222-(Xn2)-SEQ ID NO: 223 or a sequence at least 80%, 85%, 90%, 95%, 98% or 99% identical thereto that retains at least the same stability, and/or at least the same level of interaction with the respective receptor(s) of said monomeric class II cytokine),
vii) dimeric IFNA1 (which may comprise or consist of sequence SEQ ID NO: 225-(Xn1)-SEQ ID NO: 226-(NtCt)-SEQ ID NO: 227-(Xn2)-SEQ ID NO: 228 or a sequence at least 80%, 85%, 90%, 95%, 98% or 99% identical thereto that retains at least the same stability, and/or at least the same level of interaction with the respective receptor(s) of said monomeric class II cytokine),
viii) dimeric IFNα1/13 (which may comprise or consist of sequence SEQ ID NO: 230-(Xn1)-SEQ ID NO: 231-(NtCt)-SEQ ID NO: 232-(Xn2)-SEQ ID NO: 233 or a sequence at least 80%, 85%, 90%, 95%, 98% or 99% identical thereto that retains at least the same stability, and/or at least the same level of interaction with the respective receptor(s) of said monomeric class II cytokine),
ix) dimeric IFNα2 (which may comprise or consist of sequence SEQ ID NO: 235-(Xn1)-SEQ ID NO: 236-(NtCt)-SEQ ID NO: 237-(Xn2)-SEQ ID NO: 238 or a sequence at least 80%, 85%, 90%, 95%, 98% or 99% identical thereto that retains at least the same stability, and/or at least the same level of interaction with the respective receptor(s) of said monomeric class II cytokine),
x) dimeric IFNB (which may comprise or consist of sequence SEQ ID NO: 240-(Xn1)-SEQ ID NO: 241-(NtCt)-SEQ ID NO: 242-(Xn2)-SEQ ID NO: 243 or a sequence at least 80%, 85%, 90%, 95%, 98% or 99% identical thereto that retains at least the same stability, and/or at least the same level of interaction with the respective receptor(s) of said monomeric class II cytokine),
xi) dimeric IFNΩ1 (which may comprise or consist of sequence SEQ ID NO: 245-(Xn1)-SEQ ID NO: 246-(NtCt)-SEQ ID NO: 247-(Xn2)-SEQ ID NO: 248 or a sequence at least 80%, 85%, 90%, 95%, 98% or 99% identical thereto that retains at least the same stability, and/or at least the same level of interaction with the respective receptor(s) of said monomeric class II cytokine),
xii) dimeric IFNE (which may comprise or consist of sequence SEQ ID NO: 250-(Xn1)-SEQ ID NO: 251-(NtCt)-SEQ ID NO: 252-(Xn2)-SEQ ID NO: 253 or a sequence at least 80%, 85%, 90%, 95%, 98% or 99% identical thereto that retains at least the same stability, and/or at least the same level of interaction with the respective receptor(s) of said monomeric class II cytokine),
xiii) dimeric IFNκ (which may comprise or consist of sequence SEQ ID NO: 255-(Xn1)-SEQ ID NO: 256-(NtCt)-SEQ ID NO: 257-(Xn2)-SEQ ID NO: 258 or a sequence at least 80%, 85%, 90%, 95%, 98% or 99% identical thereto that retains at least the same stability, and/or at least the same level of interaction with the respective receptor(s) of said monomeric class II cytokine), or
   wherein (NtCt), (Xn1) and (Xn2) are peptide linkers.

In any such aspects and embodiments, the linker sequence (NtCt) bridging the N- and C-termini of one monomer may have any suitable sequence; and preferentially comprises a sequence that defines a conformationally restrained structure, i.e. a ‘structured’ linker. Such linkers may have from about 3 to about 20 amino acid residues; from about 3 to about 16 amino acid residues; from about 4 to about 12 amino acid residues, from about 4 to about 8 amino acid residues, from about 3 to about 8 amino acid residues or from about 3 to about 6 amino acid residues. In some embodiments, such linkers do not contain a plurality of Gly and/or Ser residues adjacent each other; for example, wherein the linker peptide contains 5 or less adjacent Gly and/or Ser residues; suitably 3 or less adjacent Gly and/or Ser residues; 2 or less Gly and/or Ser residues; contains only isolated Gly and/or Ser residues; or in some embodiments contains no Gly and/or Ser residues.

The linker can be designed by modelisation in order to interact with the receptors as the inventors found that a designed linker is better than a polyGly-Ser linker of the same length.

An exemplary structured (NtCt) linker comprises or consists of sequence ANGT (SEQ ID NO: 329), or ANGV (SEQ ID NO: 330), or TDYDSQTN (SEQ ID NO: 303).

Another exemplary structured (NtCt) linker comprises or consists of sequence EGPG (SEQ ID NO: 304), in particular where the foldikine is dimeric IFN-β.

Similarly, the engineered linkers (Xn1) and (Xn2) bridging the two monomer sequences may have any suitable sequence; and particularly comprise a sequence that defines a conformationally restrained structure, i.e. a ‘structured’ linker. Such linkers may have from about 3 to about 20 amino acid residues; from about 3 to about 16 amino acid residues; from about 4 to about 12 amino acid residues, from about 4 to about 8 amino acid residues, from about 3 to about 8 amino acid residues or from about 3 to about 6 amino acid residues. Beneficially, such linkers do not contain a plurality of Gly and/or Ser residues adjacent each other; for example, wherein the linker peptide contains 5 or less adjacent Gly and/or Ser residues; suitably 3 or less adjacent Gly and/or Ser residues; 2 or less Gly and/or Ser residues; contains only isolated Gly and/or Ser residues; or in some embodiments contains no Gly and/or Ser residues.

An exemplary pair of suitable linkers bridging the two monomer sequences comprises or consists of:
i) sequences designed with a protein design algorithm (ie FoldX, ModelX) so that the linkers interact either with themselves or with the two cyotkine domains, thereby adopting a preferred conformation; exemplary sequences include Xn1=TCMPGGSKT (SEQ ID NO: 337) and Xn2=RLELLP (SEQ ID NO: 331), or Xn1=NRLKKLMASSD (SEQ ID NO: 332) and Xn2=RLELLP (SEQ ID NO: 331);
ii) flexible sequences composed of Gly and Ser residues in different proportions, provided the length of the linkers remains short (preferably less or equal to 6, 5, 4 or 3 residues) preventing part of the split domain to fold on part of the continuous domain; exemplary sequences include Xn1=Xn2=GGG, or GGGSGG (SEQ ID NO: 273), or GGGSGGSGG (SEQ ID NO: 333), or GGGSGGSGGSGG (SEQ ID NO: 334); or
iii) Rigid sequences composed of Pro residues making a PolyPro helix of any length; exemplary sequences include Xn1=Xn2=GPG, or GPPPPPPPG (SEQ ID NO: 335), or GPPPPPPPPPG (SEQ ID NO: 336),
in particular where the foldikine is dimeric IL-22.

In some embodiments, once a class II Foldikine has been made following way A, a circular permutant variant is created where the natural Nt and Ct of the second-class II cytokine monomer are joined, and a loop is opened in the first second class II cytokine monomer.

According to some embodiments, a single chain dimeric IL-22 polypeptide is provided that comprises or consists of sequence SEQ ID NO:65, SEQ ID NO:66, SEQ ID NO: 282, SEQ ID NO: 283, SEQ ID NO: 284, SEQ ID NO: 285, SEQ ID NO: 286, SEQ ID NO: 287, or SEQ ID NO: 288, or a sequence at least 80%, 85%, 90%, 95%, 98% or 99% identical thereto that retains at least the same stability, and/or at least the same level of interaction with IL-22 receptor 1 and/or IL-22 receptor 2, as IL-22 polypeptide.

According to some embodiments, a single chain chimeric IL-10-IL-22 polypeptide is provided that comprises or consists of sequence SEQ ID NO: 76, or a sequence at least 80%, 85%, 90%, 95%, 98% or 99% identical thereto that retains at least the same stability, and/or at least the same level of interaction with IL-10 receptor and IL-22 receptor.

A foldikine comprising both a class I and a class II cytokine may be designed in the same way A as described for foldikines based on class I or class II cytokine.

According to this embodiment, in the single chain dimeric cytokine polypeptide of the invention, one of the first and second cytokine monomers is a class I cytokine monomer, and the other of the first and second cytokine monomers is a class II cytokine monomer.

In a first scheme the two cytokines are linked via two engineered peptides linkers after opening a natural loop in the type I and type II cytokines, and closing the natural N- and C-termini of the type II cytokine or type I cytokine via a linker.

### Way B of Forming Foldikines

According to this way of forming a foldikine, the natural N-terminus of cytokine monomer A is joined with the natural C-terminus of cytokine monomer B, and the natural C-terminus of cytokine monomer B is joined with the natural N-terminus of cytokine monomer A. A loop is opened in either cytokine monomer A, or cytokine monomer B.

Experiments conducted by making circular permutants of monomeric cytokines where we link the natural Nt and Ct and open the protein at different loops (see example 4) show which loops can be opened without compromising cytokine functionality.

According to this way B of making a foldikine, the single chain dimeric cytokine polypeptide comprises: (a) a first cytokine monomer domain or a functional portion thereof; and (b) a second cytokine monomer domain or a functional portion thereof, wherein the sequence of the second cytokine monomer domain or functional portion thereof is continuous in sequence while the sequence of the first cytokine monomer domain is such that the first sequence portion of the first cytokine monomer domain is arranged at the N-terminus of the second cytokine monomer domain or functional portion thereof corresponds to a C-terminal portion of a natural cytokine monomer domain, and the second sequence portion of the first cytokine monomer domain is arranged at the C-terminus of the second cytokine monomer domain or functional portion thereof corresponds to a N-terminal portion of the natural cytokine monomer domain.

Structurally, the single chain dimeric cytokine polypeptide comprises:
i) a split domain that comprises the free N- and C-termini of the single chain dimeric cytokine polypeptide, and the first and second sequence portions of the first cytokine monomer domain in an order inverted compared to their natural sequence order (i.e. the second portion which is a C-terminal portion of the first cytokine monomer domain is at the N-side of the first portion of the first cytokine monomer domain, which is a N-terminal portion of the first cytokine monomer domain); and
ii) a continuous domain that comprises the second cytokine monomer domain or a functional portion thereof in its natural sequence order (i.e. in the N-terminal to C-terminal direction);
   wherein the sequences of the first and second sequence portions of the first cytokine monomer domain, in the split domain, are separated by the sequence of the continuous domain.

In the case of class I cytokines which do not form swapped domain dimers, forming a foldikine according to Way B involves linking two monomeric class I cytokines via two linkers to bridge respectively the N- and C-termini of each cytokine monomer, followed by opening a loop in one of the monomers to create new N and C-terminals for the single chain polypeptide. The loop to be opened in the one class I cytokine monomer may be the loop between α-helices A and B, between α-helices B and C, or between α-helices C and D.

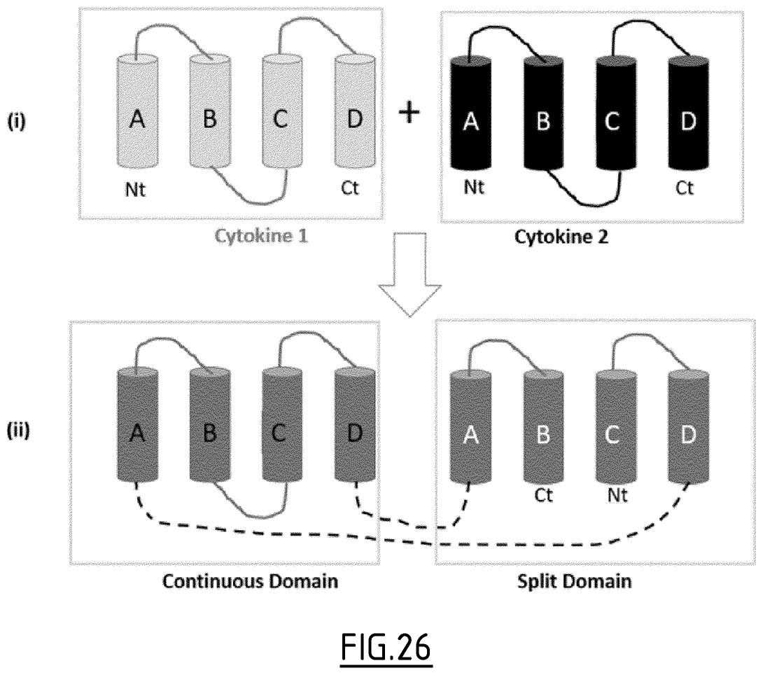

According to some embodiments, a single chain dimeric class I polypeptide is provided that comprises two class I cytokine monomer domains, or a functional portion thereof, wherein in said single chain dimeric cytokine polypeptide:
a) a linker peptide bridges the fourth α-helix (α-helix D) of a first class I cytokine monomer (e.g. ‘Cytokine 2’ on FIG. 26) with the first α-helix (α-helix A) of a second class I cytokine monomer (e.g. ‘Cytokine 1’ on FIG. 26);
b) a linker peptide bridges the fourth α-helix (C-terminal α-helix, or α-helix D) of the second class I cytokine monomer with the first α-helix (N-terminal α-helix, or α-helix A) of the first class I cytokine monomer; and
c) either α-helices A and B of said first class I cytokine monomer are not bridged, or α-helices B and C of said first class I cytokine monomer are not bridged, or α-helices C and D of said first class I cytokine monomer are not bridged.

In said single chain dimeric cytokine, the new N-terminus and C-terminus created by opening a loop in the first class I cytokine monomer are free.

According to an embodiment, the loop between α-helices B and C is opened in one cytokine monomer, and the single chain dimeric class I polypeptide comprises, from the N-terminus to the C-terminus, i) a C-terminal portion of a first class I cytokine monomer comprising α-helices C and D of said first class I cytokine monomer, ii) a linker peptide bridging α-helix D of said first class I cytokine monomer with α-helix A of a second class I cytokine monomer, iii) a portion of said second class I cytokine monomer comprising α-helices A to D of said second class I cytokine monomer, iv) a linker peptide bridging α-helix D of said second class I cytokine monomer with α-helix A of said first class I cytokine monomer; and v) a N-terminal portion of said first class I cytokine monomer comprising α-helices A and B.

According to an embodiment, the loop between α-helices A and B is opened in one cytokine monomer, and the single chain dimeric class I polypeptide comprises, from the N-terminus to the C-terminus, i) a C-terminal portion of a first class I cytokine monomer comprising α-helices B to D of said first class I cytokine monomer, ii) a linker peptide bridging α-helix D of said first class I cytokine monomer with α-helix A of a second class I cytokine monomer, iii) a portion of said second class I cytokine monomer comprising α-helices A to D of said second class I cytokine monomer, iv) a linker peptide bridging α-helix D of said second class I cytokine monomer with α-helix A of said first class I cytokine monomer; and v) a N-terminal portion of said first class I cytokine monomer comprising α-helix A.

According to an embodiment, the loop between α-helices C and D is opened in one cytokine monomer, and the single chain dimeric class I polypeptide comprises, from the N-terminus to the C-terminus, i) a C-terminal portion of a first class I cytokine monomer comprising α-helix D of said first class I cytokine monomer, ii) a linker peptide bridging α-helix D of said first class I cytokine monomer with α-helix A of a second class I cytokine monomer, iii) a portion of said second class I cytokine monomer comprising α-helices A to D of said second class I cytokine monomer, iv) a linker peptide bridging α-helix D of said second class I cytokine monomer with α-helix A of said first class I cytokine monomer; and v) a N-terminal portion of said first class I cytokine monomer comprising α-helices A to C.

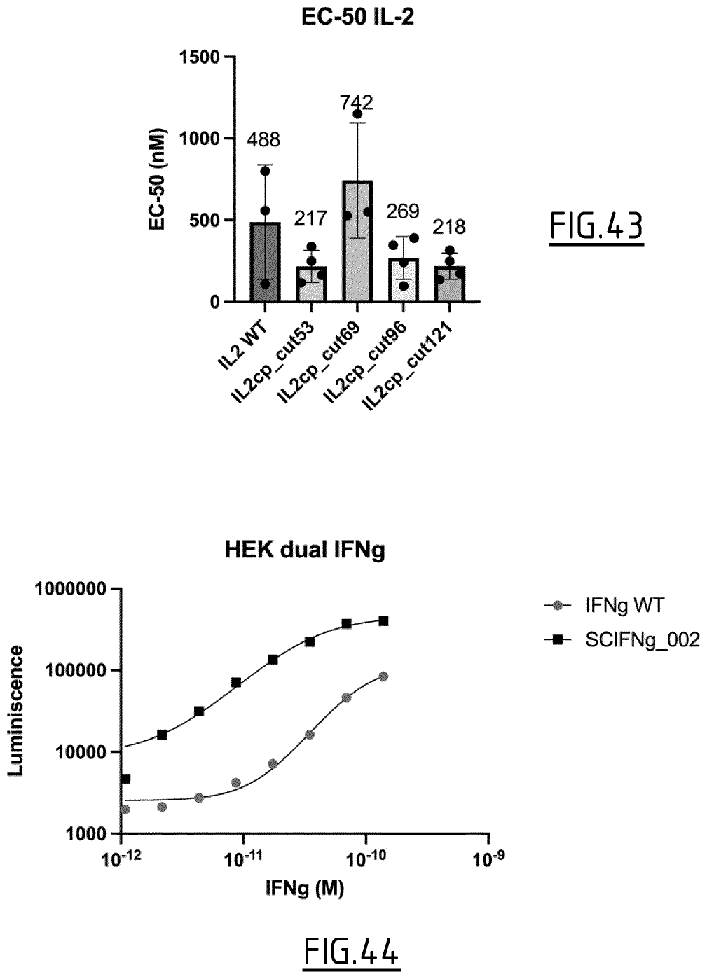

In the context of IL-2 cytokine monomer, and with reference to the sequence SEQ ID NO:79 (without signal peptide), it was shown in particular that the monomer may be opened at loop at positions 49-54 (in particular between residues at positions 52-53) or 69-76 (in particular between residues at positions 68-69) between α-helices A and B, at loop at positions 91-101 (in particular between residues at positions 96-97) between α-helices B and C, or at loop at positions 117-125 (in particular between residues at positions 120-121) between α-helices C and D (see FIG. 43).

In some embodiments, once a class I Foldikine has been made following way B, a circular permutant variant is created where the natural Nt and Ct of the second-class I cytokine monomer are joined, and a loop is opened in the first second class I cytokine monomer.

An example of single chain dimeric class I polypeptide that comprises IL-2 and IL-4 cytokine monomers (wherein IL-4 contains the continuous domain) comprises or consists of sequence SEQ ID NO: 295, or a sequence at least 80%, 85%, 90%, 95%, 98% or 99% identical thereto that retains at least the same stability, and/or at least the same level of interaction with IL-2 receptor and IL-4 receptor.

Exemplary heterodimeric Foldikine based on Class I cytokine include:

```
MYRMQLLSCIALSLALVTNSLRPRDLISNINVIVLELKGSETTFMCEYADETATIVEFLNRWITFCQSII
                                            hhhhh-hhhhhhhhhh hhhhhhhhhhhhhhhhh
                                                      IL-2 HC IL-2 HD
STLTGPPPPPGHKCDITLQEIIKTLNSLTEQKTLCTELTVTDIFAASKNTTEKETFCRAATVLRQFYSHH
H hhhhhhhhhhhh hhh hhhhhhhhhhhhhhhhhh
                                        IL-4 HA IL-4 HB
EKDTRCLGATAQQFHRHKQLIRFLKRLDRNLWGLAGLNSCPVKEANQSTLENFLERLKTIMREKYSKCSS
                  hhhhhhhhhhhhhhhhhhhhhhhhh hhhhhhhhhhhhhhhhhhhhhhhh
                                    IL-4 HC IL-4 HD
GPPPPPGTKKTQLQLEHLLLDLQMILNGINNYKNPKLTRMLTFKFYMPKKATELKHLQCLEEELKPLEEV
              hhhhhhhhhhhhhhhhhhhhhh hhhhhhhh---hhhhhh
                      IL2-HA IL-2 HB
LNLAQS*
```

Exemplary homomeric Foldikine based on short chain Class I cytokine include:
i) dimeric IL-2 (which may comprise or consist of SEQ ID NO: 89-(Xn1)-SEQ ID NO: 90-(Xn2)-SEQ ID NO:91),
ii) dimeric IL-4 (which may comprise or consist of SEQ ID NO: 93-(Xn1)-SEQ ID NO: 94-(Xn2)-SEQ ID NO:95),
iii) dimeric IL-3 (which may comprise or consist of SEQ ID NO: 97-(Xn1)-SEQ ID NO: 98-(Xn2)-SEQ ID NO:99),
iv) dimeric IL-7 (which may comprise or consist of SEQ ID NO: 103-(Xn1)-SEQ ID NO: 104-(Xn2)-SEQ ID NO:105),
v) dimeric IL-9 (which may comprise or consist of SEQ ID NO: 107-(Xn1)-SEQ ID NO: 108-(Xn2)-SEQ ID NO:109),
vi) dimeric IL-15 (which may comprise or consist of SEQ ID NO: 110-(Xn1)-SEQ ID NO: 111-(Xn22)-SEQ ID NO:112),
vii) dimeric IL-21 (which may comprise or consist of SEQ ID NO: 113-(Xn1)-SEQ ID NO: 114-(Xn)-SEQ ID NO:115),
viii) dimeric TSLP (which may comprise or consist of SEQ ID NO: 116-(Xn1)-SEQ ID NO: 117-(Xn2)-SEQ ID NO:118), and
ix) dimeric GM-CSF (which may comprise or consist of SEQ ID NO: 120-(Xn1)-SEQ ID NO: 121-(Xn2)-SEQ ID NO:12 SEQ ID NO: 101),
or a sequence at least 95%, 98% or 99% identical thereto that retains at least the same stability, and/or at least the same level of interaction with the respective receptor(s) of said monomeric short chain Class I cytokine;
wherein (Xn1) and (Xn2) are peptide linkers.

Exemplary homomeric Foldikine based on long chain Class I cytokine include:
i) dimeric IL-6 (which may comprise or consist of SEQ ID NO: 135-(Xn1)-SEQ ID NO: 136-(Xn2)-SEQ ID NO:137),
ii) dimeric IL-11 (which may comprise or consist of SEQ ID NO: 139-(Xn1)-SEQ ID NO: 140-(Xn2)-SEQ ID NO:141),
iii) dimeric IL-12a (which may comprise or consist of SEQ ID NO: 143-(Xn1)-SEQ ID NO: 144-(Xn2)-SEQ ID NO:145),
iv) dimeric IL-23a (which may comprise or consist of SEQ ID NO: 147-(Xn1)-SEQ ID NO: 148-(Xn2)-SEQ ID NO:149),
v) dimeric IL-27a (which may comprise or consist of SEQ ID NO: 151-(Xn1)-SEQ ID NO: 153-(Xn2)-SEQ ID NO:153),
vi) dimeric IL-31 (which may comprise or consist of SEQ ID NO: 154-(Xn1)-SEQ ID NO: 155-(Xn2)-SEQ ID NO:156),
vii) dimeric CLCF1 (which may comprise or consist of SEQ ID NO: 158-(Xn1)-SEQ ID NO: 159-(Xn2)-SEQ ID NO:160),
viii) dimeric ONCM (which may comprise or consist of SEQ ID NO: 161-(Xn1)-SEQ ID NO: 162-(Xn2)-SEQ ID NO:163),
ix) dimeric CTF1 (which may comprise or consist of SEQ ID NO: 165-(Xn1)-SEQ ID NO: 166-(Xn2)-SEQ ID NO:167),
x) dimeric CNTF (which may comprise or consist of SEQ ID NO: 169-(Xn1)-SEQ ID NO: 170-(Xn2)-SEQ ID NO:171),
xi) dimeric LIF (which may comprise or consist of SEQ ID NO: 173-(Xn1)-SEQ ID NO: 174-(Xn2)-SEQ ID NO:175), and
xii) dimeric CSF3 (which may comprise or consist of SEQ ID NO: 177-(Xn1)-SEQ ID NO: 178-(Xn2)-SEQ ID NO:179),
or a sequence at least 80%, 85%, 90%, 95%, 98% or 99% identical thereto that retains at least the same stability, and/or at least the same level of interaction with the respective receptor(s) of said monomeric long chain Class I cytokine;
wherein (Xn1) and (Xn2) are peptide linkers.

In any such aspects and embodiments, the linker peptides (Xn1) and (Xn2) bridging the α-helices, e.g. between the C-terminus of one monomer and the N-terminus of the other monomer, may have any suitable sequence; and preferentially comprises a sequence that defines a conformationally restrained structure, i.e. a ‘structured’ linker. Such linkers may have from about 3 to about 20 amino acid residues; from about 3 to about 16 amino acid residues; from about 4 to about 12 amino acid residues, from about 4 to about 8 amino acid residues, from about 3 to about 8 amino acid residues or from about 3 to about 6 amino acid residues. In some embodiments, such linkers do not contain a plurality of Gly and/or Ser residues adjacent each other; for example, wherein the linker peptide contains 5 or less adjacent Gly and/or Ser residues; suitably 3 or less adjacent Gly and/or Ser residues; 2 or less Gly and/or Ser residues; contains only isolated Gly and/or Ser residues; or in some embodiments contains no Gly and/or Ser residues. Preferentially such linkers contain at least two contiguous Pro residues, preferably 3, 4, 5 or 6 contiguous Pro residues, forming a PolyPro helix that is rigid. In some embodiments, the peptide linkers Xn1 and Xn2 are identical.

In some embodiments, dimeric IL-2 comprises or consists of SEQ ID NO: 89-(Xn1)-SEQ ID NO: 90-(Xn2)-SEQ ID NO:91 wherein the peptide linkers Xn1 and Xn2 are identical and comprise, or consist of, sequence GPPPPG (SEQ ID NO: 270), GPPPPPG (SEQ ID NO: 271), GPPPPPPG (SEQ ID NO: 272), GGGSGG (SEQ ID NO: 273), GGGSGGG (SEQ ID NO: 274), or GGGSGGGG (SEQ ID NO: 275). In these embodiments, dimeric IL-2 may further comprise a Lys residue immediately in N-ter of SEQ ID NO:89, and a Gln-Ser dipeptide immediately in C-ter of SEQ ID NO:91 (sequence K-SEQ ID NO: 89-(Xn1)-SEQ ID NO:90-(Xn2)-SEQ ID NO:91-QS).

In the case of class II cytokines which do not form swapped domain dimers, forming a foldikine according to Way B involves linking two monomeric class II cytokines via two linkers to bridge respectively the N- and C-termini of each cytokine monomer, followed by opening a loop in one of the monomers to create new N and C-terminals for the single chain polypeptide. The loop to be opened in the one class II cytokine monomer can be the loop between α-helices A and B, between α-helices B and C, between α-helices C and D, between α-helices D and E, or between α-helices E and F.

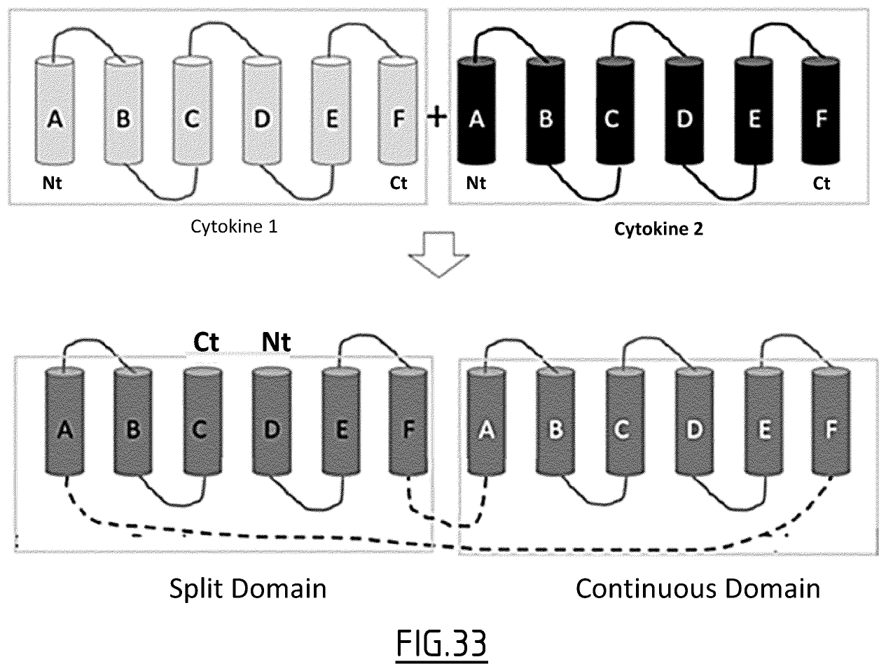

According to some embodiments, a single chain dimeric type II cytokine polypeptide is provided that comprises two cytokine monomers from class II cytokine(s) that do(es) not form swapped domain dimers, wherein in said single chain dimeric type II cytokine polypeptide:
a) a linker sequence bridges the first α-helix (N-terminal α-helix, or α-helix A) of a first class II cytokine monomer (e.g. ‘Cytokine 1’ on FIG. 33) with the sixth α-helix (C-terminal α-helix, or α-helix F) of a second class II cytokine monomer (e.g. ‘Cytokine 2’ on FIG. 33);
b) a linker sequence bridges the sixth α-helix (C-terminal α-helix, or α-helix F) of said first class II cytokine monomer with the first α-helix (C-terminal α-helix, or α-helix A) of said second class II cytokine monomer; and
c) the first and second second α-helices (α-helices A and B), the second and third α-helices (α-helices B and C), the third and fourth α-helices (α-helices C and D), the fourth and fifth α-helices (α-helices D and E), or the fifth and sixth α-helices (α-helices E and F) of said first class II cytokine monomer are not bridged.

In said single chain dimeric cytokine, the new N-terminus and C-terminus created by opening a loop in the first class II cytokine monomer are free.

According to an embodiment, the loop between α-helices B and C is opened in the first class II cytokine monomer, and the single chain dimeric class II polypeptide comprises, from the N-terminus to the C-terminus, i) a C-terminal portion of a first class II cytokine monomer comprising α-helices C to F of said first class II cytokine monomer, ii) a linker peptide bridging α-helix F of said first class II cytokine monomer with α-helix A of a second class II cytokine monomer, iii) a portion of said second class II cytokine monomer comprising α-helices A to F of said second class II cytokine monomer, iv) a linker peptide bridging α-helix F of said second class II cytokine monomer with α-helix A of said first class II cytokine monomer, and v) a N-terminal portion of said second class I cytokine monomer comprising α-helices A and B.

According to an embodiment, the loop between α-helices C and D is opened in the first class II cytokine monomer, and the single chain dimeric class II polypeptide comprises, from the N-terminus to the C-terminus, i) a C-terminal portion of a first class II cytokine monomer comprising α-helices D to F of said first class II cytokine monomer, ii) a linker peptide bridging α-helix F of said first class II cytokine monomer with α-helix A of a second class II cytokine monomer, iii) a portion of said second class II cytokine monomer comprising α-helices A to F of said second class II cytokine monomer, iv) a linker peptide bridging α-helix F of said second class II cytokine monomer with α-helix A of said first class II cytokine monomer, and v) a N-terminal portion of said first class II cytokine monomer comprising α-helices A to C.

According to an embodiment, the loop between α-helices E and F is opened in the first class II cytokine monomer, and the single chain dimeric class II polypeptide comprises, from the N-terminus to the C-terminus, i) a C-terminal portion of a first class II cytokine monomer comprising α-helix F of said first class II cytokine monomer, ii) a linker peptide bridging α-helix F of said first class II cytokine monomer with α-helix A of a second class II cytokine monomer, iii) a portion of said second class II cytokine monomer comprising α-helices A to F of said second class II cytokine monomer, iv) a linker peptide bridging α-helix F of said second class II cytokine monomer with α-helix A of said first class II cytokine monomer, and v) a N-terminal portion of said first class II cytokine monomer comprising α-helices A to E.

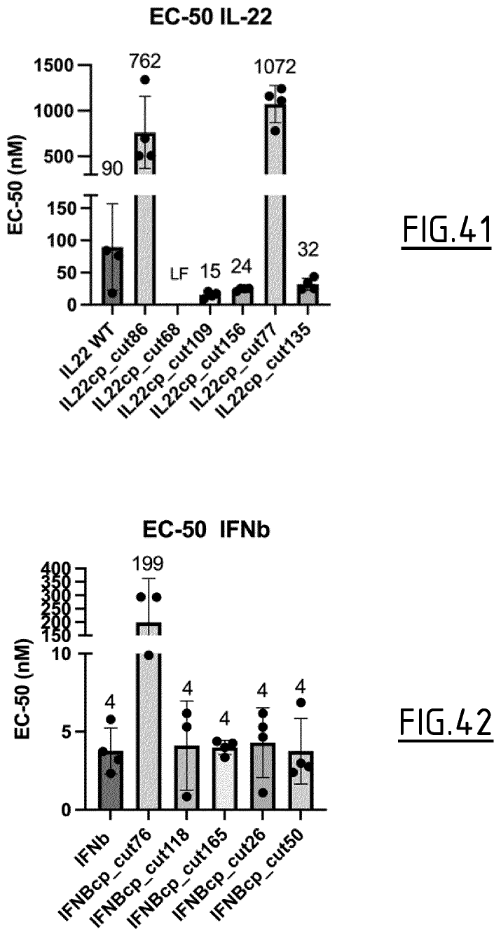

In the context of IL-22 cytokine monomer, and with reference to the sequence SEQ ID NO:291 (without signal peptide), it was shown in particular that the monomer may be opened at loop at positions 102-113 (in particular between residues at positions 108-109) between α-helices B and C, or at loop at positions 129-140 (in particular between residues at positions 134-135) between α-helices C and D, or at loop at positions 165-167 (in particular between residues at positions 164-165) between α-helices E and F (see FIG. 41).

In the context of IFNβ cytokine monomer, and with reference to the sequence SEQ ID NO:291 (without signal peptide), it was shown in particular that the monomer may be opened at loop at positions 34-51 (in particular between residues at positions 49-50) between α-helices A and B, or at loop at positions 71-81 (in particular between residues at positions 75-76) between α-helices B and C, or at loop at positions 108-121 (in particular between residues at positions 117-118) between α-helices C and D (see FIG. 42).

In any such aspects and embodiments, the linker peptides (Xn1) and (Xn2) bridging the α-helices, e.g. between the C-terminus of one monomer and the N-terminus of the other monomer, may have any suitable sequence; and preferentially comprises a sequence that defines a conformationally restrained structure, i.e. a ‘structured’ linker. Such linkers may have from about 3 to about 20 amino acid residues; from about 3 to about 16 amino acid residues; from about 4 to about 12 amino acid residues, from about 4 to about 8 amino acid residues, from about 3 to about 8 amino acid residues or from about 3 to about 6 amino acid residues. In some embodiments, such linkers do not contain a plurality of Gly and/or Ser residues adjacent each other; for example, wherein the linker peptide contains 5 or less adjacent Gly and/or Ser residues; suitably 3 or less adjacent Gly and/or Ser residues; 2 or less Gly and/or Ser residues; contains only isolated Gly and/or Ser residues; or in some embodiments contains no Gly and/or Ser residues. Preferentially such linkers contain at least two contiguous Pro residues, preferably 3, 4, 5 or 6 contiguous Pro residues, forming a PolyPro helix that is rigid.

In some embodiments, the peptide linkers Xn1 and Xn2 are identical.

A foldikine comprising both a type I and a type II cytokine monomer may be designed in the same way B as described for foldikines based on class I or class II cytokine.

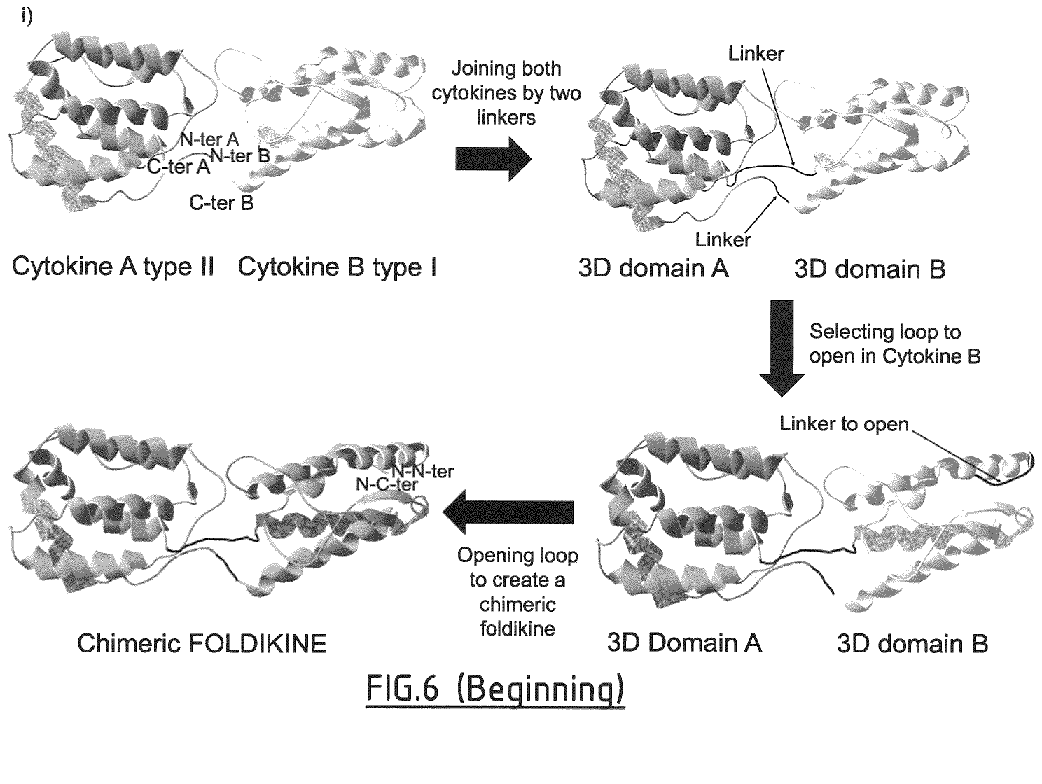

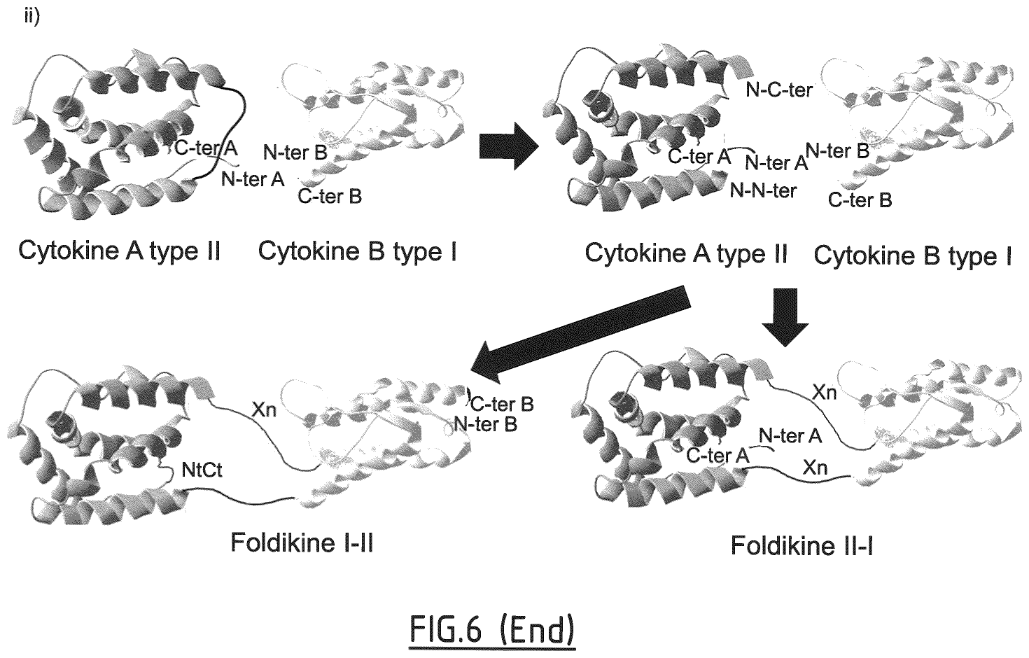

In this scheme, the two cytokines are linked via two engineered linker peptides bridging the natural N-terminus of the cytokine type I with the natural C-terminus of the cytokine type II, and the natural C-terminus of cytokine type I with the natural N-terminus of cytokine type II, and opening a loop in the cytokine type I (see e.g. FIG. 6i) or in the cytokine type II (not shown).

In some embodiments, once a class II Foldikine has been made following way B, a circular permutant variant is created where the natural Nt and Ct of the second-class II cytokine monomer are joined, and a loop is opened in the first second class II cytokine monomer.

In any such aspects and embodiments, the linker sequences bridging between the N- and C-termini of the type I and type II cytokines may have any suitable sequence; and preferentially comprises a sequence that defines a conformationally restrained structure, e.g. a ‘structured’ linker. Such linkers may have from about 3 to about 20 amino acid residues; from about 3 to about 16 amino acid residues; from about 4 to about 12 amino acid residues, from about 4 to about 8 amino acid residues, from about 3 to about 8 amino acid residues or from about 3 to about 6 amino acid residues. Beneficially, such linkers do not contain a plurality of Gly and/or Ser residues adjacent each other; for example, wherein the linker peptide contains 5 or less adjacent Gly and/or Ser residues; suitably 3 or less adjacent Gly and/or Ser residues; 2 or less Gly and/or Ser residues; contains only isolated Gly and/or Ser residues; or in some embodiments contains no Gly and/or Ser residues. Similarly, the engineered linkers bridging between the two monomer sequences may have any suitable sequence; and particularly comprise a sequence that defines a conformationally restrained structure, i.e. a ‘structured’ linker. Such linkers may have from about 3 to about 20 amino acid residues; from about 3 to about 16 amino acid residues; from about 4 to about 12 amino acid residues, from about 4 to about 8 amino acid residues, from about 3 to about 8 amino acid residues or from about 3 to about 6 amino acid residues. Beneficially, such linkers do not contain a plurality of Gly and/or Ser residues adjacent each other; for example, wherein the linker peptide contains 5 or less adjacent Gly and/or Ser residues; suitably 3 or less adjacent Gly and/or Ser residues; 2 or less Gly and/or Ser residues; contains only isolated Gly and/or Ser residues; or in some embodiments contains no Gly and/or Ser residues.

In any such aspects and embodiments, any such engineered linker peptide sequences (e.g. bridging between the N- and C-termini of cytokine type I and the opened loop termini of the type II cytokine, as well as that linking the N- and C-termini of the type II cytokine, if that option is chosen) may have any suitable sequence; and preferentially comprises a sequence that defines a conformationally restrained structure, e.g. a ‘structured’ linker. Such linkers may have from about 3 to about 20 amino acid residues; from about 3 to about 16 amino acid residues; from about 4 to about 12 amino acid residues, from about 4 to about 8 amino acid residues, from about 3 to about 8 amino acid residues or from about 3 to about 6 amino acid residues. Beneficially, such linkers do not contain a plurality of Gly and/or Ser residues adjacent each other; for example, wherein the linker peptide contains 5 or less adjacent Gly and/or Ser residues; suitably 3 or less adjacent Gly and/or Ser residues; 2 or less Gly and/or Ser residues; contains only isolated Gly and/or Ser residues; or in some embodiments contains no Gly and/or Ser residues.

### Way C of Forming Foldikines

In the case of class I cytokines, and according to way C of forming a foldikine a loop is opened in one class I cytokine monomer B, and the new N-terminus of the opened loop of cytokine monomer B is joined with the natural (or original) C-terminus of cytokine monomer A, and the new C-terminus of the opened loop of monomer B is joined with the the natural (or original) N-terminus of cytokine monomer A.

According to this way C of making a foldikine, the single chain dimeric cytokine polypeptide comprises: (a) a first class I cytokine monomer domain or a functional portion thereof; and (b) a second class I cytokine monomer domain or a functional portion thereof, wherein the sequence of the first class I cytokine monomer domain or functional portion thereof is continuous in sequence, while the sequence of second cytokine monomer domain is such that the first sequence portion of the second class I cytokine monomer which is arranged at the N-terminus of the first class I cytokine monomer or functional portion thereof corresponds to a N-terminal portion of a natural class I cytokine monomer, and the second sequence portion of the second class I cytokine monomer which is arranged at the C-terminus of the first class I cytokine monomer or functional portion thereof corresponds to a C-terminal portion of the natural class I cytokine monomer.

The single chain dimeric class I cytokine polypeptide comprises
b) a split domain that comprises the free N- and C-termini of the single chain dimeric class I cytokine polypeptide, and the first and second sequence portions of the second class I cytokine monomer domain; and
ii) a continuous domain that comprises the first class I cytokine monomer domain or a functional portion thereof;
   wherein the sequences of the first and second sequence portions of the second class I cytokine monomer domain, in the split domain, are separated by the sequence of the continuous domain.

In the case of class I cytokines, forming a foldikine according to way C may involve opening a loop in one monomer (e.g. between α-helices B and C).

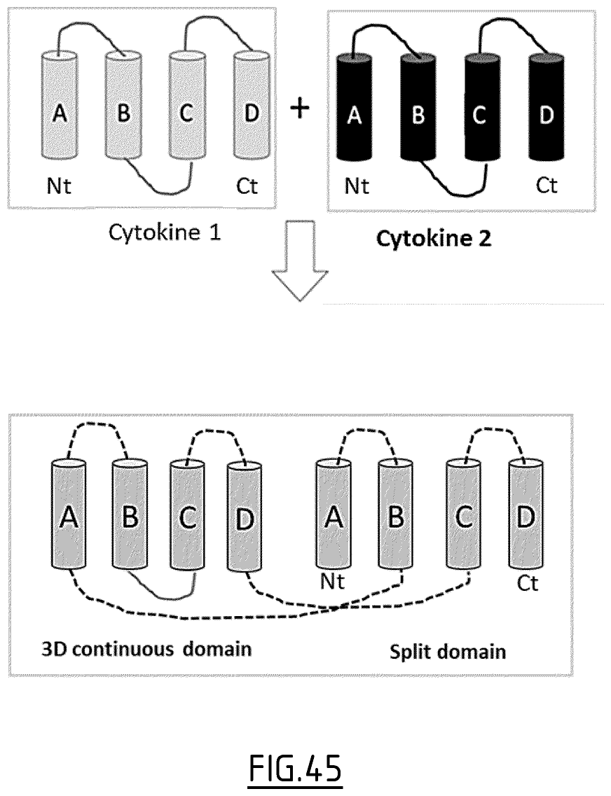

According to an embodiment, the loop between α-helices B and C is opened in one cytokine monomer, and a single chain dimeric class I polypeptide is provided that comprises two class I cytokine monomers, wherein in said single chain dimeric cytokine polypeptide:
a) a linker peptide bridges the fourth α-helix (α-helix D) of a first class I cytokine monomer (e.g. ‘Cytokine 1’ on FIG. 45) with the third α-helix (α-helix C) of the second class I cytokine monomer (e.g. ‘Cytokine 2’ on FIG. 45);
b) a linker peptide bridges the first α-helix (N-terminal α-helix, or α-helix A) of the first class I cytokine monomer with the second α-helix (α-helix B) of the second class I cytokine monomer; and
c) α-helices B and C of said second class I cytokine monomer are not bridged

In said single chain dimeric cytokine, the original N-terminus and C-terminus in the second class I cytokine monomer are free.

Altogether, the single chain dimeric class I polypeptide comprises, from the N-terminus to the C-terminus, i) a N-terminal portion of a second class I cytokine monomer comprising α-helices A and B of said second class I cytokine monomer, ii) a linker peptide (Xn1) bridging α-helix B of said second class I cytokine monomer with α-helix A of a first class I cytokine monomer, iii) a continues domain of said first class I cytokine monomer comprising α-helices A to D. iv) a linker peptide (Xn2) bridging α-helix D of the first class I cytokine monomer with α-helix C of the second class I cytokine monomer, and vii) a C-terminal portion of said second class I cytokine monomer comprising α-helices C and D of said first class I cytokine monomer.

The engineered linkers (Xn1) and (Xn2) bridging the two monomer sequences may have any suitable sequence; and particularly comprise a sequence that defines a conformationally restrained structure, i.e. a ‘structured’ linker. Such linkers may have from about 3 to about 20 amino acid residues; from about 3 to about 16 amino acid residues; from about 4 to about 12 amino acid residues, from about 4 to about 8 amino acid residues, from about 3 to about 8 amino acid residues or from about 3 to about 6 amino acid residues. Beneficially, such linkers do not contain a plurality of Gly and/or Ser residues adjacent each other; for example, wherein the linker peptide contains 5 or less adjacent Gly and/or Ser residues; suitably 3 or less adjacent Gly and/or Ser residues; 2 or less Gly and/or Ser residues; contains only isolated Gly and/or Ser residues; or in some embodiments contains no Gly and/or Ser residues.

According to some embodiments, a single chain circular permutant dimeric IL-2 polypeptide is provided that comprises or consists of sequence SEQ ID NO: X338:

```
SSSTKKTQLQLEHLLLDLQMILNGINNYKNPKLTRMLTFKFYMPKKATELKHLQCLEEELKPLEEVLNLA
      hhhhhhhhhhhhhhhhhhhhhh hhhhhhh---hhhhhhh
                            HA HB
QSKGGSGGSSSTKKTQLQLEHLLLDLQMILNGINNYKNPKLTRMLTFKFYMPKKATELKHLQCLEEELKPL
                      hhhhhhhhhhhhhhhhhhhhhh hhhhhhh---hhhh
                                    HA HB
EEVLNLAQSKNFHLRPRDLISNINVIVLELKGSETTFMCEYADETATIVEFLNRWITFCQSIISTLTGGSGG
                              hhhhh-hhhhhhhhhh hhhhhhhhhhhhhhhhh
                                              HC HD
NFLRPRDLISNINVIVLELKGSETTFMCEYADETATIVEFLNRWITFCQSIISTLTG
        hhhhh-hhhhhhhhhh hhhhhhhhhhhhhhhhh
                          HC HD
```

or a sequence at least 80%, 85%, 90%, 95%, 98% or 99% identical thereto that retains at least the same stability, and/or at least the same level of interaction with IL-2 receptor.

In some embodiments, once a class I Foldikine has been made following way C, a circular permutant variant is created where the natural Nt and Ct of the second class I cytokine monomer are joined, and a loop is opened in the first second class I cytokine monomer.

According to some embodiments, a single chain circular permutant dimeric IL-2 polypeptide is provided that comprises or consists of sequence SEQ ID NO: 296, or a sequence at least 80%, 85%, 90%, 95%, 98% or 99% identical thereto that retains at least the same stability, and/or at least the same level of interaction with IL-2 receptor.

In the case of class II cytokines, according to this way C of making a Foldikine, the single chain dimeric cytokine polypeptide comprises: (a) a first class II cytokine monomer domain or a functional portion thereof; and (b) a second class II cytokine monomer domain or a functional portion thereof, wherein the sequence of the second class II cytokine monomer domain or functional portion thereof is continuous in sequence, while the sequence of the first class II cytokine monomer domain is such that the first sequence portion of the first class II cytokine monomer domain which is arranged at the N-terminus of the second class II cytokine monomer domain or functional portion thereof corresponds to a N-terminal portion of a natural class II cytokine monomer domain, and the second sequence portion of the first class II cytokine monomer domain which is arranged at the C-terminus of the second class II cytokine monomer domain or functional portion thereof corresponds to a C-terminal portion of the natural class II cytokine monomer domain.

In the case of class II cytokines, the inserted sequence of the second-class II cytokine monomer is in its natural order.

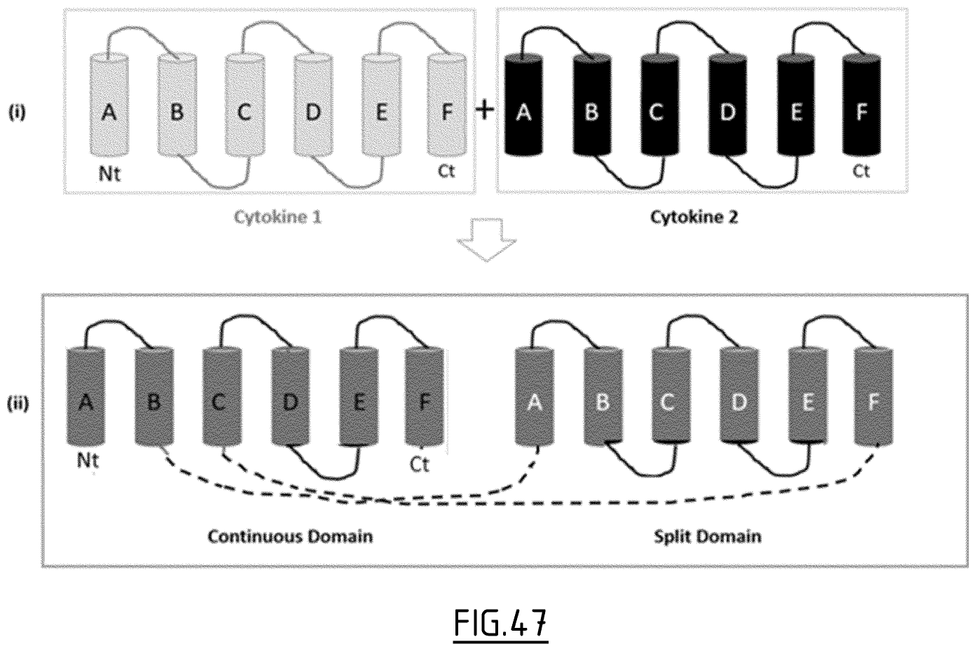

According to this way C of making a foldikine, the single chain dimeric class II cytokine polypeptide comprises (see FIG. 47):
i) a split domain that comprises the free N- and C-termini of the single chain dimeric class II cytokine polypeptide, and the first and second sequence portions of the first-class II cytokine monomer domain in their natural sequence order; and
ii) a continuous domain that comprises the second-class II cytokine monomer domain or a functional portion thereof;
   wherein the sequences of the first and second sequence portions of the first-class II cytokine monomer domain, in the split domain, are separated by the sequence of the continuous domain of the second-class II cytokine monomer domain.

In the case of class II cytokines, forming a foldikine according to way C may involve opening a loop in one monomer (in particular between α-helices B and C of one monomer) and joining the newly created C-terminus with the natural Ct of the other cytokine monomer, and joining the newly created N-terminus with the natural N-terminus of the other cytokine monomer.

According to an embodiment, the loop between α-helices B and C is opened in a first cytokine monomer, and a single chain dimeric class II polypeptide is provided that comprises two class II cytokine monomers, wherein in said single chain dimeric cytokine polypeptide:
a) a linker peptide bridges the second α-helix (α-helix B) of a first-class II cytokine monomer (‘Cytokine 1’ on FIG. 47) with the first α-helix (α-helix A) of the second-class II cytokine monomer (‘Cytokine 2’ on FIG. 47); and
b) a linker peptide bridges the third α-helix (α-helix C) of the first-class II cytokine monomer with the first α-helix (N-terminal α-helix, or α-helix A) of the second-class II cytokine monomer.

In said single chain dimeric cytokine, the natural N-terminus and C-terminus of the first-class II cytokine monomer are free, and the inserted sequence of the second-class II cytokine monomer follows its natural sequence order.

Altogether, the single chain dimeric class II polypeptide comprises, from the N-terminus to the C-terminus, i) a N-terminal portion of a first class II cytokine monomer comprising α-helices A and B of said first class II cytokine monomer, ii) a linker peptide (Xn1) bridging α-helix B of said first class II cytokine monomer with α-helix A of a second class II cytokine monomer, iii) the natural sequence of said second class II cytokine monomer comprising α-helices A to F of said second class II cytokine monomer, iv) a linker peptide (Xn2) bridging α-helix F of the second class II cytokine monomer with α-helix C of the first class II cytokine monomer, and vii) a N-terminal portion of said first class II cytokine monomer comprising α-helices C to F of said first class I cytokine monomer.

Similarly, the engineered linkers (Xn1) and (Xn2) bridging the two monomer sequences may have any suitable sequence, as disclosed above in ways A and B of making a foldikine.

In some embodiments, once a class II Foldikine has been made following way C, a circular permutant variant is created where the natural Nt and Ct of the second-class II cytokine monomer are joined and a loop is opened in the first second class II cytokine monomer.

Furthermore, a foldikine comprising both type I and type II cytokine monomer domains may be designed in the same way C as described for foldikines based on class I or class II cytokines.

In scheme C-a, the two cytokine monomers are linked via two linkers after opening a natural loop in the type II cytokine, and closing the new N- and C-termini of the opened loop via two engineered linkers to the natural N- and C-termini of the type I cytokine. In addition, a circular permutat ersion of it can be made where a natural loop in the type I cytokine may be opened and the natural N- and C-termini of the type II cytokine are closed via a linker (see FIG. 6ii), or a natural loop in the type I cytokine may be opened and the new Nt and Ct linked to the natural N- and C-termini of the type II cytokine, while the natural N- and C-termini of the type I cytokine are joined and a loop is opened in the type II cytokine to become the new Nt and Ct

### Structural Templates Used to Define the Angle Between the Two 3D Domains of a Foldikine

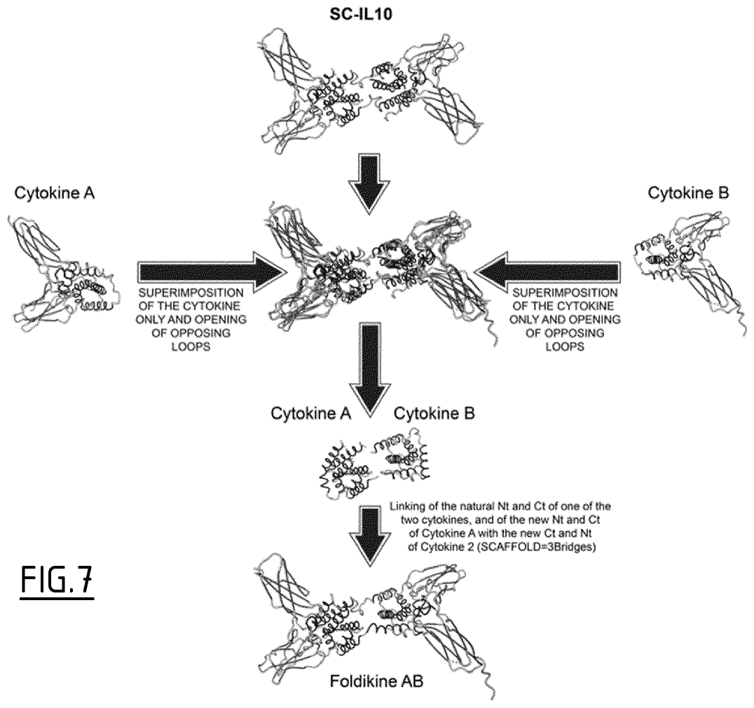

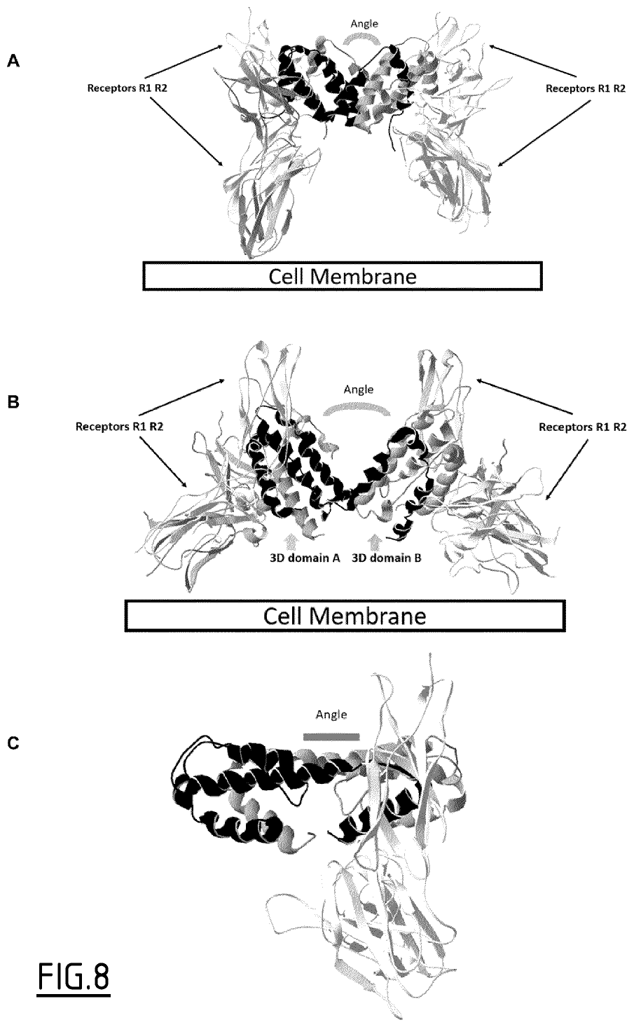

When designing a foldikine from a pair of monomeric cytokines a template/reference based on a swapped domain cytokine may be used. Beneficially, any such template/reference has a similar structure to the desired resulting foldikine, since in this way any beneficial angle between the two globular domains which could be important when binding four receptors with the synthetic foldikine may be maintained (see FIGS. 7 and 8).

### Polypeptides Comprising a Foldikine

Also provided is a polypeptide comprising a single chain dimeric polypeptide according to the invention.

In these aspects, the single chain dimeric polypeptide may be fused or covalently linked to any peptide, such as a protein tag, or other polypeptide.

### Recombinant Vectors, Host Cells and Expression

### Control Sequences/Regulatory Sequences

The terms ‘control sequence’ or ‘regulatory sequence’ are used interchangeably herein to refer to any nucleotide sequence which is capable of increasing or decreasing the expression of specific genes. This regulation may be imposed by either influencing transcription rates, translation rates, or by modification of the stability of the sequence. In embodiments, the polynucleotide sequences of the disclosure comprise regulatory elements such as but not limited to the following: promoters, enhancers, selection markers, origins of replication, linker sequences, polyA sequences, terminator sequence, and degradation sequences. In certain embodiments, a polynucleotide according to this disclosure comprises one or more suitable control sequences. In certain embodiments, the control sequences are identical for all polynucleotides of the disclosure. In alternative embodiments, different control sequences are used for or within different polynucleotides. In certain embodiments, the control sequences are control sequences naturally occurring in a target prokaryotic or eukaryotic cell. In other embodiments, the control sequences are adapted to perform their intended function in the target cell, e.g. bacteria. In some embodiments, the polynucleotides of the disclosure may encode tag sequences that ameliorate purification or localisation. Both oligonucleotide motifs and sequences that bind to other oligonucleotides or proteins and amino acid motifs or sequences are envisaged.

### Promoters

Polynucleotides of this disclosure may suitably include promoter sequences, or promoters, operably linked to a nucleotide sequence encoding a polypeptide of this disclosure. As used herein, a ‘promoter’ refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. At least one module in a promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the initiation point.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. Depending on the promoter, it appears that individual elements can function either cooperatively or independently to activate transcription.

Promoters may be constitutive, inducible, activatable, repressible, tissue-specific or any combination thereof. A promoter drives expression or drives transcription of the nucleic acid sequence that it regulates. Herein, a promoter is considered to be ‘operably linked’ when it is in a correct functional location and orientation in relation to a nucleic acid sequence it regulates to control (‘drive’) transcriptional initiation and/or expression of that sequence. The term ‘operably linked’ is used interchangeably herein with the terms ‘operatively positioned’, ‘under control’ and ‘under transcriptional control’.

A promoter may be classified as strong or weak according to its affinity for RNA polymerase (and/or sigma factor); this is related to how closely the promoter sequence resembles the ideal consensus sequence for the polymerase. The strength of a promoter may depend on whether initiation of transcription occurs at that promoter with high or low frequency. Different promoters with different strengths may be used as desired.

Suitable promoter sequences may be natural or synthetic. For example, they may be naturally associated with a gene or sequence, as may be obtained by isolating the 5′ non-coding sequences located upstream of the coding segment of a given gene or sequence. Such a promoter can be referred to as ‘endogenous’. Similarly, an activator/enhancer may be one naturally associated with a nucleic acid sequence, located either within, downstream or upstream of that sequence.

In some embodiments, a coding nucleic acid segment may be positioned under the control of a recombinant or heterologous promoter, which refers to a promoter that is not normally associated with the encoded nucleic acid sequence in its natural environment. A recombinant or heterologous enhancer refers to an enhancer not normally associated with a nucleic acid sequence in its natural environment. Such promoters or enhancers may include promoters or enhancers of other genes; promoters or enhancers isolated from any suitable eukaryotic or prokaryotic cell; and synthetic promoters or enhancers that are not ‘naturally occurring’ such as, for example, those that contain different elements of different transcriptional regulatory regions and/or mutations that alter expression through methods of genetic engineering. In addition to producing nucleic acid sequences of promoters and enhancers synthetically, sequences may be produced using recombinant cloning and/or nucleic acid amplification technology, including polymerase chain reaction (PCR).

In certain embodiments, the promoter may be a constitutive promoter. A constitutive promoter is understood by a skilled person to be a promoter whose expression is constant under the standard culturing conditions, i.e. a promoter which expresses a gene product at a substantially constant expression level.

In alternative embodiments, the promoter may be an inducible (conditional) promoter. As used herein, an ‘inducible promoter’ is one that is characterised by initiating or enhancing transcriptional activity when in the presence of, influenced by or contacted by an inducer or inducing agent. An ‘inducer’ or ‘inducing agent’ may be endogenous or a normally exogenous condition, compound or protein that contacts the promoter or transcriptional machinery in such a way as to be active in inducing transcriptional activity from the inducible promoter. Inducible promoters, and more specifically bacterial inducible promoter systems have been described in great detail in the art (e.g. in Brautaset et al., Positively regulated bacterial expression systems, Microbial biotechnology, 2009).

Inducible promoters for use in accordance with the present disclosure function in a microbial cell such as a bacterial cell. Examples of inducible promoters for use herein include, without limitation, bacteriophage promoters (e.g. Plslcon, T3, T7, SP6, PL) and bacterial promoters (e.g. Pbad, PmgrB, Ptrc2, Plac/ara, Ptac, Pm), or hybrids thereof (e.g. PLlacO, PLtetO). Examples of bacterial promoters for use in accordance with the present disclosure include, without limitation, positively regulated E. coli promoters such as positively regulated σ70 promoters (e.g., inducible pBad/araC promoter, Lux cassette right promoter, modified lamdba Prm promote, plac Or2-62 (positive), pBad/AraC with extra REN sites, pBad, P(Las) TetO, P(Las) ClO, P(Rhl), Pu, FecA, PRE, cadC, hns, pLas, pLux), as promoters (e.g. Pdps), σ32 promoters (e.g., heat shock) and σ54 promoters (e.g., glnAp2); negatively regulated E. coli promoters such as negatively regulated σ70 promoters (e.g., Promoter (PRM+), modified lamdba Prm promoter, TetR-TetR-4C P(Las) TetO, P(Las) ClO, P (Lac) IQ, RecA_DlexO_DLac01, dapAp, FecA, Pspac-hy, pel, plux-cl, plux-lac, CinR, CinL, glucose controlled, modified Pr, modified Prm+, FecA, Pcya, rec A (SOS), Rec A (SOS), EmrR_regulated, Betl_regulated, plac_lux, pTet_Lac, pLac/Mnt, pTet/Mnt, LsrA/cl, pLux/cl, Lacl, LacIQ, pLacIQI, pLas/cl, pLas/Lux, pLux/Las, pRecA with LexA binding site, reverse BBa_R0011, pLacI/ara-1, pLacIq, rrnB PI, cadC, hns, PfhuA, pBad/araC, nhaA, OmpF, RcnR), oS promoters (e.g., Lutz-Bujard LacO with alternative sigma factor 038), σ32 promoters (e.g., Lutz-Bujard LacO with alternative sigma factor σ32), and σ54 promoters (e.g., glnAp2); negatively regulated B. subtilis promoters such as repressible B. subtilis σA promoters (e.g., Gram-positive IPTG-inducible, Xyl, hyper-spank) and σ promoters. Other inducible microbial promoters and/or bacterial promoters may be used in accordance with the present disclosure.

An inducible promoter for use in accordance with the present disclosure may be induced by (or repressed by) one or more physiological condition(s), such as changes in pH, temperature, radiation, osmotic pressure, saline gradients, cell surface binding, and the concentration of one or more extrinsic or intrinsic inducing agent(s). The extrinsic inducer or inducing agent may comprise, without limitation, amino acids and amino acid analogues, saccharides and polysaccharides, nucleic acids, protein transcriptional activators and repressors, cytokines, toxins, petroleum-based compounds, metal containing compounds, salts, ions, enzyme substrate analogues, hormones or combinations thereof.

Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g. anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g. promoters derived from metallothionein (proteins that bind and sequester metal ions) genes from yeast, mouse and human), pathogenesis-regulated promoters (e.g. induced by salicylic acid, ethylene or benzothiadiazole (BTH)), temperature/heat-inducible promoters (e.g. heat shock promoters), and light-regulated promoters (e.g. light responsive promoters from plant cells). In certain embodiments, the promoter is a TetR promoter part of a Tet-On or Tet-off system (Krueger et al., Tetracycline derivatives: alternative effectors for Tet transregulators, Biotechniques, 2004; and Loew et al., Improved Tet-responsive promoters with minimized background expression, BioMedCentral Biotechnology, 2010).

Suitable promoter sequences for expression in Mycoplasma bacteria include, without limitation, the P3 promoter (48) and the synthetic promoter pSynL. Other promoters have been successfully employed in the Mycoplasma field, such as the SynMyco promoter (https://doi.org/10.1093/dnares/dsz012) or other promoters employed in intermediate versions derived from the Tn4001 transposon (10.1007/BF00382099).

Other promoters for use in accordance with the present disclosure may include any suitable promoter from any bacterial, viral or eukaryotic source; in particular, for expression in Lactococcus lactis or E. coli.

In various other embodiments, wherein the targeted cell is a mammalian cell, the human cytomegalovirus (CMV) immediate early gene promoter, the SV40 early promoter and the Rous sarcoma virus long terminal repeat may be used to obtain high-level expression of transgenes. The use of other viral or mammalian cellular or bacterial phage promoters which are well-known in the art to achieve expression of a transgene is contemplated as well, provided that the levels of expression are sufficient for a given purpose.

### Enhancers

In some embodiments of the present disclosure, a promoter may or may not be used in conjunction with an ‘enhancer’ which is also involved in transcriptional activation. An enhancer is one or more regions of DNA that can be bound with proteins (namely trans acting factors) to enhance transcription levels of a gene. The enhancer can be located at a functional region upstream or downstream of the promoter. While typically at the 5′ end of a coding region, it can also be separate from a promoter sequence, e.g. can be within an intronic region of a gene or 3′ to the coding region of the gene.

### Terminators

In some embodiments, a polynucleotide of this disclosure may contain a terminator sequence, or terminator. A ‘terminator’, as used herein, is a nucleic acid sequence that causes transcription to stop. A terminator may be unidirectional or bidirectional. It is comprised of a DNA sequence involved in specific termination of an RNA transcript by an RNA polymerase. A terminator sequence prevents transcriptional activation of downstream nucleic acid sequences by upstream promoters. Thus, in certain embodiments, a terminator that ends the production of an RNA transcript is contemplated. A terminator may be necessary in vivo to achieve desirable gene/protein expression levels.

The most commonly used type of terminator is a forward terminator. When placed downstream of a nucleic acid sequence that is usually transcribed, a forward transcriptional terminator will cause transcription to abort. In some embodiments, bidirectional transcriptional terminators are provided, which usually cause transcription to terminate on both the forward and reverse strand. In some embodiments, reverse transcriptional terminators are encompassed, which usually terminate transcription on the reverse strand only. In prokaryotic systems, terminators usually fall into two categories: (i) rho-independent terminators; and (ii) rho-dependent terminators. Rho-independent terminators are generally composed of palindromic sequence that forms a stem loop rich in G-C base pairs followed by a string of uracil bases.

Terminators for use in accordance with the present disclosure include any terminator of transcription described herein or known to one of ordinary skill in the art. Examples of terminators include, without limitation, the termination sequences of genes such as, for example, the bovine growth hormone terminator, and viral termination sequences such as, for example, the TO terminator, the TE terminator, Lambda TI and the T1T2 terminator found in bacterial systems. In some embodiments, the termination signal may be a sequence that cannot be transcribed or translated, such as those resulting from a sequence truncation.

Other genetic elements are known in the art and may be used in accordance with the present disclosure.

### Expression Vectors

Any suitable polynucleotide of the disclosure can be part of an expression vector such as a plasmid, optionally a non-replicative plasmid, a phagemid, a bacteriophage, a bacteriophage-derived vector, an artificial chromosome, a minicircle, a lentiviral vector, a retroviral vector, an adenoviral or adeno-associated viral vector, a piggyback vector, or a tol2 vector. A skilled person is aware of these different types of constructs and their generation and manipulation has been detailed at numerous instances (Sambrook et al., Molecular cloning: a laboratory manual, ISBN 0879693096, 1989 and the corresponding updated 4th Edition, Cold Spring Harbor Laboratory Press, 2012). Furthermore, it is evident to a skilled person that plasmid DNA, or (circular) recombinant DNA is commonly referred to in the art as copy DNA, complement DNA, or by the abbreviation ‘cDNA’, which may each be used interchangeably.

The term ‘expression vector’ or ‘expression construct’ means any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of an RNA into a gene product.

Particularly useful vectors are contemplated to be those vectors in which the coding portion of the DNA segment, whether encoding a full-length protein or smaller polypeptide, is positioned under the transcriptional control of a promoter, as described above.

Naturally, it will be important to employ a promoter that effectively directs the expression of the polypeptide of the disclosure in the cell type, or organism, chosen for expression. The use of promoter and cell type combinations for protein expression is generally known to those of skill in the art of molecular biology, for example, see Sambrook et al. (1989), incorporated herein by reference. Any suitable promoter may be used in a vector or plasmid of this disclosure that directs the desired level of expression of the encoded polypeptide.

The particular promoter that is employed to control the expression of a nucleic acid is not believed to be critical, so long as it is capable of expressing the desired polypeptide at a desired level in the target cell or organism. Thus, where Mycoplasma is used, it may be preferable to position the polynucleotide coding region adjacent to and under the control of a promoter that is capable of being expressed in Mycoplasma. Similarly, where it is desired to express the polypeptide in E. coli or L. lactis, an appropriate promoter suitable for expression in E. coli or L. lactis should be used.

When generating recombinant bacteria or other cells, it may be useful to include one or more ‘reporter gene’. Generally, reporter genes encode a polypeptide not otherwise produced by the host cell; or a protein or factor produced by the host cell but at much lower levels; or a mutant form of a polypeptide not otherwise produced by the host cell. The reporter gene may encode an enzyme which produces a calorimetric or fluorometric change in the host cell which is detectable by in situ analysis and is a quantitative or semi-quantitative function of transcriptional activation. Exemplary reporter genes encode esterases, phosphatases, proteases and other proteins detected by activity that generate a chromophore or fluorophore as will be known to the person skilled in the art. Well-known examples of such a reporter gene are E. coli β-galactosidase, luciferase and chloramphenicol-acetyl-transferase (CAT). Alternatively, a reporter gene may encode a selectivity marker, for example, by rendering the host cell resistant to a selection agent. For example, the gene neo renders cells resistant to the antibiotic neomycin. It is contemplated that virtually any host cell system compatible with the reporter gene cassette may be used to determine the regulatory unit. Thus, mammalian or other eukaryotic cells, insect, bacterial or plant cells may be used.

A specific initiation signal also may be required for efficient translation of coding sequences. These signals include an ATG initiation code and adjacent sequences. Exogenous translational control signals, including an ATG initiation codon, may need to be provided. One of ordinary skill in the art would readily be capable of determining this and providing the necessary signals. It is well known that the initiation codon must be ‘in-frame’ with the reading frame of the desired coding sequence to ensure translation of the entire insert. Furthermore, it is well known that certain bacteria are capable of utilising alternative (non-ATG) start sites and, thus, initiation signals including such alternative sites are also contemplated. The exogenous translational control signals and initiation codons can be either natural or synthetic. The efficiency of expression may be enhanced by the inclusion of appropriate transcription enhancer elements.

As used herein, the terms ‘engineered’ and ‘recombinant’ cells are intended to refer to a cell into which an exogenous DNA segment or gene, such a polynucleotide encoding the polypeptides (foldikines) of this disclosure has been introduced. Therefore, engineered cells are distinguishable from naturally occurring cells that do not contain a recombinantly introduced exogenous DNA segment or gene. Recombinant cells include those having an introduced cDNA or genomic gene, and also include genes positioned adjacent to a promoter not naturally associated with the particular introduced gene.

To express a foldikine polypeptide of the present invention, one may prepare an expression vector that comprises a foldikine-encoding polynucleic acid under the control of one or more promoters. To bring a coding sequence ‘under the control of’ a promoter, the 5′ end of the transcription initiation site of the transcriptional reading frame is positioned generally between about 1 and about 50 nucleotides downstream of (i.e. 3′ of) the chosen promoter. The upstream promoter stimulates transcription of the DNA and promotes expression of the encoded polypeptide. One or more enhancer elements may also be associated with the expression construct.

Many standard techniques are available to construct expression vectors containing the appropriate nucleic acids and transcriptional/translational control sequences in order to achieve protein or polypeptide expression in a variety of host-expression systems. Cell types available for expression include, but are not limited to, bacteria, such as M. pneumoniae, E. coli, L. lactis, S. aureus, and B. subtilis transformed with recombinant bacteriophage DNA, plasmid DNA or cosmid DNA expression vectors.

In general, plasmid vectors containing replicon and control sequences that are derived from species compatible with the host cell are used in connection with these hosts. The vector ordinarily carries a replication site, as well as marking sequences that are capable of providing phenotypic selection in transformed cells. For example, E. coli is often transformed using derivatives of pBR322, a plasmid derived from E. coli species. PBR322 contains genes for ampicillin and tetracycline resistance and thus provides easy means for identifying transformed cells. The pBR plasmid, or other microbial plasmid or phage must also contain, or be modified to contain, promoters which can be used by the microbial organism for expression of its own proteins.

Mycoplasmas tend to have difficulties in maintaining replicative vectors. However, transposons derived from the Tn4001 (with different resistance markers) vector may be employed. The transposon contains a transposase, placed outside a pair of inverted repeats. The exogenous DNA sequence that might be inserted into the chromosome is placed within the inverted repeats. Once the vector has been inserted into the cell, the transposase catalyses the excision of the inverted repeats and introduces the inner sequence into the chromosome in a random manner. Therefore, pieces of exogenous DNA of different lengths can be introduced into the M. pneumoniae chromosome

In addition, phage vectors containing replicon and control sequences that are compatible with the host microorganism can be used as transforming vectors in connection with these hosts. For example, the phage lambda GEM™-11 may be utilised in making a recombinant phage vector that can be used to transform host cells, such as M. pneumoniae and E. coli.

Further useful vectors include pIN vectors (Inouye and Inouye, 1985); pQE (His-tagged) vectors (Qiagen) and pGEX vectors, for use in generating glutathione-S-transferase (GST) soluble fusion proteins for later purification, separation or cleavage. Other suitable fusion proteins are those with β-galactosidase, ubiquitin and the like.

It is contemplated that a polypeptide of the disclosure may be expressed in heterologous systems, such as bacteria in which cytokines are not normally expressed; or in some cases, may be expressed in eukaryotic cells, such as human cells.

In certain embodiments, a polynucleotide of the disclosure may comprise a bicistronic expression construct. In further embodiments, the polynucleotide is incorporated, i.e. inserted, into a cellular genome, suitably a genomic sequence of a bacteria, such as M. pneumoniae, L. lactis or E. coli. In some embodiments, the polynucleotide may be part of a cellular genome, e.g. a de novo designed cellular genome or a mutagenised or synthetic bacterial genome. In further embodiments, the nucleotide arrangement is comprised in a bacterial artificial chromosome or a yeast artificial chromosome.

In certain embodiments, the polynucleotides of the disclosure may be incorporated into a genetically modified Mycoplasma strain, such as the attenuated CV2 strain (Δmpn133, Δmpn372) (58) or CV8, based on CV2 in which mpn051 gene has been replaced with the gpsA gene from Mycoplasma penetrans. These polynucleotides can also be assessed in other Mycoplasmas of interest.

In certain embodiments, the polynucleotides of the disclosure may be comprised in a bacteriophage. The term ‘bacteriophage’ as described herein is indicative for a virus that infects and optionally is able to replicate within bacteria and archaea, which may be modified for therapeutic purposes as has been described in the art (e.g. Principi et al., Advantages and Limitations of Bacteriophages for the Treatment of Bacterial Infections, Frontiers in Pharmacology, 2019).

In further embodiments, the concatenation of different sequence elements (e.g. polynucleotides of the disclosure) may be considered as an ‘operon’. An ‘operon’ as used herein refers to a functional unit of DNA containing a cluster of genes in which all genes are controlled by a single promotor. Generally, genes from an operon are therefore co-transcribed. Transcribed genes from an operon are transcribed to a single mRNA strand and may be either translated together in the cytoplasm or spliced to generate monocistronic mRNAs that may be translated separately.

Vectors and plasmids of the disclosure typically also include an ‘origin of replication’ or ‘ORI’, which refers to a sequence at which replication is initiated in either prokaryotic or eukaryotic organisms. DNA replication may proceed from this point bidirectionally or unidirectionally. Commonly used prokaryotic origins or replication include but are by no means limited to pMBI, modified pMBI, pBR322, ColEI, ColEI derivative, FI, R6K, pl5A, pSClOI, and pUC.

In order that a polynucleotide can be inserted and cloned, i.e., propagated, a vector will typically contain one or more unique restriction sites, and may be capable of autonomous replication in a defined cell or vehicle organism such that the cloned sequence is reproducible. As noted above, such a vector may also contain a selection marker, such as, e.g., an antibiotic resistance gene, to allow selection of recipient cells that contain the vector, as noted above.

### Expressed Polypeptides

In embodiments, the polypeptides of the disclosure—as encoded by the polynucleotides of this disclosure—may comprise a ‘display signal’ or ‘exposure signal’ and/or a ‘secretion signal’. Accordingly, the polynucleotides of this disclosure may comprise a nucleic acid sequence encoding one or more such sequence.

The term ‘display signal’ or ‘exposure signal’ refers to a peptide sequence that targets the fused/linked peptide (e.g. a foldikine of this disclosure) for exposure on the cell membrane. The cell may be a bacterial cell or eukaryotic cell, as desired. By ‘exposed’ or ‘displayed’, it is meant that the polypeptide of interest is still physically attached to the cell in which it was produced, preferably to the outer cell surface of the cell. Exposure signal sequences are known to the skilled person. In embodiments, the exposure signal sequence is a naturally occurring sequence in bacteria, such as E. coli, L. lactis or Mycoplasma (e.g. M. pneumoniae). In some embodiments, the exposure signal sequence is a non-naturally occurring bacterial sequence.

The term ‘secretion signal’ as used herein refers to a peptide sequence provoking or mediating secretion of a polypeptide from the cell in which it was expressed. As such, in contrast to when the polypeptide is exposed or displayed, the secreted polypeptide containing the secretion signal is no longer physically attached to the cell in which it was produced, such that it may be secreted into an extracellular space.

In embodiments, the secretion signal sequence may be a naturally occurring sequence in a bacteria, such as E. coli, L. lactis or Mycoplasma. In alternative embodiments, the secretion signal sequence is a non-naturally occurring bacteria sequence. Mycoplasma secretion signals have been described, for example, in international patent application WO2016/135281. In embodiments, concatenated secretion signals may be used to enhance secretion of the polypeptide; for example, different secretion signals may be incorporated at different locations of a polypeptide according to the disclosure.

In various embodiments, after the protein has been exposed on the cell surface (i.e. displayed), or after the protein has been secreted, the signal sequence may be removed from the linked protein by proteolytic cleavage. In certain embodiments, the exposure or secretion signal sequence is located at the N-terminus of the polypeptide.

### Diseases and Disorders

The agents, compositions, methods and uses of the present invention may be particularly suitable for the treatment of a wide range of diseases and disorders, including, for example, any disease or disorder that would benefit from a reduction or increase in an inflammatory response or in rate of cell proliferation. In particular, diseases and disorders that may be treated in accordance with the invention include cancers and/or proliferative or oncologic diseases (particularly solid tumours) and inflammatory diseases or disorders. Also, the present invention may be suitable to treat infectious diseases or to promote tissue regeneration after a tissue damage event or disease.

The skilled person will appreciate that proliferative diseases may be associated with: (1) pathological proliferation of normally quiescent or normally proliferating cells; (2) pathological migration of cells from their normal location (e.g., metastasis of neoplastic cells); (3) pathological expression of proteolytic enzymes such as the matrix metalloproteinases (e.g., collagenases, gelatinases, and elastases), which can lead to unwanted turnover of cellular matrices; and/or (4) pathological angiogenesis, as occurs in proliferative retinopathy and tumor metastasis.

Exemplary proliferative diseases include cancers, benign neoplasms, and angiogenesis that accompanies and facilitates a disease state (defined above as pathologic angiogenesis).

The compositions, agents, methods and uses of the present invention may have beneficial effects in treating a wide range of proliferative diseases and disorders and/or reducing the symptoms thereof; for example, by preventing cellular proliferation and especially in promoting cell death of pathogenic cells.

The invention may have utility in multiple cancer types, and/or have beneficial effects on tumour progression (such as, for example, reversing tumour progression) in vivo and/or in vitro. In particular, the invention may be useful in the treatment of lung cancers (in particular lung adenocarcinomas), cervical cancer, breast cancer, cardiac cancer, colon cancer, prostrate cancer, brain glioblastoma, pancreatic cancer, leukemia (e.g. acute monocytic leukemia), lymphoma, kidney cancer, colorectal cancer, bladder cancer, testicular cancer, gastrointestinal cancer, liver cancer (e.g. hepatocarcinoma), and/or glioblastoma. The invention may also be useful in the treatment of one or more of skin cancer (e.g. melanoma), head and/or neck cancer, gallbladder cancer, uterine cancer, stomach cancer, thyroid cancer, laryngeal cancer, lip and/or oral cancer, throat cancer, ocular cancer and bone cancer.

In some particular embodiments, the cancer may be selected from any one or more of the group consisting of acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, and Waldenstrom macroglobulinemia.

Furthermore, this disclosure also encompasses the therapeutic use of therapeutic agents and compositions of the invention, and methods for inhibiting or preventing local invasiveness or metastasis, or both, of any type of primary cancer. For example, the primary cancer can be melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder. In certain embodiments, the primary cancer can be liver cancer. For example, the liver cancer can be Hepatocellular carcinoma (HCC) and/or metastases to the liver.

Moreover, this disclosure can be used to prevent cancer or to treat pre-cancers or premalignant cells, including metaplasias, dysplasias, and hyperplasias. It can also be used to inhibit undesirable but benign cells, such as squamous metaplasia, dysplasia, benign prostate hyperplasia cells, hyperplastic lesions, and the like. In some embodiments, the progression to cancer or to a more severe form of cancer can be halted, disrupted, or delayed by the uses and methods of this disclosure involving the therapeutic agents disclosed herein.

As such, the invention provides agents and compositions for use in medicine and, in particular, for use in the treatment of cancers selected from lung cancers (in particular lung adenocarcinomas or lung squamous carcinoma), bladder cancer, cervical cancer, breast cancer, colon cancer, brain glioblastoma, pancreatic cancer, acute monocytic leukemia, kidney cancer, colorectal cancer, skin cancer (e.g. melanoma), stomach cancer, thyroid cancer, bone cancer and liver cancer. Methods for the treatment of such diseases are also provided. The uses and methods may comprise administering the agents according to the invention to a patient in need thereof.

Compositions and agents of this disclosure may also be useful in cancer immunotherapy and/or immuno oncology treatments.

### Inflammation

In parallel, the inflammatory response is an adaptative response to a variety of injures: physical, chemical or biological. There are different inflammation types: classical inflammation, homeostatic inflammation, low-grade inflammation, the adaptive response against stress known as para-inflammation or the metabolic-provoked inflammation. ‘Foldikines’ according to this disclosure can be used to treat inflammatory disorders, for example, in the scenario in which the immune system mistakenly attacks the cells or tissues of the own body's. Associated conditions of such immunological diseases or disorders are multiplend include pain, redness, swelling, stiffness or damage to other/neighbouring healthy tissues. The invention provides agents and compositions for use in medicine and, in particular, for use in the treatment of allergy, asthma, autoimmune diseases, coeliac disease, glomerulonephritis, hepatitis, inflammatory bowel disease, preperfusion injury, transplant rejection, diabetes, Alzheimer's disease, osteoarthritis, fibromyalgia, muscular low back pain, arthritis, muscular neck pain, myocarditis, Ankylosing Spondylitis (AS), Antiphospholipid Antibody Syndrome (APS), Myositis, Rheumatoid Arthritis, Scleroderma, Sjogren's Syndrome, fibrosis.

### Viral, Bacterial and Fungal Infections

‘Foldikines’ of this disclosure may useful be useful in the treatment of viral, bacterial or fungi infections. Any such microorganisms may trigger inflammatory responses and the release of physiological agents that alter the immune system. In other cases, such infectious agents, may produce and release toxins. To counterbalance the immune system response, foldikines might also be applied in the context of rubella, roseola, smallpox, chikungunya viral infection, measles, norovirus infection, coronavirus infection, shingles, hepatitis, herpes, dengue fever, ebola, lassa fever, Marburg hemorrhagic fever, polio, meningitis, encephalitis or rabies.

Since cytokines also play a key role in the orchestration of immune-mediated cellular repair and regenerative responses, the present invention may also provide additional benefits in boosting tissue repair after injury associated with such infections and infectious agents.

### Tissue Regeneration

The immune system plays a central role in tissue repair and regeneration. The immune response to tissue injury is crucial in determining the speed and the outcome of the healing process and the restoration of organ function. Therefore, controlling immune components via cytokines is an attractive approach in regenerative medicine. The foldikines of the present disclosure may, therefore, also have utility in methods and therapeutic uses for tissue regeneration, e.g. by promoting the proliferation and differentiation of the correct cells and preventing fibrosis. Examples of such uses are in the treatment of conditions such as colitis, wound healing, hair growth, liver regeneration and matrix synthesis.

The compositions, agents, methods and uses of the present invention may provide benefits in the treatment of any or all such diseases and disorders.

### Therapeutic Compositions

A single chain dimeric cytokine, polynucleotide or engineered bacteriophage, recombinant bacteria or cell (e.g. ‘therapeutic agents’) of the invention may be incorporated into a pharmaceutical composition for use in treating an animal; preferably a human. A therapeutic agent of the invention (or derivative thereof) may be used to treat one or more diseases or infections, depending on the activity/properties of the single chain dimeric cytokine of the invention. In various embodiments, a nucleic acid encoding the therapeutic peptide may be inserted into an expression construct/vector—particularly a bacteriophage vector, virus or bacterial genome—and incorporated into pharmaceutical formulations/medicaments for the same purpose. In the same way the foldikines of the disclosure could be delivered via conventional delivery methods, such as aerosols, intratracheal administration, injection, supositories etc. or may be administered via recombinant means.

As will be understood by the person of skill in the art, potential therapeutic agents, such as according to this disclosure, may be tested in an animal model, such as a rabbit or mouse, before they can be approved for use in human subjects. Accordingly, polypeptides, bacteriophages, bacteria or cells of this disclosure may be expressed or delivered in vivo in rabbit or mice or ex vivo in rabbit or mouse cells as well as in humans/human cells. In accordance with the invention, appropriate expression cassettes and expression constructs/vectors may be designed for each animal system specifically.

The therapeutic peptides and nucleic acids of the invention may be particularly suitable for the treatment of diseases, conditions and/or infections that can be targeted (and treated) intracellularly, for example, by targeting of bacteria or bacteriophages to a desired region of an animal (cell/tissue) as administered by conventional methods; and may also be suitable for in vitro and ex vivo applications. As used herein, the terms ‘therapeutic agent’ and ‘active agent’ encompass peptides and nucleic acids that encode a polypeptide of the invention, and also vectors, bacteriophages, viruses, bacteria and other cells that comprise peptides and/or nucleic acids as described herein. Therapeutic nucleic acids of the invention encompass modified/engineered bacteriophage, viruses or bacterial genomes.

Therapeutic uses and applications for the therapeutic agents of the invention include any disease, disorder or other medical condition that may be treatable by expressing or delivering a single chain dimeric cytokine of the invention in the subject to be treated by any delivery methodology applied in clinics.

In accordance with aspects and embodiments of the present invention, particularly preferred diseases include cancers and other proliferative diseases or disorders, inflammatory diseases or disorders, viral, bacterial and fungi infections and tissue regeneration as disclosed elsewhere herein.

One or more additional pharmaceutically acceptable ‘carrier’ (such as diluents, adjuvants, excipients or vehicles) may be combined with the therapeutic agent(s) of the invention in a pharmaceutical composition. Suitable pharmaceutical carriers are described in “Remington's Pharmaceutical Sciences” by E. W. Martin. Pharmaceutical formulations and compositions of the invention are formulated to conform to regulatory standards and can be administered orally, intravenously, topically, or via other standard routes. As used herein, the term ‘carrier’ includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase ‘pharmaceutically acceptable’ or ‘pharmacologically-acceptable’ refers to molecular entities and compositions that do not produce an allergic or similar untoward reaction when administered to a human.

In accordance with the invention, the therapeutic agent(s) may be manufactured into medicaments or may be formulated into pharmaceutical compositions. When administered to a subject, a therapeutic agent is suitably administered as a component of a composition that comprises a pharmaceutically acceptable vehicle. The molecules, compounds and compositions of the invention may be administered by any convenient route, for example, intradermal, intramuscular, intraperitoneal, intravenous, subcutaneous, intranasal, epidural, oral, sublingual, intranasal, intravaginal, transdermal, rectally, by inhalation, or topically to the skin. Administration can be systemic or local. Delivery systems that are known also include, for example, encapsulation in microgels, liposomes, microparticles, microcapsules, capsules, etc., and any of these may be used in some embodiments to administer the agents of the invention. Any other suitable delivery systems known in the art are also envisaged in use of the present invention.

Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are preferably sterile. Water is a suitable vehicle particularly when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The medicaments and pharmaceutical compositions of the invention can take the form of liquids, solutions, suspensions, lotions, gels, tablets, pills, pellets, powders, modified-release formulations (such as slow or sustained-release), suppositories, emulsions, aerosols, sprays, capsules (for example, capsules containing liquids or powders), liposomes, microparticles or any other suitable formulations known in the art. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

In some embodiments the therapeutic compositions or medicaments of the invention are formulated in accordance with routine procedures as a pharmaceutical composition adapted for oral administration (more suitably for human beings). Compositions for oral delivery may be in the form of tablets, lozenges, aqueous or oily suspensions, granules, powders, emulsions, capsules, syrups, or elixirs, for example. Thus, in various embodiments, the pharmaceutically acceptable vehicle may be a capsule, tablet or pill.

Orally administered compositions may contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. When the composition is in the form of a tablet or pill, the compositions may be coated to delay disintegration and absorption in the gastrointestinal tract, so as to provide a sustained release of active agent over an extended period of time. Selectively permeable membranes surrounding an osmotically active driving compound are also suitable for orally administered compositions. In these dosage forms, fluid from the environment surrounding the capsule is imbibed by the driving compound, which swells to displace the agent or agent composition through an aperture. These dosage forms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. For oral formulations, the location of release may be the stomach, the small intestine (the duodenum, the jejunem, or the ileum), or the large intestine. The person skilled in the art is able to prepare formulations that will not dissolve in the stomach, yet will release the material in the duodenum or elsewhere in the intestine. Suitably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 would be essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac, which may be used as mixed films.

To aid dissolution of the therapeutic agent(s) into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants, when used, could be present in the formulation of the peptide or nucleic acid or derivative either alone or as a mixture in different ratios.

Typically, compositions for intravenous administration comprise sterile isotonic aqueous buffer. Where necessary, the compositions may also include a solubilising agent.

Mixtures of the polypeptides, polynucleotides, vectors, bacteriophages, bacteria and/or other cells as described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases the form may be sterile and may be sufficiently fluid to enable injection by an appropriate syringe. Suitably, the composition is stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution may be suitably buffered if necessary and the liquid diluent first rendered isotonic with sufficient saline or glucose. These particular aqueous solutions are especially suitable for intravenous, intramuscular, subcutaneous, intratumoral and intraperitoneal administration. In this connection, sterile aqueous media that can be employed will be known to those of skill in the art.

Another suitable route of administration for the therapeutic compositions of the invention is via pulmonary or nasal delivery.

Additives may be included to enhance cellular uptake of a therapeutic agent of the invention, such as the fatty acids, oleic acid, linoleic acid and linolenic acid.

The therapeutic agents of the invention may, in some embodiments, also be formulated into compositions for topical application to the skin of a subject.

In embodiments of the invention the therapeutic compositions may include only one therapeutic agent of the invention; or may include two or more e.g. two complementary therapeutic agents of the invention. For example, inhibition of different inflammatory response pathways of the target animal may be achieved by more than one single chain dimeric cytokine of the disclosure or by single chain dimeric cytokines having two or more different receptor targeting ability. In particular, a single chain dimeric cytokine/foldikine of the invention may comprise a cytokine monomer of one type (e.g. IL-10) linked to a cytokine monomer of a different type (e.g. IL-22), such that each domain will preferentially target a different receptor and, potentially different inflammatory pathways. When two (or more) therapeutic agents are contemplated, the different peptides or encoding nucleic acid constructs such as bacteriophage, virus or bacteria may be incorporated into the same pharmaceutical composition, or may be manufactured separately. Where two (or more) pharmaceutical compositions are manufactured for administration to the same individual, it will be appreciated that the compositions may be administered simultaneously, sequentially, or separately, as directed/required.

### Methods for Delivery of Therapeutic Agents and Compositions

Any suitable delivery method for administering the therapeutic agents/therapeutic compositions of the invention may be used in accordance with the disclosure. A suitable route of administration may be determined by the skilled medical practitioner, and may be dependent on one or more factors.

Delivery of the therapeutic agents or compositions of the invention can be systemic or local. For example, the route of administration may be determined by the location and/or nature of the disease, e.g. cancer or inflammatory condition to be treated, and may include: intradermal, transdermal, parenteral, intravenous, intramuscular, intranasal, subcutaneous, regional (e.g., in the proximity of a tumor/target tissue), percutaneous, intratracheal, intraperitoneal, intraarterial, intravesical, intratumoral, inhalation, perfusion, lavage, and oral administration, for example, as described in U.S. Pat. Nos. 5,543,158; 5,641,515 and 5,399,363.

Typically, convenient forms of systemic administration may include oral administration, parenteral administration, intranasal administration, sublingual administration, rectal administration, transdermal administration, or any combinations thereof.

In various embodiments the therapeutic agents/compositions may be administered directly to the target tissue, organ or tumour. In some particularly beneficial embodiments, therapeutic compositions of the disclosure may be delivered by injection into the vasculature of the subject.

In some embodiments, (continuous) administration/administration over a prolonged period of time may be preferred and may be achieved by any suitable mechanism, for example, by implanting a catheter into a tumor, vasculature or tissue. Suitably, the dose of the therapeutic composition via continuous perfusion may be equivalent to that given by a single or multiple injections, adjusted over a period of time during which the perfusion occurs.

Injection of nucleic acid constructs, vectors and/or cells may be delivered by syringe or any other method used for injection of a solution, as long as the expression construct (e.g. bacteria) can pass through the particular gauge of needle required for injection. In some embodiments, a needleless injection system may be used, as known to the skilled person, e.g. as described in U.S. Pat. No. 5,846,233.

### Therapeutic Treatment Regimes and Doses

Treatment regimens may vary, and often depend on the type and/or location of the disease, such as a tumour, the stage/progression of the disease, and/or the health and age of the patient. For example, some tumours may respond better to treatment with localised and/or high concentration doses of a therapeutic composition of this disclosure, whereas other diseases (and individuals) may benefit from a more diffuse and/or low dose and/or prolonged administration of the therapeutic agent. The skilled clinician will be able to determine a suitable therapeutic treatment regime under each circumstance.

In various aspects and embodiments, the disclosure provides therapeutic uses and methods for treating a subject by administration of one or more therapeutic agents as disclosed herein. Accordingly, the disclosure provides uses and methods for reducing tumour size and/or growth by promoting the death of target tumour cells. The uses and methods comprise administering to an individual, or to a cancer cell or first population of target cells of the individual, a therapeutically effective amount of a therapeutic composition or agent of this disclosure, such as a single chain cytokine of the invention, or an agent that causes the single chain cytokine to be expressed. Beneficially, therefore, by use of a vector or delivery agent (e.g. bacteria, virus, cell or bacteriophage), which can replicate and cause multiple copies of the single chain cytokine to be expressed and/or released at a target site, it is not necessary to directly administer the therapeutic agent to every target cell or region (such as in a tumour) of a subject with an initial dose of the therapeutic agent or vehicle. Rather, by enabling the therapeutic agent to proliferate or to product multiple copies of the single chain cytokine of the disclosure, an enhanced therapeutic effect can be achieved.

An effective amount of a single chain cytokine or other therapeutic agent of the present disclosure or a pharmaceutical composition thereof, can include an amount sufficient to induce oncolysis/killing of a target cancer cell and/or the disruption of lysis or proliferation of a target cancer cell, or the inhibition or reduction in the growth or size of a target cancer cell or tumour. Reducing the growth of a tumour or target cancer cell may be manifested, for example, by cell death or a slower replication rate or reduced growth rate of a tumour comprising the cell, or a prolonged survival of a subject containing the cancer cell. In alternative aspects and embodiments, an effective amount of a single chain cytokine or other therapeutic agent of the present disclosure or a pharmaceutical composition thereof, can include an amount sufficient to reduce an inflammatory response in a target individual, tissue or organ, such as in the lungs.

Accordingly, in some embodiments, there is provided therapeutic uses and methods of treating a subject having a cancer or a tumor or an inflammatory disease or disorder (such as a lung disease, e.g. asthma) comprising administering, to the subject, an effective amount of a therapeutic agent of the disclosure. In some embodiments, the tumour may be entirely or substantially eradicated. In some embodiments, an inflammatory response may be entirely or substantially eradicated.

The uses and methods of the disclosure may include administering to the subject or individual the therapeutic agent. Administration may be by any suitable means, for example, by injection or ingestion. Administration may be systemic or local, e.g. directly to the site of a tumour. In one embodiment, systemic delivery is preferred.

The therapeutic compositions and/or therapies may include various ‘unit doses’, where a unit dose can be defined as containing a predetermined quantity of the therapeutic agent or composition. Identifying a suitable quantity to be administered, and the particular route of administration and formulation, are within the skill of those in the clinical arts. A unit dose may be administered as a single injection or tablet, for example; but may also be administered over a prolonged period of time, such as via a continuous infusion over a set duration.

In certain embodiments, the therapeutic agent can be administered in one or more doses over a set period of time (for example, over a 12 or 24 hour period), which together make a ‘unit dose’. In other embodiments each individual administration may be considered a ‘unit dose’.

The therapeutic compositions and/or agents of this disclosure may be administered in multiple doses (e.g., 2, 3, 4, 5, 6 or more doses), as deemed appropriate, within a suitable treatment period—for example, over a period of about 1, 2, 3, 4, 5, 6, 7, 8, 9, 10, 11 or 12 days, weeks, months or years as deemed appropriate. The frequency of administration of the therapeutic agent and/or the pharmaceutical compositions as described herein may be determined by the skilled practitioner, but may be, for example, once daily, twice daily, once every week, once every 2 weeks, once a month, once every 2 months, once every 3 months, once every 6 months and so on.

Further, in accordance with the therapeutic treatment regimes that may be devised, the unit dose may vary according to the stage or type of treatments, as well as having regard to the size and/or age of the subject, or the type of disease that it is intended to treat. For example, in some embodiments, the first dose of the therapeutic agent or pharmaceutical composition of this disclosure administered to the subject may be less than or higher than any second, third or subsequent dose that may be administered. In other embodiments, during a first administration period of time the dose may be lower or higher than the dose administered during a second, third or subsequent administration period. The skilled practitioner can determine the duration of any such administration periods, for example, about 1 day, about 1 week or about 1 month, or for any intermediate or longer period of time.

In some examples, the subject to be treated may be administered one or more additional therapeutic agent or may receive one or more additional conventional (such as radiation treatment or chemotherapy) or complementary therapy.

### Combination Therapies

In various embodiments, the uses and methods of this disclosure comprise administering a therapeutic agent of this disclosure (e.g. a polypeptide, polynucleotide, vector or cell (e.g. a recombinant bacterial cell as disclosed herein), or a pharmaceutical composition containing a therapeutic agent of this disclosure, in combination with one or more additional therapy or therapeutic agent. The one or more additional therapy or therapeutic agent may be administered simultaneously, sequentially or separately (before or after) the therapeutic agent according to this disclosure.

The additional therapeutic agent may be another therapeutic agent according to this disclosure, such that two therapeutic agents of the invention are administered in combination. Other examples of the further therapy or therapeutic agent can include, but are not limited to chemotherapy, radiation, oncolytic viral therapy with an additional virus, treatment with immunomodulatory proteins, an anti-cancer agent, anti-inflammatory agents or any combination thereof.

In particular embodiments, the additional therapeutic agent is an anti-cancer agent or cancer therapy. Anti-cancer agents can include, but are not limited to, chemotherapeutic agents, radiotherapeutic agents, cytokines, immune checkpoint inhibitors, anti-angiogenic agents, apoptosis-inducing agents, anti-cancer antibodies and/or anti-cyclin-dependent kinase agents. Cancer therapies can include chemotherapy, biological therapy, radiotherapy, immunotherapy, hormone therapy, anti-vascular therapy, cryotherapy, toxin therapy and/or surgery or combinations thereof.

In various embodiments, treatment of cancer may involve a surgical procedure. Such surgery can include resection in which all or part of a cancerous tissue is physically removed from the subject (e.g. by excision) and/or otherwise destroyed. Tumour resection refers to physical removal of at least part of a tumour. In addition to tumour resection, treatment by surgery includes laser surgery, cryosurgery, electrosurgery, and microscopically controlled surgery (Mohs' surgery). In some embodiments, it may be determined that a tumour (or part thereof) should be removed by resection, or alternatively, that the tumour is not suitable for resection. In such circumstances, embodiments of the invention may include treating the subject with therapeutic agents or compositions of this disclosure in order to improve the outcome of resection, or to enable the tumour or part thereof to be excised. Therapeutic treatments of the present disclosure may, therefore, increase the resectability of a tumour, e.g. due to shrinkage at tumour margins or by elimination of invasive portions. Additional treatments subsequent to resection will serve to eliminate microscopic residual disease at the tumour site.

### Kits

In embodiments, this disclosure provides a kit for administering a single chain dimeric cytokine, encoding polynucleotide, vector, cell or other agent (such as a bacteria or bacteriophage) as described herein. In certain embodiments, a kit of this disclosure can include a ‘therapeutic agent’, such as a single chain dimeric cytokine, encoding polynucleotide, vector, cell (e.g. bacteria) or bacteriophage or a (pharmaceutical) composition comprising such a therapeutic agent, as described above. In certain embodiments, a kit of this disclosure can further include one or more components such as instructions for use, devices and additional reagents, and components, such as tubes, containers and syringes for performing the methods and therapies disclosed herein. In various embodiments, a kit of this disclosure can further include one or more active agent, e.g., at least one selected from the group consisting of an anti-cancer agent, an immunomodulatory agent, an anti-inflammatory agent or any combinations thereof, that may be administered in combination (separately, sequentially or simultaneously) with the therapeutic agent of this disclosure.

In some embodiments, a kit according to the disclosure may comprise one or more containers containing the therapeutic agent, one or more additional active agent and/or any reagents as described herein.

In some embodiments, a kit may further include an apparatus or device for administering a therapeutic agent (such as a recombinant bacteria) of the disclosure, and/or any additional active agent to a subject. A suitable apparatus or device may include one or more of a hypodermic needle, an intravenous needle, a catheter, a needle-less injection device, an inhaler and/or a liquid dispenser.

Instructions for use of the kit may suitably include a description of what the kit should include and how it should be properly used; for example, how the various components of the kit should be administered to an individual, including timing, concentrations and quantities; proper administration methods and how/whether the individual should be monitored during use/treatment.

### EXAMPLES

### Example 1: Generation of IL-10 Variants and Single-Chain Dimeric IL-10

Unless otherwise indicated, commercially available reagents and standard techniques in molecular biological and biochemistry were used.

### Materials and Methods

The following procedures used by the Applicant are described in Sambrook, J. et al., 1989 supra.: analysis of restriction enzyme digestion products on agarose gels and preparation of phosphate buffered saline. General purpose reagents, oligonucleotides, antibodies, chemicals and solvents were purchased from Merck. Enzymes and polymerases were obtained from New England Biolabs (NEB), if not indicated otherwise.

### Methods

### Bacterial Strains and Culture Conditions

Where used, the WT Mycoplasma pneumoniae M129-B7 strain (ATTC 29342) and all the M. pneumoniae strains generated in this work are described in Table 1. Mycoplasma strains were grown at 37° C. under 5% CO₂ in tissue culture flasks (Corning) with Hayflick liquid medium. Hayflick was prepared by mixing 800 ml of non-complete medium A (20 g PPLO broth [Difco, #255420], 30 g HEPES [100 mM final], 25 ml 0.5% phenol red solution [Sigma, #P3532]), 200 ml heat-inactivated horse serum (Life Technologies, #26050088), 20 ml sterile-filtered 50% glucose and 1 ml of a 100 mg ml-1 stock of ampicillin (final concentration 100 μg ml-1, ampicillin sodium salt [Sigma, #A9518]). If growth on the plate was required, Hayflick broth was supplemented with 0.8% bacto-agar (Difco). Hayflick broth was supplemented with tetracycline or chloramphenicol (Tc; 20 μg ml-1 Cm; 20 μg ml-1) for cell selection, as needed. Pseudomonas aeruginosa PAO1 was grown in Trypticase Soy Broth (TSB, T8907) agar plates at 37° C. For cloning, the E. coli NEB® 5-alpha High-Efficiency strain (New England Biolabs) was used; it was grown at 37° C. in LB broth or on LB agar plates supplemented with ampicillin (100 μg ml⁻¹).

### Cell Lines

HAFTL cells are a foetal-liver-derived, Ha-ras-oncogene-transformed mouse pre-B cell line (47). BLaER1 are human B-cell precursor leukaemia cell lines (48); both cell lines were kindly provided by Professor Thomas Graf. The THP-1 cell line is a monocyte cell line derived from peripheral blood purchased from ATCC. Cells were grown in RPMI (12633012, GIBCO), supplemented with 2 mM L-glutamine (25030081, GIBCO), 100 U/mL penicillin+100 ng/ml streptomycin (15140122, GIBCO), and 20% FBS (10270-106, GIBCO).

### Plasmid Generation

All plasmids generated in this work were assembled following the Gibson method⁴⁷. When required, Integrated DNA Technologies (IDT) Corporation performed gene synthesis (gBlock double-stranded fragments) and oligonucleotides synthesis. Gene amplification was carried out with Phusion DNA polymerase (Thermo Fisher Scientific). In some examples, the promoter sequence for IL-10 and foldikine_10 variants was the P3 synthetic promoter⁴⁸, and the secretion signal termed S142 (U.S. Ser. No. 15/553,552, Garrido et al., 2021) was used. For IL-22 variants and IFN-Lambda the promoter used was pSynL (GTTAACGATTAAGATCAAAAAGTGCCTGGTATCGTAAATAATATTGTATAATTAAAAAAGA AT; SEQ ID NO: 339). A detailed list of plasmids, strains and their genetic parts is also given in Table 1. All plasmids were verified by PCR and Sanger sequencing (GATC biotech).

Generation of M. pneumoniae CV8 Strain

CV8 is a derivative strain of the attenuated CV2 strain (Δmpn133, Δmpn372) 58 in which mpn051 has been replaced by gpsA gene from Mycoplasma penetrans. The enzymes encoded by these two genes are involved in the oxidation of glycerol 3 phosphate, and generate the metabolic by-products of NADH in the case of the GpsA enzyme or H₂O₂ in the case of the GlpD enzyme encoded by mpn051; this H₂O₂ production is crucial for the cytotoxic effects of M. pneumoniae. The CV8 strain also carries a second gene deletion in which the puromycin-resistance gene (introduced to select the insertion of GP35 coding gene) was removed from its location in the genome (mpn560). Mutants were generated using genome editing tools adapted to mycoplasma based on ssDNA recombinase GP3549. As a substrate for recombination, long stretches of ssDNA were used, produced as previously described 50.

The sequences of the primers and the plasmids employed to generate the long stretches of ssDNA are shown in Table 4.

### Strains

For expression and secretion of the different versions of human cytokines, such as IL-10 (hIL-10), and the various foldikine constructs in bacteria, such as M. pneumoniae, transposon vectors were generated by fusing an appropriate promoter sequence to the cytokine and foldikine constructs, containing an appropriate signal peptide. For expression and secretion of IL-10 variants and foldikines having N-terminal IL10 sequences, the synthetic promoter P3 was used along with the signal peptide 142 (s142) at the N-terminus for secretion (see Table 1 for the genetic information of each construct). For the expression and secretion of IL-22 and IFN-Lambda variants and corresponding foldikine constructs, the synthetic promoter pSynL was used and fused to the signal peptide (s142). A tetracycline resistance marker was also used (see Methods herein).

To generate M. pneumoniae expression cells, the different plasmids were transformed into either WT or CV8 M. pneumoniae. Cells were grown in a 75-cm² tissue flask (Corning) containing 20-ml fresh Hayflick and incubated at 37° C. under 5% CO₂ until the late exponential phase. Cells were washed twice with a pre-cooled electroporation buffer (272 mM sucrose, 8 mM HEPES, pH 7.4), resuspended, scraped off and passed 10× through a 25-gauge (G25) syringe needle. Cell aliquots of 50 μl in 0.1-cm cuvettes with 2 μg of the desired plasmid were kept on ice for 20 min. The electroporation settings were set in 1250 V/25 μF/100Ω in a BIO-RAD Gene Pulser Xcell apparatus. After the pulse, 420-μl fresh Hayflick was added to the cells. From this culture, 80 μl were inoculated in a 25-cm² tissue flask (Corning) with 5-ml fresh Hayflick with the selection antibiotic (i.e., 20 μg ml⁻¹ chloramphenicol or tetracycline). From the different M. pneumoniae expression cells, 50-μl stock inoculum was added to a 25-cm² tissue flask (Corning) containing 5-ml fresh Hayflick and grown for 48 h. Culture media were then collected and centrifuged at 10,000 rpm for 10 min, and the supernatants were stored at −80° C. in 1 ml aliquots.

### Disulfide Identification by Mass Spectrometry

From the aliquots initially prepared and stored at −80° C., an initial inoculum of 25 μL of hIL-10, MutSC1, or MutSC2 was inoculated in a T25 cm² flask with 5 mL Hayflick medium supplemented with Cm in duplicate. After 24 h, the medium was discarded from one of the duplicates and replaced with Version 13 of the defined medium (see e.g. EP20382261). After 36 h, the supernatant of both replicates was collected and processed for proteomic analysis. No differences were observed in protein abundance under either set of conditions. For further characterisation, samples were grown in Version 13 of the defined medium.

Supernatants were collected, concentrated with MWCO 3K, and centrifuged for 15 min at 12,000 rpm with 700 μL supernatant. Then, 200 μL of supernatant was added to the same MWCO column, and the mixture was centrifuged for 60 min at 4° C. at 12,000 rpm. The final concentrated volume of approximately 100 μL was split into two: one for BCA quantification and the other for MS analysis. Each of the freshly-concentrated aliquots was resuspended in 100 μL of SDS 4%, 0.1 M HEPES. Supernatant lysates were quantified by BCA using the Pierce™ BCA Protein Assay Kit (Thermo Fisher Scientific, 23225) and sent to the UPF MS Facility for analysis. For sample preparation, the cysteines were labelled by alkylating with N-ethylmaleimide (30 nmol, 37° C., 60 min), and samples were then precipitated with 6 volumes of cold acetone. Pellets were dissolved in 6 M urea/200 mM ammonium bicarbonate, and samples were reduced with dithiothreitol (30 nmol, 37° C., 60 min) and alkylated in the dark with iodoacetamide (60 nmol, 25° C., 30 min).

Samples were then digested in the following manner, using 200 mM ammonium bicarbonate for all dilutions. For hIL-10 and MutSC2, the resulting protein extract was first diluted to 2 M urea and then incubated with the endoproteinase Lys-C(endo-LysC) (1:100 w: w, 37° C., 6 h, Wako, cat #129-02541); samples were then diluted 2-fold (1:100 w: w) and digested with i) trypsin (37° C., overnight; Promega cat #V5113); ii) chymotrypsin (25° C., overnight; Roche diagnostics cat #11418467001); or iii) GluC (25° C., overnight; Sigma Aldrich cat #P6181). The samples were first diluted to 6 M urea for trypsin digestion (1:100 w: w, 37° C., overnight, Promega cat #V5113) followed by GluC digestion (1:100 w: w, 25° C., o/n, Sigma Aldrich cat #P6181). For MutSC1, the combinatorial digestion mix was the following: i) endo-LysC plus trypsin: diluted to 2 M urea for endo-LysC (1:100 w: w, 37° C., 6 h, Wako, cat #129-02541) and then diluted 2-fold for trypsin (1:100 w: w, 37° C., overnight, Promega cat #V5113); ii) endo-LysC plus chymotrypsin: diluted to 2 M urea for endo-LysC (1:100 w: w, 37° C., 6 h, Wako, cat #129-02541) and then diluted 2-fold for trypsin digestion (1:100 w: w, 25° C., overnight, Roche diagnostics cat #11418467001); iii) LysC plus GluC: diluted to 2 M urea for endo-LysC (1:100 w: w, 37° C., 6 h, Wako, cat #129-02541), and then diluted 2-fold for GluC (1:100 w: w, 25° C., overnight, Sigma Aldrich cat #P6181); and iv) trypsin plus GluC: diluted to 6 M urea for trypsin digestion (1:100 w: w, 37° C., overnight, Promega cat #V5113) and then digested with GluC (1:100 w: w, 25° C., overnight, Sigma Aldrich cat #P6181). In all cases, the peptide samples after digestion were acidified with formic acid and desalted over a MicroSpin C18 column (The Nest Group, Inc) prior to LC-MS/MS analysis.

For chromatographic and MS analysis, the following protocol was used. Samples were analysed using an LTQ-Orbitrap Velos Pro mass spectrometer (Thermo Fisher Scientific, San Jose, CA, USA) coupled to an EASY-nLC 1000 (Thermo Fisher Scientific (Proxeon), Odense, Denmark). Peptides were loaded onto the 2 cm Nano Trap column with an inner diameter of 100 μm packed with C18 particles of 5 μm particle size (Thermo Fisher Scientific) and were separated by reversed-phase chromatography using a 25 cm column with an inner diameter of 75 μm, packed with 1.9 μm C18 particles (Nikkyo Technos Co., Ltd. Japan). Chromatographic gradients started at 93% buffer A and 7% buffer B with a flow rate of 250 nl min⁻¹ for 5 min, and gradually increased to 65% buffer A and 35% buffer B at 120 min. After each analysis, the column was washed for 15 min with 10% buffer A and 90% buffer B. Buffer A, 0.1% formic acid in water; buffer B, 0.1% formic acid in acetonitrile. The mass spectrometer was operated in positive ionisation mode with nano spray voltage set at 2.1 kV and source temperature at 300° C. Ultramark 1621 was used for external calibration of the FT mass analyser prior to analyses, and an internal calibration was performed using the background polysiloxane ion signal at m/z 445.1200. Measurements were acquired in the data-dependent acquisition (DDA) mode, and full MS scans with 1 micro scan at a resolution of 60,000 were used over a mass range of m/z 350-2000 with detection in the Orbitrap. Auto gain control (AGC) was set to 1E6, dynamic exclusion (60 sec), and a charge state filtering disqualifying singly-charged peptides was activated. In each cycle of DDA analysis, and following each survey scan, the top twenty most intense ions with multiple charged ions above a threshold ion count of 5,000 were selected for fragmentation. Fragment ion spectra were produced via collision-induced dissociation (CID) at a normalised collision energy of 35% and were acquired in the ion trap mass analyser. AGC was set to 1E4, an isolation window of 2.0 m/z, an activation time of 10 ms, and a maximum injection time of 100 ms were used. All data were acquired with Xcalibur software v2.2. Digested bovine serum albumin (New England Biolabs cat #P8108S) was analysed between each sample to avoid sample carryover and to assure stability of the instrument; QCloud (53) was used to control instrument longitudinal performance during the project.

Data were analysed using the Proteome Discoverer software suite (v2.0, Thermo Fisher Scientific) and the Mascot search engine (v2.6, Matrix Science (54)). Data were searched against a M. pneumoniae (87071 entries) plus hIL-10, MutSC1, MutSC2 and a list of common contaminants (55) and all of the corresponding decoy entries. For peptide identification, a precursor ion mass tolerance of 7 ppm was used for MS1 level, trypsin was chosen as the cleavage enzyme and up to three missed cleavages were allowed. The fragment ion mass tolerance was set to 0.5 Da for MS2 spectra. Oxidation of methionine, N-terminal protein acetylation, carbamidomethylation of cysteines and N-ethylmaleimide was used as variable modifications. The false discovery rate (FDR) in peptide identification was set to a maximum of 5%. Peptide quantification data were retrieved from the “Precursor ion area detector” node from Proteome Discoverer (v2.0) using 2 ppm mass tolerance for the peptide extracted ion current (XIC). The obtained values were used to calculate the protein's top 3 areas with the unique peptide for protein ungrouped.

For MutSC1, the PRM (parallel reaction monitoring)-chromatographic and MS analysis was performed. To confirm that the first cysteine is carbamidomethylated, the sample was digested with endo-LysC plus trypsin and analysed using an Orbitrap Eclipse (Thermo Fisher Scientific) coupled to an EASY-nanoLC 1200 UPLC system (Thermo Fisher Scientific) with a PRM method. The peptides were loaded directly onto the analytical column and were separated by reversed-phase chromatography using a 50 cm column with an inner diameter of 75 μm, packed with 2 μm C18 particles spectrometer (Thermo Scientific, San Jose, CA, USA).

Chromatographic gradients started at 95% buffer A and 5% buffer B with a flow rate of 300 nL/min for 5 min and gradually increased to 25% buffer B and 75% A over 79 min and then to 40% buffer B and 60% A over 11 min. After each analysis, the column was washed for 10 min with 10% buffer A (0.1% formic acid in water) and 90% buffer B (0.1% formic acid in 80% acetonitrile).

The mass spectrometer was operated in positive ionisation mode with an EASY-Spray nanosource at 2.4 kV and at a source temperature of 305° C. A full MS scan with 1 micro scan at resolution of 30,000 was used over a mass range of m/z 350-1400, with detection in the Orbitrap mass analyser. A PRM method was used for data acquisition with a quadrupole isolation window set to 1.4 m/z and MSMS scans over a mass range of m/z 300-2,000, with detection in the Orbitrap at resolution of 60,000. MSMS fragmentation was performed using HCD at 30 NCE, the auto gain control (AGC) was set to 1×10⁵, and maximum injection time of 502 ms. Peptide masses (m/z) were defined in the mass list and are shown in Table S2. For PRM data analysis, the Skyline-Daily software (4) (v21.1.9.353) was used to generate the libraries, which were observed as the output of the MaxQuant (1.6.10) search and predicted with Prosit (5), and to extract the fragment areas of each peptide.

### Quantifying Interleukin Expression in Bacterial Strains

To test each of the mutant candidates' expression capacity, cells were collected in 1 ml fresh Hayflick medium (e.g., cells were grown attached to the flask, the medium was aspirated and 1 ml of fresh medium was added). Cells were plated in 10 μl dots in solid Hayflick plates and grown for ten days at 37° C., 5% CO₂. The colony forming units (CFU) were counted across different dilutions ranges, from non-diluted to diluted 1×10⁻⁸. For each condition analysed, the concentration of the different expressed IL-10 variants was quantified by ELISA.

For IL-10 quantification, ELISA MAX Deluxe Set Human IL-10 (Biolegend; #430604) was performed following manufacturer's instructions. Supernatants were diluted up to 100,000-fold for this assay to enter into the detection range. As a positive control for each assay, commercial recombinant IL-10 (#200-10, Peprotech) (hIL-10r), or recombinant IL-22 (#200-22, Peprotech) was reconstituted in PBS at 10 μg ml⁻¹ and stored at −80° C. in 20 μl aliquots. The patron curve of commercial IL-10 used to infer the concentration of the mutants was fitted to a polynomial equation grade 2. The same procedure was followed for IL-22 quantification by ELISA (ELISA MAXIM Deluxe Set Human IL-22, 434504, BioLegend) or interferon lambda (LEGEND MAX™ Human IL-29 (IFN-A1) ELISA Kit, Biolegend). The SCIL10IL22 chimeras of the foldikine-22 molecules (described below) were quantified using the IL-22 ELISA kit.

### Blood Monocyte Isolation

Blood samples from four healthy donors were provided by the Banc de Sang i Teixits (Barcelona, Spain), under agreement n° 160002 approved by the Spanish Ministry of Science and Technology. Written informed consents were obtained from the donors before sample collection.

Peripheral blood mononuclear cells (PMBCs) were isolated from the buffy coat using Leucosep® tubes, according to the manufacturer's instructions. Briefly, 10 mL of blood samples were diluted with a ratio of 1:5 with phosphate-buffered saline solution (PBS), poured into the Leucosep® tubes and centrifuged for 15 min at 800 g at room temperature. PMBCs were collected using a Pasteur pipette, washed 2× with PBS and finally resuspended with 3 ml PBS. Monocytes were isolated by CD14⁺ magnetic labelling and differentiated into macrophages as previously described⁵⁴. Briefly, cells were purified using CD14 microbead-positive selection and MACS separation columns (Miltenyi Biotec), according to manufacturer's instructions.

To differentiate monocyte-derived macrophages, monocytes were adhered to glass coverslips (VWR international) in 6-well plates (Thermo Scientific), at 1.5×10⁶ cells/well for 1 h at 37° C. in warm RPMI 1640 medium (GIBCO). Medium was then supplemented to a final concentration of 10% fetal bovine serum (FBS, Sigma-Aldrich) and human recombinant macrophage colony-stimulating factor (M-CSF, Peprotech) at 20 ng ml-1. Cells were allowed to differentiate for 6-8 days.

### Culture of HEK-Blue™ Cells

HEK-blue™ IL-10, IL-22, IFN-Lambda cell lines carrying a SEAP reporter construct were purchased from InvivoGen (InvivoGen, San Diego, CA, USA). Cells were grown in DMEM (Lonza, BE12-604F) supplemented with 10% FBS, 2 mM L-glutamine. The HEK-IL-10 cells were supplemented with 100 μg ml⁻¹ normocin and selection antibiotics. HEK-blue IL-22 and HEK-blue-IFN-Lambda cells were supplemented with Blasticidin, Puromycin, Zeocin™. The cells were passaged when 70% confluence was reached, following the manufacturer's recommendation.

### Testing the Activity of Engineered Candidates in HEK-Blue™ Cells

After supernatant quantification by ELISA (see Methods), 200 μl aliquots of the different supernatants were stored at −80° C. Using fresh Hayflick medium for diluting the samples, different 500 μl ‘candidate aliquots’ of the following concentrations (30, 15, 7.5, 3.75, 1.88, 0.94, 0.47, and 0.23 ng ml-1) were prepared for each mutant candidate and as well for the recombinant protein. The volume of supernatant needed was determined by ELISA quantification, and volume was adjusted accordingly with a fresh Hayflick medium to reach the concentration stated above in the 20 μl supplement.

In parallel, HEK-blue™ IL-10, IL-22 or IFN-Lambda cell suspension was prepared at 280,000 cells ml⁻¹ in pre-warmed DMEM supplemented with 10% FBS, 2 mM L-glutamine (without antibiotics). Thereafter, 180 μl per well were seeded in a 96-well plate (Nunc Microwell, ThermoFisher Scientific, #167008). From this, 20 μl of each ‘candidate aliquot’ or the recombinant protein at a fixed concentration prepared in Hayflick medium was added, and cells were kept for 24 h at 37° C. and 5% CO₂ (induced HEK-cells). After 24 h, 180 μl of QUANTI-Blue Solution (Alkaline phosphatase detection medium, #repqbs, InvivoGen) was mixed with 20 μl induced HEK-cells in a new 96-well plate. Cells were then incubated 60 min at 37° C., and absorbance (630 nm) was measured in the spectrophotometer Tecan i-control, 2.0.10.0.

### Generation of Single-Chain (SC) Molecules Using ModelX Software

Proposed SC-IL10 variants were generated by rewiring the X-ray IL-10 dimeric structure (PDBs: 1y6k, 2ilk). The new connectivities were intelligently designed with the aid of the ModelX tool suite. The ModelX Bridging command (Cross-Linking mode) was used; it connects a pair of residues selected as anchors with all geometrically compatible fragments from a custom-made protein fragment library (PepXDB_5k). Bridging command allows the user to select different peptide lengths; the output is an ensemble of bridged models where linkers/connections with forbidden phi and psi dihedrals in the Ramachandran plot are discarded.

When peptides are longer than numeric positions between the anchors, Bridging renumbers surplus residues with res codes that are not recognised by FoldX in further modelling steps. For this reason, SC-IL10 design required a numerical rearrangement of the monomeric protein residues in the dimer, allowing numeric 18 amino acid long gaps between the regions to be connected. This is very important for the extreme case in which anchoring residues flank the numeric gap. To cover that situation, renumbering was done to create gaps of 18 numeric positions around the regions to be connected, which was enough to accept length fragments ranging from 6- to 20-residues without using the residue codes. The difference between 18 and 20 is that anchoring points are required for the algorithm to position the bridges, but they will be substituted with the terminal residues of the peptides found. Bridging also replaces all fringe residues between the anchors with the ones from the peptide. For clarity, the templates generated before using Bridging are termed ‘sewing patterns’ or ‘fusion’ patterns (e.g. FIGS. 3 to 7, 12 and 25 to 28). As the numeric gap is not a spatial gap, Bridging can also accept smaller peptide fragments, creating a discontinuous numbering.

Once the sewing/fusion patterns were created, extensive linker screening was performed by running the models through the Bridging algorithm with exhaustive combinations of anchoring points in an overlapping sliding window around the flanks of the regions to be joined. Every window was queried for different peptide lengths (6 to 20 aa). The bridged models are side chain repaired using the RepairPDB command of FoldX³⁰, and resulting models were ranked by global energy (FoldX Stability command).

The generation of MutSC1 was achieved in several steps: residues 12′-17′ (note that monomer 2 residues and atoms are depicted by ′) were deleted, as they were pointing away from the C′-terminal and were not in contact with receptor R1 in the x-ray crystal structure; and residues 18′-156′ were renumbered as 169-307 and were joined with the first monomer sequence to create a single-chain molecule, creating sewing/fusion pattern 1. As the best anchoring points, we selected the residues 147 (Ile) and 169 (Asn), which we then connected using a 17 amino acid fragment from PDB 2wyu (residues 73-89). The original sequence in 2wyu of the chosen bridge segment was LDALFAGVKEA-FGGLD-Y (SEQ ID NO: 340). Residues LDALFAGVKEA (SEQ ID NO: 341) and the last Y structurally superimposed with the wild-type IL-10 structure. Therefore, we decided to reverse all of these mutations back to the original IL-10 sequence, except when the new residue was expected to improve protein stability. In this case it was expected that stability would be improved by the Asn169 to Tyr mutation and so this mutation was maintained.

### Inferring Kinetic Data Values

Bacterial cell culture supernatants were diluted in Hayflick medium to adjust the IL-10, IL-22, IFN-Lambda or SCIL10IL22 concentration, covering a range going from 0.0243 ng ml⁻¹ to 30 ng ml⁻¹ that encompassed the dynamic range described for HEK-blue™ (see Methods). To calculate the molar concentration for each IL-10 variant, it was assumed that all IL-10 variants (including IL-10 ORF) have a molecular weight of 36 KDa, with the exception of MutM, which is a synthetic monomer described in (Josephson et al., 2001), and so has a molecular weight of 18 KDa.

For the foldikine-22 variants, the molecular weight is also 36 KDa but two active domains are considered. For the SCIL10IL22, the molecular weight is 36 KDa but 1 active site for each interleukin is considered. The changes in absorbance at 630 nm due to different interleukin tested concentrations were fitted to a saturation binding model using the following equation:

Y=B Max*Xh/(K_{D}h+Xh);

In this equation, K_{D} is the apparent dissociation constant (as the number of active receptors per cell is unknown), h is the Hill-slope, and B Max is the saturation signal. This equation assumes specific binding only; all non-specific signals were subtracted. If the saturation signal is not reached, the B Max has been set accordingly for each experiment.

At least three biological replicates (and up to six biological replicates), with two technical replicates for each condition were used. In the conditions in which saturation was not reached, Bmax was fixed to estimate the K_{D} parameters. The different experiments were analysed independently and fitted using GraphPad Prism 9 software, Excel or Kaleidagraph.

Characterisation of IL-10-Expressing CV8 and WT M. pneumoniae Cells

For the IL-10-expressing WT and CV8 cells, 50 μl of stock inoculum was added to a 25 cm² tissue flask (Corning) containing 5 ml fresh Hayflick and grown for 48 h. Culture media was then collected, centrifuged at 10,000 rpm, 10 min, and the supernatant stored at −80° C. for further quantification. Cells were collected by scraping in 1 ml PBS and centrifuged for 5 min at 10,000 rpm at 4° C.; pellets were resuspended in 1 ml PBS, centrifuged for 5 min at 10,000 rpm at 4° C., and supernatants were discarded. Finally, pellets were disaggregated in 200 μl of lysis buffer (4% SDS, 0.1 M HEPES). Protein lysates were quantified using the Pierce BCA Protein Assay kit (23227, Thermo Fisher) following the manufacturer's protocol. In parallel, the levels of the IL-10 variants in the supernatants were quantified by ELISA as previously described. The secretion capacity was normalised to biomass (protein content of the total culture) for each of the strains. Two biological replicates and two technical replicates were carried out for each assay.

### Mice Experiments/In Vivo Experiments

C57Bl/6 female or male mice (18-20 g), aged 6 to 8 weeks, were purchased from Charles River Laboratories (France) and housed under pathogen-free conditions at the PRBB animal facility (registration number B9900073). Animal handling and procedures followed the current European (Directive 86/609/EEC) and National (Real Decreto 53/2013) legislations as well as the FELASA and ARRIVE guidelines, and obtained the approval of the Animal Experimentation Ethic Committee (Comité de Ética, Experimentación Animal y Bioseguridad) of Barcelona Biomedical Research Park (PRBB) and the local Government authorisation.

M. pneumoniae Lung Infection in Mice

The M. pneumoniae WT and CV8 strains were cultured at 37° C., 5% CO₂ in T75 cm² flasks with 25 ml Hayflick liquid medium. After 3-4 days of incubation (when the colour of the medium changed), supernatant was removed, and bacterial cultures were washed three times, scraped and collected in 10 ml sterile PBS. A bacterial suspension of 100 μl (of WT or CV8) containing ˜0.5-1×10⁸ CFU ml⁻¹ (˜0.5-1×10⁷ CFU/mouse) was used for intratracheal (IT) infection in mice previously anesthetised with isoflurane 2% (ISOFLO, Covegan). At 2- or 4-days post-inoculum (dpi), animals were sacrificed by cervical dislocation, and lungs were removed and processed. The left lung was weighed and homogenised 1:10 (wt/vol) in PBS in sterile individual bags (Stomacher80, Seward Medical) for bacterial load quantification as described previously⁵⁵ (detection limit=0.7-0.9 Log 10 CFU/lung). The right lung was: (i) insufflated with 50 μl of 10% methanol stabilised formalin (Panreac) for histopathological analyses (postcaval lobe); and (ii) frozen in N₂ and stored at −80° C. until use, for RNA extraction (inferior lobe). Control animals were inoculated with 100 μl of the vehicle solution and processed in parallel. Infections were performed in groups of at least 5 mice per strain (n≥5).

Acute Lung Infection of P. aeruginosa PAO1 in the C57Bl/6 Mice Model

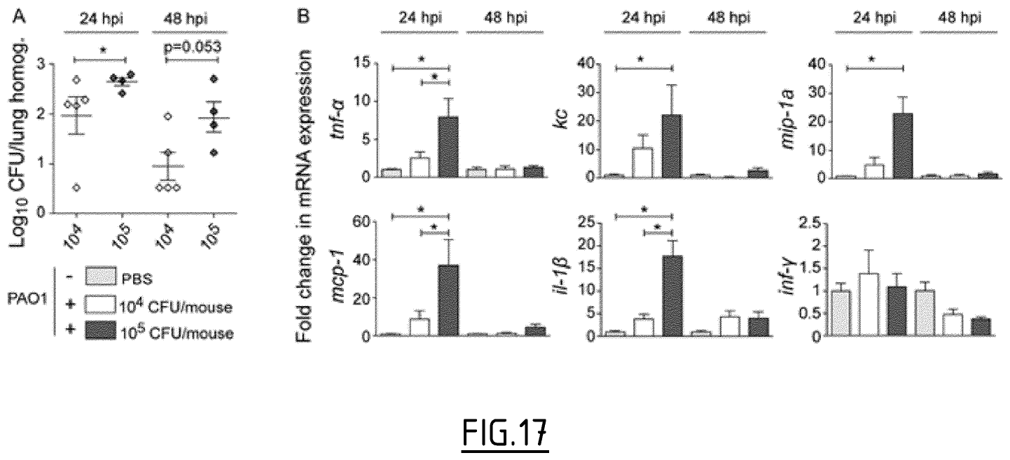

The P. aeruginosa PAO1 strain was grown at 37° C., 5% CO₂, for 14-16 h in TSB agar plates at 37° C. (#BD211825, Fisher Scientific). Bacteria were collected in PBS, and suspensions were normalised to optical density 600 nm (OD₆₀₀)=0.5. For dose-response assay, 100 μl containing ˜1×10⁵ or 1×10⁶ CFU ml⁻¹ (˜1×10⁴ or ˜1×10⁵ CFU/mouse, respectively) was used for IT infection. At 24- or 48 h post-inoculum (hpi), lungs were processed following the method described above. Control animals were inoculated with 100 μl of the vehicle solution (PBS) and processed in parallel. Infections were performed in groups of at least 5 mice per strain (n≥5). In this work we set up a Pseudomonas aeruginosa lung infection model. To determine the optimal conditions, we performed a dose-response experiment, in which lung bacterial burden and gene expression of inflammatory markers were evaluated at 24 and 48 hours post-infection (hpi) (FIG. 17). The bacterial load recovered was higher at 24 than at 48 hpi, evidencing the bacterial clearance through time (FIG. 17A). In addition, the lungs infected with 105 CFUs showed higher bacterial burden than those infected with 10⁴ CFUs, at both time points analysed (FIG. 17A). The inflammatory response was mainly localised at 24 hpi (FIG. 17B). The lungs infected with 105 CFUs showed a significant increase in the expression of tnf-a, kc, mip-1a, mcp-1 and il-1b, compared to the uninfected (PBS)- or 10⁴ CFUs-infected groups (all, p<0.05).

Immunomodulatory Effect of CV8 Expressing hIL-10 In Vivo

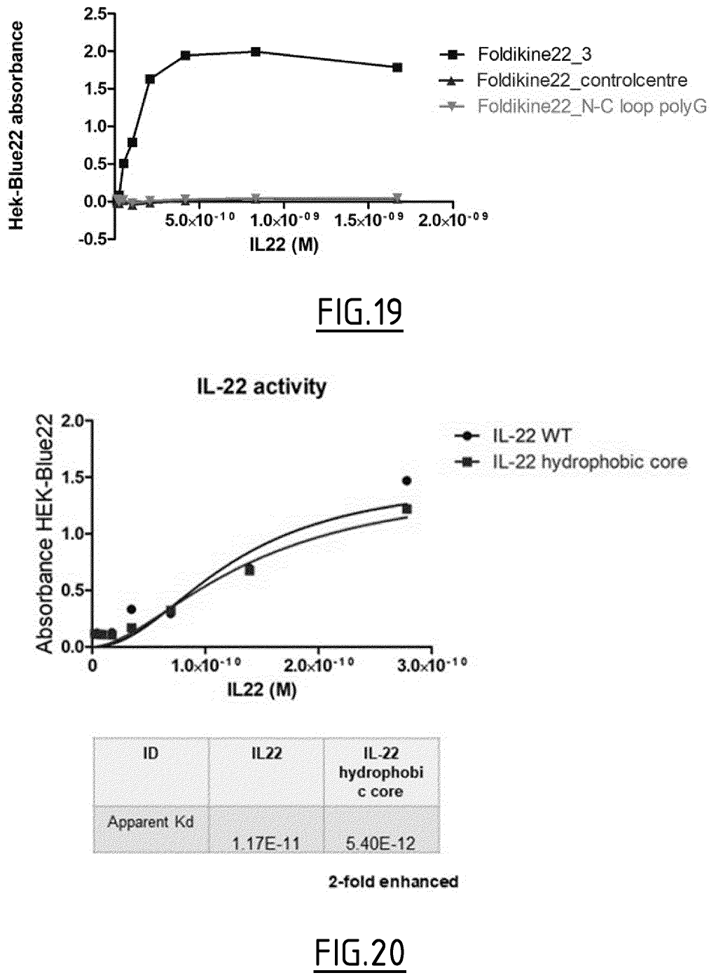

To evaluate the immunomodulatory effect of the hIL-10-encoding Mycoplasma strains, C57Bl/6 mice were infected intratracheally (IT) with 50 μl of P. aeruginosa PAO1 bacterial solution containing ˜2×10⁶ CFU ml⁻¹ (˜1× 105 CFU/mouse); and 2 h later, mice were infected IT with 50 μl of the Mycoplasma strains containing ˜2×10⁸ CFU ml⁻¹ (˜1×10⁷ CFU/mouse), following the procedures described above (FIG. 19A). Control animals were processed in parallel and treated with (i) 2 μg of human IL-10 recombinant protein (hIL-10r) (#200-10, Peprotech), or (ii) sterile PBS (vehicle solution). After 24 h of PAO1 infection, animals were sacrificed, and lungs were aseptically removed for the experiments described above.

### Whole-Lung RNA Extraction and Real-Time Quantitative PCR (RT-qPCR) Analysis

Right lungs that had been stored at −80° C. were homogenised using Ultra-Turrax (IKA), and total RNA was isolated using RNeasy® Mini Kit (Qiagen) following the manufacturer's instructions. RNA concentration was measured spectrophotometrically using Nanodrop One (Thermo-Scientific), and sample RNA integrity was confirmed by 1% agarose gel electrophoresis. RNA samples with ratio absorbance measurements at 260 nm/280 nm of 1.8-2.1 were used.

Complementary DNA (cDNA) from whole-lung cells was synthesised from total RNA (1 μg) using SuperScript II Reverse Transcriptase reagents (Invitrogen). PCR amplification was performed by using SYBR Premix Ex Taq II (Tli RNaseH Plus) (Takara), and fluorescence was analysed with AriaMx Real-Time PCR System (Agilent Technologies). Primer pairs for gapdH, tnf-a, kc, mip-1a, mcp-1, inf-y, il-1b and nf-kb2 detection are shown (Table 4). The 2^{-ΔΔCt} method was used to determine the relative abundances of mRNA in each experimental condition using gapdH as endogenous control and normalising to the values obtained in the PBS group as follows: ΔCt=mean Ct analysed gene−mean Ct gapdH; ΔΔCt=ΔCt treated group−ΔCt PBS group; fold-change in mRNA expression=2^{-ΔΔCt}.

### Histological and Immunohistochemistry (IHC) Analysis of Lung Samples

After necropsy, the postcaval lobe was fixed in formalin (Panreac), trimmed and automatically processed in ethanol series and xylene (Leica TP1020 Tissue Processor). Thereafter, tissues were embedded in paraffin (Tec2900 Histo-Line Workstation), sectioned to 4 μm (Leica RM2235 Microtome) and stained by hematoxylin and eosin (H&E) following standard procedures (Leica Autostainer XL).

Preparations were manually mounted onto glass slides and examined by light microscopy. Tissue was evaluated by a trained veterinary pathologist (Histopathology Facility Manager of Institute for Research in Biomedicine-IRB Barcelona). Alveolar cell infiltrate and perivascular-peribronchial/peribronchiolar infiltrate were used as histopathological parameters, and a quantitative score was established (0-5:0, none; 1, minimal; 2, mild; 3, moderate; 4, intense; 5 very intense); the final score of each sample was obtained by the sum of both parameters. For IHC, paraffin-embedded tissue sections were stained with the rabbit monoclonal anti-F4/80 (D2S9R) XP (#70076S, Cell signalling) as a marker of macrophages⁵⁶, or the rabbit polyclonal Anti-Neutrophil Elastase (#Ab68672, Abcam) as a marker of neutrophils⁵⁷ using the Leica Bond RX. Specificity of staining was confirmed by staining with a rabbit IgG isotype control (#Ab27478, Abcam). Brightfield images were acquired with a NanoZoomer-2.0 HT C9600 digital scanner (Hamamatsu) equipped with a 20× objective. All images were visualised with the NDP.view 2 U123888-01 software (Hamamatsu, Photonics, France), using a gamma correction set at 1.8 in the image control panel. Output results obtained from image analyses were represented as % positive cells [%=100×(positive cells/positive cells+negative cells)].

### Statistical Analyses

In all cases, statistical significance was set to p<0.05. Analyses were performed using Prism software (GraphPad Software) statistical package and are detailed in each figure legend.

### Materials

List of IL-10 variants tested and described in the following sections:
hIL-10r (commercial IL-10 protein, is a natural swapped domain dimer)
WT-IL 10/IL-10 ORF (wt swapped domain dimer IL-10 expressed and secreted by bacteria, e.g. Mycoplasma).
Mut1 to Mut3 point mutations on wt swapped domain dimer IL-10
MutSC1 foldikine_10, single chain dimeric IL-10 in which an engineered peptide linker covalently joins the N- and C-termini of the two molecules of the swapped dimer in one of the two 3D domains.
MutSC2, the same as MutSC1 but with the point mutations of Mut3 in each half of the foldikine sequence (with the exception of the mutation at residue 18 in the second half of the polypeptide
MutM, monomeric version of IL10 previously published by another group (Josephson et al., 19).
MutSC1_linkercontrol19 is a foldikine in which the engineered peptide linker of MutSC1 is replaced by a poly-Gly linker of the same length (5aa) to assess the impact of the linker composition in the functional activity of the engineered molecule.
MutSC1_controlcenter21 is a control construct based on MutSC1 in which novel N- and C-termini were generated by cutting at one of the linkers bridging the two foldikine domains (position Asn166). To keep a single chain the two original N- and C-termini present in the original swapped domain structure were bridged using the linker sequence used to bridge the original N- and C-termini in one of the foldikine domains of MutSC1. This construct is used to assess the impact of having only one of the two central linkers of the MutSC1 foldikine, such that the pair of 3D domains are only bridged by one linker sequence rather than two.
MutIL10_polygly represents a single chain version of dimeric IL-10 in which the N-terminus of one IL-10 monomer sequence is linked to the C-terminus of the other IL-10 monomer sequence with a 13 residue polyGly linker as published in (Minshawi et al., 2020; https://doi.org/10.3389/fimmu.2020.01794).
   M. pneumoniae Secrete Functional IL-10 Dimers with Disulfide Bridges

We first tested if M. pneumoniae can actively express a complex molecule, such as the human IL-10-swapped dimer with two disulfide bridges. For this, we cloned the human IL-10 fused to the mpn142 secretion signal previously described for M. pneumoniae⁵⁸.

We showed by ELISA that the M. pneumoniae WT strain expressed IL-10 in the supernatant, at a concentration of around 2 fg/cell, after 48 h in culture (equivalent to 2 μg from a culture of 1×10⁹ cells; Table 5). We confirmed by mass spectroscopy (MS) that both disulfide bridges were generated in the expressed hIL-10 (Table 2), indicating that the dimer was correctly folded. We next tested the functionality of the produced IL-10 for its ability to: (i) modulate the human primary macrophage anti-inflammatory response activation program, and (ii) become activated by phosphorylation of Tyr705 of STAT335 using primary blood, CD14⁺ monocytes isolated from four independent, healthy blood donors (Methods).

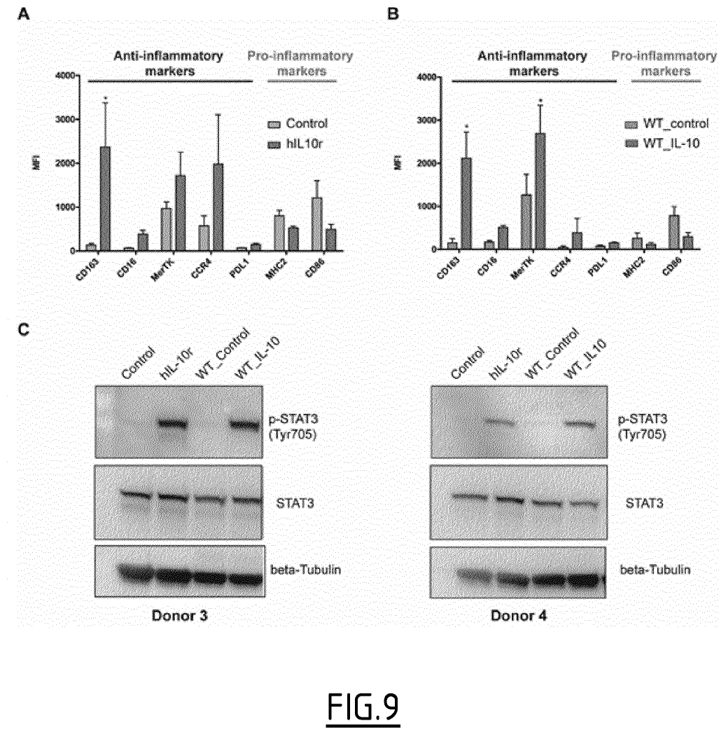

M. pneumoniae expressing wild-type (WT) IL-10 was grown for two days, and the supernatant was added to a cell culture containing approximately one million circulating monocytes (in a 1:5 ratio). As a positive control, the human IL-10 recombinant protein (hIL-10r) was added at a concentration of 20 ng ml-1. As expected, commercial hIL-10r treatment of monocytes enhanced the expression of anti-inflammatory markers (i.e., CD16, CD163 and MerTK) and decreased that of critical pro-inflammatory receptors (i.e., CD86 and MHC2) (FIG. 9A)^{36,37}, as compared to non-treated cells. Notably, the results using the IL-10 produced by M. pneumoniae (WT_IL-10 ORF) was comparable to those using hIL-10r (FIG. 9B). By Western blot, we observed phosphorylation of Tyr705 in STAT3 after 24 h exposure of freshly isolated monocytes to the supernatant of M. pneumoniae expressing IL-10 or hIL-10r (FIG. 9C). Altogether, these results demonstrated that M. pneumoniae is able to express functional human IL-10.

MutSC2 Expression in and Secretion from Eukaryotic Cells

For expression of MutSC2 in eukaryotic cells, Expi293F™ cells were chosen.

Expi293FT cells are human cells derived from the 293F cell line. They are maintained in suspension culture and grow to high density in Expi293™ Expression Medium. Expi293F™ Cells are highly transfectable and generate superior protein yields compared to standard 293 cell lines in transient protein expression.

For expression, Expi293F human cells (Thermo Fisher Scientific) were transfected with purified DNA and polyethylenimine (PEI).

To identify secreted MutSC2 protein, cells were harvested 3 days post-transfection and MutSC2-containing supernatants were collected by centrifugation at 13,000 rpm for 15 min. The MutSC2 protein was purified in Hitrap-Ni columns in an automated Fast Protein Liquid Chromatography (FPLC; Äkta avant), concentrated through 10 kDa Amicon centrifugal filter units (EMD Millipore) and resuspended in phosphate-buffered saline (PBS) and storage at −80° C.

MutSC1 Expression in and Secretion from Escherichia coli

Escherichia coli BL21 was transformed with three pCoofy vector variants (KanR) coding for IL-10 WT, MutSC1, or MutSC1-his tag, respectively. One colony transformed with each vector variant was picked and grown overnight at 37° C. in LB supplemented with kanamycin. Once the culture density reaches around 0.5 OD, the culture was cooled down to room temperature.

From the culture, 1 ml was used for CFU counting in LB plates; 4 ml of the culture was induced with IPTG at 0.5 mM and incubated overnight at 20° C. The cultures were then centrifuged at 3,500 g for 20 minutes and the supernatant was collected for ELISA quantification. Cell pellets were resuspended with 300 μl of M-PER protein extraction reagent (Thermo Scientific). IL-10 quantification was performed using a human deluxe IL-10 ELISA kit (Biolegend) and IL-10 variant functionality was checked using HEK-Blue10 reporter cells (InvivoGen).

MutSC1 Expression in and Secretion from Lactococcus lactis

The strain of Lactococcus lactis (NZ9000-pepN::nisRK;) and the transformation vector (pNZ8124) were purchased from MobiTec (Germany). Cells were grown as indicated by the manufacturer.

For electroporation, 40 μl of cells were transformed with 200 ng of the PNZ8124 vector encoding for IL-10 WT or MutSC1. The electroporation settings were set to 2,000 V, 25 μF, 200Ω in a Biorad Genepulser. Then, 1 ml of G/L-M17B+20 mM MgCl₂+2 mM CaCl₂) was added. The cuvette was kept for 5 min on ice and incubated for 1-1.5 h at 30° C. and 100 μl of the culture was plated for 2 days on M17 agar with glucose and 10 ng/ml chloramphenicol.

For expression of the different proteins, 5 ml culture was grown overnight at 30° C. and the OD of the cultures were adjusted to 0.4. 9 ml of the culture was induced with 1 ng/ml nisin (stock: 1 mg/ml nisin in 0.05% acetic acid, Thermo Fisher) and incubated for 3 hours. Cells were collected by centrifugation and expressed cytokine/foldikine in the supernatant was directly quantified by ELISA. The cell pellet was lysed with 300 μl of M-PER Extraction reagent (Thermo Scientific). Serial dilutions of the cells were plated on bacterial growth media plates for CFU counts.

### Engineering IL-10 for Increased Receptor Affinities

To overcome the limitation of the potential problem of lower protein production capacity in M. pneumoniae as compared to other bacteria²², and to beneficially be able to decrease the number of M. pneumoniae administered to a patient, the inventors proposed to engineer mutations in IL-10 with increased binding affinity to the high- and low affinity receptors R1 and R2, respectively. To generate IL-10 versions with an increased affinity towards R1, we studied the x-ray crystal structures of hIL-10 crystalised in complex with R1 with good crystal resolution (PDB 1y6k; 2.5 Å).

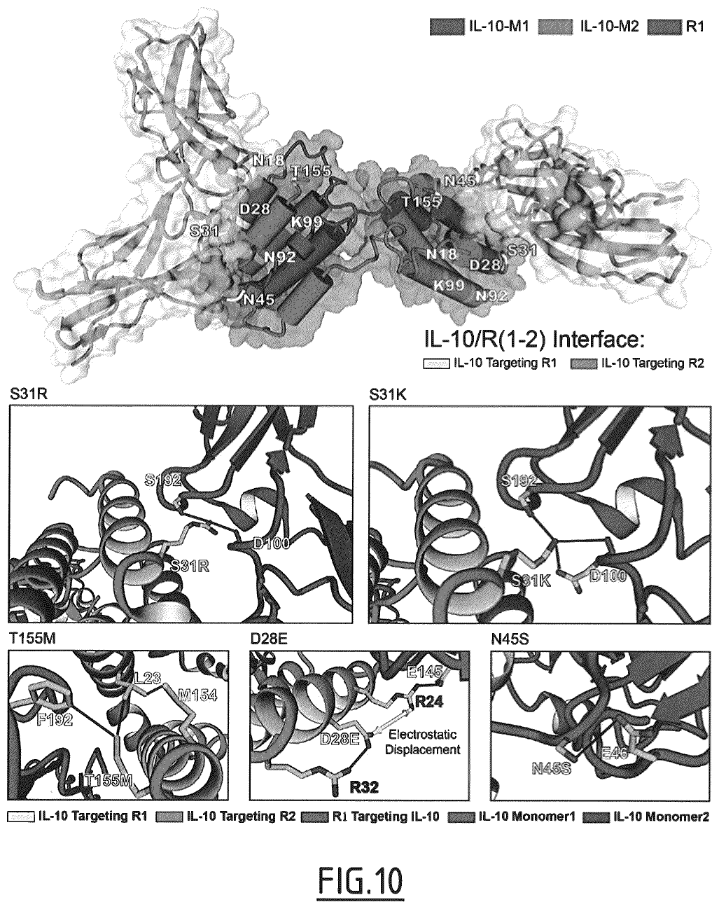

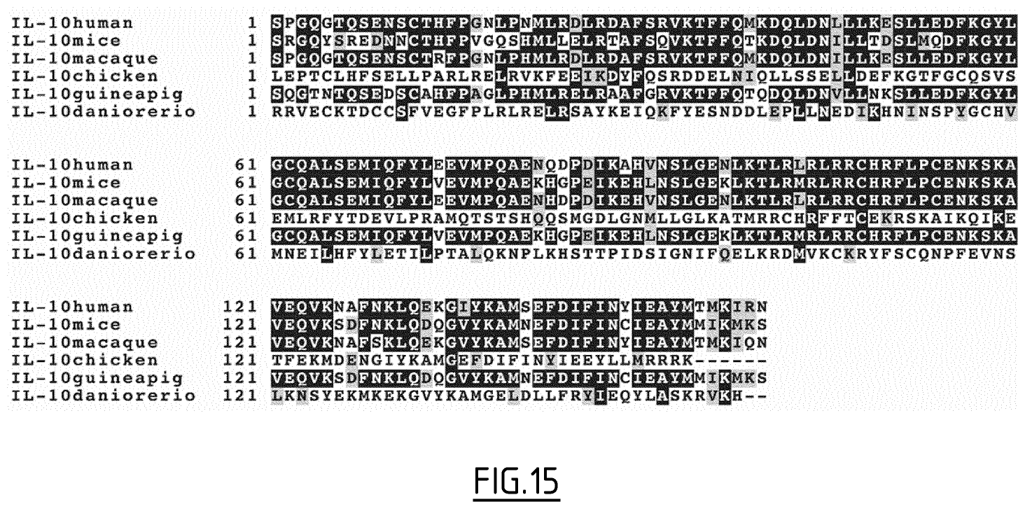

We then performed an in silico mutagenesis scanning of the complex interface residues using the PositionScan FoldX command, which individually mutates all R1-contacting IL-10 amino acids to each of the other 19 natural amino acids³¹. Based on this analysis and on sequence conservation across different species (FIG. 15), we identified a set of non-conserved residues (see e.g. Table 1) that, when mutated and modelled using the FoldX software program improved binding to R1 without compromising protein stability (Table 6). Using the selected mutations, we modelled two multiple mutants (Mut1 (SEQ ID NO: 6) and Mut2 (SEQ ID NO: 7), in which position 31 of IL-10 is changed from Ser to Lys or Arg, respectively) (FIG. 10). We further checked that the mutations were independent of the structure used by modelling them in other structures of IL-10 apo form (PDB 2ilk; 1.6 Å) or IL-10 holo form with R1 at lower resolution (PDB 1j7v, 2.9 Å) (Table 6).

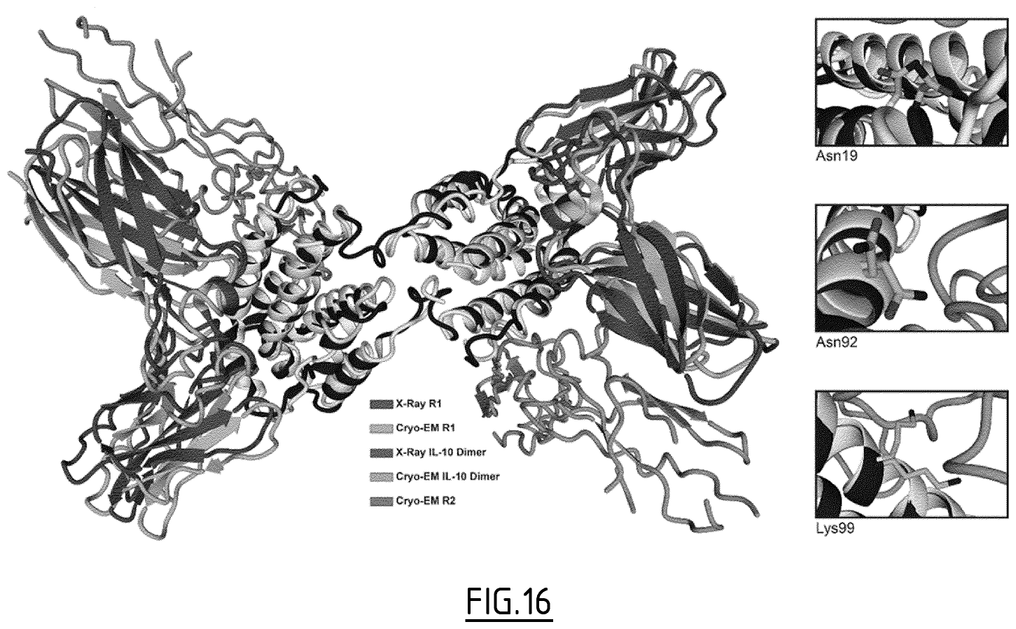

At the start of this work, no structure was available for IL-10 bound to R2. Therefore, we combined the Mut2 mutations with additional mutations of IL-10 which had previously been reported to potentially enhance the affinity of the interactions between IL-10 and R2 (e.g., N18I, N92I and K99N) 9, resulting in Mut3 (SEQ ID NO: 8). When modelling these three mutations in all IL-10 structures used here, we found that they decreased the stability of IL-10 (Table 6). Quite recently, a low-resolution structure (6×93; 3.5 Å) of the complex between IL-10 and R1/R2 became available¹⁰. Using this structure to model these mutations, we observed in fact an increase in stability of IL-10 and an increase in the predicted binding energy to R2 (Table 6). This increase can be explained by local conformational changes in the IL-10 region that interacts with R2 (FIG. 16).

Finally, we generated the mutants Mut1 to 3 and MutM (a previously-described monomeric form of IL-10²⁵) (Table 1); these were cloned into M. pneumoniae (see FIG. 10 for an overview of the mutation positions and interactions in the structures).

### Measurement of Apparent K_{D} Values for the Designed Multiple Mutants

To assess the relative affinity of the different IL-10 variants, we used a reporter cell line (HEK-blue™) that contains a secreted embryonic alkaline phosphatase (SEAP) reporter that when IL-10 is bound to R1 and R2, it triggers a JAK1/STAT3-mediated response, resulting in SEAP expression, which can be measured by absorbance at 630 nm. The intensity of the signal before saturation is proportional to the activation of STAT3, and a kinetic model³⁸ can be fitted to the data to obtain apparent K_{D} values (see Methods).

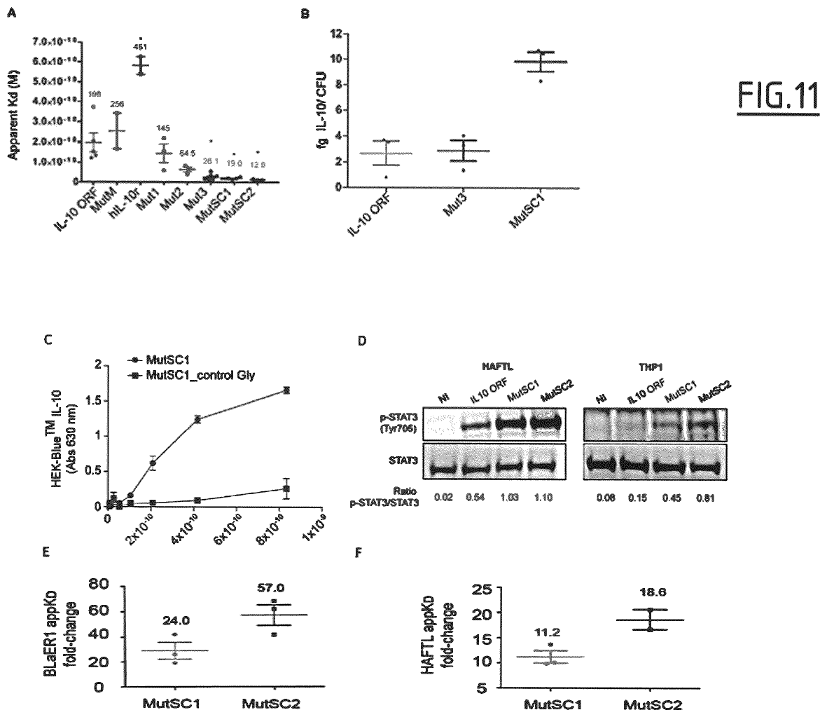

Mutants Mut1 and Mut2 (which are designed to bind better to R1) had similar apparent K_{D} values (1.45 e-¹⁰ M and 6.45 e-¹¹ M, respectively) but were better than IL-10 ORF (1.98 e-¹⁰ M), exhibiting 1.4-fold and 3.0-fold enhancement, respectively. Mut3, based on Mut2 and incorporating mutations favouring R2 binding, resulted in an apparent increase in K_{D} (2.61 e-¹¹ M) as compared to IL-10-ORF of 8-fold (FIG. 11A). We confirmed that the apparent K_{D} of the monomeric IL-10 (MutM) was lower than that of the IL-10 ORF, as previously described²⁵ (FIG. 11A and Table 7). The expression level of the best mutant (Mut3) and of the IL-10 ORF were similar (FIG. 11B).

### Single-Chain IL-10 Rational Design

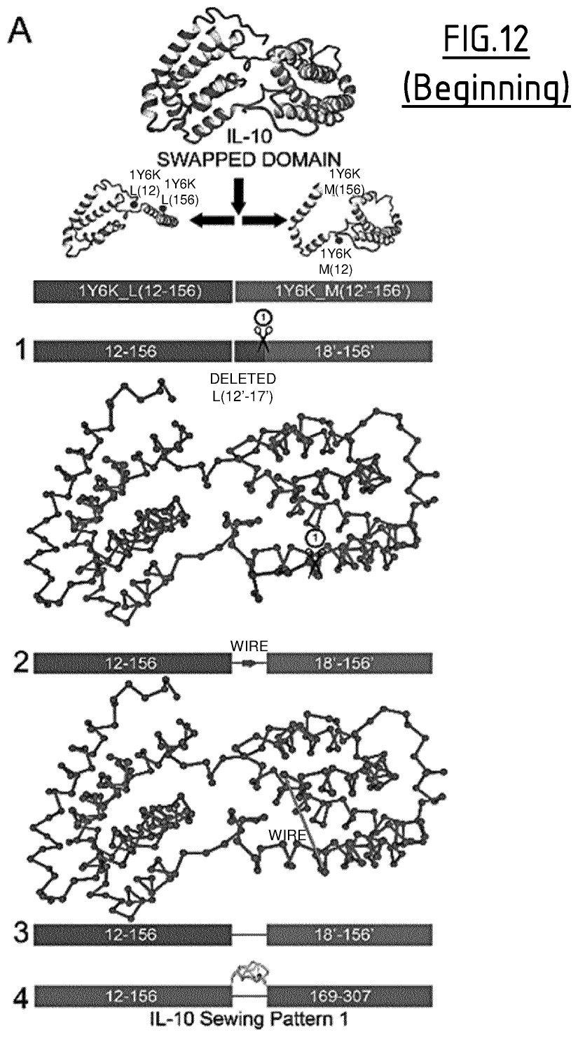

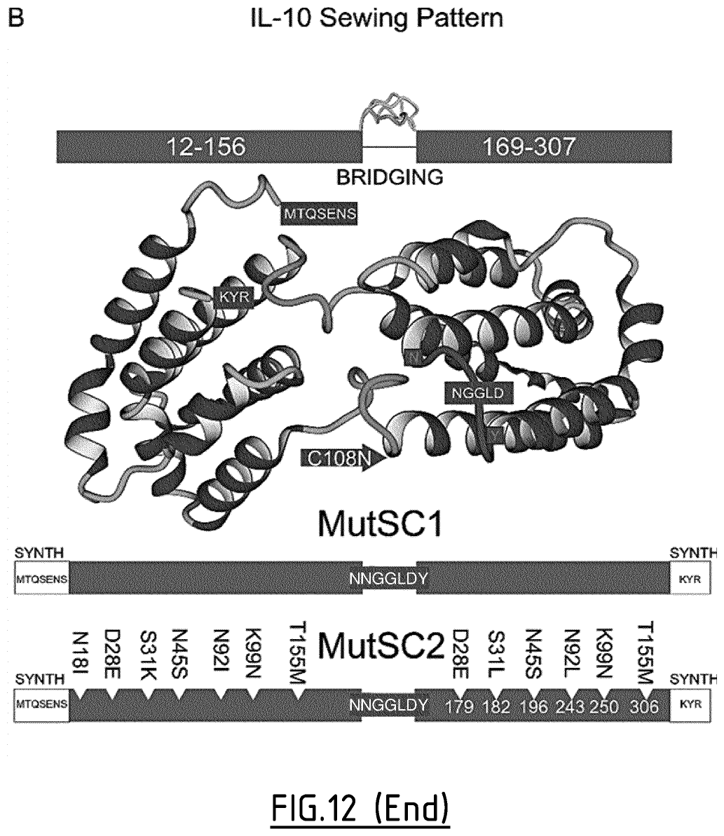

To obtain the highest possible amount/proportion of the IL-10 active form, we had to minimise potential sequestering of expressed protein due to degradation, misfolding, and/or extensive multimerisation (27). To achieve this, the N-terminal region of one monomer was linked to the C-terminal region of the other monomer in the swapped dimer (MutSC1) (FIG. 12A). After a ModelX Bridging search (Methods) using Asn18 and Lys157 as anchoring points, we obtained 541 models that were side-chain repaired; these were ranked by energy using FoldXv5 (30). It was then determined that the best linker sequence was N157-FGGLD-Y18 (SEQ ID NO: 316), whereby Asn18 was replaced by Tyr and Lys157 by Asn, and the FGGLD sequence was inserted (SEQ ID NO: 342). By introducing this linker, we deleted the N-terminal residues in one of the two monomers in the crystal structure (residues 12 to 17; CTHFPG). Afterwards, we mutated the Phe residue after N157 to Asn to improve binding to R1 (this results in NGGLD as the inserted sequence; FIG. 12A) (SEQ ID NO: 318); this improved the overall stability of the SC molecule as well as its binding to R1 (Table 6). We then renumbered the sequence. As the N-terminal sequence of one of the monomers containing Cys12 was deleted-which forms a disulfide bridge with Cys108 in the original IL-10 ORF sequence (new residue 259 was deleted)—we mutated Cys108 to Asn, to prevent the formation of potential spurious disulfide bridges. Cys12 and Cys108 remained in the structural equivalent of the other monomer of the swapped dimer. MutSC2 was designed by grafting the Mut3 mutations into MutSC1 (FIG. 12B).

The resultant polypeptide exhibited improved overall stability of the single-chain molecule as well as its binding to R1 (Table 6). This resulted in MutSC1 (Table 1). MutSC2 was designed by grafting the Mut3 mutations into MutSC1 (FIG. 12B).

### Characterisation of the Engineered IL-10 Mutants In Vitro

Quite importantly, MutSC2 had a better apparent K_{D} than the best multiple mutant in the swapped dimer (Mut3). To determine the importance of the linker sequence in the activity of MutSC1 and MutSC2, we replaced the linker sequence N-NGGLD-Y with the polyGly linker sequence N-GGGGG-Y (MutSC1_controlGly). The new control mutant MutSC1_controlGly had a very significant decrease in activity, indicating that it is important for a short linker to have a specific sequence (FIG. 11C), and potentially one which creates a particularly beneficial structural conformation—e.g. which may impart conformational stability to the folded dimer structure.

To address whether this improvement in apparent K_{D} of MutSC1 and MutSC2 was specific to the cell type used in the assay, we assessed IL-10 signalling via its activation (as shown by Western blot) in two different immune cell lines: a human monocyte cell line, THP1, and a murine B-cell line, HAFTL. In both cases, when similar molar concentrations of IL-10 ORF, MutSC1, and MutSC2 were used, a significantly higher activation for the two SC mutants as compared to the WT IL-10 ORF was observed (FIG. 11D).

To obtain a quantitative estimation of the apparent K_{D}, we assessed STAT3 phosphorylation upon ligand stimulation by FACS in two different cell lines, using murine HAFTL and human BLaER1 (B-cell precursor leukaemia cell line). In comparison to IL-10 ORF, both MutSC1 and MutSC2 mutants were significantly superior in activating p-STAT3 (FIGS. 11E and F). Altogether, the engineered mutants MutSC1 and MutSC2 resulted in enhanced affinity and maximum activity in different cell types of human and mouse origins. As in the case of the HEKblue cells MutSC2 was around two-fold better than MutSC1.

Overall, these results supported the idea that the IL-10 swapped homodimer produced by Mycoplasma is a mixture of unfolded monomeric and active dimeric forms. Thus, converting a swapped dimer into a single-chain molecule (e.g. FIG. 3) is an effective strategy for increasing the amount in bacteria not only of secreted protein but (more importantly) of secreted active protein.

### Characterization of CV8 Chassis Strain In Vivo

M. pneumoniae has been shown to induce an inflammatory response in lungs of mice 17,39. To investigate this response, we engineered an attenuated strain of M. pneumoniae, referred to here as chassis CV8 (patent application EP 20382207.7), in which the nuclease MPN133 (mpn133⁴⁰) and the CARDS toxin (mpn372)⁴¹ are deleted, and the glyceraldehyde-3-phosphate dehydrogenase GlpD (mpn051)⁴² is replaced by GpsA (see Methods), an enzyme with similar metabolic activity but that produces H₂0 rather than H₂0₂.

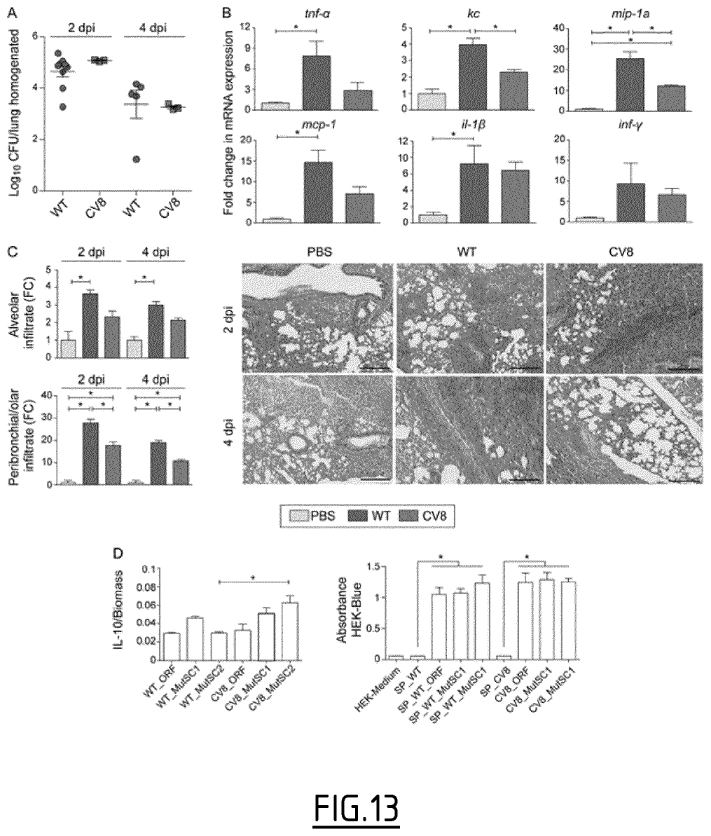

The virulence of CV8 was tested in a mouse model of intratracheal lung infection (FIG. 13). Similar bacterial loads were observed for the WT and CV8 strains at both time points analysed (FIG. 13A), suggesting that the deletion of the virulence factors did not compromise the colonisation capacity of Mycoplasma strains. The lung inflammatory profile was analysed by RT-qPCR (FIG. 13B) at 2 days post-inoculum (dpi); notably, at this time point, we have previously shown that the bacterial load is still significant but that there is sufficient time for an inflammatory response 58. Compared to the uninfected control group (using PBS), the WT strain increased the expression of tnf-a, kc, mip-1a, mcp-1 and il-1b (all, p<0.05) (FIG. 13B and Table 8). In the case of CV8, only the expression of mip-1a changed significantly as compared to the PBS control (p<0.05), but it still remained at lower levels than in the WT strain.

To confirm the attenuation of the chassis, we analysed the histological findings in lungs (FIG. 13C). The WT strain promoted the presence of alveolar and peribronchial/peribronchiolar infiltrate at both 2 and 4 dpi (p<0.05 for both), as compared with the PBS control. In the CV8-infected lungs, we also observed peribronchial/peribronchiolar infiltrate but at a lower level than that observed for the WT strain at both time points analysed (both, p<0.05). These results were in line with the attenuated phenotype of CV8. We therefore generated the CV8_IL-10 ORF, CV8_MutSC1 and CV8_MutSC2 to test their immunomodulatory effects in vivo. Critically, we observed no significant differences in either the production of a functional IL-10 ORF (FIG. 13C) by the WT or CV8 strains, or in the activity of any of the secreted mutants as compared to the WT strain (FIG. 13D).

### Engineered IL-10 Variants Exhibit Powerful Immunomodulation Effects In Vivo

We developed and used a Pseudomonas aeruginosa PAO1 infectious model (see Methods) as an in vivo tool to study the down-regulatory properties of the IL-10 variants generated in this work (FIG. 17).

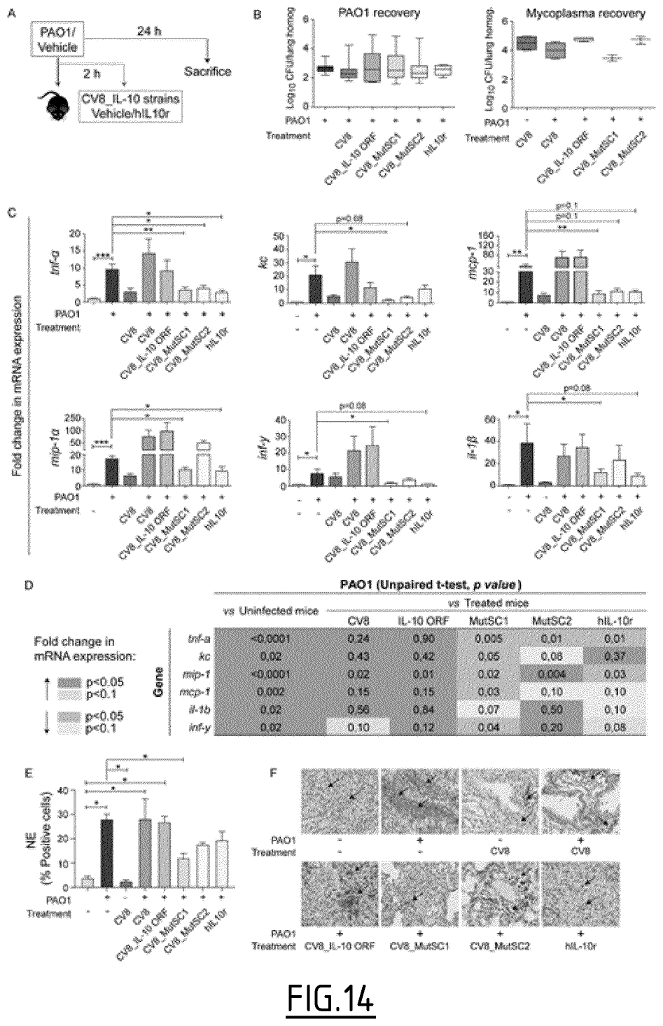

To test the anti-inflammatory properties of the engineered IL-10 mutants expressed by the CV8 chassis, we infected C57Bl/6 mice with 1×10⁵ CFU of the PAO1 strain, and 2 h later, with 1×10⁷ CFUs of a Mycoplasma strain (CV8, CV8_IL-10 ORF, CV8_MutSC1 or CV8_MutSC2). Control mice received PBS (as a negative control) or 2 μg of hIL-10r (as a positive control) (FIG. 14A).

First, we analysed the pulmonary bacterial count of PAO1 and Mycoplasma (FIG. 14B). No significant differences were observed at 24 hpi between the groups analysed, ruling out that the differences in the expression of inflammatory genes were biased by the bacterial load present in the lung.

We then analysed the inflammatory profile (FIG. 14C, 14D). Indeed, the infection with PAO1 resulted in a significant increase in the expression of inflammatory markers. Regarding the CV8 strain, we could see a minor inflammatory response when used alone (FIG. 14C), that was additive when doing a joint administration of PAO1+CV8 (FIG. 14C, 14D). Administration of 2 μg of hIL10r protein to mice infected by PAO1 (PAO1+hIL-10r) significantly reduced the expression of tnf-a and mip-1a (both, p<0.01) with a similar trend for mcp-1 (p=0.1), inf-y (p=0.08) and il-1β (p=0.06) (FIG. 14C, 14D). In contrast to the results observed for the hIL-10r treated group, the CV8 encoding IL-10 WT (CV8_IL-10 ORF) did not significantly modulate the expression of the analysed inflammatory markers (FIG. 14C, 14D). The inoculation of CV8_MutSC1 or CV8_MutSC2 reduced the expression of inflammatory markers (FIG. 14C). In contrast to what we observed in vitro, the properties of MutSC1 and MutSC2 differed in vivo, with MutSC1 proving to be the more effective candidate (FIG. 14C, 14D). The effects of MutSC1 expressed by the CV8 strain was comparable or even better than the administration of 2 μg of hIL-10r.

Notably, in a histopathological analysis of the infected lungs, we observed no major differences in the tissue parameters analysed (Table 9). We observed an important cellular infiltrate with predominance of neutrophils (PMN) in all groups infected with PAO1 (FIG. 14E, 14F), as previously described⁴³. We found a significant decrease in the number of PMN in the cellular infiltrate of animals treated with MutSC1, and a decrease (which however was not significant) with MutSC2, as compared to the PAO1+PBS group (FIG. 14E, 14F).

In summary, the combination of the M. pneumoniae CV8 chassis with the designed IL-10 variants showed its immunomodulatory potential in vivo, with MutSC1 being the most promising candidate.

### Discussion

The systemic administration of IL-10 in humans for the treatment of different pathologies, such as inflammatory bowel disease, allergic asthma, and solid tumours has been tested in different clinical trials but failed in the clinical outcome of these patients due to different problems⁴. Limitations include low concentration of IL-10 in the targeted tissue, or the precise tissue location required for IL-10 to have the desired anti-inflammatory effect yet avoid systemic toxicity.

Using live biotherapeutic products encoding IL-10 could be an effective strategy for increasing the local concentration of IL-10 in the targeted tissue, as it in essence provides a micro, self-replicating minimal machine as a drug factory that is easy to remove with antibiotics when its action is completed. Strategies relying on the local delivery of IL-10 in situ have been explored for some bacteria, such as L. lactis or Bifidobacterium bifidum in inflammatory bowel diseases (IBD), with some preliminary promising results in clinical trials^{44,45}. However, the delivery of human proteins by bacteria might also be hampered by foldability of the secreted molecule, the need to make disulphide bridges and/or to form a dimer (or a higher oligomer) in order to have a functional protein. This is the case of IL-10, which is a swapped homodimeric protein with two disulphide bridges.

In this work, we engineered human IL-10 to optimise its expression and efficacy when expressed by a bacterium in vivo. The final objective was to decrease possible bacterial pathogenicity by reducing the bacterial dose to be administered to a patient while retaining the anti-inflammatory properties of IL-10. To show the improved properties of our engineered IL-10 molecules (‘foldikines’), we selected the lung as the target organ. This choice was made based on reports showing that exogenous delivery of IL-10 ameliorates different lung diseases^{14,15,46} and because the lung is a more challenging organ than intestine, due to its reduced microbiome size²⁸.

Here, we have demonstrated that the human lung bacterium M. pneumoniae is able to secrete functional IL-10 with disulphide bridges, showing an activity comparable to that of human IL-10 recombinant protein (hIL-10r).

Infection of the lung even by non-pathogenic bacteria could trigger an inflammatory response, so the less bacteria used, the better. As M. pneumoniae has a limited protein synthesis capacity, one way to decrease the dose applied to lungs for therapy is to increase the activity of the secreted molecules. This can be done by increasing the affinity of IL-10 for its receptors, and/or improving its stability and foldability^{9,29}.

Here we engineered multiple mutant cytokines, such as IL-10 and IL-22 variants (as described herein) using a combination of intelligent design, software algorithms (ModelX, FoldX) and data from the literature (e.g. for the designed mutations present in Mut3) as well as single-chain mutants linking the N- and C-termini (foldikine MutSC1) that exhibited a significantly better apparent K_{D} in vitro, which was even better when combinations of separately beneficial sets of mutations were included (e.g. foldikine MutSC2). Moreover, MutSC1 and MutSC2 foldikines were expressed to levels around 4-times higher than IL-10 ORF or Mut3. Combining the higher expression and better apparent K_{D}, the WT Mycoplasma strain expressing MutSC2 has around 60 times relatively higher activity in vitro than the IL-10 ORF strain.

In agreement with this, the CV8 strain expressing the IL-10 MutSC1 and MutSC2 in vivo showed a significant down-regulatory effect on inflammation upon P. aeruginosa infection, as compared to CV8 expressing IL-10 ORF or to administration of 2 μg of hIL-10r protein. However, although we observed that MutSC2 performed better than MutSC1 in some assays in vitro, we see the opposite tendency in vivo. This could be due to the small difference in the apparent K_{D} between the two mutants (approx. two-fold) or more likely to the fact that the mutations engineered here were designed using the human R1 receptor, which differs at some IL-10 interacting positions as compared to the mouse R1 receptor in which the in vitro assays were performed.

Single-chain molecules with dimeric 3D structures not only result in an increase in functional protein when secreted by bacteria, they permit asymmetric molecules to be designed for a specific cell-type targeting. This can be taken into consideration when designing novel single-chain variants by structurally designing each of the two interacting surfaces of the single molecule separately.

In sum, we have generated a live biotherapeutic chassis for cargo-delivery by bacteria of optimised cytokines (such as IL-10) in lung tissue that downregulates the inflammatory response in an infectious context, thereby removing the previous constrictions on effective cytokine delivery in vivo and paving the way for clinical trial success.

By appropriate sequence and structural modifications, the foldikine concept demonstrated above for the IL-10 and IL-22 foldikines is envisaged to be applicable to many other cytokines, as described elsewhere herein, e.g. (A) Class I helical cytokines that can be sub-classified in long-chain helical (IL6, IL11, IL12A, IL23A, IL27A, IL31, CLCF1, CNTF, CTF1, LIF, OSM, and CSF3) or short-chain helical (IL2, IL3, IL4, IL5, IL7, IL9, IL13, IL15 and IL21, TSLP, CSF1 and CSF2); and (B) Class II cytokines, including the IL10-like cytokines (IL10, IL19, IL20, IL22, IL24, IL26, IL28A, IL28B, and IL29), type I interferons (IFNα (IFNA1, IFNA2, IFNA4, IFNA5, IFNA6, IFNA7, IFNA8, IFNA10, IFNA13, IFNA14, IFNA16, IFNA17 and IFNA21), IFNω (IFNW1), IFNε (IFNE), IFNκ (IFNK) and IFNβ (IFNB1).

### Tables

TABLE 1

| Genetic | | | SEQ |
|---|---|---|---|
| part | Sequence | Type | ID NO: |
| P3 | TATTGCATAATATTGTATAATATTGTATAATTAAAAATA | DNA | 2 |
| S142 | ATGAAGTCCAAGTTGAAACTCAAACGCTATTTACTCTTTCTCCCCTT | DNA | 3 |
| | GTTACCACTCGGTACCTTGAGTTTAGCTAACACTTACTTGTTACAA | | |
| IL-10 | For numbering purposes Ser at second position | prt | 4 |
| ORF | is considered residue 1 | | |
| | MSPGQGTQSENSCTHFPGNLPNMLRDLRDAFSRVKTFFQMKDQLDNL | | |
| | LLKESLLEDFKGYLGCQALSEMIQFYLEEVMPQAENQDPDIKAHVNS | | |
| | LGENLKTLRLRLRRCHRFLPCENKSKAVEQVKNAFNKLQEKGIYKAM | | |
| | SEFDIFINYIEAYMTMKIRN | | |
| MutM | For numbering purposes Ser at second position | prt | 5 |
| | is considered residue 1 | | |
| | MSPGQGTQSENSCTHFPGNLPNMLRDLRDAFSRVKTFFQMKDQLDNL | | |
| | LLKESLLEDFKGYLGCQALSEMIQFYLEEVMPQAENQDPDIKAHVNS | | |
| | LGENLKTLRLRLRRCHRFLPCENGGGSGGKSKAVEQVKNAFNKLQEK | | |
| | GIYKAMSEFDIFINYIEAYMTMKIRN* | | |
| Mut1 | For numbering purposes Ser at second position | prt | 6 |
| | is considered residue 1 | | |
| | MSPGQGTQSENSCTHFPGNLPNMLRDLREAFRRVKTFFQMKDQLDSL | | |
| | LLKESLLEDFKGYLGCQALSEMIQFYLEEVMPQAENQDPDIKAHVNS | | |
| | LGENLKTLRLRLRRCHRFLPCENKSKAVEQVKNAFNKLQEKGIYKAM | | |
| | SEFDIFINYIEAYMMMKIRN | | |
| Mut2 | For numbering purposes Ser at second position | prt | 7 |
| | is considered residue 1 | | |
| | MSPGQGTQSENSCTHFPGNLPNMLRDLREAFKRVKTFFQMKDQLDSL | | |
| | LLKESLLEDFKGYLGCQALSEMIQFYLEEVMPQAENQDPDIKAHVNS | | |
| | LGENLKTLRLRLRRCHRFLPCENKSKAVEQVKNAFNKLQEKGIYKAM | | |
| | SEFDIFINYIEAYMMMKIRN | | |
| Mut3 | For numbering purposes Ser at second position | prt | 8 |
| | is considered residue 1 | | |
| | MSPGQGTQSENSCTHFPGILPNMLRDLREAFKRVKTFFQMKDQLDSL | | |
| | LLKESLLEDFKGYLGCQALSEMIQFYLEEVMPQAENQDPDIKAHVIS | | |
| | LGENLNTLRLRLRRCHRFLPCENKSKAVEQVKNAFNKLQEKGIYKAM | | |
| | SEFDIFINYIEAYMMMKIRN | | |
| MutSC1 | For numbering purposes Met at first position is | prt | 9 |
| | considered residue 5 | | |
| | MTQSENSCTHFPGNLPNMLRDLRDAFSRVKTFFQMKDQLDNLLLKES | | |
| | LLEDFKGYLGCQALSEMIQFYLEEVMPQAENQDPDIKAHVNSLGENL | | |
| | KTLRLRLRRCHRFLPCENKSKAVEQVKNAFNKLQEKGIYKAMSEFDI | | |
| | FINYIEAYMTMNNGGLDYLPNMLRDLRDAFSRVKTFFQMKDQLDNLL | | |
| | LKESLLEDFKGYLGCQALSEMIQFYLEEVMPQAENQDPDIKAHVNSL | | |
| | GENLKTLRLRLRRNHRFLPCENKSKAVEQVKNAFNKLQEKGIYKAMS | | |
| | EFDIFINYIEAYMTMKIRN | | |
| MutSC2 | For numbering purposes Met at first position is | prt | 10 |
| | considered residue 5 | | |
| | MTQSENSCTHFPGILPNMLRDLREAFKRVKTFFQMKDQLDSLLLKES | | |
| | LLEDFKGYLGCQALSEMIQFYLEEVMPQAENQDPDIKAHVISLGENL | | |
| | NTLRLRLRRCHRFLPCENKSKAVEQVKNAFNKLQEKGIYKAMSEFDI | | |
| | FINYIEAYMMMNNGGLDYLPNMLRDLREAFKRVKTFFQMKDQLDSLL | | |
| | LKESLLEDFKGYLGCQALSEMIQFYLEEVMPQAENQDPDIKAHVISL | | |
| | GENLNTLRLRLRRNHRFLPCENKSKAVEQVKNAFNKLOEKGIYKAMS | | |
| | EFDIFINYIEAYMMMKIRN | | |

| Key: | |
|---|---|
| ID | Mutations (1y6k PDB numbering) |
| IL-10 ORF | WT human IL-10 |
| MutM | Engineered monomer IL-10 that has an inserted polyGly loop between |
| | residues Asn116 and Lys117 of IL-10 ORF |
| Mut1 | DL28E, SL31R, NL45S, TL155M |
| Mut2 | DL28E, SL31K, NL45S, TL155M |
| Mut3 | DL28E, SL31K, NL45S, TL155M, NL181, NL921, KL99N |
| MutSC1 | Single-chain generated in this work connecting the N-and C-termini by |
| | a designed FGGLDY (SEQ ID NO: 1) residue loop mutated to NGGLDY |
| | (SEQ ID NO: 13) |
| MutSC2 | D179E D28E; S182K S31K; N45S N196S; T155M T306M; N181; N2431 |
| | N921; K99N K250N (MutSC1 as background) |

This table describes various constructs generated in this disclosure as well as the
strains in which the different constructs have been implemented. For each of the genetic
constructs, we have included the amino acid or nucleotide sequence. A List of IL-10 variants
was analysed in this work. The reference molecule is the non-engineered WT IL-10 as the
starting background scaffold. For the mutants 1 (Mut1), 2 (Mut2), and 3 (Mut3), the position
and chain of the mutated residues listed corresponds to the PDB numbering. The L chain holds
IL-10. The variants MutSC1, MutSC2 are single-chain molecules generated in this work
(′foldikines′). MutSC2 is based on MutSC1 and incorporates the point mutations shown in Mut3.
Mutations in MutSC2 are duplicated, since two identical domains were merged, except for
position 18, as mutations of this position were not compatible with the loop engineered to make
a single molecule.

TABLE 2A

| | Peptide Level | | | IL10-WT | IL10-WT | IL10-WT |
|---|---|---|---|---|---|---|
| | Master | | | LysC-Trypsin | Tryp_GluC | LysC_Chym |
| | Protein | Annotated | | Areas: F12: | Areas: F7: | Areas: F6: |
| Disulfide | Accessions | Sequence | Modifications | Sample | Sample | Sample |
| | IL10WT | [K].AHVNSLGE | | 3.10E+07 | | |
| | | NLK.[T] | | | | |
| | IL10WT | [K].ESLLEDFK.[ | | 5.60E+07 | 1.90E+05 | 2.20E+06 |
| | | [G] | | | | |
| | IL10WT | [K].DQLDNLLLK. | | 7.40E+07 | 2.20E+06 | 6.40E+06 |
| | | [E] | | | | |
| | IL10WT | [E].VMPQAENQ | | | 3.20E+07 | |
| | | DPDIK.[A] | | | | |
| | IL10WT | [E].SLLEDFK.[G] | | | 7.10E+05 | |
| Cys62 | IL10WT | [E].DFKGYLGC | 1xCarbamido- | | 4.40E+06 | |
| | | QALSE.[M] | methyl [C8] | | | |
| Cys12 | IL10WT | [E].NSCTHFPG | 1xCarbamido- | | 1.60E+06 | |
| | | NLPNMLR.[D] | methyl [C3] | | | |
| | IL10WT | [K].DQLDNLLLK | | | 5.50E+06 | |
| | | E.[S] | | | | |
| | IL10WT | [Y].LEEVMPQAE | | | | |
| | | NQDPDIK.[A] | | | | |
| | IL10WT | [F].YLEEVMPQA | | | | 8.00E+06 |
| | | ENQDPDIK.[A] | | | | |

TABLE 2B

| | | | | | MutSC2 | |
|---|---|---|---|---|---|---|
| | Master | | | LysC-Trypsin | LysC_Chym | LysC_GluC |
| | Protein | Annotated | | Areas: F13: | Areas: F8 | Areas: F9 |
| Disulfide | Accessions | Sequence | Modifications | Sample | Sample | Sample |
| | MutSC2 | [Y].LEEVMPQAEN | | | 1.60E+07 | |
| | | QDPDIK.[A] | | | | |
| | MutSC2 | [Y].LEEVMPQAEN | 1xOxidation | | 1.80E+06 | |
| | | QDPDIK.[A] | [M5] | | | |
| | MutSC2 | [F].YLEEVMPQAE | | | 8.70E+07 | |
| | | NQDPDIK.[A] | | | | |
| | MutSC2 | [F].YLEEVMPQAE | 1xOxidation | | 1.10E+07 | |
| | | NQDPDIK.[A] | [M6] | | | |
| | MutSC2 | [K].ESLLEDFK.[G] | | 1.60E+08 | 4.80E+07 | 3.40E+07 |
| | MutSC2 | [K].DQLDSLLLK.[E] | | 1.80E+08 | 4.00E+07 | 2.80E+07 |
| Cys62 | MutSC2 | [Y].LGCQALSEMI | 1xCarbamido- | | 8.40E+05 | |
| | | QF.[Y] | methyl [C3] | | | |
| | MutSC2 | [K].AHVISLGENLN | | 1.90E+08 | | |
| | | TLR.[L] | | | | |
| | MutSC2 | [K].AHVISLGENLN | | 2.90E+05 | | |
| | | TLRLR.[L] | | | | |
| Cys12 | MutSC2 | [M].TQSENSCTH | 1xCarbamido- | 5.80E+06 | | |
| | | FPGILPNMLR.[D] | methyl [C7] | | | |
| | MutSC2 | [E].VMPQAENQD | | | | 5.70E+07 |
| | | PDIK.[A] | | | | |

Table 2A and 2B. Proteomics analysis of the supernatants of selected Mycoplasma strains (WT IL-10 and MutSC2 expressed by M. pneumoniae WT for the identification of disulfide bridges. The details of the protocol followed in the proteomics facility using three different digestion procedures are described in the Methods section: Table 2A—sulfide WT IL—10, and Table 2B —sulfide MutSC2. In each of them, we showed the different peptides found after the digestion and their possible chemical modifications if found. The presence of a carbamidomethyl implies that the peptide cysteine is part of a disulfide bridge. Peptide sequences in Table 2A from top to bottom are SEQ ID NOs: 19 to 28, respectively; peptide sequences in Table 2B from top to bottom are SEQ ID NOs: 27, 27, 28, 28, 20, 21, 29 to 32, and 22. Amino acid residues indicated in square brackets do not form part of the peptide sequence. LysC digested with LysC protease. Chym, digested with Chymotrypsin. GluC digested with GluC protease. Tryp, digested with Trypsin. When the two names appear together, it means both proteases were used. A comparable proteomics analysis was performed for sulfide MutSC1 and potential disulfide bridge cysteine residues were identified at positions Cys12 and Cys62 (data not shown).

TABLE 3
List of antibodies used in this work. For each of them,
we include the clone description and the supplier.

| Antigen | Clone number | Supplier |
|---|---|---|
| CD16 | 3GS | Biolegend |
| MerTK | 590H1 1G183 | Biolegend |
| CD163 | GH1/61 | Biolegend |
| B7-H1 PD-L1 | MIH2 | Biolegend |
| CD86 | IT2.2 | Biolegend |
| HLA-DR | I.243 | Biolegend |
| CD16/32 (FcBlock) | 93 | Biolegend |
| STAT-3 | D1A5 | Cell Signaling |
| | | Technology |
| P-STAT3 | D3A7 | Cell Signaling |
| | | Technology |
| anti-actin | 20-33 | Sigma-Aldrich |

TABLE 4

| Gene | Orientation | Sequence (5′-3′) | SEQ ID NO: | Reference |
|---|---|---|---|---|
| gapdH | F | CCCACTAACATCAAATGGGG | 33 | Regueiro et al., |
| | R | CCTTCCACAATGCCAAAGTT | 34 | 2011 |
| tnf-α | F | AGGCACTCCCCCAAAAGA | 35 | Euba et al., 2017 |
| | R | GCTCCTCCACTTGGTGGTTT | 36 | |
| kc | F | GACAGACTGCTCTGATGGCA | 37 | Euba et al., 2017 |
| | R | TGCACTTCTTTTCGCACAAC | 38 | |
| mip-1a | F | CCCAGCCAGGTGTCATTTTCC | 39 | Wang et al., |
| | R | GCATTCAGTTCCAGGTCAGTG | 40 | 2019 |
| mcp-1 | F | CTTCTGGGCCTGCTGTTCA | 41 | Kei Goto et al., |
| | R | CCAGCCTACTCATTGGGATCA | 42 | 2016 |
| inf-γ | F | CTGGCAAAAGGATGGTGAC | 43 | Software |
| | R | GCTGATGGCCTGATTGTCTT | 44 | Primer3 |
| tlr2 | F | CTCCTGAAGCTGTTGCGTTAC | 45 | Wang et al., |
| | R | GCTCCCTTACAGGCTGAGTTC | 46 | 2019 |
| il-1ß | F | GCTGCTTCCAAACCTTTGAC | 47 | Shen et al., 2018 |
| | R | AGCTTCTCCACAGCCACAAT | 48 | |
| il-4 | F | GCAACGAAGAACACCACAGA | 49 | Software |
| | R | GCAACGAAGAACACCACAGA | 50 | Primer3 |
| il-6 | F | TGATGCACTTGCAGAAAACA | 51 | Leili et al., 2019 |
| | R | GGTCTTGGTCCTTAGCCACTC | 52 | |
| il- | F | CAGCACCAGCTTCTTCATCAG | 53 | Happel et al., |
| 12p40 | R | TCTTCAAAGGCTTCATCTGCAA | 54 | 2005 |
| il-18 | F | ACTGTACAACCGCAGTAATACG | 55 | Brieland et al., |
| | R | AGTGAACATTACAGATTTATCCC | 56 | 2000 |
| il-23 | F | TGCTGGATTGCAGAGCAGTAA | 57 | Kei Goto et al., |
| | R | GCATGCAGAGATTCCGAGAGA | 58 | 2016 |
| ΔglpDv2 | F | G*T*T*G*CCGTCTTGTAACAGG | 59 | This work |
| | R | [BtnTg]- | 60 | |
| | | AAAATAAGGGTTAATTGGTAAACC | | |
| ΔPuro | F | G*T*G*A*GCACAATCAGCGCTCAAACTG | 61 | This work |
| | R | [BtnTg]-GTAAGTTATCGAGGTGACTTCAC | 62 | |

List of the primers used in this work. For each of them, we include the orientation, sequence,
source reference and show any chemical modification present (*: phosphorotioated base, BtnTg:
biotinylated 5′ end).

TABLE 5
IL-10 protein secretion capacity per cell (M. pneumoniae WT) of WT and variants
(IL-10 ORF, Mut3, and MutSC1) across different biological replicates (R2, R3,
and R4). For each of them, we have integrated the colony-forming units (CFU)
counted for each flask with the IL-10 concentration estimated for each of the
supernatants by ELISA (see Methods). We have corrected the expression values
selecting as reference the MutSC1 expression per cell (fg/CFU) in R2.

| | | R3 normalised | R4 normalised | | | |
|---|---|---|---|---|---|---|
| Data | R2 | (factor | (factor | | | Fold- |
| normalised | reference | applied *1/60) | applied *2) | Average | Stdev | change |
| IL-10 ORF | 3.542092857 | 0.810555702 | 3.71E+00 | 2.6869789 | 1.328565237 | 1 |
| (fg/cell) | | | | | | |
| Mut3 | 4.043921687 | 1.372270192 | 3.30E+00 | 2.9040929 | 1.125366579 | 1.0808 |
| (fg/cell) | | | | | | |
| MutSC1 | 10.49550595 | 10.7686619 | 8.34E+00 | 9.8666104 | 1.088271667 | 3.67201 |
| (fg/cell) | | | | | | |

TABLE 6
Energy calculations using FoldXv5 (see Methods). Values are provided
in Kcal/mol. For each of the mutants (Mut1, Mut2, and Mut3),
interaction energies with R1 and R2 independently and stability
energy in the apo form and the complex has been calculated (Summary
analysis sheet). Energies have been calculated in three independent
PDB models (2ilk, 1y6k, and 6x93).

| | | DDGSta- | DDGInter- | DDGInter- | DDGSta- |
|---|---|---|---|---|---|
| Mutant | Model | bility | action_R1 | action_R2 | bilityComplex |
| Mut1 | 2ilk | −5.92 | | | |
| Mut2 | 2ilk | −5.25 | | | |
| Mut3 | 2ilk | −1.45 | | | |
| Mut1 | 1y6k | −5.58 | −7.64 | | −10.22 |
| Mut2 | 1y6k | −4.25 | −2.66 | | −6.71 |
| Mut3 | 1y6k | −1.30917 | −2.2395 | | −2.83119 |
| Mut1 | 6x93 | −2.05981 | 0.74141 | −0.4357 | −3.13052 |
| Mut2 | 6x93 | −2.23692 | 1.3284 | −0.2881 | −1.55588 |
| Mut3 | 6x93 | −2.4908 | −0.2762 | −1.3807 | −4.46315 |

TABLE 7
Ex vivo characterisation of the kinetic parameters of IL-10 variants generated in this
work expressed and secreted by M. pheumoniae. Top we show the average and standard
deviation of different kinetic parameters from several biological replicas (bottom).

| | | | | | Apparent | sd | Fold | |
|---|---|---|---|---|---|---|---|---|
| | Bmax | sd | Hill | sd | Kd (M) | App. | change- | Biological |
| | average | Bmax | average | Hill | average | Kd | apparentKD | replicates |
| IL-10 | 1.89 | 0.43 | 1.55 | 0.77 | 1.98E−10 | 9.24E−11 | 1.00E+00 | 5 |
| ORF | | | | | | | | |
| MutM | 1.63 | 0.37 | 3.38 | 2.48 | 2.56E−10 | 8.77E−11 | 7.73E−01 | 2 |
| Mut1 | 2.19 | 0.47 | 1.25 | 0.50 | 1.45E−10 | 6.59E−11 | 1.37E+00 | 3 |
| Mut2 | 1.57 | 0.32 | 1.21 | 0.22 | 6.45E−11 | 1.73E−11 | 3.07E+00 | 3 |
| Mut3 | 1.97 | 0.44 | 1.63 | 0.75 | 2.61E−11 | 1.49E−11 | 7.59E+00 | 6 |
| MutSC1 | 1.73 | 0.18 | 1.40 | 0.33 | 1.90E−11 | 5.12E−12 | 1.04E+01 | 4 |
| MutSC2 | 1.76 | 0.15 | 1.26 | 0.21 | 1.20E−11 | 2.80E−12 | 1.65E+01 | 3 |
| rIL-10 | 1.51 | 0.01 | 2.12 | 0.83 | 4.61E−10 | 1.67E−10 | 4.30E−01 | 2 |

TABLE 8
Gene expression of inflammatory markers in lungs infected with M. pneumoniae WT or
CV8. C57BI/6 mice were infected with 100 μl containing 107 CFU/mouse and sacrificed
at 2- or 4 days post-infection (dpi). The control group (PBS) was treated with 100
μl of vehicle solutions readouts of these reporter cells are measured as absorbance
at 630 nm (see Methods) and are proportional to the capacity of each of the IL-10 variants
to activate IL-10 receptors (R1 and R2). The ELISA quantification and the HEK-blue
derived absorbance values are displayed in different sheets for each replica. All the
kinetics obtained derived from these results (Bmax, hill constant (h), and KD) have
been calculated with a kinetic model (see Methods) using the GraphPad Prism software.
Each of the parameters calculated is shown in the ‘Integrated Table’ sheet. In
those experiments where saturation has not been reached, the Bmax has been set to the
maximum Bmax in the same biological replicate and displayed with an asterisk.

| | Fold change of mRNA gene expression (2^{-ΔΔCt}) | | | | | |
|---|---|---|---|---|---|---|
| ID | 2 dpi | | | 4 dpi | | |
| Gene | PBS | WT | CV8 | PBS | WT | CV8 |
| tnf-a | 1 ± 0.39 | 7.89 ± 4.32 * | 2.35 ± 1.61 | 1 ± 0.21 | 0.74 ± 0.62 | 0.64 ± 0.3 |
| kc | 1 ± 0.58 | 3.94 ± 0.73 * | 3.86 ± 0.56 # | 1 ± 0.27 | 1.48 ± 0.39 | 0.83 ± 0.1 |
| mip-1a | 1 ± 0.6 | 25.3 ± 7.02 * | 12.05 ± 0.53 | 1 ± 0.13 | 7.24 ± 4.41 | 2.54 ± 0.74 |
| mcp-1 | 1 ± 0.6 | 14.7 ± 6.65 * | 7.11 ± 2.95 | 1 ± 0.36 | 6.03 ± 2.96 | 2.20 ± 0.34 |
| il-1b | 1 ± 0.76 | 7.24 ± 4.93 * | 6.43 ± 1.47 | 1 ± 0.54 | 3.76 ± 2.32 | 4.02 ± 0.85 |
| inf-γ | 1 ± 0.78 | 9.27 ± 13.46 | 6.66 ± 2.61 | 1 ± 0.54 | 5.47 ± 3.65 | 3.78 ± 0.4 |

Statistical analysis was performed using One-Way ANOVA + Tukey’s Multiple Comparison Test
* p < 0.05 vs Ctrl, at equivalent time point
# p < 0.05 WT vs CV8, at equivalent time point

TABLE 9
Histological findings were observed in lungs infected with PAO1 and treated with CV8 encoding IL-10 variants.
Control mice were inoculated with PBS or hIL-10r protein. Hematoxylin and Eosin (H&E) staining was used
for evaluation of cell infiltration (score: 0-5), perivascular/peribronchial infiltrated (score: 0-5) and
PMN in bronchial/bronchiolar lumen (scored 0-5). The total score (score: 0-15) was calculated by the sum
of all parameters. Immunohistochemistry was used for the quantitative determination of neutrophils (neutrophil
elastase) or macrophages (FA). The lower score means a less inflammatory or pathogenic phenotype.

| | | Groups | | | | | | | |
|---|---|---|---|---|---|---|---|---|---|
| Histological | | | PAO + | PBS + | PAO1+ | PAO1 + | PAO1 + | PAO1 + | PAO1 + |
| finding | | PBS | PBS | CV8 | CV8 | CV8_ORF | CV8_MutSC1 | CV8_MutSC2 | hIL10h |
| H&E | Cell | 2.1 ± | 3.33 ± | 2.88 ± | 3.25 ± | 3.75 ± | 3.0 ± | 3.63 ± | 3.33 ± |
| | infiltration | 0.2 | 0.47 * | 0.22 | 0.56 * | 0.43 * | 0.35 | 0.41 * | 0.47 * |
| | Perivascular/ | 0 ± | 3.5 ± | 2.0 ± | 3.63 ± | 3.63 ± | 2.75 ± | 3.75 ± | 3.33 ± |
| | Peribronchial | 0 | 0.41 * | 0.35 *, # | 0.41 * | 0.41 * | 0.56 * | 1 * | 0.47 * |
| | cell infiltrated | | | | | | | | |
| | PMN in | 0 ± | 3 ± | 0.25 ± | 0.63 ± | 1.88 ± | 1 ± | 1 ± | 1.67 ± |
| | Bronchial/ | 0 | 0 * | 0.63 # | 0.65 # | 0.74 * | 0.71 *, # | 0.71 *, # | 0.47 * |
| | bronchiolar | | | | | | | | |
| | lumen | | | | | | | | |
| | Total score | 2.1 ± | 9.83 ± | 5.13 ± | 7.50 ± | 9.25 ± | 6.75 ± | 8.38 ± | 8.33 ± |
| | (A + B + C) | 0.2 | 0.85 * | 0.74 *, # | 1.27 * | 1.09 * | 1.03 *, # | 0.96 * | 1.25 * |
| IHC | NE (% | 4.9 ± | 28.02 ± | 2.56 ± | 20.81 ± | 26.81 ± | 12.01 ± | 17.63 ± | 19.46 ± |
| | Positive | 1.15 | 2.44 * | 0.95 # | 8.74 * | 8.74 * | 2.3 # | 1.18 | 3.92 |
| | cells) | | | | | | | | |
| | FA (% | 10.77 ± | 61.86 ± | 11.13 ± | 44.3 ± | 51.19 ± | 40.68 ± | 42.30 ± | 44.48 ± |
| | Positive | 5.4 | 5.89 * | 0.38 # | 0.47 * | 3.21 * | 5.44 | 4.43 * | 11.09 * |
| | cells) | | | | | | | | |

Statistical comparison was done using ONE-Way ANOVA + Tukey post-hoc test
* p < 0.05, vs PBS;
# p < 0.05, vs PAO1 + PBS

### Assessing the Impact of the Engineered Linkers in the Foldikine-10

### Constructs and Designs

Above, we have described a novel molecule termed MutSC1 with the following amino acid sequence:

```
MutSC1
(SEQ ID NO: 9)
MTQENSCTHFPGNLPNMLRDLRDAFSRVKTFFQMKDQLDNLLLKESLLEDFKGYLGCQALSEMIQFYLEEVMPQ
                        hhh hhhhhhhhhhh hhhhhh hhhhhhhhhhhhhhhhhhh
                                                HA HB
AENQDPDIKAHVNSLGENLKTLRLRLRRCHRFLPCENKSKAVEQVKNAFNKLQEKGIYKAMSEFDIFINY
hhh hhhhhhhhhhhhhhhhhhh hhhhhhhhhhhh hhhhhhh hhhhhh
                              HC HD HE
IEAYMTMNGGLDYLPNMLRDLRDAFSRVKTFFQMKDQLDNLLLKESLLEDFKGYLGCQALSEMIQFYLEEV
hhhhhhhh hhhhhhhhhhh hhhhhh hhhhhhhhhhhhhhhh
      HF HA HB
MPQAENQDPDIKAHVNSLGENLKTLRLRLRRNHRFLPCENKSKAVEQVKNAFNKLQEKGIYKAMSEFDIF
hhhhhh hhhhhhhhhhhhhhhhhhh hhhhhhhhhhhh hhhhhhh hhh
                                HC HD HE
```

The sequence underscored in bold corresponds to the peptide linker that was designed to connect the C- and N-termini of the two monomers domains. In—bold italics—we show the sequence being part of the two wild type structured linkers (CENKSKA; SEQ ID NO: 168) between the 3D domains. In the case of IL-10, which is a natural ‘swapped dimer’, these linker sequences are natural, structured linkers and impart the characteristic fold angle (V-form) between the two domains of the foldikine (or between the left and right domains of a wild-type IL-10 dimer. We have generated a control in which the NGGLD sequence (SEQ ID NO:318) was replaced by a poly Gly linker of the same length (5aa) to assess the impact of the linker composition in the functional activity of the engineered molecule (underlined). This results in a molecule termed MutSC1_polyglycontrol_19.

```
>MutSC1_polyglycontrol_19
(SEQ ID NO: 63)
MTQENSCTHFPGNLPNMLRDLRDAFSRVKTFFQMKDQLDNLLLKESLLEDFKGYLGCQALSEMIQFYLEEVMPQ
                        hhh hhhhhhhhhhh hhhhhh hhhhhhhhhhhhhhhhh
                                                HA HB
AENQDPDIKAHVNSLGENLKTLRLRLRRCHRFLPCENKSKAVEQVKNAFNKLQEKGIYKAMSEFDIFINY
hhh hhhhhhhhhhhhhhhhhhh hhhhhhhhhhhh hhhhhhh hhhhhh
                            HC HD HE
IEAYMTMGGGGGYLPNMLRDLRDAFSRVKTFFQMKDQLDNLLLKESLLEDFKGYLGCQALSEMIQFYLEEV
hhhhhhhh hhhhhhhhhhh hhhhhh hhhhhhhhhhhhhhhh
      HF HA HB
MPQAENQDPDIKAHVNSLGENLKTLRLRLRRNHRFLPCENKSKAVEQVKNAFNKLQEKGIYKAMSEFDIF
hhhhhh hhhhhhhhhhhhhhhhhhh hhhhhhhhhhhh hhhhhhh hhh
                                HC HD HE
INYIEAYMTMKIRN
Hhhhhhhhhh
HF
```

To assess the importance of having two linkers between each 3D domain compared to a single linker and the importance of the rigidity of the V-form at the center of the molecule the following control polypeptides were synthesised and assayed: A) We have duplicated the N—C termini linker of one 3D domain into the other foldikine domain, such that each domain is formed from a continuous primary sequence, and to create new N- and C-termini for the polypeptide we have cut at position Asn166 in one of the two linkers between the 3D domains (see the underlined sequences in MutSC1 and MutSC1_control_center21 showing the novel terminus and in bold italics and the two non-natural N- to C-terminal linkers (bold). Thus, in contrast to the foldikines of the invention (as described elsewhere herein), the foldikine ‘MutSC1_control_center21’ only has one structural linker spanning between the left and right 3D domains in the folded protein.

```
>MutSC1_controlcenter_21
(SEQ ID NO: 64)
KSKAVEQVKNAFNKLQEKGIYKAMSEFDIFINYIEAYMTMNFGGLDYLPNMLRDLRDAFSR
    hhhhhhhhhhhh hhhhhhh hhhhhhhhhhhhhh hhhhhhhh
            HA HE HF HA
VKTFFQMKDQLDNLLLKESLLEDFKGYLGCOALSEMIQFYLEEVMPQAENQDPDIKAHVNS
hhh hhhhhh hhhhhhhhhhhhhhhhhhhhhh hhhhhhhh
                                                                          HB HC
LGENLKTLRLRLRRCHRFLPCENKSKAVEQVKNAFNKLQEKGIYKAMSEFDIFINYIEAYM
hhhhhhhhhhhh hhhhhhhhhhhh hhhhhhh hhhhhhhhhhh
                                                              HD HE HF
TMNFGGLDYLPNMLRDLRDAFSRVKIFFQMKDQLDNLLLKESLLEDFKGYLGCQALSEMIQ
hhh hhhhhhhhhh hhhhh hhhhhhhhhh
                                    HA HB
FYLEEVMPQAENQDPDIKAHVNSLGENLKTLRLRLRRCHFLPCEN
hhhhhhhhhhhh hhhhhhhhhhhhhhhhhhh
                                    HC
```

### Results

The three different constructs were inserted into the Mycoplasma pneumoniae genome via transposon (Tn4001, CmR) under the control of a synthetic promoter (p3) (https://doi.org/10.1038/s41467-017-00239-7 and a secretion signal (s142_OPT) (https://doi.org/10.15252/msb.202010145). M. pneumoniae was grown in Hayflick medium 48 hours and after this, the supernatants were collected and the concentration of the different engineered molecules calculated by ELISA. In all three cases, the concentration of the supernatant was higher than 30 ng/ml, the maximum concentration that enters in the dynamic range of the HEK-BLUE-IL10 cells.

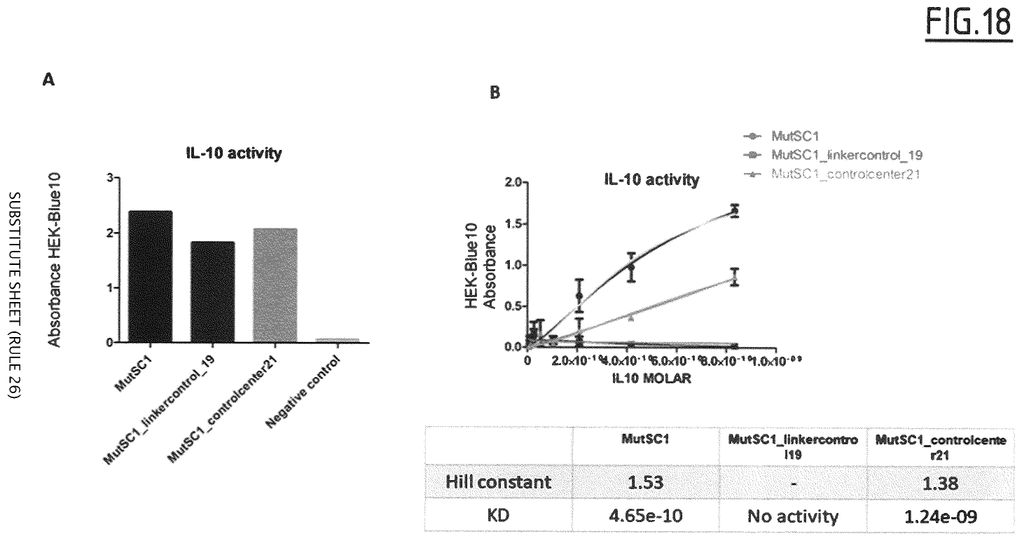

A first approximation of the functionality of the molecules was performed by assessing the activity of non-diluted supernatant in the reporter HEK-blue cells. All three constructs activated the STAT3 signaling pathway (see FIG. 18).

Then, we performed 8-serial dilutions starting from 30 ng/ml (and diluting 0.5× each) and assessed the activation of HEK blue cells (see FIG. 18B). We fitted a Michaelis-Menten model to the experimental data (saturation binding, specific binding with Hill slope, fixing Bmax at 2.3) and obtained the apparent KD. When the engineered loop was substituted by a polyGly linker of the same size, this mutant had a very significant drop in activity (MutSC1_linkercontrol19/MutSC1_polyglycontrol_19). When the N- and C-terminal linker was introduced into both 3D domains (with new N- and C-termini formed between the foldikine domains) there was a drop in activity of around 2.5-fold (MutSC1_linkercontrol 21/MutSC1_controlcenter_21).

The above results suggest that both the correct amino acid composition of the N- and C-termini linker introduced and the presence of two linker peptides bridging between the adjacent 3D domains is very important for the optimal functionality of the foldikines.

### Example 2: Generation of Foldikines from Monomeric Cytokines of Type Class II with Three Engineered Linkers

List of IL-22 variants tested and described in the following sections. IL-22 foldikine scaffolds are based on the scheme depicted in FIGS. 5 and 27.
IL22 WT, monomeric IL22 based on the Il22 xtal expressed in bacteria e.g. M. pneumoniae (PDB=3dlq).
Hydroph_core-1, monomeric IL22 with mutations T56L, A66M, V95I, T99F, S173L.
Hydroph_core-2, monomeric IL22 with mutations T56M, A66M, V95I, T99F, S173L, N68Q.
CtoA, IL22 WT with Cys at position 7 replaced by Ala
T56M point mutation in monomeric IL22
A66M point mutation in monomeric IL22
V95I point mutation in monomeric IL22
T99F point mutation in monomeric IL22
S173L point mutation in monomeric IL22
foldikine-22_1 (SCIL22_1) (SEQ ID NO: 65) a single-chain IL-22 dimer in which a linker is engineered to connect the N- and C-termini of one of the IL-22 monomer sequences and a dimeric unit was then created by fusing that monomer to another IL-22 monomer sequence by breaking a loop sequence in each monomer sequence and connecting the respective new N- and C-termini to form interdomain bridging linkers (as illustrated in FIGS. 5 and 27, for example), as further explained below.
foldikine-22_3 (SCIL22_3) (SEQ ID NO: 66) a single chain foldikine similar to foldikine-22_1, but having a different engineered linker sequence between one of the new N- and C-termini, and in which the Cys residue at position 2 of SEQ ID NO: 65 is replaced with a Val residue.
foldikine-22_3_linkerNCpolygly 1 foldikine-22_3_NCpolygly includes two engineered linkers bridging between left and right 3D domains, but replaces the designed/engineered N—C terminus linker with polyGly-Ser. The exact length of the engineered linker was maintained in the new poly-Gly-Ser linker.
foldikine-22_3_centrallinkers keeps the N—C terminus linker of foldikine-22_3 (SCIL22_3), but changes the two central linkers bridging left and right 3D domains to poly Gly-Ser. We maintained the exact length of the engineered linker when changing it to poly-Gly-Ser.

### Constructs and Designs: Generation of a Single-Chain IL-22

Human IL-22 is naturally, a monomeric interleukin. We have converted the IL-22 into a foldikine (termed ‘foldikine-22’) using a combination of intelligent design and the ModelX and FoldX software as described when designing a foldikine based on IL-10 (termed ‘foldikine-10’ herein). Since IL-22 is not naturally a swapped dimer, to generate the foldikine-22, it was necessary to create two non-natural bridges between the separate monomers, and also to link the N- and C-termini of one of the monomers. The two bridges between the separate monomers are created by opening a loop in each monomer and cross-linking the monomers at each of the open sequences (between respective new N- and C-termini), as described and illustrated with respect to FIGS. 5 and 27. The two new bridges between monomers are indicated by underline and double underline in the sequences below. Since these bridges are not located in identical positions and do not fuse identical sequences, each linker sequence may suitably be different, as illustrated in the embodiments below. Two different mutant designs were generated differing at one of the linker sequences (single underlined), as well as having Cys2 mutated to Val in one case (bold, underlined). In bold, we label the designed sequence ANGT (SEQ ID NO: 329) linking the N- and C-termini of one of the 3D domains. The bridging linkers between left and right foldikine domains are: TCMPGGSKT (SEQ ID NO: 337) and RLELLP (SEQ ID NO: 331) in foldikine-22_1 and NRLKKLMASSD (SEQ ID NO: 332) and RLELLP (SEQ ID NO: 331) in foldikine-22_3. For ease of reference, structurally conserved helices in the foldikine 22 are labelled by the consecutive order in which they are found in the original IL-22 WT sequence (i.e. HA, HB . . . ), and the helical segments by h. When a helical segment is a slightly distorted helix and therefore some residues are not assigned as h in swisspdb but is surrounded by regular helical segments this is indicated as h-Nres-h, where Nres could be any number of residues shown as -. The helical segments are defined using the swisspdb software and could vary slightly in length depending on the software used to define them, as well as of the crystal structure

```
>SCIL22_1 (foldikine-22_1)
(SEQ ID NO: 65)
GCRLDKSNFQQPYITNRTFMLAKEASLADNNTDVRLIGEKLFHGVSMSERCYLMKQVLNFT
                      hhhhhhhhhhhhhhhhh hhh hhhhhhhhhhhh
                                      HA HB
LEEVLFPQSDRFQPYMQEVVPFLARLSNRLSTCMPGGSKTHIQRNVQKLKDTVKKLGESGE
h---hhhh hhhhhhhhhhhhhhhhh hhhhhhhhhhhhhh hhh
                                        HC HD
IKAIGELDLLFMSLRNACIANGTLDKSNFQQPYITNRTFMLAKEASLADNNTDVRLIGEKL
hhhh hhhhhhhhhhh hhhhhhhhhhhhhhhhh hh
HE HF HA
FHGVSMSERCYLMKQVLNFTLEEVLFPQSDRFQPYMQEVVPFLARLSNRLSRLELLPHIQR
h hhhhhhhhhhhhh hhh hhhhhhhhhhhhhhhhh hhhh
                          HB HC
NVQKLKDTVKKLGESGEIKAIGELDLLFMSLRNACI
hhhhhhhhhhh hhhhhhh hhhhhhhhhhh
    HD HE HF
>SCIL22_3 (foldikine-22_3)
(SEQ ID NO: 66)
GVRLDKSNFQQPYITNRTFMLAKEASLADNNTDVRLIGEKLFHGVSMSERCYLMKQVLNFT
                      hhhhhhhhhhhhhhhhh hhh hhhhhhhhhhhh
                                      HA HB
LEEVLFPQSDRFQPYMQEVVPFLARLSNRLKKLMASSDHIQRNVQKLKDTVKKLGESGEIK
h---hhhh hhhhhhhhhhhhhhhhh hhhhhhhhhhhhhhh hhh
                                        HC HD
AIGELDLLFMSLRNACIANGTLDKSNFQQPYITNRTFMLAKEASLADNNTDVRLIGEKLFH
hhhh hhhhhhhhhhh hhhhhhhhhhhhhhhhh hh
HE HF HA
GVSMSERCYLMKQVLNFTLEEVLFPQSDRFQPYMQEVVPFLARLSNRLSRLELLPHIQRNV
h hhhhhhhhhhhh hhh hhhhhhhhhhhhhhhhh hhhh
                          HB HC
QKLKDTVKKLGESGEIKAIGELDLLFMSLRNACI
hhhhhhhhhhh hhhhhhh hhhhhhhhhhh
    HD HE HF
```

To further investigate the importance of the engineered linker sequences we generated a number of additional mutant foldikine constructs based on the foldikine-22_3 framework: the first one, foldikine-22_3_linkerNCpolygly retains the sequence of the two engineered linker bridges of foldikine-22_3, but changes the N—C terminus linker to polyGly. The other construct (foldikine-22_3_centrallinkers) keeps the N—C terminus linker (bold) but changes the two central linker bridges between 3D domains to poly Gly-Ser (grey). In both cases, we maintained the exact length of the engineered linker when changing it to poly-Gly or to poly Gly-Ser respectively.

>foldikine-22_3_linkerNCpolygly
(SEQ ID NO: 67)
GVRLDKSNFQQPYITNRTFMLAKEASLADNNTDVRLIGEKLFHGVSMSE
RCYLMKQVLNFTLEEVLFPQSDRFQPYMQEVVPFLARLSNRLKKLMASS
DHIQRNVQKLKDTVKKLGESGEIKAIGELDLLFMSLRNACIGGGGLDKS
NFQQPYITNRTFMLAKEASLADNNTDVRLIGEKLFHGVSMSERCYLMKQ
VLNFTLEEVLFPQSDRFQPYMQEVVPFLARLSNRLSRLELLPHIQRNVQ
KLKDTVKKLGESGEIKAIGELDLLFMSLRNACI
>foldikine-22_3_centrallinkers
(SEQ ID NO: 68)
GVRLDKSNFQQPYITNRTFMLAKEASLADNNTDVRLIGEKLFHGVSMSE
RCYLMKQVLNFTLEEVLFPQSDRFQPYMQEVVPFLARLSGGSGGSGGSG
GHIQRNVQKLKDTVKKLGESGEIKAIGELDLLFMSLRNACI-ANGTLDK
SNFQQPYITNRTFMLAKEASLADNNTDVRLIGEKLFHGVSMSERCYLMK
QVLNFTLEEVLFPQSDRFQPYMQEVVPFLARLSNRLSGGSGGSHIQRNV
QKLKDTVKKLGESGEIKAIGELDLLEMSLRNACI

### Stabilising the Hydrophobic Core of IL-22 in the Bound State

In parallel, we engineered on to the IL-22 framework some mutations that, based on modelling of the hydrophobic core, were predicted to stabilise the hydrophobic core in the bound conformation. Those amino acid replacements that were expected to provide a stability enhancement and that were not 100% conserved by evolution across different species were particularly desirable. Each potential mutation was assessed individually and in combination.

The crystallographic structure and sequence that was selected as a reference for the calculation of the stability changes when mutating the hydrophobic core was the 3dlq.PDB. We identified residues T56, A66, V95, T99 and S193 as the ones that upon mutation could potentially stabilise the IL22 protein. These residues are marked in grey on the WT Pdb sequence (SEQ ID NO: 69).

```
3dlq. Pdb sequence
(SEQ ID NO: 69)
ARLDKSNFQQPYITNRTFMLAKEASLADNNTDVRLIGEKLFHGVSMSERCYLMKQVLNFTL
                    hhhhhhhhhhhhhhhhh hhh hhhhhhhhhhhhh
                                  HA HB
EEVLFPQSDRFQPYMQEVVPFLARLSNRLSTCHIEGDDLHIQRNVQKLKDTVKKLGESGEI
---hhhh hhhhhhhhhhhhhhhhh hhhhhhhhhhhhhhhh hhhh
                                      HC HD HE
KAIGELDLLFMSLRNACI
hhh hhhhhhhhhhhh
                                                    HF
```

In Table 10 below, we list the 5 mutants predicted with FoldX to stabilize the hydrophobic core of IL22.

TABLE 10
stabilising mutations in the hydrophobic core of IL-22

| Name | Mutant |
|---|---|
| 56 | T56M |
| 66 | A66M |
| 95 | V95I |
| 99 | T99L |
| 173 | S173L |

Then we introduced the hydrophobic mutations individually and collectively in the foldikine-22 constructs described above.

```
3dlq. Pdb sequence-Hydrophobic core stabilised protein (56)
(SEQ ID NO: 343)
ARLDKSNFQQPYITNRMFMLAKEASLADNNTDVRLIGEKLFHGVSMSERCYLMKQVLNFTL
                    hhhhhhhhhhhhhhhhh hhh hhhhhhhhhhhhh
                                  HA HB
EEVLFPQSDRFQPYMQEVVPFLARLSNRLSTCHIEGDDLHIQRNVQKLKDTVKKLGESGEI
---hhhh hhhhhhhhhhhhhhhhh hhhhhhhhhhhhhhhh hhhh
                                      HC HD HE
KAIGELDLLFMSLRNACI
hhh hhhhhhhhhhhh
                      HF
3dlq. Pdb sequence-Hydrophobic core stabilised protein (66)
(SEQ ID NO: 344)
ARLDKSNFQQPYITNRTFMLAKEMSLADNNTDVRLIGEKLFHGVSMSERCYLMKQVLNFTL
                    hhhhhhhhhhhhhhhhh hhh hhhhhhhhhhhhh
                                  HA HB
EEVLFPQSDRFQPYMQEVVPFLARLSNRLSTCHIEGDDLHIQRNVQKLKDTVKKLGESGEI
---hhhh hhhhhhhhhhhhhhhhh hhhhhhhhhhhhhhhh hhhh
                                      HC HD HE
KAIGELDLLFMSLRNACI
hhh hhhhhhhhhhhh
                      HF
3dlq. Pdb sequence-Hydrophobic core stabilised protein (95)
(SEQ ID NO: 345)
ARLDKSNFQQPYITNRTFMLAKEASLADNNTDVRLIGEKLFHGVSMSERCYLMKQILNFTL
                    hhhhhhhhhhhhhhhhh hhh hhhhhhhhhhhhh
                                  HA HB
EEVLFPQSDRFQPYMQEVVPFLARLSNRLSTCHIEGDDLHIQRNVQKLKDTVKKLGESGEI
---hhhh hhhhhhhhhhhhhhhhh hhhhhhhhhhhhhhhh hhhh
                                      HC HD HE
KAIGELDLLFMSLRNACI
hhh hhhhhhhhhhhh
                      HF
3dlq. Pdb sequence-Hydrophobic core stabilised protein (99)
(SEQ ID NO: 346)
ARLDKSNFQQPYITNRTFMLAKEASLADNNTDVRLIGEKLFHGVSMSERCYLMKQVLNFML
                    hhhhhhhhhhhhhhhhh hhh hhhhhhhhhhhhh
                                  HA HB
EEVLFPQSDRFQPYMQEVVPFLARLSNRLSTCHIEGDDLHIQRNVQKLKDTVKKLGESGEI
---hhhh hhhhhhhhhhhhhhhhh hhhhhhhhhhhhhhhh hhhh
                                      HC HD HE
KAIGELDLLFMSLRNACI
hhh hhhhhhhhhhhh
                      HF
3dlq. Pdb sequence-Hydrophobic core stabilised protein (173)
(SEQ ID NO: 347)
ARLDKSNFQQPYITNRTFMLAKEASLADNNTDVRLIGEKLFHGVSMSERCYLMKQVLNFTL
                    hhhhhhhhhhhhhhhhh hhh hhhhhhhhhhhhh
                                  HA HB
EEVLFPQSDRFQPYMQEVVPFLARLSNRLSTCHIEGDDLHIQRNVQKLKDTVKKLGEMGEI
---hhhh hhhhhhhhhhhhhhhhh hhhhhhhhhhhhhhhh hhhh
                                      HC HD HE
KAIGELDLLFMSLRNACI
hhh hhhhhhhhhhhh
                      HF
3dlq. Pdb sequence-Hydrophobic core stabilised protein (IL-22hydC)
(SEQ ID NO: 70)
ARLDKSNFQQPYITNRMFMLAKEMSLADNNTDVRLIGEKLFHGVSMSERCYLMKQILNFML
                    hhhhhhhhhhhhhhhhh hhh hhhhhhhhhhhhh
                                  HA HB
EEVLFPQSDRFQPYMQEVVPFLARLSNRLSTCHIEGDDLHIQRNVQKLKDTVKKLGELGEI
---hhhh hhhhhhhhhhhhhhhhh hhhhhhhhhhhhhhhh hhhh
                                      HC HD HE
KAIGELDLLFMSLRNACI
hhh hhhhhhhhhhhh
                      HF
```

### Engineering IL-22 Interface Mutations

A crystallographic structure of mouse IL-22 bound to receptors R1 and R2 is available (6we0.pdb). We have generated a humanised model of this crystallographic structure using FoldX software to mutate the residues that are different between mouse and human, and ModelX software to model small backbone moves to better accommodate, if necessary, the amino acid differences. After, having human and mouse IL-22 structures with the R1 and R2 receptors we performed a position scan analysis with FoldX for each of the positions in contact with the receptors. Then we analysed the interaction energy with both receptors and the stability of the complex. We selected those positions that may favour binding to both human and mouse receptors.

In Table 11 below, we list the different positions analysed in the human structure, the DDG in the stability of the complex, and the DDG of interaction with R1 and R2 respectively. The values provided are in kcal/mol.

TABLE 11
residue positions in human IL-22 that affect
stability and interaction with R1 and R2.

| | DDG | IL22-R1 | IL22-R2 |
|---|---|---|---|
| | Stability | DDG | DDG |
| Mutant | Complex | Interaction | Interaction |
| A123Y | −2.16 | 0.00 | −2.32 |
| D43D, S45H | 3.21 | 0.00 | 0.13 |
| D43D, S45K | −0.07 | 0.00 | −0.32 |
| D43D, S45N | 0.26 | 0.00 | −0.51 |
| D43D, S45Q | 0.88 | 0.00 | 0.10 |
| D43D, S45R | 0.49 | 0.00 | −0.33 |
| D43E, S45H | 3.89 | 0.00 | −0.09 |
| D43E, S45K | 1.76 | −0.02 | −0.47 |
| D43E, S45N | 1.87 | −0.02 | −1.01 |
| D43E, S45Q | 2.67 | −0.02 | 0.01 |
| D43E, S45R | 2.13 | −0.05 | −0.53 |
| D43H | 4.28 | 0.00 | −0.20 |
| D43H, S45H | 7.22 | 0.00 | −0.37 |
| D43H, S45K | 3.27 | 0.00 | −0.56 |
| D43H, S45N | 4.27 | 0.00 | −1.23 |
| D43H, S45Q | 3.96 | 0.00 | −0.15 |
| D43H, SZ45R | 3.82 | 0.00 | −0.82 |
| D43N, S45H | 3.40 | 0.00 | −0.01 |
| D43N, S45K | 1.90 | −0.01 | −1.08 |
| D43N, S45N | 3.10 | 0.00 | −0.10 |
| D43N, S45Q | 1.44 | 0.00 | −1.22 |
| D43N, S45R | 2.24 | 0.01 | −1.19 |
| D43Q, S45H | 4.43 | 0.00 | 0.03 |
| D43Q, S45K | 3.11 | −0.02 | −0.71 |
| D43Q, S45N | 3.10 | 0.00 | −1.44 |
| D43Q, S45Q | 3.43 | 0.00 | −0.02 |
| D43Q, S45R | 3.52 | −0.03 | −0.93 |
| E77Y | −0.56 | −1.74 | 0.00 |
| K162K | −0.05 | 0.01 | 0.00 |
| K61E | 0.50 | −0.94 | 0.04 |
| K61H | −0.30 | −0.90 | −0.01 |
| K61W | −0.73 | −1.18 | 0.02 |
| N176E | 0.47 | 0.14 | −0.01 |
| N68Q | 0.62 | −0.03 | 0.00 |
| N97Q | −0.21 | 0.00 | 0.01 |
| Q116R | −0.06 | 0.00 | 0.21 |
| Q116W | 0.11 | 0.00 | −1.08 |
| Q116Y | 0.44 | 0.00 | 0.08 |
| Q48L | −0.75 | −0.26 | −0.11 |
| Q49S | −0.47 | −0.05 | 0.57 |
| R128K | −0.48 | 0.00 | 0.41 |
| S45R | 0.49 | 0.00 | −0.36 |
| S64E | 0.07 | −2.56 | 0.00 |
| S64M | −1.11 | −0.65 | 0.00 |
| T70I | −0.98 | −1.55 | 0.00 |
| K61W, S64K | | 2.24 | |
| K61W, S64M | | −1.94 | |

In Table 12, below, we list the different positions analyzed in the mice structure, the DDG in Stability of the complex, and the DDG of interaction with R1 and R2 respectively. The values provided are in kcal/mol.

TABLE 12
residue positions in mouse IL-22 that affect
stability and interaction with R1 and R2.

| | DDG | IL22-R1 | IL22-R2 |
|---|---|---|---|
| | Stability | DDG | DDG |
| Mutant | Complex | Interaction | Interaction |
| Q48L | −1.39 | −0.57 | −0.11 |
| K61E | 0.50 | −0.80 | −0.18 |
| K61H | −0.42 | −1.25 | 0.01 |
| K61W | −0.94 | −1.52 | 0.03 |
| S64M | −1.74 | −0.82 | 0.00 |
| Q116W | 0.35 | 0.00 | −0.36 |
| Q116Y | 0.35 | 0.00 | 0.10 |
| Q116R | −0.51 | 0.00 | −0.09 |
| N176E | 0.46 | 0.25 | −0.05 |
| E43H | 1.15 | −0.03 | −0.78 |
| S45R | −0.33 | −0.08 | −1.17 |
| Q49S | −1.14 | −0.03 | −0.84 |
| N68Q | 0.00 | 0.09 | 0.00 |
| N97Q | −0.15 | 0.00 | 0.00 |
| Q128K | 0.18 | 0.00 | −0.17 |
| S64E | 1.68 | 0.22 | −0.01 |
| T70I | 0.09 | −0.21 | 0.00 |
| E77Y | −0.22 | −1.06 | 0.00 |
| K162K | 0.00 | 0.00 | 0.00 |
| Q128Y | 0.60 | 0.00 | 0.00 |
| E43D, S45H | −0.17 | −0.07 | −1.46 |
| E43D, S45R | −0.63 | 0.00 | 0.48 |
| E43D, S45Q | −0.65 | −0.02 | 0.19 |
| E43D, S45K | −1.03 | 0.00 | 0.65 |
| E43D, S45N | 0.17 | −0.02 | 1.93 |
| E43E, S45H | 1.01 | −0.07 | −1.70 |
| E43E, S45R | −1.00 | −0.03 | −1.77 |
| E43E, S45Q | 0.77 | 0.00 | −0.28 |
| E43E, S45K | 0.29 | 0.00 | 0.80 |
| E43E, S45N | 1.70 | 0.00 | 0.18 |
| E43H, S45H | 2.73 | −0.10 | −1.00 |
| E43H, S45R | 0.49 | −0.08 | −1.83 |
| E43H, S45Q | 1.80 | −0.04 | −0.44 |
| E43H, S45K | 0.95 | −0.04 | −0.27 |
| E43H, S45N | 2.36 | −0.04 | −1.04 |
| E43Q, S45H | 1.88 | −0.09 | −2.04 |
| E43Q, S45R | 0.52 | −0.11 | −2.24 |
| E43Q, S45Q | 1.55 | −0.03 | −1.02 |
| E43Q, S45K | 0.91 | −0.07 | −1.70 |
| E43Q, S45N | 1.75 | −0.03 | −1.01 |
| E43N, S45R | 1.24 | −0.04 | −0.59 |
| E43N, S45Q | 1.43 | −0.04 | −0.38 |
| E43N, S45K | 0.67 | −0.04 | −0.39 |
| E43N, S45N | 1.30 | −0.04 | −1.54 |
| E43N, S45H | 1.60 | −0.10 | −2.10 |

With these elements, we selected a series of combinations of mutations to be explored first in the IL-22 WT. Below, we can find the sequence of the interface-engineered mutants.

Table 13 (below) indicates the mutations made to the wild type IL-22 sequence that increase both stability of IL-22 and the interaction with its receptor. The mutations were implemented over the IL-22 WT, and further included in the foldikine-22 scaffolds (partial sequence alignments of wild-type and mutants C to E shown below).

TABLE 13
mutations in II-22 that increase stability

| | | |
|---|---|---|
| mutantC_IL22 | A132Y, D43E, S45R, E77Y, T70I; | SEQ ID NO: 71 |
| mutantD_IL22 | K61W, S64M | SEQ ID NO: 72 |
| mutantE_IL22 | A132Y, D43E, S45R, E77Y, T70I, | SEQ ID NO: 73 |
| | K61W, S64M; | |

WT22
(SEQ ID NO: 317)
APISSHCRLDKSNFQQPYITNRTFMLAKEASLADNNTDVRLIGEK
LFHGVSMSERCYLMKQVLNFTLEEVLFPQSDRFQPYMQEVVPFLA
RLSNRLSTCHIEGDDLHIQRNVQKLKDTVKKLGESGEIKAIGELD
LLFMSLRNACI
mutantC_IL22
(SEQ ID NO: 71)
APISSHCRLEKRNFQQPYITNRTFMLAKEASLADNNIDVRLIGYK
LFHGVSMSERCYLMKQVLNFTLEEVLFPQSDRFQPYMQEVVPFLY
RLSNRLSTCHIEGDDLHIQRNVQKLKDTVKKLGESGEIKAIGELD
LLFMSLRNACI
mutantD_IL22
(SEQ ID NO: 72)
APISSHCRLDKSNFQQPYITNRTFMLAWEAMLADNNTDVRLIGEK
LFHGVSMSERCYLMKQVLNFTLEEVLFPQSDRFQPYMQEVVPFLA
RLSNRLSTCHIEGDDLHIQRNVQKLKDTVKKLGESGEIKAIGELD
LLFMSLRNACI
mutantE_IL22
(SEQ ID NO: 73)
APISSHCREDRSNFQQPYITNRTFMLAWEAMLADNNIDVRLIGYK
LFHGVSMSERCYLMKQVLNFTLEEVLFPQSDRFQPYMQEVVPFLY
RLSNRLSTCHIEGDDLHIQRNVQKLKDTVKKLGESGEIKAIGELD
LLFMSLRNACI

### Experimental Results

### Analysis of the Impact of Linker Protein Composition in IL-22 Protein Functionality

The substitution of the N—C termini linker of the IL-22 foldikine with a polygly linker (foldikine-22_3_linkerNCpolygly) or the central linkers (foldikine-22_3_centrallinkers) resulted in a complete loss of function in the protein, supporting the idea that engineered linker composition greatly impacts protein affinity (see FIG. 19).

### Analysis of Mutations Stabilising the Hydrophobic Core of IL-22

For the mutations introduced over the IL-22 scaffold (Kd=1.7e-11), we observed an enhancement in the measured KD, which is postulated to be due to the stabilisation of this protein in the bound state when the core-stabilising mutations are introduced into the hydrophobic scaffold (Kd=5.4e-12) (FIG. 20).

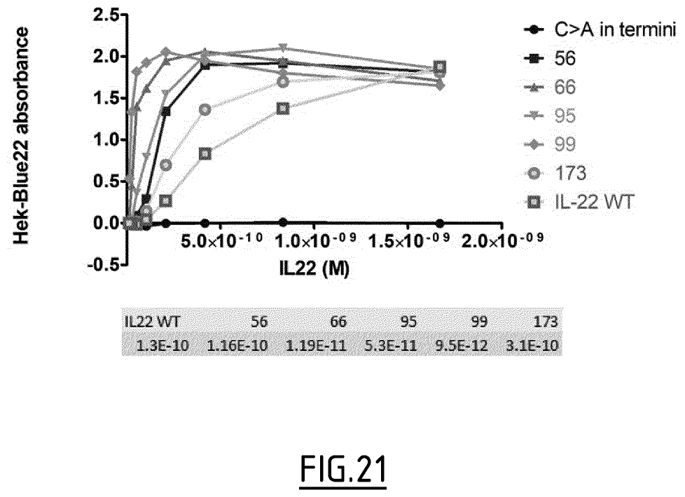

We have also analysed the impact of each of the mutants separately and compared versus IL-22 WT. When the Cys2 amino acid in the N-terminus of the foldikine-22 sequence was mutated to prevent formation of a disulphide bridge, we observed a complete loss of function. Notwithstanding, when different single mutations in the hydrophobic core were assessed, we observed an enhancement in the affinity of the protein for three of the five mutations at positions 66, 95 and 99 (FIG. 21). We observed that the mutation at position 173 is deleterious and the one at 56 appears to be neutral. This may explain why only a two-fold gain in affinity was achieved in the multiple hydrophobic mutant. Therefore, the future version of IL-22 and foldikine-22 may beneficially include the three mutations at 66, 95 and 99. Our results show that stabilising the hydrophobic core of a foldikine (or monomeric) protein in the bound state can be a successful strategy to enhance protein affinity (FIG. 21).

Analysis of Mutations Improving Interaction with R1 and R2 Receptors.

The impact of mutations designed to improve interaction between IL-22 and R1 (mutantC_IL22, SEQ ID NO: 71), R2 (mutantD_IL22, SEQ ID NO: 72) and both (mutantE_IL22, SEQ ID NO: 73) were analysed. A 10-fold increase in affinity for the mutantE_IL22 over wt (app KD 3.37E-11 vs 1.28E-10), and a two-fold increase in affinity for mutantD_IL22 was observed.

### Analysis of the Foldikine-22 Variants.

We inserted the different constructs into a bacterial genome via transposon (Tn4001, TcR) as described in Methods. The bacterial cells (e.g. M. pneumoniae, L. lactis or E. coli) were grown in Hayflick medium for 48 hours and after this, the supernatants were collected and the concentration of the different IL-22 engineered molecules calculated by ELISA. In all three cases, the concentration of the supernatant was higher than 30 ng/ml, the maximum concentration that enters in the dynamic range of the HEK-BLUE-22 cells.

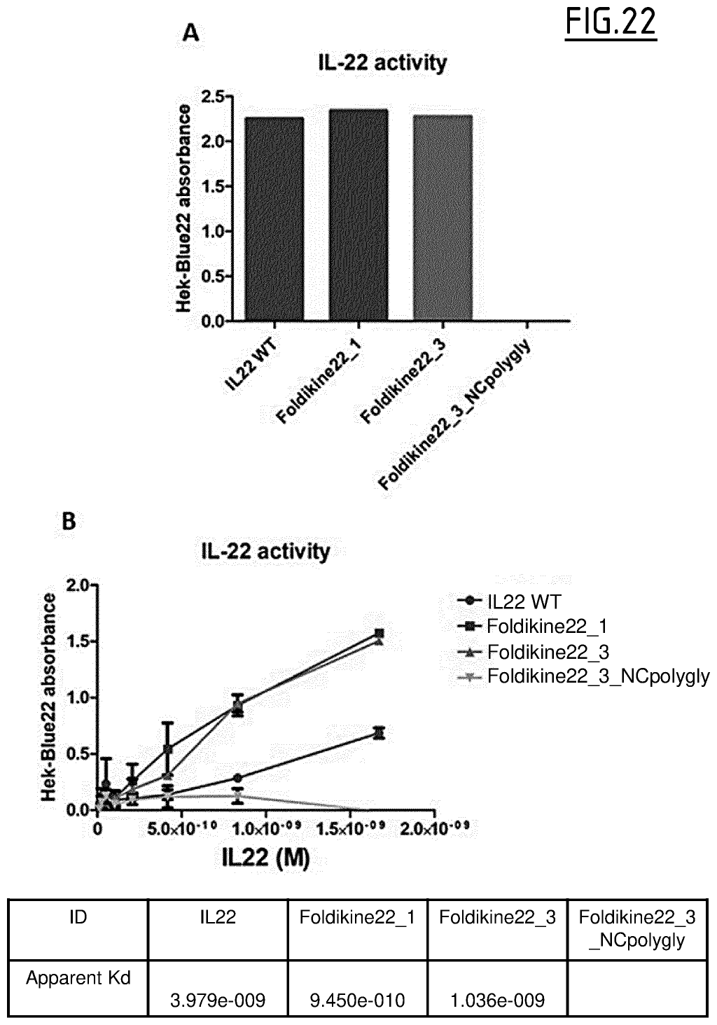

The first approximation of the functionality of the molecules was performed by assessing the activity of non-diluted supernatant in the reporter HEK-blue cells (see FIG. 22A). From these molecules, IL-22, foldikine-22-1 and foldikine-22_3 showed a capacity to activate IL-22 mediated signaling pathway via STAT3. The construct in which foldikine22_linker N—C termini was substituted by a poly-Gly linker (foldikine-22_3_linkerNCpolygly) did not activate the signaling pathway, suggesting that it is not active (FIGS. 19 and 22B).

Next, we performed 8-serial dilutions starting from 30 ng/ml (and diluting 0.5× each) and assessed the activation of HEK blue cells (see FIG. 22B). We fitted a Michaelis-Menten model to the experimental data (Saturation binding, specific binding with Hill slope, fixing Bmax at 2.2) and obtained the apparent Kd. The apparent Kds are displayed in panel B. Both foldikines showed an increased Kd of around 4-fold in comparison with IL22 WT, validating the improvement in activity of foldikine-22 over wild-type IL-22.

### Chimeric IL-10/IL-22

List of chimeric IL-10/IL-22 chimeric foldikine variants tested and described in the following sections:
Foldikine10-22. Chimeric foldikine joining IL-10 and IL-22, where the IL-10 corresponds to the closed/continuous foldikine 3D domain and IL-22 to the open/split domain. (SEQ ID NO: 74).
Chimera 3/Foldikine10-22_3 is based on Foldikine10-22 in which the Cys residue at position 1 is replaced with an Ala residue. (SEQ ID NO: 75).
Chimera 5/Foldikine10-22_5 is based on Foldikine10-22_3 in which a group of 5 additional amino acid substitutions have been made in an attempt to increase the stability of the IL-22 split 3D domain to improve IL-22 activity of the protein (SEQ ID NO: 76).
Heterodimer10-22. Control in which the engineered monomeric IL-10 described in (Josephson et al., 2000) is fused to IL-22 by a short gly-ser linker of six amino acids joining the C-termini of IL-10 to the N-termini of IL-22.
Heterodimer22-10 Control were the engineered monomeric IL-22 is fused to the monomeric IL-10 described in (Josephson et al., 2000) by a short gly-ser linker of six amino acids joining the C-termini of IL-22 to the N-termini of IL-10.

### Constructs and Designs

We started by modelling an IL-22 dimer structure (3dlq.pdb) over the IL-10 swapped domain structure 1y6k.pdb). To form the ‘Sewing1022’ construct, (SEQ ID NO: 74) we removed the IL-22 loop between helices C and D of the natural IL-22 sequence (cutting after residue Leu129 and before Arg142) and removed the loop between Lys88 and Cys114 of IL-10 (helices C and D), and connected Leu129 of IL-22 with Lys88 of IL-10 with an engineered DPKAAFKS linker (SEQ ID NO: 314), and Cys114 of IL-10 with Arg143 of IL-22 via a DSKVNR linker (SEQ ID NO: 315). At the same time the N- and C-terminal ends of IL-10 were closed by an engineered peptide linker NFGGLDY (SEQ ID NO: 316). In italic we show the residues corresponding to IL-22, and in underscored those of IL-10. Bold italic indicates the two central linkers introduced to join the two halves of IL-22 to IL-10. In bold non-italic is the linker peptide linking the N- and C-termini of IL-10.

```
>sewing1022/Foldikine10-22
(SEQ ID NO: 74)
CRLDKSNFQQPYITNRTFMLAKEASLADNNTDVRLIGEKLFHGVSMSERCYLMKQVLNFTL
                    hhhhhhhhhhhhhhhhh hhh hhhhhhhhhhhhh
                              HA-IL22 HB-IL22
EEVLFPQSDRFQPYMQEVVPFLARLSNRLDPKAAFKSKAVEQVKNAFNKLQEKGIYKAMSE
            hhhhhhhhhhhhhhhhh Xn hhhhhhhhhhhh hhhhhhh
                                HC HD-IL10 HE-IL10
FDIFINYIEAYMTMNFGGLDYLPNMLRDLRDAFSRVKTFFQMKDQLDNLLLKESLLEDEKG
  hhhhhhhhhhh NtCt hhhhhhhhhh hhhhhh
          HF-IL10 HA-IL10
YLGCQALSEMIQFYLEEVMPQAENQDPDIKAHVNSLGENLKTLRLRLRRCHRFLPCDSKVN
    hhhhhhhhhhhhhhhhhhhhhh hhhhhhhhhhhhhhhhhhhh Xn
                      HB-IL10 HC-IL10
RRNVQKLKDTVKKLGESGEIKAIGELDLLFMSLRNACI
  Hhhhhhhhhhhh hhhhhhh hhhhhhhhhhh
        HD-IL22 HE_IL22 HF-IL22
```

This initial sewing pattern of the foldikine-10/22 molecule demonstrated IL-10 activity in assays, but not significant IL-22 activity. The same was observed with Foldikine10-22_3/Chimera 3 (SEQ ID 75) in which the first Cys residue was mutated to Ala. This result could be due to a lack of stability of the IL-22 split domain, or a problem with the designed linkers. To investigate and solve this issue we generated a set of mutations for increasing the stability of the hydrophobic core of the split domain IL-22 to attempt to rescue functionality (Foldikine1022_5/Chimera 5; SEQ ID NO: 76). In the table below, we list the set of mutations performed. The mutations are shown in bold and underlined in SEQ ID NOS: 75 and 76, below).

> Foldikine10-22_3
(SEQ ID NO: 75)
ARLDKSNFQQPYITNRTFMLAKEASLADNNTDVRLIGEKLFHGVS
MSERCYLMLQVLNFTLEEVLFPQSDRFQPYMQEVVPFLARLSNRL
DPKAAFKCKAVEQVKNAFNKLQEKGIYKAMSEFDIFINYIEAYMT
MNFGGLDYLPNMLRDLRDAFSRVKTFFQMKDQLDNLLLKESLLED
EKGYLGCLCLSEMIQFYLEEVMPQAENQDPDIKAHVNSLGENLKT
LRLRLRRCHRFLPCISKVLRRNVQKLKDTVKKLGESGEIKAIGEL
DLLFMSLRNACI*
>Foldikine10-22_5
(SEQ ID NO: 76)
ARLDKSNFQQPYITNRLFMLAKEASLMDNNTDVRLIGEKLFHGVS
MSERCYLMLQILNFELEEVLFPQSDRFQPYMQEVVPFLARLSNR
LDPKAAFKCKAVEQVKNAFNKLQEKGIYKAMSEFDIFINYIEAY
MTMNFGGLDYLPNMLRDLRDAFSRVKTFFQMKDQLDNLLLKESL
LEDEKGYLGCLCLSEMIQFYLEEVMPQAENQDPDIKAHVNSLGE
NLKTLRLRLRRCHRFLPCISKVLRRNVQKLKDTVKKLGESGEIK
AIGELDLLFMLLRNACI

The mutations of this molecule are the following

| Identifier | Mutations over the starting SCIL1022 |
|---|---|
| Foldikine10-22_3 | C1A |
| Foldikine10-22_5 | C1A, S314L, T60F, V56I, A27M, T17L |

As controls, we generated two different protein fusions. The engineered monomeric IL-10 described in (Josephson et al., 2000) (underscored below) fused to IL-22 (italics) by a short gly-ser linker of six amino acids (heterodimer10-22) (SEQ ID NO: 77). In parallel, we generated the inverse construct (heterodimer22-10) in which IL-22 (italics) C-terminus was fused to IL-10 (underscored)N-terminus via a short gly-ser linker of six amino acids (SEQ ID NO: 78). The linker is indicated in bold underlined in SEQ ID NO: 78 below.

>heterodimer10-22
(SEQ ID NO: 77)
MSPGQGTQSENSCTHFPGNLPNMLRDLRDAFSRVKTFFQMKDQLD
NLLLKESLLEDFKGYLGCQALSEMIQFYLEEVMPQAENQDPDIKA
HVNSLGENLKTLRLRLRRCHRFLPCENGNGGKSKAVEQVKNAFNK
LQEKGIYKAMSEFDIFINYIEAYMTMKIRNGGSGGSAPISSHCRL
DKSNFQQPYITNRTFMLAKEASLADNNTDVRLIGEKLFHGVSMSE
RCYLMKQVLNFTLEEVLFPQSDRFQPYMQEVVPFLARLSNRLSTC
HIEGDDLHIQRNVQKLKDTVKKLGESGEIKAIGELDLLFMSLRNA
CI
>heterodimer22-10
(SEQ ID NO: 78)
APISSHCRLDKSNFQQPYITNRTFMLAKEASLADNNTDVRLIGEK
LFHGVSMSERCYLMKQVLNFTLEEVLFPQSDRFQPYMQEVVPFLA
RLSNRLSTCHIEGDDLHIQRNVQKLKDTVKKLGESGEIKAIGELD
LLFMSLRNACIGGSGGSMSPGQGTQSENSCTHFPGNLPNMLRDLR
DAFSRVKTFFQMKDQLDNLLLKESLLEDFKGYLGCQALSEMIQFY
LEEVMPQAENQDPDIKAHVNSLGENLKTLRLRLRRCHRFLPCENG
NGGKSKAVEQVKNAFNKLQEKGIYKAMSEFDIFINYIEAYMTMKI
RN*

### Results

For assessing the functionality of the engineered molecules we first started by quantifying by ELISA the concentration in the supernatant of the cell culture of the different versions upon expression and secretion by the bacterium (e.g. M. pneumoniae, L. lactis or E. coli). For the quantification of the different chimeras, we used antibodies recognising IL-22 by ELISA, because we found that the commercial antibody we used to recognise human IL-10 (Deluxe human IL-10 ELISA kit, Biolegend) does not recognise it in the IL-10/IL-22 chimera.

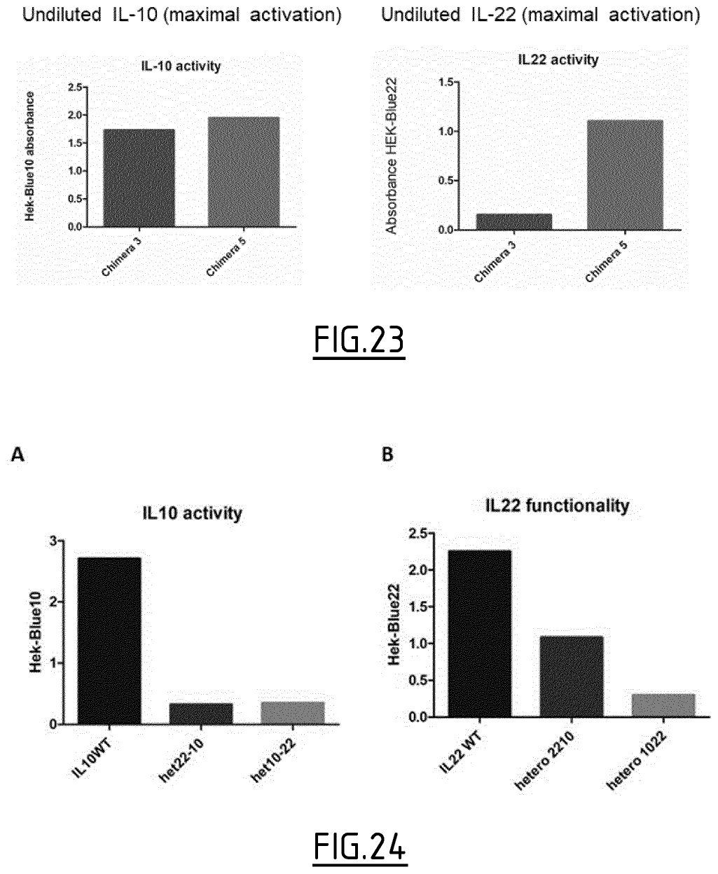

Then, we assessed the functionality of the molecules using two reporter cell lines in parallel. The first, HEK-Blue™ IL-22 cells derive from embryonic kidney HEK293 cell line to detect bioactive human and murine IL-22. The second reporter cell line, HEK-Blue™ IL-10 also detects bioactive human and mouse interleukin 10 (IL-10). In FIG. 23 we show the experimental results

For IL-10 activity, we demonstrated that Chimera 3 and Chimera 5 were active for IL-10 functionality. Plus, we demonstrated that Chimera 5 had also IL-22 functionality. Therefore, Foldikine1022_5 (Chimera 5) demonstrates that it is possible to generate a single-molecule cytokine with a dual functionality. Moreover, it is evident that one or more of the mutations in the IL-22 first half split domain, i.e. T60F, V56I, A27M, T17L, and/or second half split domain, i.e. S314L, are responsible for rescuing the activity of IL-22.

Regarding the control tests, i.e. based on fusion proteins of monomeric IL-10 to IL-22 and the inverse fusion, i.e. IL-22 to IL-10 linked via flexible linkers, resulted in near complete loss of IL-10 function, and only partial IL-22 activity, observed in the fusion 22-10 (‘heterodimer22-10’, see FIG. 24). This result supports the foldikine concept and the importance of the two structured linkers joining the IL-10 3D/foldikine domain to the split IL-22 3D/foldikine domain, which bridge the left and right 3D domains of the folded single-chain protein.

### Example 3: Construction of Foldikines Based on Cytokine Helical Bundle Families

At the structural level, two main cytokine helical bundle families can be used to make foldikines

As stated above we can classify these cytokines into either Class I or Class II, which foldikine linkers and bridges inserted accordingly, as described herein.

Below, we propose the homomeric human foldikines class I helical cytokines. As will be clear to the skilled person, the concept of foldikines can be expanded to any other non-human interleukin that presents structural compatibility.

### Class I Helical Cytokines

### A) Short-Chain Helical, Sequences and Corresponding Homomeric Foldikines

### Human Sequences of IL2, IL3, IL4, IL5, IL7, IL9, IL15, IL21, TSLP and GMCS-F

We have removed the signal peptide in the below sequences, but the skilled person will appreciate that similar constructs containing the signal peptide also fall within the scope of the invention.

>sp|P60568|IL2_HUMAN Interleukin-2
(SEQ ID NO: 79)
MYRMQLLSCIALSLALVINSAPTSSSTKKTQLQLEHLLLDLQMIL
NGINNYKNPKLTRMLTFKFYMPKKATELKHLQCLEEELKPLEEVL
NLAQSKNFHLRPRDLISNINVIVLELKGSETTFMCEYADETATIV
EFLNRWITFCQSIISTLT
>sp|P08700|IL3_HUMAN Interleukin-3
(SEQ ID NO: 80)
RPGLQAPMTQTTPLKTSWVNCSNMIDEIITHLKQPPLPLLDENNL
NGEDQDILMENNLRRPNLEAFNRAVKSLQNASAIESILKNLLPCL
PLATAAPTRHPIHIKDGDWNEFRRKLTFYLKTLENAQAQQTTLSL
AIF
>sp|P05112|IL4_HUMAN Interleukin-4
(SEQ ID NO: 81)
GNFVHGHKCDITLQEIIKTLNSLTEQKTLCTELTVTDIFAASKNT
TEKETFCRAATVLRQFYSHHEKDTRCLGATAQQFHRHKQLIRFLK
RLDRNLWGLAGLNSCPVKEANQSTLENFLERLKTIMREKYSKCSS
>sp|P05113|IL5_HUMAN Interleukin-5
(SEQ ID NO: 82)
TEIPTSALVKETLALLSTHRILLIANETLRIPVPVHKNHQLCTEE
IFQGIGTLESQTVQGGTVERLFKNLSLIKKYIDGQKKKCGEERRR
VNQFLDYLQEFLGVMNTEWIIES
>sp|P13232|IL7_HUMAN Interleukin-7
(SEQ ID NO: 83)
PVASSDCDIEGKDGKQYESVLMVSIDQLLDSMKEIGSNCLNNEFN
FFKRHICDANKEGMELFRAARKLRQFLEC-50NSTGDFDLHLLKV
SEGTTILLNCTGQVKGRKPAALGEAQPTKSLEENKSLKEQKKLND
LCFLKRLLQEIKTCWNKILMGTKEH
>sp|P15248|IL9_HUMAN Interleukin-9
(SEQ ID NO: 84)
QGCPTLAGILDINFLINEC-
50QEDPASKCHCSANVTSCLCLGIPSDNCTRPCFSERLSQMTNTT
MQTRYPLIFSRVKKSVEVLKNNKCPYFSCEQPCNQTTAGNALTFL
KSLLEIFQKEEC-50RGMRGKI
>sp|P40933|IL15_HUMAN Interleukin-15
(SEQ ID NO: 85)
NSHELTEAGIHVFILGCFSAGLPKTEANWVNVISDLKKIEDLIQS
MHIDATLYTESDVHPSCKVTAMKCFLLELQVISLESGDASIHDTV
ENLIILANNSLSSNGNVTESGCKECEELEEKNIKEFLQSFVHIVQ
MFINTS
>sp|Q9HBE4|IL21_HUMAN Interleukin-21
(SEQ ID NO: 86)
HKSSSQGQDRHMIRMRQLIDIVDQLKNYVNDLVPEFLPAPEDVET
NCEWSAFSCFQKAQLKSANTGNNERIINVSIKKLKRKPPSTNAGR
RQKHRLTCPSCDSYEKKPPKEFLERFKSLLQEC-50IHQHLSSRT
HGSED
>sp|Q969D9|TSLP_HUMAN Thymic stromal lymphopoietin
(SEQ ID NO: 87)
YDFTNCDFEKIKAAYLSTISKDLITYMSGTKSTEFNNTVSCSNRP
HCLTEIQSLTENPTAGCASLAKEMFAMKTKAALAIWCPGYSETQI
NATQAMKKRRKRKVTTNKCLEQVSQLQGLWRRFNRPLLKQQ
>sp|P04141|CSF2_HUMAN Granulocyte-macrophage
colony-stimulating factor
(SEQ ID NO: 88)
QPWEHVNAIQEARRLLNLSRDTAAEMNETVEVISEMFDLQEPTCL
QTRLELYKQGLRGSLTKLKGPLTMMASHYKQHCPPTPETSCATQI
ITFESFKENLKDELLVIPFDCWEPVQE

For each of the above interleukins, we propose a methodology for the generation of homomeric foldikines generally following the scheme indicated in FIG. 26—as explained below. For ‘domain swapped’ dimers the foldikine strategy is different, as indicated by FIG. 25, for example.

### Proposed Homomeric Foldikine Class I: Short Helix Bundle Subclass.

In bold double-underlined we highlight the loop sequence that was opened in the other of the cytokine monomers in order to form a split domain. In italics the split molecule/domain fused to the N- and C-termini of the intact monomer. (Xn) are the connecting linkers engineered to link one foldikine domain to the other foldikine domain. Linker sequences are designed to provide a desired structural conformation or are simply a combination of Gly and Ser of different lengths. Beneficially, however, the engineered linker sequences are ‘structured’ linkers that prefer a particular 3D conformation and, therefore, in advantageous embodiments of the disclosure, the engineered linkers do not contain Gly or Ser residues, or beneficially contain 3 or less, 2 or less, or 1 or less Gly or Ser residue. XX_and_YY indicate that extra residues could be added to the N- and C-termini of the split domain at the position of the removed, partially removed or simply opened loop sequence. Furthermore, up to 10 residues right or left respectively at the new N- and C-terminals may be deleted without affecting activity, depending on the design and structural requirements. Thus, XX and YY are optional sequence elements, which may comprise all of part of the opened loop sequences, and do not form part of the sequences identified by the corresponding SEQ ID NOs below.

It should be appreciated that the illustrated position of these linkers represents an embodiment of the disclosure; but that it may be possible to change the position of these linkers—in particular the position of the linker bridges may change by between 1 and 10 or between 1 and 5 amino acids, such as by 1, 2, 3, 4 or 5 amino acids in either the N-terminal direction or the C-terminal direction without preventing the formation of a functional foldikine. As such, the indicated position of these linkers should be considered to be approximate and in each case could be expanded to incorporate (or remove) some of the surrounding residues. For example, up to 10 amino acids could be deleted on each side of the (Xn) position.

For ease of reference, structurally conserved helices in each family are labelled by the consecutive order they are found in the original WT sequence (i.e. HA, HB . . . ), and the helical segments by h. When a helical segment is a slightly distorted helix and, therefore, some residues are not assigned as h in swisspdb but is surrounded by regular helical segments we indicate it as h-Nres-h, where Nres could be any number of residues shown as -. The helical segments are defined using the swisspdb software and could vary slightly in length depending on the software used to define them, as well as of the crystal structure.

```
Foldikine-2(Interleukin-2)(XX_SEQ ID NO: 89-(Xn)-SEQ ID NO: 90-(Xn)-SEQ ID NO: 91_YY)
Sequence opened in one of the cytokine monomers QSKNFHLR, SEQ ID NO: 92
XX_PRDLISNINVIVLELKGSETTFMCEYADETATIVEFLNRWITFCQSIISTLT(Xn)TSSSTKKTQLQLEH
      hhhhh-hhhhhhhhhh hhhhhhhhhhhhhhhhh hhhhhhhhhhhhh
                HC HD HA
LLLDLQMILNGINNYKNPKLTRMLTFKFYMPKKATELKHLQCLEEELKPLEEVLNLAQSKNFHLRPRDLI
hhhhhhhhhhhh hhhhhh hhh hhhhhhh---hhhhhh hhhh
                                                                                        HB
SNINVIVLELKGSETTFMCEYADETATIVEFLNRWITFCQSIISTLT(Xn)TSSSTKKTQLQLEHLLLDLQM
hhhhhhhhhh hhhhhhhhhhhhhhhhhh hhhhhhhhhhhhhhhhhhh
        HC HD HA
ILNGINNYKNPKLTRMLTFKFYMPKKATELKHLQCLEEELKPLEEVLNLA_YY
hhhhh hhhhhh hhhhhhh---hhhhhhh
                                                                          HB
Foldikine-4(Interleukin-4)(XX_SEQ ID NO: 93-(Xn)-SEQ ID NO: 94-(Xn)-SEQ ID NO: 95_YY)
Sequence opened in one of the cytokine monomers, GATA, SEQ ID NO: 96
XX_QQFHRHKQLIRFLKRLDRNLWGLAGLNSCPVKEANQSTLENFLERLKTIMREKYSKCSS(Xn)GNFVHGH
        hhhhhhhhhhhhhhhhhhhhhhh hhhhhhhhhhhhhhhhhhhhhhh
                          HC HD
KCDITLQEIIKTLNSLTEQKTLCTELTVTDIFAASKNTTEKETFCRAATVLRQFYSHHEKDTRCLGATAQ
        hhhhhhhhhhhh hhh hhhhhhhhhhhhhhhhhh hhh hh
                  HA HB
QFHRHKQLIRFLKRLDRNLWGLAGLNSCPVKEANQSTLENFLERLKTIMREKYSKCSS(Xn)GNFVHGHKCD
hhhhhhhhhhhhhhhhhhhhhhh hhhhhhhhhhhhhhhhhhhhhhh
              HC HD
ITLQEIIKTLNSLTEQKTLCTELTVTDIFAASKNTTEKETFCRAATVLRQFYSHHEKDTRCL_YY
hhhhhhhhhhhh hhh hhhhhhhhhhhhhhhhhhh hhh
    HA HB
Foldikine-3(Interleukin-3)(XX_SEQ ID NO: 97-(Xn)-SEQ ID NO: 98-(Xn)-SEQ ID NO: 99_YY)
Sequence opened in one of the cytokine monomers, KSLQNA, SEQ ID NO: 100
XX_SAIESILKNLLPCLPLATAAPTRHPIHIKDGDWNEFRRKLTFYLKTLENAQA(Xn)
      hhhhhhh hh hhhhhhhhhhhhhhhhhh
            HC HD
SWVNCSNMIDEIITHLKQPPLPLLDENNLNGEDQDILMENNLRRPNLEAFNRAV
        hhhhhhhhhhhh hhhhhhh hhhhhhhhhhh
                  HA HB
KSLQNASAIESILKNLLPCLPLATAAPTRHPIHIKDGDWNEFRRKLTFYLKTLENAQAQ(Xn)
            hhhhhhh hh hhhhhhhhhhhhhhhhhh
                HC HD
SWVNCSNMIDEIITHLKQPPLPLLDENNLNGEDQDILMENNLRRPNLEAFNRAV-YY
        hhhhhhhhhhhh hhhhhh hhhhhhhhhhhh
                  HA HB
Foldikine-5(Interleukin-5; swapped dimer)(XX_SEQ ID NO: 101-(NtCt)-SEQ ID NO: 102_YY)
XX_TEIPTSALVKETLALLSTHRTLLIANETLRIPVPVHKNHQLCTEEIFQGIGTLESQTVQGGIVERLFK
              hhhhhhhhhhhhh hhhh hhhhhhhhhhhh hhhhhh
                          HA HB
NLSLIKKYIDGQKKKCGEERRRVNQFLDYLQEFLGVMNTEWIIES(NtCt)
hhhhhhhhhhhh hhhhhhhhhhhhhhhhh
        HC HD
TEIPTSALVKETLALLSTHRTLLIANETLRIPVPVHKNHQLCTEEIFQGIGTLESQTVQGGTVERLEKNL
        hhhhhhhhhhhhh hhhh hhh hhhhhhhhhh hhhhhhhh
                  HA HB HC
SLIKKYIDGQKKKCGEERRRVNQFLDYLQEFLGVMNTEWIIES_YY
hhhhhhhhhh hhhhhhhhhhhhhhhhh
                                                    HD
Foldikine-7(Interleukin-7)(XX_SEQ ID NO: 103-(Xn)-SEQ ID NO: 104-
(Xn)-SEQ ID NO: 105_YY)
Sequence opened in one of the cytokine monomers, KMNSTG, SEQ ID NO: 106
XX_DFDLHLLKVSEGTTILLNCTGQVKGRKPAALGEAQPTKSLEENKSLKEQKKLNDLCFLKRLLQEIKTC
      hhhhhhhhhhhhhh hhhhhhhhhhhhhh
                  HC HD
WNKILMGTKEH(Xn)PVASSDCDIEGKDGKQYESVLMVSIDQLLDSMKEIGSNCLNNEFNFFKRHICDANKE
hhhhh hhh---hhhhhhhhh hhhh
                                                                  HA
GMFLFRAARKLRQFLKMNSTGDFDLHLLKVSEGTTILLNCTGQVKGRKPAALGEAQPTKSLEENKSLKEQ
hhhhhhhhhh hhhhhhhhhhhhhh
        HB HC
KKLNDLCFLKRLLQEIKTCWNKILMGTKEH(Xn)PVASSDCDIEGKDGKQYESVLMVSIDQLLDSMKEIGSN
          hhhhhhhhhhhhhhhhhhh hhh---hhhhhhhhh
                          HD HA
CLNNEFNFFKRHICDANKEGMFLFRAARKLRQFL_YY
              hhhh hhhhhhhhh
                                            HB
Foldikine-9(Interleukin-9)(XX_SEQ ID NO: 107-(Xn)-SEQ ID NO: 108-(Xn)-SEQ ID NO: 109_YY)
Sequence opened in one of the cytokine monomers, RYP
XX_LIFSRVKKSVEVLKNNKCPYFSCEQPCNQTTAGNALTFLKSLLEIFQKEKMRGMRGKI(Xn)QGCPTLAG
      hhhhhhhhhhhhhhh hhhhhhhhhhhhhhhh hhh
                  HC HD
ILDINFLINKMQEDPASKCHCSANVTSCLCLGIPSDNCTRPCFSERLSQMTNTTMQTRYPLIFSRVKKSV
hhhhhhhhhhhh hhhhhh hhhh hhhhhhhhhh
        HA HB HC
EVLKNNKCPYFSCEQPCNQTTAGNALTFLKSLLEIFQKEKMRGMRGKI(Xn)QGCPTLAGILDINFLINKMQ
hhhhh hhhhhhhhhhhhhhhh hhhhhhhhhhhhhhh
                                                            HD HA
EDPASKCHCSANVTSCLCLGIPSDNCTRPCFSERLSQMTNTTMQT_YY
                                                              hhhhhh hhhh
                                                                    HB
Foldikine-15(Interleukin-15)(XX_SEQ ID NO: 110-(Xn)-SEQ ID NO: 111-(Xn)-SEQ ID
NO: 112_YY)
Sequence opened in one of the cytokine monomers, SGD
XX_ASIHDTVENLIILANNSLSSNGNVTESGCKECEELEEKNIKEFLQSFVHIVOMFINTS(Xn)
      hhhhhhhhhhhhhhhhh hhhhhhhhhhhhhhh
                HC HD
TEANWVNVISDLKKIEDLIQSMHIDATLYTESDVHPSCKVTAMKCFLLELQVIS
        hhhhhhhhhhhhhhhh hhhhhhhhhhhhhhhh
                      HA HB
LESGDASIHDTVENLIILANNSLSSNGNVTESGCKECEELEEKNIKEFLQSFVHIVQMFINTS(Xn)NSHFL
hh hhhhhhhhhhhhhhhhh hhhhhhhhhhhhhhh
                        HC HD
TEANWVNVISDLKKIEDLIQSMHIDATLYTESDVHPSCKVTAMKCFLLELQVISLE_YY
        hhhhhhhhhhhhhhh hhhhhhhhhhhhhhhhhh
                    HA HB
Foldikine-21(Interleukin-21)(XX_SEQ ID NO: 113-(Xn)-SEQ ID NO: 114-(Xn)-SEQ ID
NO: 115_YY)
Sequence opened in one of the cytokine monomers, NTG
XX_NERIINVSIKKLKRKPPSTNAGRRQKHRLTCPSCDSYEKKPPKEFLERFKSLLQKMIHQHLSSRTHGS
        hhhhhhhhhhhh hhhhhhhhhhhhhhhhhh
                HC HD
ED(Xn)HKSSSQGQDRHMIRMRQLIDIVDQLKNYVNDLVPEFLPAPEDVETNCEWSAFSCFQKAQLKSANTGN
                            hhhhhhhhhh---hhhhh hhhhhhhhhh
                                          HA HB
NERIINVSIKKLKRKPPSTNAGRRQKHRLTCPSCDSYEKKPPKEFLERFKSLLQKMIHQHLSSRTHGSE
hhhhhhhhhhhh hhhhhhhhhhhhhhhhhh
          HC HD
D(Xn)HKSSSQGQDRHMIRMRQLIDIVDQLKNYVNDLVPEFLPAPEDVETNCEWSAFSCFQKAQLKSA_YY
                      hhhhhhhhhh---hhhhh hhhhhhhhhh
                                          HC HD
Foldikine-TSL(Thymic stromal lymphopoietin)(XX_SEQ ID NO: 116-(Xn)-SEQ ID NO: 117-
(Xn)-SEQ ID NO: 118_YY)
Sequence opened in one of the cytokine monomers, SLAK, SEQ ID NO: 119)
XX_EMFAMKTKAALAIWCPGYSETQINATQAMKKRRKRKVTTNKCLEQVSQLQGLWRRFNRPLLKQQ(Xn)
        hhhhhhhhhhhhh hhhhhhhhhhhhhhh
                  HC HD
NCDFEKIKAAYLSTISKDLITYMSGTKSTEFNNTVSCSNRPHCLTEIQSLTFNPTAGCASLAKEMFAM
      Hhhhhhhhh---hhhhhhhh hhhhhhhhhhhh hhhh
                        HA HB
KTKAALAIWCPGYSETQINATQAMKKRRKRKVTTNKCLEQVSQLQGLWRRENR(Xn)NCDFE
hhhhhhhhh hhhhhhhhhhhhhhh hh
        HC HD
KIKAAYLSTISKDLITYMSGTKSTEFNNTVSCSNRPHCLTEIQSLTFNPTAGCA_YY
hhhhhhh---hhhhhhhh hhhhhhhhhhhh
                HA HB
Foldikine-GMCSF(Granulocyte-macrophage colony-stimulating factor)(XX_SEQ ID NO: 120-
(Xn)-SEQ ID NO: 121-(Xn)-SEQ ID NO: 122_YY)
Sequence opened in one of the cytokine monomers, RGS
XX_LTKLKGPLIMMASHYKQHCPPTPETSCATQIITFESFKENLKDFLLVIPFDCW(Xn)QPWEHVNA
      hh hhhhhhhhhhhhh hhhhhhhhhhhhhhhhhhhh hhhh
                          HA HB
IQEARRLLNLSRDTAAEMNETVEVISEMFDLQEPTCLQTRLELYKQGLRGSLTKLKGPLTMMASHYKQHC
hhhhhhhhhh hhhh hhhhhhhhhhh hh hhhhhhhhhhhhh
          HA HB HC
PPTPETSCATQIITFESFKENLKDELLVIPFDCW(Xn)QPWEHVNAIQEARRLLNLSRDTAAEMNET
                hhhhhhhhhhhhhhhhhhhhh hhhhhhhhhhhhhh hhhh
                                HD HA
VEVISEMFDLQEPTCLQTRLELYKQGL_YY
                              hhhhhhhhhhh
                                        HB
```

### B) Long-Chain Helical, Sequences and Corresponding Homomeric Foldikines

### Human Sequences of IL6, IL11, IL12A, IL23A, IL27A, IL31, CLCF1, CNTF, CTF1, LIF, OSM and CSF3.

Again, we have removed the signal peptide in each construct, but the signal peptide could of course be retained in embodiments.

>sp|P05231|IL6_HUMAN Interleukin-6 OS = Homo sapiens
OX = 9606 GN = IL6 PE = 1 SV = 1
(SEQ ID NO: 123)
PVPPGEDSKDVAAPHRQPLISSERIDKQIRYILDGISALRKETCNKSNMCESSKEALAENN
LNLPKMAEKDGCFQSGFNEETCLVKIITGLLEFEVYLEYLQNRFESSEEQARAVQMSTKVL
IQFLQKKAKNLDAITTPDPTTNASLLTKLQAQNQWLQDMTTHLILRSFKEFLQSSLRALRQ
M
>tr|A8K3F7|A8K3F7_HUMAN Interleukin 11 OS = Homo sapiens
OX = 9606 GN = IL11 PE = 2 SV = 1
(SEQ ID NO: 124)
PGPPPGPPRVSPDPRAELDSTVLLTRSLLADTRQLAAQLRDKFPADGDHNLDSLPTLAMSA
GALGALQLPGVLTRLRADLLSYLRHVQWLRRAGGSSLKTLEPELGTLQARLDRLLRRLQLL
MSRLALPQPPPDPPAPPLAPPSSAWGGIRAAHAILGGLHLTLDWAVRGLLLLKTRL
>sp|P29459|IL12A_HUMAN Interleukin-12 subunit alpha OS = Homo sapiens
OX = 9606
GN = IL 12A PE = 1 SV = 2
(SEQ ID NO: 125)
RNLPVATPDPGMFPCLHHSQNLLRAVSNMLQKARQTLEFYPCTSEEIDHEDITKDKTSTVE
ACLPLELTKNESCLNSRETSFITNGSCLASRKTSFMMALCLSSIYEDLKMYQVEFKTMNAK
LLMDPKRQIFLDQNMLAVIDELMQALNENSETVPQKSSLEEPDFYKTKIKLCILLHAFRIR
AVTIDRVMSYLNAS
>sp|Q9NPF7|IL23A_HUMAN Interleukin-23 subunit alpha OS = Homo sapiens
OX = 9606 GN = IL23A PE = 1 SV = 1
(SEQ ID NO: 126)
RAVPGGSSPAWTQCQQLSQKLCTLAWSAHPLVGHMDLREEGDEETTNDVPHIQCGDGCDPQ
GLRDNSQFCLQRIHQGLIFYEKLLGSDIFTGEPSLLPDSPVGQLHASLLGLSQLLQPEGHH
WETQQIPSLSPSQPWQRLLLRFKILRSLQAFVAVAARVFAHGAATLSP
>sp|Q8NEV9|IL27A_HUMAN Interleukin-27 subunit alpha OS = Homo sapiens
OX = 9606 GN = IL27 PE = 1 SV = 2
(SEQ ID NO: 127)
FPRPPGRPQLSLQELRREFTVSLHLARKLLSEVRGQAHRFAESHLPGVNLYLLPLGEQLPD
VSLTFQAWRRLSDPERLCFISTTLQPFHALLGGLGTQGRWTNMERMQLWAMRLDLRDLQRH
LRFQVLAAGFNLPEEEEEEEEEEEEERKGLLPGALGSALQGPAQVSWPQLLSTYRLLHSLE
LVLSRAVRELLLLSKAGHSVWPLGFPTLSPQP
>sp|Q6EBC2|IL31_HUMAN Interleukin-31 OS = Homo sapiens
OX = 9606 GN = IL31 PE = 1 SV = 1
(SEQ ID NO: 128)
SHTLPVRLLRPSDDVQKIVEELQSLSKMLLKDVEEEKGVLVSQNYTLPCLSPDAQPPNNIH
SPAIRAYLKTIRQLDNKSVIDEIIEHLDKLIFQDAPETNISVPTDTHECKRFILTISQQFS
ECMDLALKSLTSGAQQATT
>sp|Q9UBD9|CLCF1_HUMAN Cardiotrophin-like cytokine factor 1
OS = Homo sapiens OX = 9606 GN = CLCF1 PE = 1 SV = 1
(SEQ ID NO: 129)
LNRTGDPGPGPSIQKTYDLTRYLEHQLRSLAGTYLNYLGPPFNEPDENPPRLGAETLPRAT
VDLEVWRSLNDKLRLTQNYEAYSHLLCYLRGLNRQAATAELRRSLAHFCTSLQGLLGSIAG
VMAALGYPLPQPLPGTEPTWTPGPAHSDFLQKMDDFWLLKELQTWLWRSAKDENRLKKKMQ
PPAAAVTLHLGAHGF
>sp|P13725|ONCM_HUMAN Oncostatin-M OS = Homo sapiens
OX = 9606 GN = OSM PE = 1 SV = 2
(SEQ ID NO: 130)
MAAIGSCSKEYRVLLGQLQKQTDLMQDTSRLLDPYIRIQGLDVPKLREHCRERPGAFPSEE
TLRGLGRRGFLQTLNATLGCVLHRLADLEQRLPKAQDLERSGLNIEDLEKLQMARPNILGL
RNNIYCMAQLLDNSDTAEPTKAGRGASQPPTPTPASDAFQRKLEGCRFLHGYHRFMHSVGR
VFSKWGESPNRSRRHSPHQALRKGVRRTRPSRKGKRLMTRGQLPR
>sp|Q16619| CTF1_HUMAN Cardiotrophin-1 OS = Homo sapiens
OX = 9606 GN = CTF1 PE = 1 SV = 1
(SEQ ID NO: 131)
MSRREGSLEDPQTDSSVSLLPHLEAKIRQTHSLAHLLTKYAEQLLQEYVQLQGDPFGLPSF
SPPRLPVAGLSAPAPSHAGLPVHERLRLDAAALAALPPLLDAVCRRQAELNPRAPRLLRRL
EDAARQARALGAAVEALLAALGAANRGPRAEPPAATASAASATGVFPAKVLGLRVCGLYRE
WLSRTEGDLGQLLPGGSA
>sp|P26441|CNTF_HUMAN Ciliary neurotrophic factor OS = Homo sapiens
OX = 9606 GN = CNTF PE = 1 SV = 1
(SEQ ID NO: 132)
MAFTEHSPLTPHRRDLCSRSIWLARKIRSDLTALTESYVKHQGLNKNINLDSADGMPVAST
DQWSELTEAERLQENLQAYRIFHVLLARLLEDQQVHFTPTEGDFHQAIHTLLLQVAAFAYQ
IEELMILLEYKIPRNEADGMPINVGDGGLFEKKLWGLKVLQELSQWTVRSIHDLRFISSHQ
TGIPARGSHYIANNKKM
>sp|P15018|LIF_HUMAN Leukemia inhibitory factor OS = Homo sapiens
OX = 9606 GN = LIF PE = 1 SV = 1
(SEQ ID NO: 133)
SPLPITPVNATCAIRHPCHNNLMNQIRSQLAQLNGSANALFIL_YYTAQGEPFPNNLDKLC
GPNVTDFPPFHANGTEKAKLVELYRIVVYLGTSLGNITRDQKILNPSALSLHSKLNATADI
LRGLLSNVLCRLCSKYHVGHVDVTYGPDTSGKDVFQKKKLGCQLLGKYKQIIAVLAQAF
>sp|P09919|CSF3_HUMAN Granulocyte colony-stimulating factor
OS = Homo sapiens OX = 9606 GN = CSF3 PE = 1 SV = 1
(SEQ ID NO: 134)
TPLGPASSLPQSFLLKCLEQVRKIQGDGAALQEKLVSECATYKLCHPEELVLLGHSLGIPW
APLSSCPSQALQLAGCLSQLHSGLFLYQGLLQALEGISPELGPTLDTLQLDVADFATTIWQ
QMEELGMAPALQPTQGAMPAFASAFQRRAGGVLVASHLQSFLEVSYRVLRHLAQP

### Proposed Homomeric Foldikine Class I: Long Helix Bundle Subclass.

The foldikines according to these embodiments of the disclosure are synthesised following the scheme depicted in FIG. 26.

In bold, double-underlined we highlight the loop sequence that was opened in the other of the cytokine monomers in order to form a split domain. In italics the split domain molecule fused to the N- and C-termini of the intact monomer. (Xn) are the connecting linkers engineered to link one foldikine domain to the other foldikine domain. Linker sequences are designed to provide a desired structural conformation or are simply a combination of Gly and Ser of different lengths. Beneficially, however, the engineered linker sequences are ‘structured’ linkers that prefer a particular 3D conformation and, therefore, in advantageous embodiments of the disclosure, the engineered linkers do not contain Gly or Ser residues, or beneficially contain 3 or less, 2 or less, or 1 or less Gly or Ser residue. XX_and_YY indicate that extra residues could be added to the N- and C-termini of the split domain at the position of the removed, partially removed or simply opened loop sequence. Furthermore, up to 10 residues right or left respectively at the N- and C-terminals may be removed without affecting activity, depending on the design and structural requirements. Thus, XX and YY are optional sequence elements, which may comprise all of part of the opened loop sequences, and do not form part of the sequences identified by the corresponding SEQ ID NOs below.

It should be appreciated that the illustrated position of these linkers represents an embodiment of the disclosure; but that it may be possible to change the position of these linkers—in particular the position of the linker bridges may change by between 1 and 10 or between 1 and 5 amino acids, such as by 1, 2, 3, 4 or 5 amino acids in either the N-terminal direction or the C-terminal direction without preventing the formation of a functional foldikine. As such, the indicated position of these linkers should be considered to be approximate and in each case could be expanded to incorporate (or remove) some of the surrounding residues. For example, up to 10 amino acids could be deleted on each side of the (Xn) position.

For ease of reference, structurally conserved helices in the family are labelled by the consecutive order they are found in the original WT sequence (ie HA, HB . . . ), and the helical segments by h. When a helical segment is a slightly distorted helix and, therefore, some residues are not assigned as h in swisspdb but is surrounded by regular helical segments we indicate it as h-Nres-h, where Nres could be any number of residues shown as -. The helical segments are defined using the swisspdb software and could vary slightly in length depending on the software used to define them, as well as of the crystal structure.

```
Foldikine-6(IL6_HUMAN Interleukin-6)(XX_SEQ ID NO: 135-(Xn)-SEQ ID NO: 136-(Xn)-
SEQ ID NO: 137_YY)
Sequence opened in one of the cytokine monomers, ESSE, SEQ ID NO: 138
XX_SEEQARAVQMSTKVLIQFLQKKAKNLDAITTPDPTTNASLLTKLQAQNQWLQDMTTHLILRSFKEFLQ
                    hhhhhhhhhhhhhh hhhhhhhhhhhh hhhhhhhhhhhhhhhhhhhh
                                HC HD
SSLRALRQM(Xn)LTSSERIDKQIRYILDGISALRKETCNKSNMCESSKEALAE
hhhhhh hhhhhhhhhhhhhhhhhhhhhhhhhhh
                                                  HA
NNLNLPKMAEKDGCFQSGFNEETCLVKIITGLLEFEVYLEYLQNRFESSEEQARAVQMSTKVLIQFLQKK
                                        hhhhhhhhhhhhhhhhhhhhhhhhh hhhhhhhhhhhhhh
                                                              HB HC
AKNLDAITTPDPTTNASLLTKLQAQNQWLQDMTTHLILRSFKEFLQSSLRALRQM(Xn)
                      hhhhhhhhhhhh hhhhhhhhhhhhhhhhhhhhhhhhh
                                                                        HD
LTSSERIDKQIRYILDGISALRKETCNKSNMCESSKEALAENNLNLPKMAEKDGCFQSGFNEETC
    hhhhhhhhhhhhhhhhhhhhhhhhhhh hhhh
                        HA
LVKIITGLLEFEVYLEYLQNRFES_YY
hhhhhhhhhhhhhhhhhhhh
                HB
Foldikine-11(HUMAN Interleukin 11)(XX_SEQ ID NO: 139-(Xn)-SEQ ID NO: 140-(Xn)-SEQ ID
NO: 141_YY)
Sequence opened in one of the cytokine monomers GSSL, SEQ ID NO: 142
XX_SLKTLEPELGTLQARLDRLLRRLQLLMSRLALPQPPPDPPAPPLAPPSSAWGGIRAAHAILGGLHLTL
                    hhhhhhhhhhhhhhhhhhhhh hhhhhhhhhhhhhhhhhhh
                                      HC HD
DWAVRGLLLLKTRL(Xn)SPDPRAELDSTVLLTRSLLADTRQLAAQLRDKFPADGDHNLDSL
hhhhhhhhhhhhhh hhhhhhhhhhhhhhhhhhhhhhhhhhhhh
                                                                HA
PTLAMSAGALGALQLPGVLTRLRADLLSYLRHVQWLRRAGGSSLKTLEPELGTLQARLDRLLRRLQLLMS
                              hhhhhhhhhhhhhhhhhhhhhhh hhhhhhhhhhhhhhhhhhhhh
                                                      HB HC
RLALPQPPPDPPAPPLAPPSSAWGGIRAAHAILGGLHLTLDWAVRGLLLLKTRL(Xn)SPD
h hhhhhhhhhhhhhhhhhhhhhhhhhhhhhhh
                                                                          HD
PRAELDSTVLLTRSLLADTRQLAAQLRDKFPADGDHNLDSLPTLAMSAGALGALQLPGVLTRLRADLLSYL
hhhhhhhhhhhhhhhhhhhhhhhhhhhhh hhhhhhhhhhhhhhh
                          HA HB
RHVQWLRRAG_YY
hhhhhhhh
Foldikine-12a(HUMAN Interleukin-12 subunit Alpha)(XX_SEQ ID NO: 143-(Xn)-SEQ ID
NO: 144-(Xn)-SEQ ID NO: 145_YY)
Sequence opened in one of the cytokine monomers MDPK, SEQ ID NQ: 146
XX_RQIFLDQNMLAVIDELMQALNENSETVPQKSSLEEPDFYKTKIKLCILLHAFRIRAVTIDRVMSYLNA
            hhhhhhhhhhhhhhh hhhhhhhhhhhhhhhhhhhhhhhhhh
                        HC HD
S(Xn)RNLPVATPDPGMFPCLHHSQNLLRAVSNMLQKARQTLEFYPCTSEEIDHEDITKDKTSTVEACLPLE
                                                    hhhhhhhhhhhhh hhhh
                                                                HA
LTKNESCLNSRETSFITNGSCLASRKTSFMMALCLSSIYEDLKMYQVEFKTMNAKLLMDPKIFLDQNM
                                                        hhhhhhhhhhhhhhhhhhhhhhhhhhhhh hhhhh
                                                                                  HB
LAVIDELMQALNFNSETVPQKSSLEEPDFYKTKIKLCILLHAFRIRAVTIDRVMSYLNAS(Xn)RNLPVATP
hhhhhhhhh hhhhhhhhhhhhhhhhhhhhhhhhhhh
      HC HD
DPGMFPCLHHSQNLLRAVSNMLQKARQTLEFYPCTSEEIDHEDITKDKISTVEACLPLELTKNESCLNSR
                          hhhhhhhhhhhhh hhhh
                                      HA
ETSFITNGSCLASRKTSFMMALCLSSIYEDLKMYQVEFKTMNAKLL_YY
                                  hhhhhhhhhhhhhhhhhhhhhhhhhhhhh
                                                              HB
Foldikine-23a(HUMAN Interleukin-23 subunit Alpha)(XX_SEQ ID NO: 147-(Xn)-SEQ ID
NO: 148-(Xn)-SEQ ID NO: 149_YY)
Sequence opened in one of the cytokine monomers TGEPSLL, SEQ ID NO: 150
XX_LPDSPVGQLHASLLGLSQLLQPEGHHWETQQIPSLSPSQPWQRLLLRFKILRSLQAFVAVAARVFAHG
              hhhhhhhhhhhhhhh hhhhhhhhhhhhhhhhhhhhhhhhhh
AATLSP(Xn)SPAWTQCQQLSQKLCTLAWSAHPLVGHMDLREEGDEETTNDVPHIQCGDGCDPQG
hh hhhhhhhhhhhhhhhh hhh
                                      HA
LRDNSQFCLQRIHQGLIFYEKLLGSDIFTGEPSLLPDSPVGQLHASLLGLSQLLQPEGHHWETQQIPSLS
hh hhhhhhhhhhhhhhhhhhh hhhhhhhhhhhhhhh
                            HB HC
PSQPWQRLLLRFKILRSLQAFVAVAARVFAHGAATLS(Xn)SPAWTQCQQLSQKLCTLAWSAHP
            hhhhhhhhhhhhhhhhhhhhhhhhhhhh hhhhhhhhhhhhhhhh
                                        HD HA
LVGHMDLREEGDEETTNDVPHIQCGDGCDPQGLRDNSQFCLQRIHQGLIFYEKLLGSDIFT_YY
                                                          hhhh hhhhhhhhhhhhhhhhhh
                                                                                            HB
Foldikine-27a(HUMAN Interleukin-27 subunit Alpha)(XX_SEQ ID NO: 151-(Xn)-SEQ ID
NO: 152-(Xn)-SEQ ID NO: 153_YY)
Sequence opened in one of the cytokine monomers QGR
XX_WTNMERMQLWAMRLDLRDLQRHLRFQVLAAGFNLPEEEEEEEEEEEEERKGLLPGALGSALQGPAQVS
          hhhhhhhhhhhhhhhhhhhhhhhhhhhh
WPQLLSTYRLLHSLELVLSRAVRELLLLSKAGH(Xn)LRREFTV
hhhhhhhhhhhhhhhhhhhhhhhhhhhhhh hhhhh
                              HD
SLHLARKLLSEVRGQAHRFAESHLPGVNLYLLPLGEQLPDVSLTFQAWRRLSDPERLCFISTTLQPFHAL
hhhhhhhhhhhhhhhhhhhhhh hhhhh hhhhhhhhhhhhhhhhhh
                    HA HB
LGGLGTQGRWTNMERMQLWAMRLDLRDLQRHLRFQVLAAGENLPEEEEEEEEEEEEERKGLLPGALGSAL
hhhhh hhhhhhhhhhhhhhhhhhhhhhhhhhhh
                                            HC
QGPAQVSWPQLLSTYRLLHSLELVLSRAVRELLLLSKAGH(Xn)
              hhhhhhhhhhhhhhhhhhhhhhhhhhhhhh
                                    HD
LRREFTVSLHLARKLLSEVRGQAHRFAESHLPGVNLYLLPLGEQLPDVSLTFQAWRRLSDPERLCFISTT
    hhhhhhhhhhhhhhhhhhhhhhhhhhh hhhhh hhhhhhhhhhh
                            HA HB
LQPFHALLGGLGT_YY
hhhhhhhhhhhhh
Foldikine-31(HUMAN Interleukin-31)(XX_SEQ ID NO: 154-(Xn)-SEQ ID NO: 155-(Xn)-SEQ ID
NO: 156_YY)
Sequence opened in one of the cytokine monomers DNKS, SEQ ID NO: 157
XX_VIDEIIEHLDKLIFQDAPETNISVPTDTHECKRFILTISQQFSECMDLALKSLTSGAQQATT(Xn)SHTL
      hhhhhhhhhhhh hhhhhhhhhhhhhhhhhhhhhhhhhhhhhhh
                HC HD
PVRLLRPSDDVQKIVEELQSLSKMLLKDVEEEKGVLVSQNYTLPCLSPDAQPPNNIHSPAIRAYLKTIRQL
              hhhhhhhhhhhhhhhhhhhhhhh hhhhhhhhhhhhhh
                                    HA HB
DNKSIDEIIEHLDKLIFQDAPETNISVPTDTHECKRFILTISQQFSECMDLALKSLTSGAQQATT(Xn)S
        hhhhhhhhhh hhhhhhhhhhhhhhhhhhhhhhhhhhhhhhh
                HC HD
HTLPVRLLRPSDDVQKIVEELQSLSKMLLKDVEEEKGVLVSQNYTLPCLSPDAQPPNNIHSPAIRAYLKT
                    hhhhhhhhhhhhhhhhhhhhhhh hhhhhhhhhh
                                          HA HB
IRQL_YY
Hhhh
Foldikine-CLCF1(HUMAN Cardiotrophin-like cytokine factor 1)(XX_SEQ ID NO: 158-(Xn)-
SEQ ID NO: 159-(Xn)-SEQ ID NO: 160_YY)
Sequence opened in one of the cytokine monomers, AAT
XX_AELRRSLAHFCTSLQGLLGSIAGVMAALGYPLPQPLPGTEPTWTPGPAHSDFLQKMDDFWLLKELQTW
      hhhhhhhhhhhhhhhhhhhhhhhhhhhh hhhhhhhhhhhhhhhhhhh
                                HC HD
LWRSAKDFNRLKKKMQPP(Xn)PGPGPSIQKTYDLTRYLEHQLRSLAGTYLNY
hhhhhhhhhhhhhhh hhhhhhhhhhhhhhhhhhhhhhhhhhhh
                                                                        HA
LGPPFNEPDFNPPRLGAETLPRATVDLEVWRSLNDKLRLTQNYEAYSHLLCYLRGLNRQAATAELRRSLA
                                                    hhhhh hhhhhhhhhhhhhhhhhhhhhhhhh hhhhhhhh
                                                                                          HB
HFCTSLQGLLGSIAGVMAALGYPLPQPLPGTEPTWTPGPAHSDFLQKMDDFWLLKELQTWLWRSAKDENR
hhhhhhhhhhhhhhhhhhhh hhhhhhhhhhhhhhhhhhhhhhhhhhh
                HC HD
LKKKMQPP(Xn)PGPGPSIQKTYDLTRYLEHQLRSLAGTYLNYLGPPFNEPDF
hhhhh hhhhhhhhhhhhhhhhhhhhhhhh
                                                        HA
NPPRLGAETLPRATVDLEVWRSLNDKLRLTQNYEAYSHLLCYLRGLNR_YY
                                hhhhh hhhhhhhhhhhhhhhhhhhhhhhh
                                                                      HB
Foldikine-ONCM(HUMAN Oncostatin-M)(XX_SEQ ID NO: 161-(Xn)-SEQ ID NO: 162-(Xn)-SEQ
ID NO: 163_YY)
Sequence opened in one of the cytokine monomers KAQDLERSGLN, SEQ ID NO: 164
XX_NIEDLEKLQMARPNILGLRNNIYCMAQLLDNSDTAEPTKAGRGASQPPTPTPASDAFQRKLEGCRFLH
          hhhhhhhhhhhhhhhhhhhhhhhhhh hhhhhhhhhhhhh
                                  HC HD
GYHRFMHSVGRVFSKWGE(Xn)SCSKEYR
hhhhhhhhhhhhhhh hh
VLLGQLQKQTDLMQDTSRLLDPYIRIQGLDVPKLREHCRERPGAFPSEETLRGLGRRGFLQTLNATLGCV
hhhhhhhhhhhhh hhhhhhh hhhhhhhhh hhhhhh hhhhhhhhhhhhhh
              HA HB
LHRLADLEQRLPKAQDLERSGLNIEDLEKLQMARPNILGLRNNIYCMAQLLDNSDTAEPTKAGRGASQPP
hhhhhhhhhh hhhhhhhhhhhhhhhhhhhhhhhhhh
                                                                        HC
TPTPASDAFQRKLEGCRFLHGYHRFMHSVGRVFSKWGE(Xn)SCSKEYRVLLGQLQKQTDLMQDTSRLLDPYIRIQGLD
              hhhhhhhhhhhhhhhhhhhhhhhhhhhh hhhhhhhhhhhhhhh hhhhhh
                                      HD HA
VPKLREHCRERPGAFPSEETLRGLGRRGFLQTLNATLGCVLHRLADLEQRLPK_YY
  hhhhhhhhh hhhhh hhhhhhhhhhhhhh
                                                                HB
Foldikine-CTF1(HUMAN Cardiotrophin-1)(XX_SEQ ID NO: 165-(Xn)-SEQ ID NO: 166-(Xn)-SEQ
ID NO: 167_YY)
Sequence opened in one of the cytokine monomers NPRA, SEQ ID NO: 168
XX_PRLLRRLEDAARQARALGAAVEALLAALGAANRGPRAEPPAATASAASATGVFPAKVLGLRVCGLYRE
        hhhhhhhhhhhhhhhhhhhhhhhhhhhhhhh hhhhhhhhhhhhhhhhh
                                  HC
WLSRTEGDLGQLLP(Xn)HLEAKIRQTHSLAHLLTKYAEQLLQEYVQL
hhhhhhhhhhh hhhhhhhhhhhhhhhhhhhhhhhhhh
                                                                    HA
QGDPFGLPSFSPPRLPVAGLSAPAPSHAGLPVHERLRLDAAALAALPPLLDAVCRRQAELNPRAPRLLRR
                                                  Hhh hhhhhhhhhhhhhhhhhhhhhhhhhhhhh hhhhh
                                                                                    HB
LEDAARQARALGAAVEALLAALGAANRGPRAEPPAATASAASATGVFPAKVLGLRVCGLYREWLSRTEGD
hhhhhhhhhhhhhhhhhhhhhhhh hhhhhhhhhhhhhhhhhhhhhhhhh
                    HC HD
LGQLLP(Xn)HLEAKIRQTHSLAHLLTKYAEQLLQEYVQLQGDPFGL
hhh hhhhhhhhhhhhhhhhhhhhhhhhhh
                                                      HA
PSFSPPRLPVAGLSAPAPSHAGLPVHERLRLDAAALAALPPLLDAVCRRQAEL_YY
                                                hhhhhhhhhhhhhhhhhhhhhhhhhhhhh
                                                                          HB
Foldikine-CNTF(HUMAN Ciliary neurotrophic factor)(XX_SEQ ID NO: 169-(Xn)-SEQ ID
NO: 170-(Xn)-SEQ ID NO: 171_YY)
Sequence opened in one of the cytokine monomers TPTEGD, SEQ ID NO: 172
XX_FHQAIHTLLLQVAAFAYQIEELMILLEYKIPRNEADGMPINVGDGGLFEKKLWGLKVLQELSQWTVRS
      hhhhhhhhhhhhhhhhhhhhhhhhhh hhhhhhhhhhhhhhhhhhhhh
                              HC HD
IHDLRFISSHQTGIPARGSHYIANNKKM(Xn)PHRRDLCSRSIWLARKIRSDLTALTESYVK
hhhhhhhhh hhhhhhhhhhhhhhhhhhhhhhhhhhhh
                                                                                            HA
HQGLNKNINLDSADGMPVASTDQWSELTEAERLQENLQAYRTFHVLLARLLEDQQVHFTPTEGDFHQAIH
hh hhhhhhhhhhhhhhhhhhhhhhhhhhh hhhhhh
                                                                                  HB
TLLLQVAAFAYQIEELMILLEYKIPRNEADGMPINVGDGGLFEKKLWGLKVLQELSQWTVRSIHDLRFIS
hhhhhhhhhhhhhhhhhhhh hhhhhhhhhhhhhhhhhhhhhhhhhhhhh
                        HC HD
SHQ(Xn)PHRRDLCSRSIWLARKIRSDLTALTESYVKHQGLNKNI
h hhhhhhhhhhhhhhhhhhhhhhhhhhhhhh
                                              HA
NLDSADGMPVASTDQWSELTEAERLQENLQAYRIFHVLLARLLEDQQVHF_YY
                                        hhhhhhhhhhhhhhhhhhhhhhhhhhh
                                                              HB
Foldikine-LIF(HUMAN Leukemia inhibitory factor)(XX_SEQ ID NO: 173-(Xn)-
SEQ ID NO: 174-(Xn)-SEQ ID NO: 175_YY)
Sequence opened in one of the cytokine monomers NPSA, SEQ ID NO: 176
XX_LSLHSKLNATADILRGLLSNVLCRLCSKYHVGHVDVTYGPDTSGKDVFQKKKLGCQLLGKYKQIIAVL
          hhhhhhhhhhhhhhhhhhhhhhhhhh hhhhhhhhhhhhhhhhhhhhh
                                      HC HD
AQAF(Xn)HPCHNNLMNQIRSQLAQLNGSANALFILYYTAQGEPFPNNLDKLCGPNV
                            hhhhhhhhhhhh---hhhhhhhhhhhh
                                                    HA
TDFPPFHANGTEKAKLVELYRIVVYLGTSLGNITRDQKILNPSALSLHSKLNATADILRGLLSNVLCRLC
                      hhhhhhhhhhhhhhhhhhhhhhhhhhhhh hhhhhhhhhhhhhhhhhhhhhhhhhh
                                              HB HC
SKYHVGHVDVTYGPDTSGKDVFQKKKLGCQLLGKYKQIIAVLAQAF(Xn)HPCHNNL
hhh hhhhhhhhhhhhhhhhhhhhh h
                                                              HD
MNQIRSQLAQLNGSANALFILYYTAQGEPFPNNLDKLCGPNVTDFPPFHANGTEKAKLVELYRIVVYLGT
hhhhhhhhhhh---hhhhhhhhhhhh hhhhhhhhhhhhhhhhhh
                      HA HB
SLGNITRDQKIL_YY
hhhhhhhhhhhh
Foldikine-CSF3(HUMAN Granulocyte colony-stimulating factor)(XX_SEQ ID NO: 177-(Xn)-
SEQ ID NO: 178-(Xn)-SEQ ID NO: 179_YY)
Sequence opened in one of the cytokine monomers EGIS, SEQ ID NO: 180
XX_ELGPTLDTLQLDVADFATTIWQQMEELGMAPALQPTQGAMPAFASAFQRRAGGVLVASHLQSFLEVSY
      hhhhhhhhhhhhhhhhhhhhhhhhhhh hhhhhhhhhhhhhhhhhhhhhhh
RVLRHLAQP(Xn)SSLPQSFLLKCLEQVRKIQGDGAALQEKLVSECATYKLCHPEELVLLGHSLGI
hhhhhhh hhhhhhhhhhhhhhhhhhhhhhhhhhhhhhhh
                                                            HA
PWAPLSSCPSQALQLAGCLSQLHSGLFLYQGLLQALEGISPELGPTLDTLQLDVADFATTIWQQMEELGM
                              hhhhhhhhhhhhhhhhhhhh hhhhhhhhhhhhhhhhhhhhhhhhhhhh
                                                HB HC
APALQPTQGAMPAFASAFQRRAGGVLVASHLQSFLEVSYRVLRHLAQP(Xn)SSLPQSFLLKCLEQ
                                hhhhhhhhhhhhhhhhhhhhhhhhhhhhhh hhhhhhhhhhh
                                                        HD
VRKIQGDGAALQEKLVSECATYKLCHPEELVLLGHSLGIPWAPLSSCPSQALQLAGCLSQLHSGLFLYQG
hhhhhhhhhhhhhhhhhhhhh hhhhhhhhhhhhhhhh
                HA HB
LLQAL_YY
hhhhh
```

### Class II Helical Cytokines

The IL10-like class of proteins include IL10, IL19, IL20, IL22, IL24, and IL26. IL28A, IL28B and IL29 (IFNλ), type I interferon (IFNα (IFNA1, IFNA2, IFNA4, IFNA5, IFNA6, IFNA7, IFNA8, IFNA10, IFNA13, IFNA14, IFNA16, IFNA17 and IFNA21), IFNω (IFNW1), IFNε (IFNE), IFNκ (IFNK) and IFNβ (IFNB1)), IFNγ.

Below we list each of the interleukins included in this structural family.

>sp|P22301|IL10_HUMAN Interleukin-10 OS = Homo sapiens
OX = 9606 GN = IL10 PE = 1 SV = 1
(SEQ ID NO: 181)
QSENSCTHFPGNLPNMLRDLRDAFSRVKTFFQMKDQLDNLLLKESLLEDFKGYLGCQALSE
MIQFYLEEVMPQAENQDPDIKAHVNSLGENLKTLRLRLRRCHRFLPCENKSKAVEQVKNAF
NKLQEKGIYKAMSEFDIFINYIEAYMTMKIRN
>sp|Q9UHDO| IL19_HUMAN Interleukin-19 OS = Homo sapiens
OX = 9606 GN = IL 19 PE = 1 SV = 2
(SEQ ID NO: 182)
VDNHGLRRCLISTDMHHIEESFQEIKRAIQAKDTFPNVTILSTLETLQIIKPLDVCCVTKN
LLAFYVDRVFKDHQEPNPKILRKISSIANSFLYMQKTLRQCQEQRQCHCRQEATNATRVIH
DNYDQLEVHAAAIKSLGELDVFLAWINKNHEVMESA
>sp|Q9N_YY1| IL20_HUMAN Interleukin-20 OS = Homo sapiens
OX = 9606 GN = IL20 PE = 1 SV = 2
(SEQ ID NO: 183)
KTLNLGSCVIATNLQEIRNGFSEIRGSVQAKDGNIDIRILRRTESLQDTKPANRCCLLRHL
LRLYLDRVFKNYQTPDHYTLRKISSLANSFLTIKKDLRLCHAHMTCHCGEEAMKKYSQILS
HFEKLEPQAAVVKALGELDILLQWMEETE
>sp|Q9GZX6| IL22_HUMAN Interleukin-22 OS = Homo sapiens
OX = 9606 GN = IL22 PE = 1 SV = 1
(SEQ ID NO: 184)
APISSHCRLDKSNFQQPYITNRTFMLAKEASLADNNTDVRLIGEKLFHGVSMSERCYLMKQ
VLNFTLEEVLFPQSDRFQPYMQEVVPFLARLSNRLSTCHIEGDDLHIQRNVQKLKDTVKKL
GESGEIKAIGELDLLEMSLRNACI
>sp|Q9NPH9|IL26_HUMAN Interleukin-26 OS = Homo sapiens
OX = 9606 GN = IL26 PE = 1 SV = 1
(SEQ ID NO: 185)
HKQSSFTKSCYPRGTLSQAVDALYIKAAWLKATIPEDRIKNIRLLKKKTKKQFMKNCQFQE
QLLSFFMEDVFGQLQLQGCKKIRFVEDFHSLRQKLSHCISCASSAREMKSITRMKRIFYRI
GNKGIYKAISELDILLSWIKKLLESSQ
>sp|Q8IZJQ|IFNL2_HUMAN Interferon lambda-2 OS = Homo sapiens
OX = 9606 GN = IFNL2 PE = 2 SV = 1
(SEQ ID NO: 186)
RGCHIAQFKSLSPQELQAFKRAKDALEESLLLKDCRCHSRLFPRTWDLRQLQVRERPMALE
AELALTLKVLEATADTDPALVDVLDQPLHTLHHILSQFRACIQPQPTAGPRTRGRLHHWLY
RLQEAPKKESPGCLEASVTFNLFRLLTRDLNCVASGDLCV
>sp|Q8IZI9|IFNL3_HUMAN Interferon lambda-3 OS = Homo sapiens
OX = 9606 GN = IFNL3 PE = 1 SV = 2
(SEQ ID NO: 187)
VPVARLRGALPDARGCHIAQFKSLSPQELQAFKRAKDALEESLLLKDCKCRSRLFPRIWDL
RQLQVRERPVALEAELALTLKVLEATADTDPALGDVLDQPLHTLHHILSQLRACIQPQPTA
GPRTRGRLHHWLHRLQEAPKKESPGCLEASVTFNLFRLLTRDLNCVASGDLCV
>sp|Q8IU54|IFNL1_HUMAN Interferon lambda-1 OS = Homo sapiens
OX = 9606 GN = IFNL1 PE = 1 SV = 1
(SEQ ID NO: 188)
GPVPTSKPTTTGKGCHIGRFKSLSPQELASFKKARDALEESLKLKNWSCSSPVFPGNWDLR
LLQVRERPVALEAELALTLKVLEAAAGPALEDVLDQPLHTLHHILSQLQACIQPQPTAGPR
PRGRLHHWLHRLQEAPKKESAGCLEASVTFNLFRLLTRDLKYVADGNLCLRTSTHPEST
>sp|P01562|IFNA1_HUMAN Interferon alpha-1/13 OS = Homo sapiens
OX = 9606 GN = IFNA1 PE = 1 SV = 1
(SEQ ID NO: 189)
DLPETHSLDNRRTLMLLAQMSRISPSSCLMDRHDFGFPQEEFDGNQFQKAPAISVLHELIQ
QIFNLFTTKDSSAAWDEDLLDKFCTELYQQLNDLEACVMQEERVGETPLMNADSILAVKKY
FRRITLYLTEKKYSPCAWEVVRAEIMRSLSLSTNLQERLRRKE
>sp|P01574|IFNB_HUMAN Interferon beta OS = Homo sapiens
OX = 9606 GN = IFNB1 PE = 1 SV = 1
(SEQ ID NO: 190)
MSYNLLGFLQRSSNFQCQKLLWQLNGRLEYCLKDRMNFDIPEEIKQLQQFQKEDAALTIYE
MLQNIFAIFRQDSSSTGWNETIVENLLANVYHQINHLKTVLEEKLEKEDFTRGKLMSSLHL
KR_YYGRILHYLKAKEYSHCAWTIVRVEILRNFYFINRLTGYLRN
>sp|P05000|IFNW1_HUMAN Interferon omega-1 OS = Homo sapiens
OX = 9606 GN = IFNW1 PE = 1 SV = 2
(SEQ ID NO: 191)
LGCDLPQNHGLLSRNTLVLLHQMRRISPFLCLKDRRDFRFPQEMVKGSQLQKAHVMSVLHE
MLQQIFSLFHTERSSAAWNMTLLDQLHTGLHQQLQHLETCLLQVVGEGESAGAISSPALTL
RRYFQGIRVYLKEKKYSDCAWEVVRMEIMKSLFLSTNMQERLRSKDRDLGSS
>sp|Q86WN2|IFNE_HUMAN Interferon epsilon OS = Homo sapiens
OX = 9606 GN = IFNE PE = 2 SV = 1
(SEQ ID NO: 192)
LDLKLIIFQQRQVNQESLKLLNKLQTLSIQQCLPHRKNFLLPQKSLSPQQYQKGHTLAILH
EMLQQIFSLFRANISLDGWEENHTEKFLIQLHQQLEYLEALMGLEAEKLSGTLGSDNLRLQ
VKMYFRRIHDYLENQDYSTCAWAIVQVEISRCLFFVFSLTEKLSKQGRPLNDMKQELTTEF
RSPR
>sp|Q9POWO| IFNK_HUMAN Interferon kappa OS = Homo sapiens
OX = 9606 GN = IFNK PE = 1 SV = 2
(SEQ ID NO: 193)
LDCNLLNVHLRRVTWQNLRHLSSMSNSFPVECLRENIAFELPQEFLQYTQPMKRDIKKAFY
EMSLQAFNIFSQHTFKYWKERHLKQIQIGLDQQAEYLNQCLEEDKNENEDMKEMKENEMKP
SEARVPQLSSLELRRYFHRIDNFLKEKKYSDCAWEIVRVEIRRCL_YYFYKFTALFRRK
>sp|P01579|IFNG_HUMAN Interferon gamma OS = Homo sapiens
OX = 9606 GN = IFNG PE = 1 SV = 1
(SEQ ID NO: 194)
QDPYVKEAENLKKYFNAGHSDVADNGTLFLGILKNWKEESDRKIMQSQIVSFYFKLFKNFK
DDQSIQKSVETIKEDMNVKFFNSNKKKRDDFEKLTNYSVTDLNVQRKAIHELIQVMAELSP
AAKTGKRKRSQMLERGRRASQ

### Proposed Homomeric Foldikine Class II

Homodimeric foldikines of class II cytokines are suitably assembled following the scheme of FIG. 27. In italics the split domain/molecule fused to the N- and C-termini of the intact monomer. (Xn) are the connecting linkers.

In swapped domain proteins such as IL-10 and IFNγ, Xn corresponds to the residues in bold, double-underlined that is the natural sequence equivalent to the designed Sn loops introduced in the monomeric cytokines. Swapped domain foldikines of class II cytokines are assembled following the scheme of FIG. 28.

In non-swapped domain cytokines, (Xn) are the connecting linkers engineered to link one foldikine domain to the other foldikine domain. Linker sequences are designed to provide a desired structural conformation or are simply a combination of Gly and Ser of different lengths. Beneficially, however, the engineered linker sequences are ‘structured’ linkers that prefer a particular 3D conformation and, therefore, in advantageous embodiments of the disclosure the engineered linkers do not contain Gly or Ser residues, or contain 5 or less, beneficially 3 or less, 2 or less, or 1 or less Gly or Ser residue. XX_and_YY indicate that extra residues could be added to the N- and C-termini, or alternatively up to 10 residues right or left respectively of the XX_ and YY_positions at the new N- and C-terminals could be deleted without affecting activity, depending on the design and structural requirements. Thus, XX and YY are optional sequence elements and do not form part of the sequences identified by the corresponding SEQ ID NOs below.

It should be appreciated that the illustrated position of these linkers represents an embodiment of the disclosure; but that it may be possible to change the position of these linkers—in particular the position of the linker bridges may change by between 1 and 10 or between 1 and 5 amino acids, such as by 1, 2, 3, 4 or 5 amino acids in the N-terminal direction, the C-terminal direction, or both without preventing the formation of a functional foldikine. As such, the indicated position of these linkers should be considered to be approximate and in each case could be expanded to incorporate (or remove) some of the surrounding residues. For example, up to 10 amino acids could be deleted on each side of the (Xn) position.

For ease of reference, structurally conserved helices in the family are labelled by the consecutive order they are found in the original WT sequence (i.e. HA, HB . . . ), and the helical segments by h. When a helical segment is a slightly distorted helix and therefore some residues are not assigned as h in swisspdb but is surrounded by regular helical segments we indicate it as h-Nres-h, where Nres could be any number of residues shown as -. The helical segments are defined using the swisspdb software and could vary slightly in length depending on the software used to define them, as well as of the crystal structure. (NtCt) a linker designed to join the N- and C-termini of one monomer to the other.

```
Foldikine-10(IL10_HUMAN Interleukin-10)(XX_SEQ ID NO: 11-(NtCt)-SEQ ID NO: 12-YY)
No loop is removed since it is a natural swapped domain cytokine. Boxes in bold, double-
underlined correspond to the linker sequences that may be swapped with inter-helix linkers of
monomeric cytokines (as indicated below) to make foldikines. Thus, (Xn)in the sequences
below may be the natural sequence indicated below, or may be another designed bridging
sequence.
XX_CTHFPGNLPNMLRDLRDAFSRVKTFFQMKDQLDNLLLKESLLEDFKGYLGCQALSEMIQFYLEEVMPQ
                  hhh hhhhhhhhhhh hhhhhh hhhhhhhhhhhhhhhhhhh
                                          HA HB
AENQDPDIKAHVNSLGENLKTLRLR [symbol] KAVEQVKNAFNKLQEKGIYKAMSEFDIFINY
hhh hhhhhhhhhhhhhhhhhhh hhhhhhhhhhhh hhhhhhh hhhhhh
                              HC HD HE
IEAYMTMKIRN(NtCt)PNMLRDLRDAFSRVKTFFQMKDQLDNLLLKESLLEDFKGYLGCQALSEMIQFYLEEV
hhhhhhhh hhhhhhhhhhh hhhhhh hhhhhhhhhhhhhhhh
      HF HA HB
MPQAENQDPDIKAHVNSLGENLKTLRLR [symbol] KAVEQVKNAFNKLQEKGIYKAMSEFDIF
hhhhhh hhhhhhhhhhhhhhhhhhh hhhhhhhhhhhh hhhhhhh hhh
                                HC HD HE
INYIEAYMTM_YY
Hhhhhhhhhh
HF
Foldikine-19(HUMAN Interleukin-19)(XX_SEQ ID NO: 195-(Xn)-SEQ ID NO: 196-(NtCt)-SEQ ID
NO: 197-(Xn)-SEQ ID NO: 198_YY)
(loop removed CQQCR; SEQ ID NO: 199)
XX_VDNHGLRRCLISTDMHHIEESFQEIKRAIQAKDTFPNVTILSTLETLQIIKPLDVCCVTKNLLAFYVD
                                    hhhhhhhh----hhhh hh hhhhhhhhhhhhhhhhh
                                                    HA HB
RVFKDHQEPNPKILRKISSIANSFLYMQKTLRQ(Xn)QEATNATRVIHDNYDQLEVHAAAIKSLGELDVFLA
hhhhhh hhhhhhhhhhhhhhhhhhhhhhh hhhhhhhhhhhhhhh hhhhhhhhh hhhhh
                                            HC HD HE HF
WINKNHEVMFSA(NtCt)VDNHGLRRCLISTDMHHIEESFQEIKRAIQAKDTFPNVTILSTLETLQIIKP
hhhhh hhhhhhh----hhhh hh h
                                                                                HA
LDVCCVTKNLLAFYVDRVFKDHQEPNPKILRKISSIANSFLYMQKTLRQ(Xn)QEATNATRVIHDNYDQLEV
hhhhhhhhhhhhhhhhhhhhh hhhhhhhhhhhhhhhhhhhhhhh hhhhhhhhhhhhhhh h
                    HB HC HD
HAAAIKSLGELDVFLAWINKNHEVMFSA_YY
hhhhhhh hhhhhhhhhh
      HE HF
Foldikine-20(HUMAN Interleukin-20)(XX_SEQ ID NO: 200-(Xn)-SEQ ID NO: 201-(NtCt)-SEQ ID
NO: 202-(Xn)-SEQ ID NO: 203_YY)
(loop removed MTCHC; SEQ ID NO: 204)
XX_KTLNLGSCVIATNLQEIRNGFSEIRGSVQAKDGNIDIRILRRTESLQDTKPANRCCLLRHLLRLYLDR
                                  hhhhhhhh---hhhhh hhhhhhhhhhhhhhhhh
                                                  HA HB
VFKNYQTPDHYTLRKISSLANSFLTIKKDLRLCHAH(Xn)GEEAMKKYSQILSHFEKLEPQAAVVKALGELD
                  hhhhhhhhhhhhhhhhhhhhhhhhhhh hhhhhhhhhhhhhhh hhhhhhh hh
                                              HC HD HE
ILLQWMEETE(NtCt)KTLNLGSCVIATNLQEIRNGFSEIRGSVQAKDGNIDIRILRRTESLQDTKPANR
hhhhhhh hhhhhhhh---hhhhh hhhh
      HF HA
CCLLRHLLRLYLDRVFKNYQTPDHYTLRKISSLANSFLTIKKDLRLCHAH(Xn)GEEAMKKYSQILSHFEKL
hhhhhhhhhhhhh hhhhhhhhhhhhhhhhhhhhhhhhhhh hhhhhhhhhhhhhhhh
          HB HC HD
EPQAAVVKALGELDILLOWMEETE_YY
    hhhhhhh hhhhhhhhh
          HE HF
Foldikine-22(HUMAN Interleukin-22)(XX_SEQ ID NO: 205-(Xn)-SEQ ID NO: 206-(NtCt)-SEQ ID
NO: 207-(Xn)-SEQ ID NO: 208_YY)
(loop removed STCHIEGDDL; SEQ ID NO: 209)
XX_CRLDKSNFQQPYITNRTFMLAKEASLADNNTDVRLIGEKLFHGVSMSERCYLMKQVLNFTLEEVLFPQ
                          hhhhhhhhhhhhhhhhh hhh hhhhhhhhhhhhh---hhhh
                                        HA HB
SDRFQPYMQEVVPFLARLSNRL(Xn)HIQRNVQKLKDTVKKLGESGEIKAIGELDLLFMSLRNACI(NtCt)
          hhhhhhhhhhhhhhhhh hhhhhhhhhhhhhhh hhhhhhh hhhhhhhhhhh
                          HC HD HE HF
APISSHCRLDKSNFQQPYITNRTFMLAKEASLADNNTDVRLIGEKLFHGVSMSERCYLMKQVLNFTLEE
                                hhhhhhhhhhhhhhhhh hhh hhhhhhhhhhhhh
                                              HA HB
VLFPQSDRFQPYMQEVVPFLARLSNRL(Xn)HIQRNVQKLKDTVKKLGESGEIKAIGELDLLFMSLRNACI-YY
  hhhh hhhhhhhhhhhhhhhh hhhhhhhhhhhhhhh hhhhhhh hhhhhhhhhhh
                                    HC HD HE HF
Foldikine-26(HUMAN Interleukin-26)(XX_SEQ ID NO: 210-(Xn)-SEQ ID NO: 211-(NtCt)-SEQ ID
NO: 212-(Xn)-SEQ ID NO: 213_YY)
(loop removed SCASSARE; SEQ ID NO: 214)
XX_HKQSSFTKSCYPRGTLSQAVDALYIKAAWLKATIPEDRIKNIRLLKKKTKKQFMKNCQFQEQLLSFFM
                              hhhhhhhhhhhhhhhhhhhh hhhhhhh hhhhhhhhhhhh
                                              HA HB
EDVFGQLQLQGCKKIRFVEDFHSLRQKLSHCI(Xn)MKSITRMKRIFYRIGNKGIYKAISELDILLSWIKKL
hhhhhh hhhhhhhhhhhhhhhhhhh hhhhhhhhhhhh hhhhhhh hhhhhhhhhh
                                      HC HD HE HF
LESSQ(NtCt)HKQSSFTKSCYPRGTLSQAVDALYIKAAWLKATIPEDRIKNIRLLKKKTKKQFMKNCQF
hhh hhhhhhhhhhhhhhhhhhhh hhhhhhh hhh
                                                            HA
QEQLLSFFMEDVFGQLQLOGCKKIRFVEDFHSLRQKLSHCI(Xn)MKSITRMKRIFYRIGNKGIYKAISELD
hhhhhhhhhhhhhhh hhhhhhhhhhhhhhhhhhh hhhhhhhhhhhh hhhhhhh h
            HB HC HD HE
ILLSWIKKLLESSQ_YY
hhhhhhhhhhhh
        HF
Foldikine-L2(HUMAN Interferon lambda-2)(XX_SEQ ID NO: 215-(Xn)-SEQ ID NO: 216-
(NtCt)-SEQ ID NO: 217-(Xn)-SEQ ID NO: 218_YY)
(loop removed IQPQPTAGPRTRG; SEQ ID NO: 219)
XX_RGCHIAQFKSLSPQELQAFKRAKDALEESLLLKDCRCHSRLFPRTWDLRQLQVRERPMALEAELALTL
            hhh hhhhhhhhhhhhhhhhhhh hhhhhhhhhhhhhhhh
                                              HA HB
KVLEATADTDPALVDVLDQPLHTLHHILSQFRAC(Xn)RLHHWLYRLQEAPKKESPGCLEASVTFNLFRLLTR
hhhhhhhh hhhhhhhhhhhhhhhhhhhhhhh hhhhhhhhhhhhhh hhhhhhhhhhhhhhhhhh
                                          HC HD HE HF
DLNCVASGDLCV(NtCt)RGCHIAQFKSLSPQELQAFKRAKDALEESLLLKDCRCHSRLFPRTWDLRQLQ
hhhhhh hhh hhhhhhhhhhhhhhhhhhh
                                                                            HA
VRERPMALEAELALTLKVLEATADTDPALVDVLDQPLHTLHHILSQFRAC(Xn)RLHHWLYRLQEAPKKESPG
hhhhhhhhhhhhhhhhhhhhhhhh hhhhhhhhhhhhhhhhhhhhhhh hhhhhhhhhhhhhh hh
                    HB HC HD
CLEASVTFNLFRLLTRDLNCVASGDLCV_YY
hhhhhhhhhhhhhhhhhhhhhhh
      HE HF
Foldikine-L3(HUMAN Interferon lambda-3)(XX_SEQ ID NO: 220-(Xn)-SEQ ID NO: 221-
(NtCt)-SEQ ID NO: 222-(Xn)-SEQ ID NO: 223_YY)
(loop removed IQPQPTAGPRTRGR; SEQ ID NO: 224)
XX_RGCHIAQFKSLSPQELQAFKRAKDALEESLLLKDCKCRSRLFPRTWDLRQLQVRERPVALEAELALTL
            hhh hhhhhhhhhhhhhhhhhhh hhhhhhhhhhhhhhhh
                                              HA HB
KVLEATADTDPALGDVLDQPLHTLHHILSQLRAC(Xn)RLHHWLHRLQEAPKKESPGCLEASVTFNLFRLLTR
hhhhhhhh hhhhhhhhhhhhhhhhhhhhhhh hhhhhhhhhhhhhh hhhhhhhhhhhhhhhhhh
                                          HC HD HE-HF
DLNCVASGDLCV(NtCt)RGCHIAQFKSLSPQELQAFKRAKDALEESLLLKDCKCRSRLFPRTWDLRQLQ
hhhhhh hhh hhhhhhhhhhhhhhhhhhh
                                                                          HA
VRERPVALEAELALTLKVLEATADTDPALGDVLDQPLHTLHHILSQLRAC(Xn)RLHHWLHRLQEAPKKESPG
hhhhhhhhhhhhhhhhhhhhhhhh hhhhhhhhhhhhhhhhhhhhhhh hhhhhhhhhhhhhh hh
                    HB HC HD
CLEASVTFNLFRLLTRDLNCVASGDLCV_YY
hhhhhhhhhhhhhhhhhhhhhhh
                HE-HF
Foldikine-L1(HUMAN Interferon lambda-1)(XX_SEQ ID NO: 225-(Xn)-SEQ ID NO: 226-
(NtCt)-SEQ ID NO: 227-(Xn)-SEQ ID NO: 228_YY)
(loop removed IQPQPTAGPRTRGR; SEQ ID NO: 229)
XX_TGKGCHIGRFKSLSPQELASFKKARDALEESLKLKNWSCSSPVFPGNWDLRLLQVRER
                                  hhhhhhhhhhhhhhhhh
                                                HA
PVALEAELALILKVLEAAAGPALEDVLDQPLHTLHHILSQLQAC(Xn)LHHWLHRLQEAPKKESAGCLEASV
hhhhhhhhhhhhhhhhh hhhhhhhhhhhhhhhhhhhhhh hhhhhhh hhhhhhhh
              HB HC HD HE
TFNLFRLLTRDLKYVADGNLCLRTSTHPEST(NtCt)TGKGCHIGRFKSLSPQELASFKK
h hhhhhhhhhhhh hhhhhhhhh
                HF
ARDALEESLKLKNWSCSSPVFPGNWDLRLLQVRERPVALEAELALTLKVLEAAAGPALEDVLDQPLHTLH
hhhhhhhhh hhhhhhhhhhhhhhhhh hhhhhhhhhhhhhhh
        HA HB HC
HILSQLQAC(Xn)LHHWLHRLQEAPKKESAGCLEASVTFNLFRLLTRDLKYVADGNLCLRTSTHPEST_YY
hhhhhhhh hhhhhhh hhhhhhhhh hhhhhhhhhhhh
                                HD HE HF
Foldikine-A1(HUMAN Interferon alpha-1/13)(XX_SEQ ID NO: 230-(Xn)-SEQ ID NO: 231-
(NtCt)-SEQ ID NO: 232-(Xn)-SEQ ID NO: 233_YY)
(loop removed MQEERVGETPLMN; SEQ ID NO: 234)
XX_SLDNRRTLMLLAQMSRISPSSCLMDRHDFGFPQEEFDGNQFQKAPAISVLHELIQQIFNLFT
          hhhhhhhhhhh hhhhhhhhhhhhh
                    HA HB
TKDSSAAWDEDLLDKFCTELYQQLNDLEACV(Xn)ADSILAVKKYFRRITLYLTEKKYSPCAWEVVRAEIMR
hhhhhh hhhhhhhhhhhhhhhhhhh hhhhhhhhhhhhhhhhhhh hhhhhhhhhhhhh
                                  HC HD HE-HF
SLSLSTNLQERLRRKE(NtCt)SLDNRRTLMLLAQMSRISPSSCLMDRHDFGFPQEEFDGNQFQ
hhhhh hhhhhhhhhhh
                                                        HA
KAPAISVLHELIQQIFNLFTTKDSSAAWDEDLLDKFCTELYQQLNDLEACV(Xn)ADSILAVKKYFRRITLY
          hhhhhhhhhhhh hhhhh hhhhhhhhhhhhhhhhhhh hhhhhhhhhhhhhhh
                    HB HC HD
LTEKKYSPCAWEVVRAEIMRSLSLSINLQERLRRKE_YY
hhhh hhhhhhhhhhhhhhhhhh
                          HE-HF
Foldikine-A2(HUMAN Interferon alpha-2 OS = Homo sapiens
OX = 9606 GN = IFNA2 PE = 1
SV = 1(XX_SEQ ID NO: 235-(Xn)-SEQ ID NO: 236-(NtCt)-SEQ ID NO: 237-(Xn)-SEQ ID NO:
238_YY)
(loop removed IQGVGVTETPLMK; SEQ ID NO: 239)
XX_SLGSRRILMLLAQMRKISLFSCLKDRHDFGFPQEEFGNQFQKAETIPVLHEMIQQIFNLFSTKDSSAA
          hhhhhhhhhhh hhhhhhhhhhhhh hhhhhh
                  HA HB
WDETLLDKFYTELYQQLNDLEACV(Xn)EDSILAVRKYFQRITLYLKEKKYSPCAWEVVRAEIMRSFSLSIN
    hhhhhhhhhhhhhhhhhhh hhhhhhhhhhhhhhhhhhh hhhhhhhhhhhhhhhhhh
                  HC HD HE-HF
LQESLRSKE(NtCt)SLGSRRTLMLLAQMRKISLFSCLKDRHDFGFPQEEFGNQFQKAETIPVLHEMIQQ
                                  hhhhhhhhhhh hhhhhhhhh
                                            HA HB
IFNLFSTKDSSAAWDETLLDKFYTELYQQLNDLEACV(Xn)EDSILAVRKYFQRITLYLKEKKYSPCAWEVV
hhh hhhhh hhhhhhhhhhhhhhhhhhh hhhhhhhhhhhhhhhhhhh hhhhhhh
                                                HC HD HE-HF
RAEIMRSFSLSIN _YY
hhhhhhhhh
Foldikine-IFNB(HUMAN Interferon beta)(XX_SEQ ID NO: 240-(Xn)-SEQ ID NO: 241-(NtCt)-
SEQ ID NO: 242-(Xn)-SEQ ID NO: 243_YY)
(loop removed KEDFTRGK; SEQ ID NO: 244)
XX_MSYNLIGFLQRSSNFQCQKLLWQLNGRLEYCLKDRMNFDIPEEIKQLQQFQKEDAALTIYEMLONIFA
          hhhhhhhhhhhhhhhhhhh hhhh hhhh hhhhhhhhhhhhhhhhh
                            HA HB
IFRQDSSSIGWNETIVENLLANVYHQINHLKTVLEEKLE(Xn)LMSSLHLKRYYGRILHYLKAKEYSHCAWTIVRVE
hh hh hhhhhhhhhhhhhhhhhhhhhhhhhh hhhhhhhhhhhhhhh hhhhhhhhhh
                                                  HC HD
ILRNFYFINRLTGYLRN(NtCt)MSYNLLGFLQRSSNFQCQKLLWQLNGRLEYCLKDRMNFDIPEEIKQL
hhhhhhhhhhhhh hhhhhhhhhhhhhhhhhhh hhhh hhhh
        HE-HF HA
QQFQKEDAALTIYEMLQNIFAIFRQDSSSTGWNETIVENLLANVYHQINHLKTVLEEKLE(Xn)LMSSLHLKRYYG
        hhhhhhhhhhhhhhhhhhh hhhhhhhhhhhhhhhhhhhhhhhhhh hhhhhh
                        HB HC
RILHYLKAKEYSHCAWTIVRVEILRNFYFINRLIGYLRN_YY
hhhhhhhhh hhhhhhhhhhhhhhhhhhhhhhh
        HD HE-HF
Foldikine-IFNW1(HUMAN Interferon omega-1)(XX_SEQ ID NO: 245-(Xn)-SEQ ID NO:
246-(NtCt)-SEQ ID NO: 247-(Xn)-SEQ ID NO: 248_YY)
(loop removed LLQVVGEGESAGAIS; SEQ ID NO: 249)
XX_LGCDLPQNHGLLSRNTLVLLHQMRRISPFLCLKDRRDFRFPQEMVKGSQLQKAHVMSVLHEMLQQIFS
                        hhhhhhhhhhhhh hhhhhhhhhhhhhhhhh
                                  HA HB
LFHTERSSAAWNMTLLDQLHTGLHQQLQHLETC(Xn)SPALTLRRYFQGIRVYLKEKKYSDCAWEVVRMEIM
      hhhhh hhhhhhh---hhhh hhhhhhhhhhhhhhhhh hhhhhhhhhhhh
                                        HC HD HE-HF
KSLFLSTNMQ(NtCt)CDLPQNHGLLSRNTLVLLHQMRRISPFLCLKDRRDFRFP
hhhhhhh hhhhhhhhhhhhh
                                                            HA
QEMVKGSQLQKAHVMSVLHEMLQQIFSLFHTERSSAAWNMTLLDQLHTGLHQQLQHLETC(Xn)SPALTLRR
                    hhhhhhhhhhhhhhhhh hhhhh hhhhhhh---hhhh hhhhh
                                    HB HC
YFQGIRVYLKEKKYSDCAWEVVRMEIMKSLFLSINMQ_YY
hhhhhhhhhhhh hhhhhhhhhhhhhhhhhhh
          HD HE-HF
Foldikine-IFNE(HUMAN Interferon epsilon)(XX_SEQ ID NO: 250-(Xn)-SEQ ID NO: 251-
(NtCt)-SEQ ID NO: 252-(Xn)-SEQ ID NO: 253_YY)
(loop removed GLEAEKLSGTLG; SEQ ID NO: 254)
XX_LDLKLIIFQQRQVNQESLKLLNKLQTLSIQQCLPHRKNELLPQKSLSPQQYQKGHTLAILHEMLQQIF
      hhhhhhhhhhhhhhhhhhhhhh hhhhhhhhhhhhhhhh
SLFRANISLDGWEENHTEKFLIQLHQQLEYLEALM(Xn)SDNLRLQVKMYFRRIHDYLENQDYSTCAWAIVQ
hhhh hhhhhhhhhhhhhhhhhhhh hhhhhhhhhhhhhhhhhhhhh hhhhhhhh
VEISRCLFFVFSLTEKLSKQGRPLNDMKQELTTEFRSPR(NtCt)LDLKLIIFQQRQVNQESLKLLNKLQ
hhhhhhhhhhhhhhhhhhhh hhhhhhhhhh hhhhhhhhhhhhhhhhhhhhh
TLSIQQCLPHRKNFLLPQKSLSPQQYQKGHTLAILHEMLQQIFSLFRANISLDGWEENHTEKFLIQLHQQ
                                                    Hhhhhhhhhhhhhhhhhhh hhhhhhhhhhhh
LEYLEALM(Xn)SDNLRLQVKMYFRRIHDYLENQDYSTCAWAIVQVEISRCLFFVFSLTEKLSKQGRPLNDM
hhhhhhhh hhhhhhhhhhhhhhhhhhhhh hhhhhhhhhhhhhhhhhhhhhhhhhh hhh
KQELTTEFRSPR_YY
hhhhhhh
Foldikine-IFNK(HUMAN Interferon kappa)(XX_SEQ ID NO: 255-(Xn)-SEQ ID NO: 256-
(NtCt)-SEQ ID NO: 257-(Xn)-SEQ ID NO: 258_YY)
(loop removed EEDKNENEDMKEMKENEMKPSEAR; SEQ ID NO: 259)
XX_LDCNLLNVHLRRVTWQNLRHLSSMSNSFPVECLRENIAFELPQEFLQYTQPMKRDIKKAFYEMSLQAF
                hhhhhhhhhhhhhhhhh hhh hhhhhhhhhhhhhhhh
                              HA HB
NIFSQHTFKYWKERHLKQIQIGLDQQAEYLNQCL(Xn)VPQLSSLELRRYFHRIDNFLKEKKYSDCAWEIVR
hhh hhhhhhhhhhhhhhhhhhhhh hhhhhhhhhhhhhhhhhhhhhh hhhhhhhh
                                              HC HD
VEIRRCL_YYFYKFTALFRRK(NtCt)LDCNLLNVHLRRVTWQNLRHLSSMSNSFPVECLRENIAFELPQE
hhhhhhhhhhhhhhhh hhhhhhhhhhhhhhhhh hhh
        HE-HF HA
FLQYTQPMKRDIKKAFYEMSLQAFNIFSQHTFKYWKERHLKQIQIGLDQQAEYLNQCL(Xn)VPQLSSLELR
              hhhhhhhhhhhhhhhhhhh hhhhhhhhhhhhhhhhhhhhh hhhhhhhhh
                              HB HC
RYFHRIDNFLKEKKYSDCAWEIVRVEIRRCL_YYFYKFTALFRRK_YY
hhhhhhhhhhhhh hhhhhhhhhhhhhhhhhhhhhhhh
          HD HE-HF
Foldikine-IFNG(HUMAN Interferon gamma)(XX-SEQ ID NO: 14-(NtCt)-SEQ ID NO: 15-YY)
NO LOOP REMOVED SINCE IT IS A NATURAL SWAPPED DOMAIN IL
XX_QDPYVKEAENLKKYFNAGHSDVADNGTLFLGILKNWKEESDRKIMQSQIVSFYFKLFKNFKDDQSIQK
          hhhhhhhhhhhh hh hhhhh hhhhhhhhhhhhhhhhhhhh hh
                    HA HB
SVETIKEDMNVKFFNSNKKKRDDFEKLTNYSVTDLNVQRKAIHELIQVMAELSPMLF(NtCt)
hhhhhhhhhhhhhh hhhhhhhhhhh hhhhhhhh hhhhhh
            HC HD HE HF
MQDPYVKEAENLKKYFNAGHSDVADNGTLFLGILKNWKEESDRKIMQSQIVSFYFKLFKNFKDD
          hhhhhhhhhhhhh hhhhh hhhhhhhhhhhhhhhhhhhh
                    HA HB
QSIQKSVETIKEDMNVKFFNSNKKKRDDFEKLINYSVTDLNVQRKAIHELIQVMAELSPAAKTGKRKRSQ
      hhhhhhhhhhhhhhhh hhhhhhhhhh hhhhhhh---hhhhhh
                      HC HD HE HF
MLFRGRRASQ_YY
Foldikine-IFNG(HUMAN Interferon gamma)(ORKIFNg-002)(SEQ ID NO: 18)
GSQDPYVKEAENLKKYFNAGHSDVADNGTLFLGILKNWKEESDRKIMQSQIVSFYFKLFKNFKDDQSIQK
          hhhhhhhhhhhh hh hhhhh hhhhhhhhhhhhhhhhhhhh hh
                      HA HB
SVETIKEDMNVKFFNSNKKKRDDFEKLTNYSVTDLNVQRKAIHELIQVMASKPHPGQLWA
hhhhhhhhhhhhhh hhhhhhhhhhh hhhhhhhh hhhhhh
          HC HD HE HF
YVKEAENLKKYFNAGHSDVADNGTLFLGILKNWKEESDRKIMQSQIVSFYFKLFKNFKDDQSIQKSVETI
hhhhhhhhhhhh hh hhhhh hhhhhhhhhhhhhhhhhhhh hhhhhhh
      HA HB HC
KEDMNVKFFNSNKKKRDDFEKLTNYSVTDLNVQRKAIHELIQVMAELSPAA
hhhhhhhhh hhhhhhhhhhh hhhhhhhh hhhhhh
HC HD HE HF
```

### Class I and Class II Heterodimeric Foldikines

Single molecule heterodimeric foldikines may be formed between a class I cytokine and a class II cytokine following the schemes indicated in FIGS. 6A and 6B.

A) Class I cytokines that may be combined with class II cytokines to form heterodimeric foldikines according to various embodiments of the construction scheme of FIG. 6A include short-helix bundle cytokines, such as IL2, IL3, IL4, IL7, IL9, IL15, IL21, TSLP, GMCS-F, CSF1, and CSF2; and long-helix bundle cytokines, such as of IL6, IL11, IL12A, IL23A, IL27A, IL31, CLCF1, CNTF, CTF1, LIF, OSM and CSF3.

Class II cytokines that may be combined with class I cytokines to form heterodimeric foldikines according to various embodiments of the construction scheme of FIG. 6A include the IL10-like class of proteins IL19, IL20, IL22, IL24, and IL26; IL28A, IL28B and IL29 (IFNλ); type I interferon (IFNα (IFNA1, IFNA2, IFNA4, IFNA5, IFNA6, IFNA7, IFNA8, IFNA10, IFNA13, IFNA14, IFNA16, IFNA17 and IFNA21); IFNω (IFNW1); IFNE (IFNE); IFNκ (IFNK); IFNβ (IFNB1)) and IFNγ.

### Class I/Class II Foldikine Heterodimers

Foldikines according to this aspect and various embodiments of the disclosure are formed by linking the N-terminus of one monomer covalently to the C-terminus of the other monomer and vice versa to create a single polypeptide (see black lines, top right panel; FIG. 6A). In addition, a linker region between adjacent α-helices in one of the original monomer domains (see black line; bottom right panel; FIG. 6A) is opened to create new N- and C-termini (N_N-ter and N-C-ter; bottom left panel; FIG. 6A) in the foldikine. New N- and C-termini are created in the original cytokine type I sequence.

By way of example, a heterodimeric IL-2/IL-22 polypeptide according to one embodiment of this aspect is illustrated below (SEQ ID NO: 197). In this sequence, underscored we show the split IL-2 polypeptide sequence and in italics the ‘continuous’ IL-22 sequence. In bold the loop of IL-2 that will be opened to create new N- and C-termini for the new foldikine protein. The exact position of the opening and the residues to be kept could vary and include any number of the bold residues, as described previously.

```
IL-2/IL-22(Foldikine2-22_6A; XX_SEQ ID NO: 260-(Xn)-SEQ ID NO: 261-(Xn)-
SEQ ID NO: 262_YY)
XX-FHLRPRDLISNINVIVLELKGSETTFMCEYADETATIVEFLNRWITFCQSIISTLT(Xn)
                hhhhhhhhhhhhhh hhhhhhhhhhhhhhhhhh
                            HC HD
CRLDKSNFQQPYITNRTFMLAKEASLADNNTDVRLIGEKLFHGVSMSERCYLMKQVLNFTLEEVLFPQ
                    hhhhhhhhhhhhhhhhh hhh hhhhhhhhhhhhh---hhhh
SDRFQPYMQEVVPFLARLSNRLSTCHIEGDDLHIQRNVQKLKDTVKKLGESGEIKAIGELDLLFMSLRNACI
          Hhhhhhhhhhhhhhhhh hhhhhhhhhhhhhhhh hhhhhhh hhhhhhhhhhh
                        HC HD HE HF
(Xn)TSSSTKKTQLQLEHLLLDLQMILNGINNYKNPKLTRMLTFKFYMPKKATELKHLQCLEEELKPLEEVLNLAQSKN-YY
            hhhhhhhhhhhhhhhhhhhhhhhhh hhhhhh hhh hhhhhhh---hhhhhh
                                HA HB
```

B) Class I cytokines that may be combined with class II cytokines to form heterodimeric foldikines according to various embodiments of the construction scheme of FIG. 6B include short-helix bundle cytokines, such as IL2, IL3, IL4, IL7, IL9, IL15, IL21 and GMCS-F; and long-helix bundle cytokines, such as of IL6, IL11, IL12A, IL23A, IL27A, IL31, CLCF1, CNTF, CTF1, LIF, OSM and CSF3.

Class II cytokines that may be combined with class I cytokines to form heterodimeric foldikines according to various embodiments of the construction scheme of FIG. 6B include the IL10-like class of proteins IL19, IL20, IL22, IL24, and IL26; IL28A, IL28B and IL29 (IFNλ); type I interferon (IFNα (IFNA1, IFNA2, IFNA4, IFNA5, IFNA6, IFNA7, IFNA8, IFNA10, IFNA13, IFNA14, IFNA16, IFNA17 and IFNA21); IFNω (IFNW1); IFNE (IFNE); IFNκ (IFNK); IFNβ (IFNB1)) and IFNγ.

Two different schemes may be employed according to whether the split domain cytokine monomer is provided by the class I or the class II cytokine.

### Class I/Class II Foldikine Heterodimers

In the first example a class I foldikine is split and a continuous class II cytokine is inserted between the split N- and C-terminal portions of the class I cytokine. According to these embodiments, the class I and the class II cytokine are inverted in comparison to the structure of the wild type class I and class II cytokines, respectively, in that the C-terminal portion of the wild type cytokine forms the N-terminal portion of the corresponding foldikine portion/domain, and the N-terminal portion of the wild type cytokine forms the C-terminal portion of the corresponding foldikine portion/domain.

Foldikines according to this aspect and various embodiments of the disclosure are formed by opening a linker region/loop in the class I cytokine to create new N- and C-termini; for example, between the second and third α-helices. Similarly, a linker region/loop in the class II cytokine is opened to create new N- and C-terminal ends for fusing to the original N- and C-terminal ends of the class I cytokine. Next, linkers (Xn) are used to connect the original C-terminal end of the class I cytokine to the new N-terminal end of the class II cytokine, and the original N-terminal end of the class I cytokine to the new C-terminal end of the class II cytokine. The loop that is opened in the class I cytokine is shown in bold in the sequence below.

By way of example, a heterodimeric IL-2/IL-22 polypeptide according to this embodiment is illustrated below (SEQ ID NO: 198). In this sequence, in italics we show the split IL-2 polypeptide sequence and in underscored the ‘continuous’ (inverted) IL-22 sequence. In bold the loop of IL-2 that will be opened to create new N- and C-termini for the new foldikine protein. The exact position of the opening and the residues to be kept could vary and include any number of the bold residues, as described previously.

```
IL-2/IL-22 (Foldikine2-22_6B; XX_SEQ ID NO: 263-(Xn)-SEQ ID NO: 264-(NtCt)-
SEQ ID NO: 265-(Xn)-SEQ ID NO: 266_YY) [symbol]
X-FHLRPRDLISNINVIVLELKGSETTFMCEYADETATIVEFLNRWITFCQSIISTLT(Xn)LHIQRNVQKLKDTVKK
                hhhhhhhhhhhhhh hhhhhhhhhhhhhhhhhh hhhhhhhhhhhhhhhh
                              HC HD HD
LGESGEIKAIGELDLLFMSLRNACI(NtCt)CRLDKSNFQQPYITNRTFMLAKEASLADNNTDVRLIGEKLFHGVSMSE
      hhhhhhh hhhhhhhhhhh hhhhhhhhhhhhhhhhh hhh
            HE HF HA
RCYLMKQVLNFTLEEVLFPQSDRFQPYMQEVVPFLARLSNRL(Xn)TSSSTKKTQLQLEHLLLDLQMILNGINNYKNP
hhhhhhhhhhhhh hhhh hhhhhhhhhhhhhhhh hhhhhhhhhhhhhhhhhhhhhhhhh
            HB HC HA
KLTRMLTFKFYMPKKATELKHLQCLEEELKPLEEVLNLA [symbol]
hhhhhh hhh hhhhhhh---hhhhhh
                            HB
```

In the second example a class II foldikine is split and a continuous class I cytokine is inserted between the split N- and C-terminal portions of the class II cytokine. According to these embodiments, neither the class I nor the class II cytokine is inverted, such that the order of α-helices is according to that of the corresponding wild type cytokines (with the exception of the split monomer domain).

Foldikines according to this aspect and various embodiments of the disclosure are formed by opening a linker region/loop in the class II cytokine to create new N- and C-termini; for example, between the third and fourth α-helices. Next, linkers (Xn) are used to connect the new C- and N-termini of the split class II cytokine sequences to the original N- and C-termini, respectively of the continuous class I cytokine domain.

By way of example, a heterodimeric IL-22/IL-2 polypeptide according to this embodiment is illustrated below (SEQ ID NO: 199). In this sequence, in italics we show the split IL-22 polypeptide sequence and in underscored the ‘continuous’ IL-2 sequence.

```
IL-22/IL-2 (Foldikine 22-2_6B; XX_SEQ ID NO: 267-(Xn)-SEQ ID NO: 268-(Xn)-
SEQ ID NO: 269_YY)
IL-22 is split and shown as italic residues.
IL-2 is continuous and showed as underscored residues.
XX_CRLDKSNFQQPYITNRTFMLAKEASLADNNTDVRLIGEKLFHGVSMSERCYLMKQVLNFTLEEVLFPQ
                          hhhhhhhhhhhhh hhhh hhhhhhhhhhhhh---hhhh
                                          HA HB
SDRFQPYMQEVVPFLARLSNRL(Xn)TSSSTKKTQLQLEHLLLDLQMILNGINNYKNPKLTRMLTFKFYMPKKATEL
          hhhhhhhhhhhhhhhhh hhhhhhhhhhhhhhhhhhhhhhhhh hhhhhh hhh
                        HC HA
KHLQCLEEELKPLEEVLNLAQSKNFHLRPRDLISNINVIVLELKGSETTFMCEYADETATIVEFLNRWITFCQSIISTLT(Xn)
hhhhhhh---hhhhhh hhhhhhhhhhhhhh hhhhhhhhhhhhhhhhhh
                HB HC HD
LHIQRNVQKLKDTVKKLGESGEIKAIGELDLLFMSLRNACI-YY
hhhhhhhhhhhhhhhh hhhhhhh hhhhhhhhhhh
              HD HE HF
```

### Example 4: Design of Linkers in Different Foldikine Scaffolds and Identification of Loops to be Opened

### Methods

### Generation of Circular Permutants

The general protocol to generate circular permutants for interleukins is done by, first IntraLinker design over the corresponding structure, closing natural NC termini (using Bridging command from ModelX). This first step is common for each different interleukin and it creates a structural model represented by a continuous sequence. A circular permutant is then created by breaking a peptidic-bond on the continuous sequence and renumbering it starting from the new Nterm (breaking residue). Selection of different breaking residues results in different permutants. Breaking residues selected are those having smaller stabilizing properties in each crystallographic structure.

### PDB Structures Used:

For IL-2 we used structure PDB for protein modelling 5lqb, for IL-22 6weo, for IFNb 1au1 and for GMCSF 2gmf.

### IntraLinker Design Using ModelX and FoldX Software:

The new connectivity linking the natural Nt and Ct of the target ILs (IntraLinker) were designed with the aid of the ModelX tool suite. The ModelX Bridging command (Cross-Linking mode) was used; it connects a pair of residues selected as anchors with all geometrically compatible fragments from a custom-made protein fragment library (PepXDB_5k). Bridging command allows the user to select different peptide lengths; the output is an ensemble of bridged models where linkers/connections with forbidden phi and psi dihedrals in the Ramachandran plot or having backbone clashes are discarded.

When peptides are longer than numeric positions between the anchors, Bridging renumbers surplus residues with res codes that are not recognised by FoldX in further modelling steps. For this reason, the design of a circular permutant required a numerical rearrangement of the monomeric protein residues after opening at an indicated position and linking the natural Nt and Ct. Bridging also replaces all fringe residues between the anchors with the ones from the peptide.

For closing NC termini of a given structure, extensive linker screening was performed by running the models through the Bridging algorithm with exhaustive combinations of anchoring points in an overlapping sliding window around the flanks of the regions to be joined. Every window was queried for different peptide lengths (6 to 20 aa). The bridged models are side chain repaired using the RepairPDB command of FoldX (Delgado et al, 2019), and resulting models were ranked by global energy (FoldX Stability command). Then a position scan mutagenesis (PSSM) from FoldX was run on the linker and adjacent residues to identify mutations that optimize packing with the IL and the corresponding receptors.

### Plasmid Generation

All plasmids generated in this work were assembled following the Gibson method (Gibson et al, Nat Methods 6, 343-345 (2009). When required, Integrated DNA Technologies (IDT) Corporation performed gene synthesis (gBlock double-stranded fragments) and oligonucleotides synthesis. Gene amplification was carried out with Phusion DNA polymerase (Thermo Fisher Scientific) and transformed in Escherichia coli DH5-alpha competent cells (NEB).

For mammalian protein expression in ExpiCHO cells, the vector used was pcDNA3.1 (V790-20, Invitrogen). For bacterial protein expression using Mycoplasma pneumoniae M129 cells, transposons derived from Tn4001 (Lyon et al., Mol Gen Genet. 1984; 193 (3): 554-6) were used. In the case of Mycoplasma pneumoniae we used the P3 synthetic promoter (Yus et al, Nat Commun. 2017 Aug. 28; 8 (1): 368) and the secretion signal for M. pneumoniae (s142) (patent U.S. Pat. No. 10,745,450) was used for all to all variants.

All the plasmids were verified by Sanger sequencing (Eurofins Genomics).

### Protein Expression Using ExpiCHO System

ExpiCHO protein expression kit cells were purchased from Thermofisher (A29133). For a low-scale production (2.5 mL), 24 deep well plates were used (AXYPDW10ML24CS, Merck) and covered by gas-permeable film (ThermoFisher). The day-1 of production, ExpiCHO-cells were split to a final density of 3×10⁶ viable cells/mL. Grown at 37° C., 8% CO₂ and shaking at 110 rpm. The Day 0 of production, ExpiCHO cells were split to a final density of 3×10⁶ viable cells/ml per sample and aliquots of 2.5 mL of the cell suspension were plated per plate well. Per sample, we prepared 2 μg of DNA in 200 μl of cold OptiPRO SFM with 9.0 μL ExpiFectamine CHO Reagent (complexation mixture) and added to each plate well. The plate was covered with a gas permeable seal and incubated at 37° C., 8% CO₂ and 225 rpm. The day 1 after 18-22 hours post-transfection, ExpiFectamine CHO Enhancer and ExpiCHO Feed was added to each well. For an entire plate of 24 wells, 400 μL ExpiFectamine CHO Enhancer and 16 mL ExpiCHO Feed in a conical tube were mixed. From this mixture, 600 μL were added to each well of the plate. Then the plates were incubated at 37° C., 8% CO₂ and 225 rpm for 4 days and then harvested.

### Protein Quantification by ELISA

Protein production of the proteins of interest for Mycoplasma pneumoniae M129-expressed or ExpiCHO-expressed was quantified by ELISA. Each of the different experiments were performed following the manufacturer instructions for IFN-beta (Human IFN-beta DuoSet ELISA, Biotechne), IL-10 (ELISA MAX™ Deluxe Set Human IL-10, Biolegend), IL-22 (ELISA MAX™ Deluxe Set Human IL-22, Biolegend), and IL-2 (ELISA MAX™ Deluxe Set Human IL-2, Biolegend).

### Protein Quantification by Mass Spectroscopy

For IFN-β variants and IL-22 variants, ELISA results for protein quantification have been verified by mass spectrometry. For sample preparation, In solution digestion samples were reduced with dithiothreitol (30 nmols, 1 h, 37° C.) and alkylated in the dark with iodoacetamide (60 nmol, 30 min, 25° C.). The resulting protein extract was first diluted 1/3 with 200 mM NH₄HCO₃ and digested with 1 μg LysC (Wako, cat #129-02541) overnight at 37° C. and then diluted 1/2 and digested with 1 μg of trypsin (Promega, cat #V5113) for eight hours at 37° C. After digestion, peptide mix was acidified with formic acid and desalted with a MicroSpin C18 column (The Nest Group, Inc) prior to LC-MS/MS analysis. For chromatographic and mass spectrometric analysis, samples were analyzed using a Orbitrap Eclipse mass spectrometer (Thermo Fisher Scientific, San Jose, CA, USA) coupled to an EASY-nLC 1200 (Thermo Fisher Scientific (Proxeon), Odense, Denmark). Peptides were loaded directly onto the analytical column and were separated by reversed-phase chromatography using a 50-cm column with an inner diameter of 75 μm, packed with 2 μm C18 particles spectrometer (Thermo Scientific, San Jose, CA, USA).

Chromatographic gradients started at 95% buffer A and 5% buffer B with a flow rate of 300 nl/min and gradually increased to 25% buffer B and 75% A in 79 min and then to 40% buffer B and 60% A in 11 min. After each analysis, the column was washed for 10 min with 100% buffer B. Buffer A: 0.1% formic acid in water. Buffer B: 0.1% formic acid in 80% acetonitrile (January 2019).

The mass spectrometer was operated in positive ionization mode with nanospray voltage set at 2.4 kV and source temperature at 305° C. The acquisition was performed in data-dependent adquisition (DDA) mode and full MS scans with 1 micro scans at resolution of 120,000 were used over a mass range of m/z 350-1400 with detection in the Orbitrap mass analyzer. Auto gain control (AGC) was set to ‘standard’ and injection time to ‘auto’. In each cycle of data-dependent acquisition analysis, following each survey scan, the most intense ions above a threshold ion count of 10000 were selected for fragmentation. The number of selected precursor ions for fragmentation was determined by the “Top Speed” acquisition algorithm and a dynamic exclusion of 60 seconds. Fragment ion spectra were produced via high-energy collision dissociation (HCD) at normalized collision energy of 28% and they were acquired in the ion trap mass analyzer. AGC was set to 2E4, and an isolation window of 0.7 m/z and a maximum injection time of 12 ms were used. Digested bovine serum albumin (New England biolabs cat #P8108S) was analyzed between each sample to avoid sample carryover and to assure stability of the instrument and QCloud (Chiva et al. (2018) PLOS One. January 11; 13 (1)) has been used to control instrument longitudinal performance during the project. For data Analysis, the acquired spectra were analysed using the Proteome Discoverer software suite (v2.0, Thermo Fisher Scientific) and the Mascot search engine (v2.6, Matrix Science; Perkins, et al. (1999) Electrophoresis. December; 20 (18): 3551-67). The data were searched against a Swiss-Prot Chinese Hamster reference database (as in November 2022, 23885 entries) plus a list (Beer et al (2017) Methods Mol Biol. 1619:339-352) of common contaminants and all the corresponding decoy entries. For peptide identification a precursor ion mass tolerance of 7 ppm was used for MS1 level, trypsin was chosen as enzyme, and up to three missed cleavages were allowed. The fragment ion mass tolerance was set to 0.5 Da for MS2 spectra. Oxidation of methionine and N-terminal protein acetylation were used as variable modifications whereas carbamidomethylation on cysteines was set as a fixed modification. False discovery rate (FDR) in peptide identification was set to a maximum of 5%.

Peptide quantification data were retrieved from the “Precursor Ion Quantifier” node from Proteome Discoverer (v2.5) using 2 ppm mass tolerance for the peptide extracted ion current (XIC).

### HEK Blue IL Reporter Cell Culture

The HEK-Blue™ cell lines carrying a SEAP reporter construct were purchased from InvivoGen (InvivoGen, San Diego, CA, USA). In this work we used IFN-α/B Reporter HEK 293 Cells (hkb-ifnab), Human IL-2 & IL-15 Reporter Cells (hkb-il2bg), Human & Mouse IL-22 Reporter Cells (hkb-il22) and Human IL-10 Reporter Cells (hkb-il10). We used HEK-Dual cells (two reporter: SEAP and Lucia) for IFN-gamma detection (Human IFN-γ SEAP/Luciferase Reporter Cells, hkd-ifng).

Cells were grown in DMEM (Lonza, BE12-604F) supplemented with 10% FBS, 2 mM L-glutamine and specified selection antibiotics by the manufacturer in each of the different reporter cells. Cells were passed when 70% confluence was reached, following the manufacturer's recommendation.

### Colorimetric Analysis in HEK-Blue™ Cells

After supernatant quantification by ELISA, samples were adjusted to fit into the dynamic range of the HEK reporter cells using DMEM media for performing the different dilutions. Each of the top concentration was set for each reporter cell to match saturation, and 8 serial dilutions were performed (0.5× each) for each sample. Then, HEK reporter cells were prepared according to manufacturer instructions. Briefly, 180 μL of cells per well were seeded in a 96-well plate (Nunc Microwell, ThermoFisher Scientific, #167008). From this, 20 μL of each supernatant with adjusted concentration was added, and cells were kept between 20-24 h at 37° C. and 5% CO₂ (induced HEK-Blue™). After, 180 μL of QUANTI-Blue Solution (Alkaline phosphatase detection medium, #repqbs, InvivoGen) was mixed with 20-μL induced HEK-Blue™ cells in a new 96-well plate. Cells were then incubated 60 min at 37° C., and absorbance (630 nm) was measured in the spectrophotometer Tecan i-control, 2.0.10.0.

### Inferring EC-50 Data

For each of the conditions, the concentration in molar was calculated taking into consideration the molecular weight. For the ‘Foldikines’, it was considered that both subunits of the protein were active and therefore we used the molecular weight of the monomer when it is a homo-foldikine (two domains the same). When the foldikine is a hetero-foldikine we determined the abundance using an antibody against one of the foldikines and we considered the molecular weight of each IL to determine the molarity. The different absorbance measures with subtracted background were fitted to a Michaelis Menten equation using GraphPad Prism 9 Software

### Results

Way B of Making Foldikine—Class I Cytokine (e.g. IL-2)

The list of variants tested was as follows:
IL2 WT (wild-type IL-2 expressed and secreted by eukaryote cells, i.e. CHO cells)
ORK2-006: foldikine-2 connected via two 6 aa InterLinkers with the aa sequence GPPPPG (SEQ ID NO:270).
ORK2-007: foldikine-2 connected via two 7 aa InterLinkers with the aa sequence GPPPPPG (SEQ ID NO: 271).
ORK2-008: foldikine-2 connected via two 8 aa InterLinkers with the aa sequence GPPPPPPG (SEQ ID NO: 272).
ORK2-009: foldikine-2 connected via two 6 aa InterLinkers with the aa sequence GGGSGG (SEQ ID NO: 273).
ORK2-010: foldikine-2 connected via two 7 aa InterLinkers with the aa sequence GGGSGGG (SEQ ID NO: 274).
ORK2-011: foldikine-2 connected via two 8 aa InterLinkers with the aa sequence GGGSGGGG (SEQ ID NO: 275).

The amino acid sequences used were the following (canonical α-helices, only, are labelled; underlined sequence is secretion signal for M. pneumoniae):

```
>IL2 WT
  (SEQ ID NO: 79)
MYRMQLLSCIALSLALVTNSAPTSSSTKKTQLQLEHLLLDLQMILNGINNYKNPKLTRMLTFKFYMPKKA
                                                    hhhhhhhhhhhhhhhhhhhhhh hhhhhh
                                                                        HA
TELKHLQCLEEELKPLEEVLNLAQSKNFHLRPRDLISNINVIVLELKGSETTFMCEYADETATIVEFLNR
      hhhhhhh--- hhhhhhhh hhhhh-hhhhhhhhh hhhhhhh
                  HB HC HD
WITFCQSIISTLT*
hhhhhhhhhhh
        HD
>ORK2-006 PolyPro 6 aa linker
  (SEQ ID NO: 313)
MYRMQLLSCIALSLALVTNSLRPRDLISNINVIVLELKGSETTFMCEYADETATIVEFLNRWITFCQSII
                                            hhhhh-hhhhhhhhhh hhhhhhhhhhhhhhhh
                                                            HC HD
STLIGPPPPGTKKTQLQLEHLLLDLQMILNGINNYKNPKLTRMLTFKFYMPKKATELKHLQCLEEELKP
h hhhhhhhhhhhhhhhhhhhhhh hhhhhhh---hhh
                                          HA HB
LEEVLNLAQSKNFHLRPRDLISNINVIVLELKGSETTFMCEYADETATIVEFLNRWITFCQSIISTLIGP
hhhh hhhhh-hhhhhhhhhh hhhhhhhhhhhhhhhhh
  HB HC HD
PPPGTKKTQLQLEHLLLDLQMILNGINNYKNPKLTRMLTFKFYMPKKATELKHLQCLEEELKPLEEVLN
          Hhhhhhhhhhhhhhhhhhhhhh hhhhhhhhhh---hhhh
                                  HA HB
LAQS*
>ORK2-007 PolyPro 7 aa linker
  (SEQ ID NO: 276)
MYRMQLLSCIALSLALVTNSLRPRDLISNINVIVLELKGSETTFMCEYADETATIVEFLNRWITFCQSII
                                            hhhhh-hhhhhhhhhh hhhhhhhhhhhhhhhh
                                                        HC HD
STLIGPPPPPGTKKTQLQLEHLLLDLQMILNGINNYKNPKLTRMLTFKFYMPKKATELKHLQCLEEELKP
h hhhhhhhhhhhhhhhhhhhhhh hhhhhhh---hhh
                                          HA HB
LEEVLNLAQSKNFHLRPRDLISNINVIVLELKGSETTFMCEYADETATIVEFLNRWITFCQSIISTLIGP
hhhh hhhhh-hhhhhhhhhh hhhhhhhhhhhhhhhhh
  HB HC HD
PPPPGTKKTQLQLEHLLLDLQMILNGINNYKNPKLTRMLTFKFYMPKKATELKHLQCLEEELKPLEEVLN
          Hhhhhhhhhhhhhhhhhhhhhh hhhhhhhhhh---hhhh
                                  HA HB
LAQS*
>ORK2-008 PolyPro 8 aa linker
(SEQ ID NO: 277)
MYRMQLLSCIALSLALVTNSLRPRDLISNINVIVLELKGSETTFMCEYADETATIVEFLNRWITFCQSII
                                            hhhhh-hhhhhhhhhh hhhhhhhhhhhhhhhh
                                                            HC HD
STLIGPPPPPPGTKKTQLQLEHLLLDLQMILNGINNYKNPKLTRMLTFKFYMPKKATELKHLQCLEEELK
h hhhhhhhhhhhhhhhhhhhhhh hhhhhhh---hh
                                            HA HB
PLEEVLNLAQSKNFHLRPRDLISNINVIVLELKGSETTFMCEYADETATIVEFLNRWITFCQSIISTLTG
hhhhh hhhhh-hhhhhhhhhh hhhhhhhhhhhhhhhhh
      HB HC HD
PPPPPPGTKKTQLQLEHLLLDLQMILNGINNYKNPKLTRMLTFKFYMPKKATELKHLQCLEEELKPLEEV
              hhhhhhhhhhhhhhhhhhhhhh hhhhhhh---hhhhhhh
                                HA HB
LNLAQS*
>ORK2-009 PolyGly 6aa linker
  (SEQ ID NO: 278)
MYRMQLLSCIALSLALVTNSLRPRDLISNINVIVLELKGSETTFMCEYADETATIVEFLNRWITFCQSII
                                            hhhhh-hhhhhhhhhh hhhhhhhhhhhhhhhh
                                                            HC HD
STLIGGGSGGTKKTQLQLEHLLLDLQMILNGINNYKNPKLTRMLTFKFYMPKKATELKHLQCLEEELKPL
h hhhhhhhhhhhhhhhhhhhhhh hhhhhhh---hhhh
                                            HA HB
EEVLNLAQSKNFHLRPRDLISNINVIVLELKGSETTFMCEYADETATIVEFLNRWITFCQSIISTLIGGG
hhh hhhhh-hhhhhhhhhh hhhhhhhhhhhhhhhhh
  HB HC HD
SGGTKKTQLQLEHLLLDLQMILNGINNYKNPKLTRMLTFKFYMPKKATELKHLQCLEEELKPLEEVLNLA
      hhhhhhhhhhhhhhhhhhhhhh hhhhhhh---hhhhhhh
                                HA HB
QS*
>ORK2-010 PolyGly 7aa linker
  (SEQ ID NO: 279)
MYRMQLLSCIALSLALVTNSLRPRDLISNINVIVLELKGSETTFMCEYADETATIVEFLNRWITFCQSII
                                            hhhhh-hhhhhhhhhh hhhhhhhhhhhhhhhh
                                                            HC HD
STLIGGGSGGGTKKTQLQLEHLLLDLQMILNGINNYKNPKLTRMLTFKFYMPKKATELKHLQCLEEELKP
h hhhhhhhhhhhhhhhhhhhhhh hhhhhhh---hh
                                            HA HB
LEEVLNLAQSKNFHLRPRDLISNINVIVLELKGSETTFMCEYADETATIVEFLNRWITFCQSIISTLIGG
hhhh hhhhh-hhhhhhhhhh hhhhhhhhhhhhhhhhh
  HB HC HD
GSGGGTKKTQLQLEHLLLDLQMILNGINNYKNPKLTRMLTFKFYMPKKATELKHLQCLEEELKPLEEVLN
          hhhhhhhhhhhhhhhhhhhhhh hhhhhhh---hhhhhhh
                                HA HB
LAQS*
>ORK2-011 PolyGly 8 aa linker
  (SEQ ID NO: 280)
MYRMQLLSCIALSLALVTNSLRPRDLISNINVIVLELKGSETTFMCEYADETATIVEFLNRWITFCQSII
                                            hhhhh-hhhhhhhhhh hhhhhhhhhhhhhhhh
                                                            HC HD
STLTGGGSGGGGTKKTQLQLEHLLLDLQMILNGINNYKNPKLTRMLTFKFYMPKKATELKHLQCLEEELK
h hhhhhhhhhhhhhhhhhhhhhh hhhhhhh---h
                                            HA HB
PLEEVLNLAQSKNFHLRPRDLISNINVIVLELKGSETTFMCEYADETATIVEFLNRWITFCQSIISTLIG
hhhhh hhhhh-hhhhhhhhhh hhhhhhhhhhhhhhhhh
  HB HC HD
GGSGGGGTKKTQLQLEHLLLDLQMILNGINNYKNPKLTRMLTFKFYMPKKATELKHLQCLEEELKPLEEV
              hhhhhhhhhhhhhhhhhhhhhh hhhhhhh---hhhhhhh
                                HA HB
LNLAQS*
```

### Results

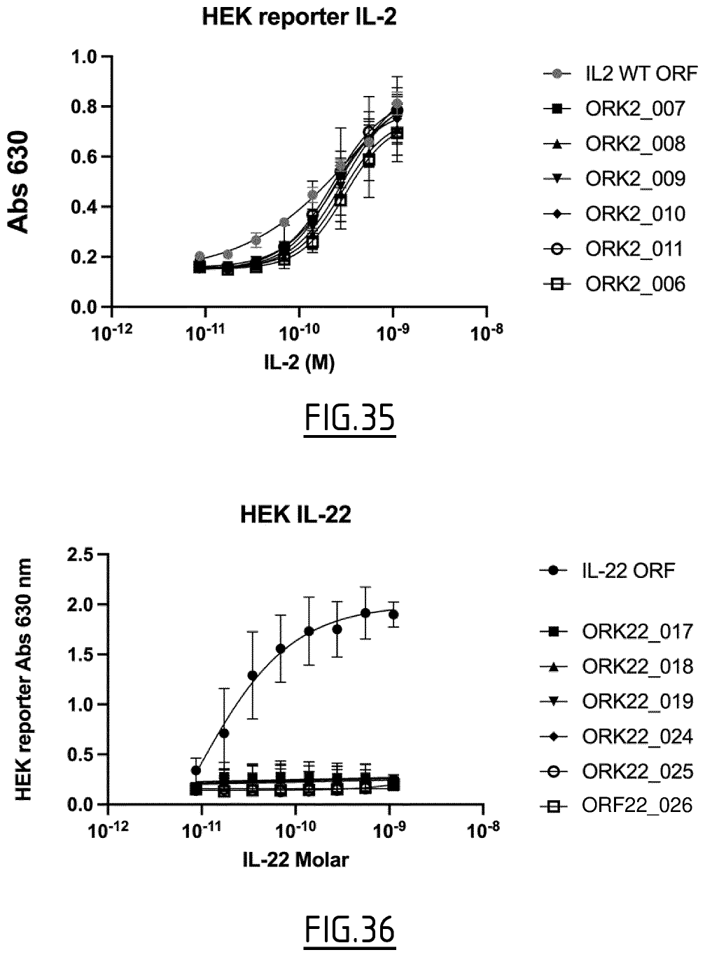

The different constructs were transiently transformed into ExpiCHO cells as described in Methods. ExpiCHO cells were grown as described in Methods and after 4 days the supernatants were collected and the concentration of the different engineered molecules calculated by ELISA. Then, we performed serial dilutions starting from 20 ng/ml (and diluting 0.5× each) and assessed the activation of IL-2 reporter HEK blue cells (see FIG. 35). We fitted a Michaelis Menten mathematical model to the experimental data (see methods) and obtained the EC-50 (Table 14).

TABLE 14
Fitting parameters for each of the mutants. Proteins have been expressed
using CHO as expression system. Vmax has been set to 0.9.

| | IL2 WT | ORK2_007 | ORK2_008 | ORK2_009 | ORK2_010 | ORK2_011 | ORK2_006 |
|---|---|---|---|---|---|---|---|
| Vmax | =0.9000 | =0.9000 | =0.9000 | =0.9000 | =0.9000 | =0.9000 | =0.9000 |
| EC-50EC-50 | 1.211E−10 | 1.837E−10 | 2.507E−10 | 2.114E−10 | 1.903E−10 | 1.706E−10 | 2.848E−10 |

The above results suggest that, in the case of IL-2, a non-designed flexible (polyGly) or rigid linker (PolyPro) does not seem to be important.

Way A of Making Foldikine—Class II Cytokine (e.g. IL-22)

The amino acid sequences used were the following (canonical α-helices, only, are labelled; IL-22 signal peptide has been replaced by M. pneumoniae signal peptide—see Methods):

```
>ORK22-016 short 1aa Gly linker, Nt-Ct linker in bold italic
(SEQ ID NO: 281)
                                                                  ARLDKSNFQQPYITNRTFMLAKEASLADNNTDVRLIG
                                                                                      hhhhhhhhhhhhhhhhh
                                                                                                      HA
EKLFHGVSMSERCYLMKQVLNFTLEEVLFPQSDRFQPYMQEVVPFLARLSGHIQRNVQKLKDTVKKLGES
                        hhhhhhhhhhhhh hhhhhhhhhhhhh hhhhhhhhhhhhhhh h
                                    HB HC HD
GEIKAIGELDLLFMSLRNACIANGVLDKSNFQQPYITNRTFMLAKEASLADNNTDVRLIGEKLFHGVSMS
hhhhhh hhhhhhhhhhhh hhhhhhhhhhhhhhhhh
    HE HF HA
ERCYLMKQVLNFTLEEVLFPQSDRFQPYMQEVVPFLARLSNRLSGHIQRNVQKLKDTVKKLGESGEIKAI
    hhhhhhhhhhhhh hhhhhhhhhhhhhhhhh hhhhhhhhhhhhhhh hhhhhhh
                  HB HC HD HE
GELDLLFMSLRNACI*
      hhhhhhhhhhhh
                HF
>ORK22-017 short 3aa Gly linker, Nt-Ct linker in bold italic underlined
(SEQ ID NO: 282)
                                                                  ARLDKSNFQQPYITNRTFMLAKEASLADNNTDVRLIG
                                                                                      hhhhhhhhhhhhhhhhh
                                                                                                    HA
EKLFHGVSMSERCYLMKQVLNFTLEEVLFPQSDRFQPYMQEVVPFLARLSGGGHIQRNVQKLKDTVKKLG
                        hhhhhhhhhhhhh hhhhhhhhhhhhhh hhhhhhhhhhhhhhh
                                    HB HC HD
ESGEIKAIGELDLLFMSLRNACIANGVLDKSNFQQPYITNRTFMLAKEASLADNNTDVRLIGEKLFHGVS
  hhhhhhh hhhhhhhhhhhh hhhhhhhhhhhhhhhhh
        HE HF HA
MSERCYLMKQVLNFTLEEVLFPQSDRFQPYMQEVVPFLARLSNRLSGGGHIQRNVQKLKDTVKKLGESGE
        Hhhhhhhhhhhhh hhhhhhhhhhhhhhhhh hhhhhhhhhhhhhhh hhh
                      HB HC HD HE
IKAIGELDLLFMSLRNACI*
hhhh hhhhhhhhhhh
HE HF
>ORK22-018 long 6aa polyGly linker, Nt-Ct linker in bold italic underlined
(SEQ ID NO: 283)
                                                                  ARLDKSNFQQPYITNRTFMLAKEASLADNNTDVRLIG
                                                                                      hhhhhhhhhhhhhhhhh
                                                                                                    HA
EKLFHGVSMSERCYLMKQVLNFTLEEVLFPQSDRFQPYMQEVVPFLARLSGGGSGGHIQRNVQKLKDTVK
                        hhhhhhhhhhhhh hhhhhhhhhhhhhh hhhhhhhhhhhhhh
                                      HB HC HD
KLGESGEIKAIGELDLLFMSLRNACIANGVLDKSNFQQPYITNRTFMLAKEASLADNNTDVRLIGEKLFH
h hhhhhhh hhhhhhhhhhh hhhhhhhhhhhhhhhhh
            HE HF HA
GVSMSERCYLMKQVLNFTLEEVLFPQSDRFQPYMQEVVPFLARLSNRLSGGGSGGHIQRNVQKLKDTVKK
              hhhhhhhhhhhh hhhhhhhhhhhhhhhhh hhhhhhhhhhhhhhh
                      HB HC HD
LGESGEIKAIGELDLLFMSLRNACI*
      hhhhhhh hhhhhhhhhhh
            HE HF
>ORK22-019 long 9aa polyGly linker, Nt-Ct linker in bold italic underlined
(SEQ ID NO: 284)
                                                                  ARLDKSNFQQPYITNRTFMLAKEASLADNNTDVRLIG
                                                                                      hhhhhhhhhhhhhhhhh
                                                                                                          HA
EKLFHGVSMSERCYLMKQVLNFTLEEVLFPQSDRFQPYMQEVVPFLARLSGGGSGGSGGHIQRNVQKLKD
                        hhhhhhhhhhhhh hhhhhhhhhhhhhh hhhhhhhhhhh
                                      HB HC HD
TVKKLGESGEIKAIGELDLLFMSLRNACIANGVLDKSNFQQPYITNRTFMLAKEASLADNNTDVRLIGEK
hhhh hhhhhhh hhhhhhhhhhh hhhhhhhhhhhhhhhhh
HD HE HF HA
LFHGVSMSERCYLMKQVLNFTLEEVLFPQSDRFQPYMQEVVPFLARLSNRLSGGGSGGSGGHIQRNVQKL
                    Hhhhhhhhhhhhh hhhhhhhhhhhhhhhhh hhhhhhhhh
                                HB HC HD
KDTVKKLGESGEIKAIGELDLLFMSLRNACI*
hhhhhh hhhhhhh hhhhhhhhhhh
      HD HE HF
>ORK22-020 long 12aa polyGly linker, Nt-Ct linker in bold italic underlined
(SEQ ID NO: 285)
                                                                  ARLDKSNFQQPYITNRTFMLAKEASLADNNTDVRLIG
                                                                                      hhhhhhhhhhhhhhhhh
                                                                                                HA
EKLFHGVSMSERCYLMKQVLNFTLEEVLFPQSDRFQPYMQEVVPFLARLSGGGSGGSGGSGGHIQRNVQK
                        Hhhhhhhhhhhhh hhhhhhhhhhhhhh hhhhhhhh
                                    HB HC HD
LKDTVKKLGESGEIKAIGELDLLFMSLRNACIANGVLDKSNFQQPYITNRTFMLAKEASLADNNTDVRLI
hhhhhhh hhhhhhh hhhhhhhhhhh hhhhhhhhhhhhhhhhh
      HD HE HF HA
GEKLFHGVSMSERCYLMKQVLNFTLEEVLFPQSDRFQPYMQEVVPFLARLSNRLSGGGSGGSGGSGGHIQ
                          hhhhhhhhhhhhh hhhhhhhhhhhhhhhhh hhh
                                        HB HC HD
RNVQKLKDTVKKLGESGEIKAIGELDLLFMSLRNACI*
hhhhhhhhhhhhs hhhhhhh hhhhhhhhhhh
        HD HE HF
>ORK22-024 PolyPro 3aa linker. Nt-Ct linker in bold italic and underlined
(SEQ ID NO: 286)
                                                                  ARLDKSNFQQPYITNRTFMLAKEASLADNNTDVRLIG
                                                                                      hhhhhhhhhhhhhhhhh
                                                                                                    HA
EKLFHGVSMSERCYLMKQVLNFTLEEVLFPQSDRFQPYMQEVVPFLARLSGPGHIQRNVQKLKDTVK
                        Hhhhhhhhhhhhh hhhhhhhhhhhhh hhhhhhhhhhhhhh
                                        HB HC HD
KLGESGEIKAIGELDLLFMSLRNACIANGVLDKSNFQQPYITNRTFMLAKEASLADNNTDVRLIGEKLFH
h hhhhhhh hhhhhhhhhhh hhhhhhhhhhhhhhhhh
          HE HF HA
GVSMSERCYLMKQVLNFTLEEVLFPQSDRFQPYMQEVVPFLARLSNRLSGPGHIQRNVQKLKDTVKK
              hhhhhhhhhhhhh hhhhhhhhhhhhhhhhh hhhhhhhhhhhhhhh
                            HB HC HD
LGESGEIKAIGELDLLFMSLRNACI*
      hhhhhhh hhhhhhhhhhh
            HE HF
>ORK22-025 PolyPro 9aa linker Nt-Ct linker in bold italic and underlined
(SEQ ID NO: 287)
                                                                  ARLDKSNFQQPYITNRTFMLAKEASLADNNTDVRLIG
                                                                                      hhhhhhhhhhhhhhhhh
                                                                                                      HA
EKLFHGVSMSERCYLMKQVLNFTLEEVLFPQSDRFQPYMQEVVPFLARLSGPPPPPPPGHIQRNVQKLKD
                        hhhhhhhhhhhhh hhhhhhhhhhhhhh hhhhhhhhhhh
                                      HB HC HD
TVKKLGESGEIKAIGELDLLFMSLRNACIANGVLDKSNFQQPYITNRTFMLAKEASLADNNTDVRLIGEK
hhhh hhhhhhh hhhhhhhhhhh hhhhhhhhhhhhhhhhh
HD HE HF HA
LFHGVSMSERCYLMKQVLNFTLEEVLFPQSDRFQPYMQEVVPFLARLSNRLSGPPPPPPPGHIQRNVQKL
                    hhhhhhhhhhhhh hhhhhhhhhhhhhhhhh hhhhhhhhh
                                  HB HC HD
KDTVKKLGESGEIKAIGELDLLFMSLRNACI*
dhhhhh hhhhhhh hhhhhhhhhhh
      HD HE HF
>ORK22-026 PolyPro 11aa linker Nt-Ct linker in bold italic and underlined
(SEQ ID NO: 288)
                                                                  ARLDKSNFQQPYITNRTFMLAKEASLADNNTDVRLIG
                                                                                      hhhhhhhhhhhhhhhhh
                                                                                                  HA
EKLFHGVSMSERCYLMKQVLNFTLEEVLFPQSDRFQPYMQEVVPFLARLSGPPPPPPPPPGHIQRNVQKL
                        hhhhhhhhhhhhh hhhhhhhhhhhhh hhhhhhhhh
                                  HB HC HD
KDTVKKLGESGEIKAIGELDLLFMSLRNACIANGVLDKSNFQQPYITNRTFMLAKEASLADNNTDVRLIG
hhhhhh hhhhhhh hhhhhhhhhhh hhhhhhhhhhhhhhhhh
    HD HE HF HA
EKLFHGVSMSERCYLMKQVLNFTLEEVLFPQSDRFQPYMQEVVPFLARLSNRLSGPPPPPPPPPGHIQRN
                        hhhhhhhhhhhhh hhhhhhhhhhhhhhhhh hhhhh
                                    HB HC HD
VQKLKDTVKKLGESGEIKAIGELDLLFMSLRNACI*
hhhhhhhhhh hhhhhhh hhhhhhhhhhh
          HD HE HF
```

### Results

Results obtained with class A foldikine 22 with linkers of different lengths and/or different compositions (Gly-Ser or proline based) are shown in FIG. 36 (ORK22-17 to ORK22-19, and ORK22-24 to ORK22-26). None of these Foldikines with PolyGly or PolyPro linkers of different length were found to be active when expressed in M. pneumomoniae. Only foldikine 22 class A with engineered linkers by design (MutSC1 and MutSC2) resulted in an enhanced function.

Way B of Making Foldikine—Class II Cytokine (e.g. IL-22)

The list of variants tested is as follows:
ORK22-012: Foldikine-22 linked with two InterLinkers of 10aa with the aa sequence GGPPPPPPGG (SEQ ID NO: 289) connecting the natural Nt and Ct of the two IL-22 molecules.
ORK22-014: Foldikine-22 linked with two InterLinkers of 10aa with the aa sequence GGGGGGGGGGA (SEQ ID NO: 290) connecting the natural Nt and Ct of the two IL-22 molecules.

The amino acid sequences used were the following (canonical α-helices, only, are labelled; IL-22 signal peptide has been replaced by M. pneumoniae signal peptide—see Methods):

```
>IL22_WT
  (SEQ ID NO: 291)
MAALQKSVSSFLMGTLATSCLLLLALLVQGGAAAPISSHCRLDKSNFQQPYITNRTFMLAKEASLADNNT
                                                                                                  hhhhhhhhhhhhhhhhh
                                                                                                                  HA
DVRLIGEKLFHGVSMSERCYLMKQVLNFTLEEVLFPQSDRFQPYMQEVVPFLARLSNRLSTCHIEGDDLH
                                    hhhhhhhhhhhhh hhhhhhhhhhhhhhh
                                                HB HC
IQRNVQKLKDTVKKLGESGEIKAIGELDLLFMSLRNACI
hhhhhhhhhhhhhh hhhhhhh hhhhhhhhhhh
            HD HE HF
>ORK22-012 PolyPro linker 10aa
  (SEQ ID NO: 292)
MAALQKSVSSFLMGTLATSCLLLLALLVQGGAAAPISESGEIKAIGELDLLFMSLRNACIGGPPPPPPGG
                                                                            hhhhhhh hhhhhhhhhhhh
                                                                                HE HF
APISSHCRLDKSNFQQPYITNRTFMLAKEASLADNNTDVRLIGEKLFHGVSMSERCYLMKQVLNFTLEEV
                                hhhhhhhhhhhhhhhhh hhh hhhhhhhhhhhhh
                                              HA HB
LFPQSDRFQPYMQEVVPFLARLSNRLSTCHIEGDDLHIQRNVQKLKDTVKKLGESGEIKAIGELDLLFMS
                  hhhhhhhhhhhhhhhhh hhhhhhhhhhhhhhh hhhhhhh hhhhhh
                            HC HD HE HF
LRNACIGGPPPPPPGGAPISSHCRLDKSNFQQPYITNRTFMLAKEASLADNNTDVRLIGEKLFHGVSMSE
hhhhhh hhhhhhhhhhhhhhhhh hhh
  HF HA
RCYLMKQVLNFTLEEVLFPQSDRFQPYMQEVVPFLARLSNRLSTCHIEGDDLHIQRNVQKLKDTVKKLG
  hhhhhhhhhhhhh hhhhhhhhhhhhhhhhh hhhhhhhhhhhhhhh
                HB HC HD
>ORK22-014 PolyGly linker 10aa
  (SEQ ID NO: 293)
MAALQKSVSSFLMGTLATSCLLLLALLVQGGAAAPISESGEIKAIGELDLLFMSLRNACIGGGGGGGGGG
                                                                            hhhhhhh hhhhhhhhhhhh
                                                                                  HE HF
APISSHCRLDKSNFQQPYITNRTFMLAKEASLADNNTDVRLIGEKLFHGVSMSERCYLMKQVLNFTLEEV
                                hhhhhhhhhhhhhhhhh hhhhhhhhhhhhh
                                                HA HB
LFPQSDRFQPYMQEVVPFLARLSNRLSTCHIEGDDLHIQRNVQKLKDTVKKLGESGEIKAIGELDLLFMS
                  hhhhhhhhhhhhhhhhh hhhhhhhhhhhhhhh hhhhhhh hhhhhh
                                HC HD HE HF
LRNACIGGGGGGGGGGAPISSHCRLDKSNFQQPYITNRTFMLAKEASLADNNTDVRLIGEKLFHGVSMSE
hhhhhh hhhhhhhhhhhhhhhhh
    HF HA
RCYLMKQVLNFTLEEVLFPQSDRFQPYMQEVVPFLARLSNRLSTCHIEGDDLHIQRNVQKLKDTVKKLG
hhhhhhhhhhhhhh hhhhhhhhhhhhhhhhh hhhhhhhhhhhhhhh
            HB HC HD
```

### Results

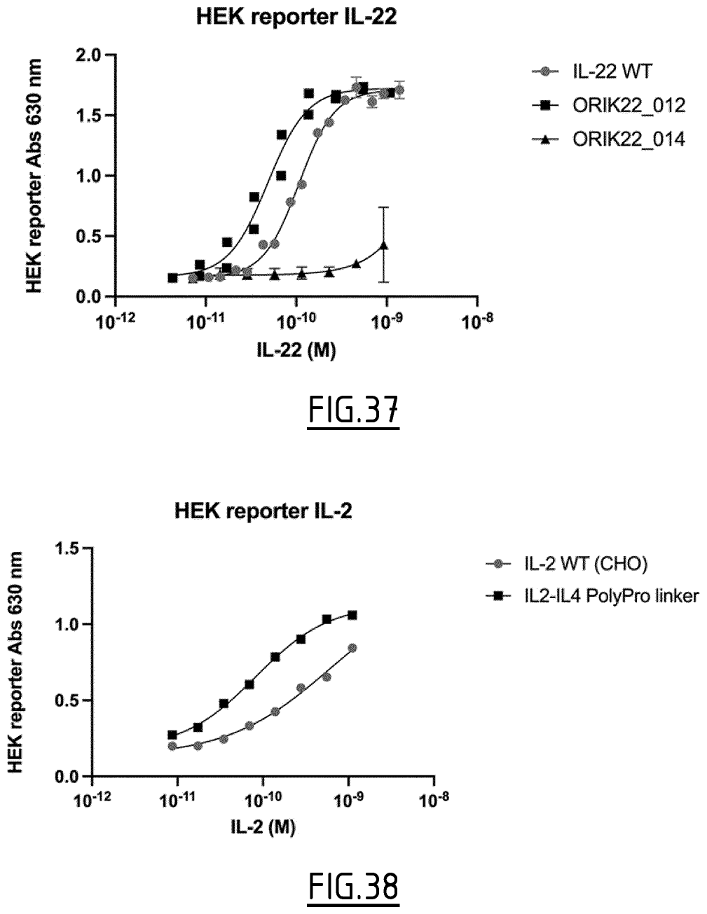

The different constructs were transiently transformed into ExpiCHO cells as described in Methods. ExpiCHO cells were grown as described in Methods and after 4 days the supernatants were collected and the concentration of the different engineered molecules calculated by ELISA. Then, we performed serial dilutions starting from 20 ng/ml (and diluting 0.5× each) and assessed the activation of IL-22 reporter HEK blue cells (see FIG. 37).

We fitted a Michaelis Menten mathematical model to the experimental data (see methods) and obtained the apparent EC-50 (Table 15).

TABLE 15
Fitting parameters for each of the mutants. Vmax was fixed
to 1.8, the saturation value obtained for IL-22WT.

| | IL-22 WT | ORIK22_012 | ORIK22_014 |
|---|---|---|---|
| Vmax | =1.800 | =1.800 | =1.800 |
| EC-50 | 9.762E−11 | 4.328E−11 | 2.419E−09 |

We found that the foldikine ORIK-12 with polyPro Interlinkers had an increased activation of the IL-22 HEK blue cells compared to IL-22 WT, while the one with a flexible polyGly linker has a strongly reduced activation.

These results indicate that, for IL-22, making a Class B foldikine, the rigidity of the InterLinkers is important.

### Way B of Making Foldikine—Class II Cytokines (Chimera IL-4-IL-2)

The variant tested is IL2-IL4 PolyPro linker, a hetero foldikine type I comprising IL-2 and IL-4, wherein IL-4 contains the continuous domain. The two ILs are linked by two InterLinkers of 7 aa with the following aa composition: GPPPPPG (SEQ ID NO: 271).

The sequences used are as follows (canonical α-helices, only, are labelled; signal peptide has been replaced by M. pneumoniae signal peptide—see Methods):

```
>IL2 WT
(SEQ ID NO: 79)
MYRMQLLSCIALSLALVTNSAPTSSSTKKTQLQLEHLLLDLQMILNGINNYKNPKLTRMLTFKFYMPKKA
                                                    hhhhhhhhhhhhhhhhhhhhhh hhhhhh
                                                                        HA
TELKHLQCLEEELKPLEEVLNLAQSKNFHLRPRDLISNINVIVLELKGSETTFMCEYADETATIVEFLNR
      hhhhhhh---hhhhhhhh hhhhh-hhhhhhhhh hhhhhhh
                  HB HC HD
WITFCQSIISTLT*
hhhhhhhhhhh
        HD
>IL-4
  (SEQ ID NO: 294)
MGLTSQLLPPLFFLLACAGNFVHGHKCDITLQEIIKTLNSLTEQKTLCTELTVTDIFAASKNTTEKETFCRAA
                                                          hhhhhhhhhhhh hhh hhhhhhhhh
                                                                      HA HB
TVLRQFYSHHEKDTRCLGATAQQFHRHKQLIRFLKRLDRNLWGLAGLNSCPVKEANQSTLENFLERLKTIMRE
hhhhhhhhh hhhhhhhhhhhhhhhhhhhhhhhhh hhhhhhhhhhhhhhhhhhh
      HB HC HD
KYSKCSS*
hhhhh
  HD
>IL2-IL4 PolyPro linker
  (SEQ ID NO: 295)
MYRMQLLSCIALSLALVTNSLRPRDLISNINVIVLELKGSETTFMCEYADETATIVEFLNRWITFCQSII
                                            hhhhh-hhhhhhhhhh hhhhhhhhhhhhhhhhh
                                                      IL-2 HC IL-2 HD
STLIGPPPPPGHKCDITLQEIIKTLNSLTEQKTLCTELTVTDIFAASKNTTEKETFCRAATVLRQFYSHH
H hhhhhhhhhhhh hhh hhhhhhhhhhhhhhhhhh
                                        IL-4 HA IL-4 HB
EKDTRCLGATAQQFHRHKQLIRFLKRLDRNLWGLAGLNSCPVKEANQSTLENFLERLKTIMREKYSKCSS
                  hhhhhhhhhhhhhhhhhhhhhhhhh hhhhhhhhhhhhhhhhhhhhhhhh
                                    IL-4 HC IL-4 HD
GPPPPPGTKKTQLQLEHLLLDLQMILNGINNYKNPKLTRMLTFKFYMPKKATELKHLQCLEEELKPLEEV
              hhhhhhhhhhhhhhhhhhhhhh hhhhhhhh---hhhhhh
                                IL2-HA IL-2 HB
LNLAQS*
```

### Results

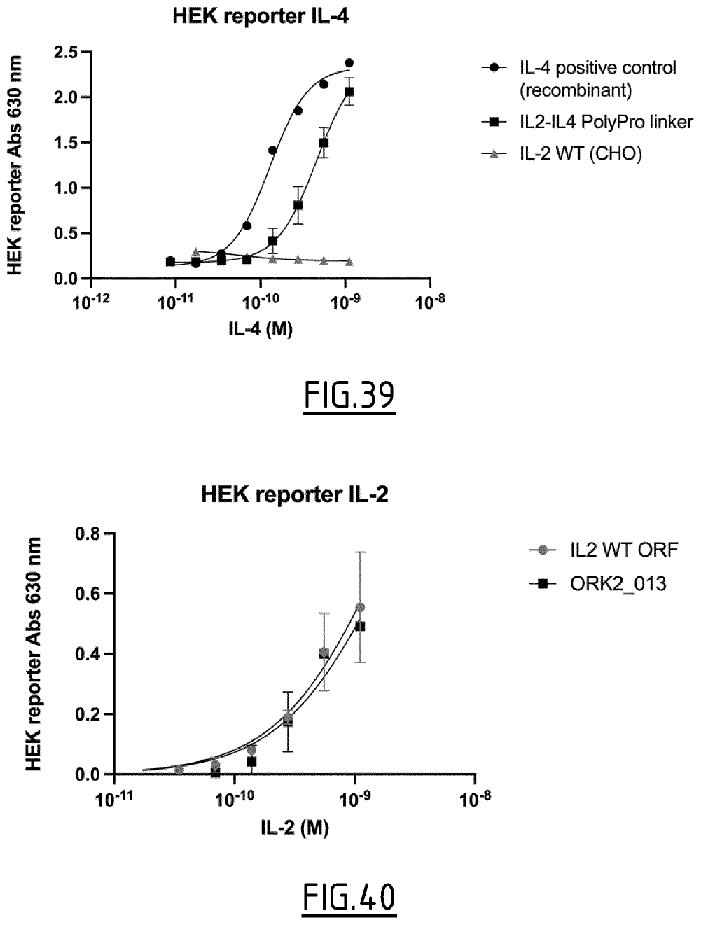

The different constructs were transiently transformed into ExpiCHO cells as described in Methods. ExpiCHO cells were grown as described in Methods and after 4 days the supernatants were collected and the concentration of the different engineered molecules calculated by ELISA. Then, we performed serial dilutions starting from 20 ng/ml (and diluting 0.5× each) and assessed the activation of IL-2 and IL-4 reporter HEK blue cells (see FIGS. 38-39).

We fitted a Michaelis Menten mathematical model to the experimental data (see methods) and obtained the apparent EC-50 (Table 16).

TABLE 16
Fitting parameters. Vmax has been set to 1.0 for
HEK IL-2 data fitting and 2.4 for HEK IL-4 data.

| IL-2 HEK | IL-2 WT (CHO) | IL2-IL4 PolyPro linker |
|---|---|---|
| Vmax | =1.000 | =1.000 |
| EC-50 | 1.719E−10 | 3.527E−11 |
| | IL-4 positive control | |
| IL-4 HEK | (recombinant) | IL2-IL4 PolyPro linker |
| Vmax | =2.400 | =2.400 |
| EC-50 | 1.232E−10 | 4.074E−10 |

We found that the chimeric Foldikine has superior activity to IL-2 WT cytokine and slightly less than commercially purchased IL-4 (in this case the difference is not significant since in one case the foldikine was expressed in CHO cells and quantified by ELISA while the other was purchased from Biotechne (6507-IL-010/CF).

Way C of Making Foldikine—Class I Cytokine (e.g. IL-2)

The Variants used and their sequences were as follows:

```
>IL2 WT
  (SEQ ID NO: 79)
MYRMQLLSCIALSLALVTNSAPTSSSTKKTQLQLEHLLLDLQMILNGINNYKNPKLTRMLTFKFYMPKKA
                                                    hhhhhhhhhhhhhhhhhhhhhh hhhhhh sss
                                                                        HA
TELKHLQCLEEELKPLEEVLNLAQSKNFHLRPRDLISNINVIVLELKGSETTFMCEYADETATIVEFLNR
      hhhhhhh---hhhhhhhh hhhhh-hhhhhhhhh sss hhhhhhh
                  HB HC HD
WITFCQSIISTLT*
hhhhhhhhhhh
        HD
```

ORK_013: A Way C circular permutant of IL-2 foldikine where one monomer has a loop opened to link it with the natural Nt and Ct of the other II-2 monomer via two InterLinkers of length 3 with the sequence GTS.

```
>ORK2_013
  (SEQ ID NO: 296)
MYRMQLLSCIALSLALVTNSNFLRPRDLISNINVIVLELKGSETTFMCEYADETATIVEFLNRWITFCQS
                                                hhhhh-hhhhhhhhhh hhhhhhhhhhhhhh
                                                            HC HD
IISTLIGGSGGNFLRPRDLISNINVIVLELKGSETTFMCEYADETATIVEFLNRWITFCQSIISTLIGGT
hhh hhhhh-hhhhhhhhhh hhhhhhhhhhhhhhhhs
HD HC HD
SSSTKKTQLQLEHLLLDLQMILNGINNYKNPKLTRMLTFKFYMPKKATELKHLQCLEEELKPLEEVLNLA
      hhhhhhhhhhhhhhhhhhhhhh hhhhhhh---hhhhhhh
                            HA HB
QSKGGSGGTSSSTKKTQLQLEHLLLDLQMILNGINNYKNPKLTRMLTFKFYMPKKATELKHLQCLEEELK
                        hhhhhhhhhhhhhhhhhhhhhh hhhhhhh---hh
                                          HA HB
PLEEVLNLAQSK
hhhhh
```

### Results

The different constructs were transiently transformed into ExpiCHO cells as described in Methods. ExpiCHO cells were grown as described in Methods and after 4 days the supernatants were collected and the concentration of the different engineered molecules calculated by ELISA. Then, we performed serial dilutions starting from 20 ng/ml (and diluting 0.5× each) and assessed the activation of IL-2 reporter HEK blue cells (see FIG. 40).

We fitted a Michaelis Menten mathematical model to the experimental data (see methods) and obtained the apparent EC-50 (Table 17).

TABLE 17
Fitting parameters. Hillslope & Bottom parameters after curve
fitting for each of the mutant. Proteins have been expressed
using CHO as expression system. Vmax was fixed to 0.7

| | IL2 WT ORF | ORK2_013 |
|---|---|---|
| Vmax | =0.7000 | =0.7000 |
| EC-50 | 5.298E−10 | 6.371E−10 |

We found that the chimeric Way C Foldikine is active although with slightly lower EC-50.

### IFNgamma (Class II Cytokine Forming Swapped Domain)

### List of Variants

### ORKIFNg-002

### List of Sequences

```
Nt-Ct linker in bold italic ASKPHPGQLWY (SEQ ID NO: 17)
Mutations in bold underlined
As IFNg WT we used the crystallised version of IFNg of PDB 6e3k
>IFNg WT
  (SEQ ID NO: 312)
SQDPYVKEAENLKKYFNAGHSDVADNGTLFLGILKNWKEESDRKIMQSQIVSFYFKLFKNFKDDQSIQKSVET
        hhhhhhhhhhhh hh hhhhhh hhhhhhhhhhhhhhhhh hhhhhh
                  HA HB
IKEDMNVKFFNSNKKKRDDFEKLTNYSVTDLNVQRKAIHELIQVMAELSPAA*
hhhhhhhhhh hhhhhhhhhhh hhhhhhhh hhhhhh
        HC HD HE HF
>ORKIFNg-002
  (SEQ ID NO: 18)
GSQDPYVKEAENLKKYFNAGHSDVADNGTLFLGILKNWKEESDRKIMQSQIVSFYFKLFKNFKDDQSIQK
          hhhhhhhhhhhh hh hhhhhh hhhhhhhhhhhhhhhhhhhh hh
                      HA HB
SVETIKEDMNVKFFNSNKKKRDDFEKLTNYSVTDLNVQRKAIHELIQVMASKPHPGQLWA
hhhhhhhhhhhhhh hhhhhhhhhhh hhhhhhhh hhhhhh
          HC HD HE HF
YVKEAENLKKYFNAGHSDVADNGTLFLGILKNWKEESDRKIMQSQIVSFYFKLFKNFKDDQSIQKSVETI
hhhhhhhhhhhh hh hhhhh hhhhhhhhhhhhhhhhhhhh hhhhhhh
        HA HB HC
KEDMNVKFFNSNKKKRDDFEKLTNYSVTDLNVQRKAIHELIQVMAELSPAA
hhhhhhhhh hhhhhhhhhhh hhhhhhhh hhhhhh
  HC HD HE HF
```

### Results

The different constructs were transiently transformed into ExpiCHO cells as described in Methods. ExpiCHO cells were grown as described in Methods and after 4 days the supernatants were collected and the concentration of the different engineered molecules calculated by ELISA (FIG. 43)

We fitted a Michaelis-Menten model to the experimental data (see methods) and obtained the apparent EC-50 (Table 18).

TABLE 18
Fitting parameters. Hillslope & Bottom parameters after curve
fitting for each of the mutant. Proteins have been expressed
using Mycoplasma pneumoniae as expression vector.

| | IFNg WT | SCIFNg_002 |
|---|---|---|
| Bottom | 2549 | 9151 |
| Hillslope | Unstable | Unstable |
| Top | Unstable | Unstable |
| EC50 | 8.661E−11 | 3.282E−11 |

We found that the foldikine IFNG has a lower EC-50 than the WT IFNg.

### Determining Suitable Loops to Open: Circular Permutants

These are very important to determine which loop to open for Class B Foldikines where we are linking the natural Nt and Ct of the two molecules and we need to open at a loop.

For class A, and Class C foldikines, they are less important since a loop that when opened in a circular permutation could destabilize the protein could be rescued by linking the new Nt and Ct resulting from the loop opened to another IL via its natural Nt and Ct (Class C) or via another loop opened (Class A).

Despite this, if geometrically feasible and compatible with receptor binding, it is always better to open a loop that does not destabilize the protein.

### IL-22

(Using structure 3dlq.pdb and SwissPDB viewer for definition of secondary structure). Depending of the crystal and complex the length of the canonical helices could vary.

#IL-22 specific Nt-Ct linker is TDYDSQTN (SEQ ID NO: 303)

#IL-22 signal peptide
(SEQ ID NO: 348)
MAALQKSVSSFLMGTLATSCLLLLALLVQGGAA

Loops in gray boxes

h alpha helix

s Strand

We only show loops and all secondary structure elements aside from the canonical HA, HB, HC, HD, HE and HF alpha helices for the WT sequence (IL22_WT)
IL22_cutasn68 Loop opened between 67-68 (loop 67-88, IAB)
IL22_cut77 Loop opened between 76-77 (loop 66-88, IaB)
IL22_cut86 Loop opened between 85-86 (loop 66-88, IAB)
IL22_cut109 Loop opened between 108-109 (loop 102-113, IBC)
IL22_cut135 Loop opened between 134-135 (loop 129-140, ICD)
IL22_cut156 Loop opened between 164-165 (loop 165-167, IEF)

loop 155-157 (IDE) has a very unfavorable orientation that will preclude joining to another cytokine and was not tested.

```
>IL22_WT
  (SEQ ID NO: 291)
                                                                                                                                      1AB
MAALQKSVSSFLMGTLATSCLLLLALLVQGGAAAPISSHCRLDKSNFQQPYITNRTFMLAKEASLADNNT
                                                                                                  hhhhhhhhhhhhhhhhh
                                                                                                                  HA
            1AB 1BC 1CD
DVRLIGEKLFHGVSMSERCYLMKQVLNFTLEEVLFPQSDRFQPYMQEVVPFLARLSNRLSTCHIEGDDLH
                                    hhhhhhhhhhhhh hhhhhhhhhhhhhhh
                                                HB HC
                            1DE 1EF
IQRNVQKLKDTVKKLGESGEIKAIGELDLLFMSLRNACI
hhhhhhhhhhhhhh hhhhhhh hhhhhhhhhhh
            HD HE HF
>IL22_cutasn68 (loop open between residues 67-68)
  (SEQ ID NO: 297)
MAALQKSVSSFLMGTLATSCLLLLALLVQGGAANNTDVRLIGEKLFHGVSMSERCYLMKQVLNFTLEEVL
                                                                                                            hhhhhhhhhhhhh
                                                                                                                        hB
FPQSDRFQPYMQEVVPFLARLSNRLSTCHIEGDDLHIQRNVQKLKDTVKKLGESGEIKAIGELDLLFMSL
H hhhhhhhhhhhhhhh hhhhhhhhhhhhhh hhhhhhh hhhhhhh
                            HC HD HE HF
RNACTDYDSQTNCRLDKSNFQQPYITNRTFMLAKEASLAD
hhhh hhhhhhhhhhhhhhhhh
HF HA
>IL22_cut77 (loop open between residues 76-77)
  (SEQ ID NO: 298)
MAALQKSVSSFLMGTLATSCLLLLALLVQGGAAEKLFHGVSMSERCYLMKQVLNFTLEEVLFPQSDRFQP
                                                                                          hhhhhhhhhhhhh
                                                                                                      HB
YMQEVVPFLARLSNRLSTCHIEGDDLHIQRNVQKLKDTVKKLGESGEIKAIGELDLLFMSLRNACTDYDS
Hhhhhhhhhhhhhhh hhhhhhhhhhhhhh hhhhhhh hhhhhhhhhhh
            HC HD HE HF
QTNCRLDKSNFQQPYITNRTFMLAKEASLADNNTDVRLIG*
                          hhhhhhhhhhhhhhhhh
                                          HA
>IL22_cut86 (loop open between residues 85-86)
  (SEQ ID NO: 299)
MAALQKSVSSFLMGTLATSCLLLLALLVQGGAASERCYLMKQVLNFTLEEVLFPQSDRFQPYMQEVVPFL
                                                                        hhhhhhhhhhhhh hhhhhhhhh
                                                                                    HB HC
ARLSNRLSTCHIEGDDLHIQRNVQKLKDTVKKLGESGEIKAIGELDLLFMSLRNACTDYDSQTNCRLDKS
hhhhhh hhhhhhhhhhhhhh hhhhhhh hhhhhhhhhhh
      HC HD HE HF
NFQQPYITNRTFMLAKEASLADNNTDVRLIGEKLFHGVSM*
        hhhhhhhhhhhhhhhhh
                        HA
>IL22_cut109 (loop open between residues 108-109)
  (SEQ ID NO: 300)
MAALQKSVSSFLMGTLATSCLLLLALLVQGGAADRFQPYMQEVVPFLARLSNRLSTCHIEGDDLHIQRNV
                                                                            hhhhhhhhhhhhhhh hhhhh
                                                                                      HC HD
QKLKDTVKKLGESGEIKAIGELDLLFMSLRNACTDYDSQTNCRLDKSNFQQPYITNRTFMLAKEASLADN
hhhhhhhhh hhhhhhh hhhhhhhhhhh hhhhhhhhhhhhhhhhh
      HD HE HF HA
NTDVRLIGEKLFHGVSMSERCYLMKQVLNFTLEEVLFPQS*
                                        hhhhhhhhhhhhh
                                                  HB
>IL22_cut135 (loop open between residues 134-135)
  (SEQ ID NO: 301)
MAALQKSVSSFLMGTLATSCLLLLALLVQGGAAEGDDLHIQRNVQKLKDTVKKLGESGEIKAIGELDLLF
                                                                              hhhhhhhhhhhhhh hhhhhhh hhhh
                                                                                            HD HE HF
MSLRNACTDYDSQTNCRLDKSNFQQPYITNRTFMLAKEASLADNNTDVRLIGEKLFHGVSMSERCYLMKQ
hhhhhhh hhhhhhhhhhhhhhhhh hhhhhh
      HF HA HB
VLNFTLEEVLFPQSDRFQPYMQEVVPFLARLSNRLSTCHI*
Hhhhhhh hhhhhhhhhhhhhhh
    HB HC
>IL22_cut156 (loop open between residues 164-165)
  (SEQ ID NO: 302)
MAALQKSVSSFLMGTLATSCLLLLALLVQGGAAGESGEIKAIGELDLLFMSLRNACTDYDSQTNCRLDKS
                                                                      hhhhhhh hhhhhhhhhhh
                                                                            HE HF
NFQQPYITNRTFMLAKEASLADNNTDVRLIGEKLFHGVSMSERCYLMKQVLNFTLEEVLFPQSDRFQPYM
        hhhhhhhhhhhhhhhhh hhhhhhhhhhhhh hh
                    HA HB HC
QEVVPFLARLSNRLSTCHIEGDDLHIQRNVQKLKDTVKKL*
Hhhhhhhhhhhhh hhhhhhhhhhhhhh
            HC HD
```

### IFNbeta

(Using structure 1au1.pdb and SwissPDB viewer for definition of secondary structure). Depending of the crystal and complex the length of the canonical helices could vary.

(SEQ ID NO: 304)
#IFNb specific linker is EGPG
(SEQ ID NO: 305)
#IFNb signal peptide MTNKCLLQIALLLCFSTTALS

Loops in gray boxes
h alpha helix
s Strand

We only show loops and all secondary structure elements aside from the canonical HA, HB, HC, HD, HE and HF alpha helices for the WT sequence (IL22_WT+sp)

### List of Variants

IFNb_cut26 Loop opened between 25-26 (loop 22-29, IAB1)
IFNb_cut50 Loop opened between 49-50 (loop 34-51, IAB2)
IFNb_cut76 Loop opened between 75-76 (loop 71-81, IBC)
IFNb_cut112 Loop opened between 117-118 (loop 108-121, ICD)
IFNb_cut165 adding the Nt-Ct linker to the Nt and Ct of the WT

```
>IFNbWT
  (SEQ ID NO: 306)
                                                                                    1AB1 1AB2 1AB3
MTNKCLLQIALLLCFSTTALSYNLIGFLQRSSNFQCQKLLWQLNGRLEYCLKDRMNFDIPEEIKQLQQFQ
                                          hhhhhhhhhhhhhhhhhhh hhhh hhhh
                                                            HA
                                        1BC1 1BC2 1CD
KEDAALTIYEMLQNIFAIFRQDSSSTGWNETIVENLLANVYHQINHLKTVLEEKLEKEDFTRGKLMSSLH
hhhhhhhhhhhhhhhhhhh hh hhhhhhhhhhhhhhhhhhhhhhhhhh
              HB HC
                              1EF
LKRYYGRILHYLKAKEYSHCAWTIVRVEILRNFYFINRLTGYL*
hhhhhhhhhhhhhhh hhhhhhhhhhhhhhhhhhhhhhh
                          D
HE-HF
>IFNb_cut165 (adding the split Nt-Ct IntraLinker (in bold) to the Nt and Ct
of the WT)
  (SEQ ID NO: 307)
MTNKCLLQIALLLCFSTTALSGPGYNLLGFLQRSSNFQCQKLLWQLNGRLEYCLKDRMNFDIPEEIKQLQ
                                                hhhhhhhhhhhhhhhhhhh hhhh hhhh
                                                                  HA
QFQKEDAALTIYEMLQNIFAIFRQDSSSTGWNETIVENLLANVYHQINHLKTVLEEKLEKEDFTRGKLMS
      hhhhhhhhhhhhhhhhhh hhhhhhhhhhhhhhhhhhhhhhhhhh
                      HB HC
SLHLKRYYGRILHYLKAKEYSHCAWTIVRVEILRNFYFINRLTGYLE*
      hhhhhhhhhhhhhhh hhhhhhhhhhhhhhhhhhhhhhh
                  HD HE-HF
>IFNb_cut26 (loop opened between 25-26)
  (SEQ ID NO: 308)
MTNKCLLQIALLLCFSTTALSGRLEYCLKDRMNFDIPEEIKQLQQFQKEDAALTIYEMLQNIFAIFRQDS
                                                  hhhh hhhh hhhhhhhhhhhhhhhhhhh
                                                                                                                  HB
SSTGWNETIVENLLANVYHQINHLKTVLEEKLEKEDFTRGKLMSSLHLKRYYGRILHYLKAKEYSHCAWT
              hhhhhhhhhhhhhhhhhhhhhhhhhh hhhhhhhhhhhhhhh hhhhh
                                      HC HD HE-HF
IVRVEILRNFYFINRLTGYLEGPGYNLLGFLQRSSNFQCQKLLWQLN*
hhhhhhhhhhhhhhhhhh hhhhhhhhhhhhhhhhhhh
      HE-HF HA
>IFNb_cut50 (loop opened between 49-50)
  (SEQ ID NO: 309)
MTNKCLLQIALLLCFSTTALSFQKEDAALTIYEMLQNIFAIFRQDSSSTGWNETIVENLLANVYHQINHL
                                              hhhhhhhhhhhhhhhhhhh hhhhhhhhhhhhhhhhh
                                                              HB HC
KTVLEEKLEKEDFTRGKLMSSLHLKRYYGRILHYLKAKEYSHCAWTIVRVEILRNFYFINRLTGYLEGPG
Hhhhhhhhh hhhhhhhhhhhhhhh hhhhhhhhhhhhhhhhhhhhhhh
          HC HD HE-HF
YNLLGFLQRSSNFQCQKLLWQLNGRLEYCLKDRMNFDIPEEIKQLQQ*
hhhhhhhhhhhhhhhhhhh hhhh hhhh
                  HA
>IFNb_cut76 (loop opened between 75-76)
  (SEQ ID NO: 310)
MTNKCLLQIALLLCFSTTALSSTGWNETIVENLLANVYHQINHLKTVLEEKLEKEDFTRGKLMSSLHLKR
                                                      hhhhhhhhhhhhhhhhhhhhhhhhhh hhh
                                                                                HC HD
YYGRILHYLKAKEYSHCAWTIVRVEILRNFYFINRLTGYLEGPGYNLLGFLQRSSNFQCQKLLWQLNGRL
hhhhhhhhhhhh hhhhhhhhhhhhhhhhhhhhhhh hhhhhhhhhhhhhhhhhhh
            HD HE-HF HA
EYCLKDRMNFDIPEEIKQLQQFQKEDAALTIYEMLQNIFAIFRQDSS*
  hhhh hhhh hhhhhhhhhhhhhhhhhhh
                                                              HB
>IFNb_cut112 (loop opened between 117-118)
  (SEQ ID NO: 311)
MTNKCLLQIALLLCFSTTALSSSLHLKRYYGRILHYLKAKEYSHCAWTIVRVEILRNFYFINRLTGYLEG
                                                  hhhhhhhhhhhhhhh hhhhhhhhhhhhhhhhhhhhhhh
                                                            HD HE-HF
PGYNLLGFLQRSSNFQCQKLLWQLNGRLEYCLKDRMNFDIPEEIKQLQQFQKEDAALTIYEMLQNIFAIF
    hhhhhhhhhhhhhhhhhhh hhhh hhhh hhhhhhhhhhhhhhhhhhh
                    HA HB
RQDSSSTGWNETIVENLLANVYHQINHLKTVLEEKLEKEDFTRGKLM*
                      hhhhhhhhhhhhhhhhhhhhhhhhhh
                                              HC
```

### IL-2

(Using structure 2b5i.pdb and SwissPDB viewer for definition of secondary structure). Depending of the crystal and complex the length of the canonical helices could vary.

(SEQ ID NO: 319)
#IL-2 specific Nt-Ct linker is LPKLGM
(SEQ ID NO: 320)
#IL-2 signal peptide MYRMQLLSCIALSLALVTNS

Loops in gray boxes

h alpha helix

s Strand

We only show loops and all secondary structure elements aside from the canonical HA, HB, HC, HD, HE and HF alpha helices for the WT sequence (IL22_WT+sp)

### List of Variants

IL2_cut53 Loop opened between 32-33 (loop 29-34;IAB1)
IL2_cut69 Loop opened between 48-49 (loop 69-76;IAB3)
IL2_cut96 Loop opened between 76-77 (loop 91-101;IBC)
IL2_cut121 Loop opened between 100-101 (loop 97-105;ICD1)

```
>IL2 WT
  (SEQ ID NO: 79)
                                                                                                    1AB1 1AB2
MYRMQLLSCIALSLALVTNSAPTSSSTKKTQLQLEHLLLDLQMILNGINNYKNPKLTRMLTFKFYMPKKA
                                                    hhhhhhhhhhhhhhhhhhhhhh hhhhhh sss
                                                                        HA
1AB3 1BC 1CD1 1CD2
TELKHLQCLEEELKPLEEVLNLAQSKNFHLRPRDLISNINVIVLELKGSETTFMCEYADETATIVEFLNR
      hhhhhhh---hhhhhhhh hhhhh-hhhhhhhhh sss hhhhhhh
                  HB HC HD
WITFCQSIISTLT*
hhhhhhhhhhh
        HD
>IL2_cut53 (loop open between residues 32-33)
  (SEQ ID NO: 321)
MYRMQLLSCIALSLALVTNSNPKLTRMLTFKFYMPKKATELKHLQCLEEELKPLEEVLNLAQSKNFHLRP
                                                                                hhhhhhh---hhhhhh hh
                                                                                                HB HC
RDLISNINVIVLELKGSETTFMCEYADETATIVEFLNRWITFSQSIISLLPKLGMNKKTQLQLEHLLLDL
Hhh-hhhhhhhhhhhh hhhhhhhhhhhhhhhhh hhhhhhhhhhhhhh
          HC HD HA
QMILNGINNYK*
hhhhhhh
    HA
>IL2_cut69 (loop open between residues 48-49)
(SEQ ID NO: 322)
MYRMQLLSCIALSLALVTNSKATELKHLQCLEEELKPLEEVLNLAQSKNFHLRPRDLISNINVIVLELKG
                                                  hhhhhhh---hhhhhh hhhhh-hhhhhhhhhh
                                                                HB HC
SETTFMCEYADETATIVEFLNRWITFSQSIISLLPKLGMNKKTQLQLEHLLLDLQMILNGINNYKNPKLT
                              hhhhhhhhhhhhhhhhh hhhhhhhhhhhhhhhhhhhhh
                                            HD HA
RMLTFKFYMPK*
>IL2_cut96 (loop open between residues 76-77)
  (SEQ ID NO: 323)
MYRMQLLSCIALSLALVTNSNFHLRPRDLISNINVIVLELKGSETTFMCEYADETATIVEFLNRWITFSQ
                                                  hhhhh-hhhhhhhhhh hhhhhhhhhhhhh
                                                                HC HD
SIISLLPKLGMNKKTQLQLEHLLLDLQMILNGINNYKNPKLTRMLTFKFYMPKKATELKHLQCLEEELKP
hhhh hhhhhhhhhhhhhhhhhhhhh hhhhhhh---hhh
  HD HA HB
LEEVLNLAQSK*
hhh
HB
>IL2_cut121 (loop open between residues 100-101)
  (SEQ ID NO: 324)
MYRMQLLSCIALSLALVTNSTTFMCEYADETATIVEFLNRWITFSQSIISLLPKLGMNKKTQLQLEHLLL
                                                                  hhhhhhhhhhhhhhhhh hhhhhhhhhhhh
                                                                                HD HA
DLQMILNGINNYKNPKLTRMLTFKFYMPKKATELKHLQCLEEELKPLEEVLNLAQSKNFHLRPRDLISNI
hhhhhhhhh hhhhhhh---hhhhhh hhhhh-hhh
        HA HB HC
NVIVLELKGSE*
hhhhhhhhh
      HC
```

### Results

The different constructs were transiently transformed into ExpiCHO cells as described in Methods. ExpiCHO cells were grown as described in Methods and after 4 days the supernatants were collected and the concentration of the different engineered molecules calculated by ELISA. Then, we performed serial dilutions starting from 20 ng/ml (and diluting 0.5× each) and assessed the activation of IL-22, IFNb, IL-2 and IL-4 reporter HEK blue cells (see FIGS. 41-43).

We fitted a Michaelis-Menten model to the experimental data (see methods) and obtained the apparent EC-50. We found that depending of the IL some loops were quite tolerant to opening them while other resulted in a significant decrease in EC-50. Interestingly in the case of IL-22 and IL-2 and we found that some circular permutants had beeter EC-50 than the corresponding IL WT. This could suggest that InterLinker introduced to join the natural Nt and Ct could have favourable interactions with the receptor that increase affinity.

### REFERENCES

1. Chan, I. H. et al. The Potentiation of IFN-γ and Induction of Cytotoxic Proteins by Pegylated IL-10 in Human CD8 T Cells. J. Interferon Cytokine Res. 35, 948-955 (2015).
2. Ouyang, W. & O'Garra, A. IL-10 Family Cytokines IL-10 and IL-22: from Basic Science to Clinical Translation. Immunity vol. 50 871-891 (2019).
3. Fillatreau, S. & O'Garra, A. Interleukin-10 in Health and Disease. (Springer, 2014).
4. Saraiva, M., Vieira, P. & O'Garra, A. Biology and therapeutic potential of interleukin-10. Journal of Experimental Medicine vol. 217 (2020).
5. van Deventer, S. J., Elson, C. O. & Fedorak, R. N. Multiple doses of intravenous interleukin 10 in steroid-refractory Crohn's disease. Crohn's Disease Study Group. Gastroenterology vol. 113 383-389 (1997).
6. Saxena, A. et al. Interleukin-10 paradox: A potent immunoregulatory cytokine that has been difficult to harness for immunotherapy. Cytokine 74, 27-34 (2015).
7. Terai, M. et al. Human interleukin 10 receptor 1/IgG1-Fc fusion proteins: immunoadhesins for human IL-10 with therapeutic potential. Cancer Immunol. Immunother. 58, 1307-1317 (2009).
8. Duncan, S. A. et al. Prolonged Release and Functionality of Interleukin-10 Encapsulated within PLA-PEG Nanoparticles. Nanomaterials (Basel) 9, (2019).
9. Gorby, C. et al. Engineered IL-10 variants elicit potent immunomodulatory effects at low ligand doses. Sci. Signal. 13, (2020).
10. Saxton, R. A. et al. Structure-based decoupling of the pro- and anti-inflammatory functions of interleukin-10. Science 371, (2021).
11. Steidler, L. et al. Treatment of murine colitis by Lactococcus lactis secreting interleukin-10. Science 289, 1352-1355 (2000).
12. Cardoso, A. et al. The Dynamics of Interleukin-10-Afforded Protection during Dextran Sulfate Sodium-Induced Colitis. Front. Immunol. 9, 400 (2018).
13. Braat, H. et al. A Phase I Trial With Transgenic Bacteria Expressing Interleukin-10 in Crohn's Disease. Clinical Gastroenterology and Hepatology vol. 4 754-759 (2006).
14. Cypel, M. et al. Functional repair of human donor lungs by IL-10 gene therapy. Sci. Transl. Med. 1, 4ra9 (2009).
15. Shamskhou, E. A. et al. Hydrogel-based delivery of II-10 improves treatment of bleomycin-induced lung fibrosis in mice. Biomaterials 203, 52-62 (2019).
16. Wang, X., Wong, K., Ouyang, W. & Rutz, S. Targeting IL-10 Family Cytokines for the Treatment of Human Diseases. Cold Spring Harbor Perspectives in Biology vol. 11 a028548 (2019).
17. Waites, K. B. & Talkington, D. F. Mycoplasma pneumoniae and Its Role as a Human Pathogen. Clinical Microbiology Reviews vol. 17 697-728 (2004).
18. Wodke, J. A. H. Dissecting the energy metabolism in Mycoplasma pneumoniae through genome-scale metabolic modeling. Mol. Syst. Biol. 9, 653 (2013).
19. Inamine, J. M., Ho, K. C., Loechel, S. & Hu, P. C. Evidence that UGA is read as a tryptophan codon rather than as a stop codon by Mycoplasma pneumoniae, Mycoplasma genitalium, and Mycoplasma gallisepticum. J. Bacteriol. 172, 504-506 (1990).
20. Sluijter, M. et al. The Mycoplasma genitalium MG352-encoded protein is a Holliday junction resolvase that has a non-functional orthologue in Mycoplasma pneumoniae. Molecular Microbiology vol. 77 1261-1277 (2010).
21. Maier, T. et al. Quantification of mRNA and protein and integration with protein turnover in a bacterium. Molecular Systems Biology vol. 7 511 (2011).
22. Kühner, S. et al. Proteome Organization in a Genome-Reduced Bacterium. Science vol. 326 1235-1240 (2009).
23. Scott, M., Gunderson, C. W., Mateescu, E. M., Zhang, Z. & Hwa, T. Interdependence of cell growth and gene expression: origins and consequences. Science 330, 1099-1102 (2010).
24. Bennett, M. J., Choe, S. & Eisenberg, D. Domain swapping: entangling alliances between proteins. Proceedings of the National Academy of Sciences vol. 91 3127-3131 (1994).
25. Josephson, K. et al. Design and analysis of an engineered human interleukin-10 monomer. J. Biol. Chem. 275, 13552-13557 (2000).
26. Syto, R. et al. Structural and biological stability of the human interleukin 10 homodimer. Biochemistry 37, 16943-16951 (1998).
27. Westerhof, L. B. et al. 3D Domain Swapping Causes Extensive Multimerisation of Human Interleukin-10 When Expressed In Planta. PLOS ONE vol. 7 e46460 (2012).
28. Huffnagle, G. B., Dickson, R. P. & Lukacs, N. W. The respiratory tract microbiome and lung inflammation: a two-way street. Mucosal Immunol. 10, 299-306 (2017).
29. Minshawi, F. et al. The Generation of an Engineered Interleukin-10 Protein With Improved Stability and Biological Function. Front. Immunol. 11, 1794 (2020).
30. Delgado, J., Radusky, L. G., Cianferoni, D. & Serrano, L. FoldX 5.0: working with RNA, small molecules and a new graphical interface. Bioinformatics 35, 4168-4169 (2019).
31. Schymkowitz, J. et al. The FoldX web server: an online force field. Nucleic Acids Res. 33, W382-8 (2005).
32. Delgado Blanco, J., Radusky, L. G., Cianferoni, D. & Serrano, L. Protein-assisted RNA fragment docking (RnaX) for modeling RNA-protein interactions using ModelX. Proc. Natl. Acad. Sci. U.S.A 116, 24568-24573 (2019).
33. Cianferoni, D., Radusky, L. G., Head, S. A., Serrano, L. & Delgado, J. ProteinFishing: a protein complex generator within the ModelX toolsuite. Bioinformatics 36, 4208-4210 (2020).
34. Blanco, J. D., Radusky, L., Climente-González, H. & Serrano, L. FoldX accurate structural protein—DNA binding prediction using PADA1 (Protein Assisted DNA Assembly 1). Nucleic Acids Research vol. 46 3852-3863 (2018).
35. Yu, H., Pardoll, D. & Jove, R. STATs in cancer inflammation and immunity: a leading role for STAT3. Nat. Rev. Cancer 9, 798-809 (2009).
36. Lastrucci, C. et al. Tuberculosis is associated with expansion of a motile, permissive and immunomodulatory CD16 (+) monocyte population via the IL-10/STAT3 axis. Cell Res. 25, 1333-1351 (2015).
37. Zizzo, G., Hilliard, B. A., Monestier, M. & Cohen, P. L. Efficient clearance of early apoptotic cells by human macrophages requires M2c polarization and MerTK induction. J. Immunol. 189, 3508-3520 (2012).
38. Heck, H. D. Statistical theory of cooperative binding to proteins. Hill equation and the binding potential. Journal of the American Chemical Society vol. 93 23-29 (1971).
39. Tamiya, S., Yoshikawa, E., Suzuki, K. & Yoshioka, Y. Susceptibility Analysis in Several Mouse Strains Reveals Robust T-Cell Responses After Mycoplasma pneumoniae Infection in DBA/2 Mice. Frontiers in Cellular and Infection Microbiology vol. 10 (2021).
40. Somarajan, S. R., Kannan, T. R. & Baseman, J. B. Mycoplasma pneumoniae Mpn133 is a cytotoxic nuclease with a glutamic acid-, lysine- and serine-rich region essential for binding and internalization but not enzymatic activity. Cell. Microbiol. 12, 1821-1831 (2010).
41. Segovia, J. A. Mycoplasma Pneumoniae Cards Toxin Regulates NLRP3 Inflammasome Activation. Journal of Allergy and Clinical Immunology vol. 135 AB153 (2015).
42. Hames, C., Halbedel, S., Hoppert, M., Frey, J. & Stülke, J. Glycerol metabolism is important for cytotoxicity of Mycoplasma pneumoniae. J. Bacteriol. 191, 747-753 (2009).
43. Sun, L. et al. Effect of IL-10 on neutrophil recruitment and survival after Pseudomonas aeruginosa challenge. Am. J. Respir. Cell Mol. Biol. 41, 76-84 (2009).
44. Mauras, A. et al. A New Bifidobacteria Expression SysTem (BEST) to Produce and Deliver Interleukin-10 in Bifidobacterium bifidum. Frontiers in Microbiology vol. 9 (2018).
45. Steidler, L. et al. Treatment of murine colitis by Lactococcus lactis secreting interleukin-10. Science 289, 1352-1355 (2000).
46. Lindner, H. A., Velasquez, S. Y., Thiel, M. & Kirschning, T. Lung Protection vs. Infection Resolution: Interleukin 10 Suspected of Double-Dealing in COVID-19. Front. Immunol. 12, 602130 (2021).
47. Gibson, D. G. et al. Enzymatic assembly of DNA molecules up to several hundred kilobases. Nat. Methods 6, 343-345 (2009).
48. Yus, E., Yang, J.-S., Sogues, A. & Serrano, L. A reporter system coupled with high-throughput sequencing unveils key bacterial transcription and translation determinants. Nat. Commun. 8, 368 (2017).
49. Piñero-Lambea, C. et al. Genome Editing Based on Oligo Recombineering and Cas9-Mediated Counterselection. ACS Synth. Biol. 9, 1693-1704 (2020).
50. Burgos, R., Weber, M., Martinez, S., Lluch-Senar, M. & Serrano, L. Protein quality control and regulated proteolysis in the genome-reduced organism Mycoplasma pneumoniae. Mol. Syst. Biol. 16, e9530 (2020).
51. Chiva, C. et al. QCloud: A cloud-based quality control system for mass spectrometry-based proteomics laboratories. PLOS One 13, e0189209 (2018).
52. Perkins, D. N., Pappin, D. J., Creasy, D. M. & Cottrell, J. S. Probability-based protein identification by searching sequence databases using mass spectrometry data. Electrophoresis 20, 3551-3567 (1999).
53. Beer, L. A., Liu, P., Ky, B., Barnhart, K. T. & Speicher, D. W. Efficient Quantitative Comparisons of Plasma Proteomes Using Label-Free Analysis with MaxQuant. Methods Mol. Biol. 1619, 339-352 (2017).
54. Troegeler, A. et al. An efficient siRNA-mediated gene silencing in primary human monocytes, dendritic cells and macrophages. Immunol. Cell Biol. 92, 699-708 (2014).
55. Grillo, M.-J., Blasco, J. M., Gorvel, J. P., Moriyon, I. & Moreno, E. What have we learned from brucellosis in the mouse model? Vet. Res. 43, 29 (2012).
56. Kamei, A. et al. Exogenous remodeling of lung resident macrophages protects against infectious consequences of bone marrow-suppressive chemotherapy. Proc. Natl. Acad. Sci. U.S.A 113, E6153-E6161 (2016).
57. Hirche, T. O. et al. Neutrophil elastase mediates innate host protection against Pseudomonas aeruginosa. J. Immunol. 181, 4945-4954 (2008).
58. Victoria Garrido & Carlos Piñero-Lambea, Irene Rodriguez-Arce, Bernhard Paetzold, Tony Ferrar, Marc Weber, Eva Garcia-Ramallo, Carolina Gallo, María Collantes, Iván Peñuelas, Luis Serrano, María-Jesús Grillo, Maria Lluch-Senar. Engineering a genome-reduced bacterium to eliminate Staphylococcus aureus biofilms in vivo. Molecular Systems Biology, in press.

### CLAUSES

The following single chain dimeric cytokine polypeptides make part of the invention:
A1. A single chain dimeric cytokine polypeptide comprising: (a) a first cytokine monomer domain or a functional portion thereof; and (b) a second cytokine monomer domain or a functional portion thereof, wherein the first cytokine monomer domain or a functional portion thereof is fused to the second cytokine monomer domain or a functional portion thereof by an engineered peptide linker sequence comprises 5 or less, 3 or less, or 2 or less adjacent Gly and/or Ser residues.
A1A. The single chain dimeric cytokine polypeptide of Clause A1, wherein the peptide linker has a sequence of from:
   (i) about 3 to about 20 amino acids;
   (ii) about 3 to about 16 amino acids;
   (iii) about 3 to about 12 amino acids;
   (iv) about 3 to about 8 amino acids;
   (v) about 4 to about 8 amino acids; or
   (vi) about 4 to about 6 amino acids.
A2. The single chain dimeric cytokine polypeptide of Clause A1 or Clause A1A, wherein the single chain dimeric cytokine polypeptide has a tertiary structure comprising a left 3D cytokine domain and a right 3D cytokine domain, and wherein the left 3D cytokine domain is connected to the right 3D cytokine domain by two bridging linker peptides.
A3. The single chain dimeric cytokine polypeptide of Clause A2, wherein the left 3D cytokine domain is formed from a portion of the first cytokine monomer and a portion of the second cytokine monomer; and the right 3D cytokine domain is formed from a portion of the first cytokine monomer and a portion of the second cytokine monomer.
A4. The single chain dimeric cytokine polypeptide of Clause A2 or Clause A3, wherein the two bridging linker peptides are structured linkers having from 3 to 20 amino acids, wherein the bridging peptide linker sequences comprise 5 or less, suitably 3 or less adjacent Gly and/or Ser residues.
A5. The single chain dimeric cytokine polypeptide of any of Clauses A1 to A4, wherein: (a) the first and second cytokine monomers are of the same structural type; or (b) wherein the first and second cytokine monomers are of different types.
A6. The single chain dimeric cytokine polypeptide of any of Clauses A1 to A5, wherein the first cytokine monomer is selected from or derived from a cytokine of the group consisting of: IL-10, IL-5 and IFNγ; and wherein the second cytokine monomer is selected from or derived from a cytokine of the group consisting of: IL-10, IL-5 and IFNγ.
A7. The single chain dimeric cytokine polypeptide of any of Clauses A1 to A6, wherein the first cytokine monomer and the second cytokine monomer are IL-10 monomers; optionally wherein the IL-10 is derived from a human or a mouse sequence.
A8. The single chain dimeric cytokine polypeptide of any of Clauses A1 to A7, wherein the first and second cytokine monomers comprise the sequence of IL-10 ORF (SEQ ID NO: 4), SEQ ID NO: 181, SEQ ID NO: 349 or SEQ ID NO: 350 or a functional fragment thereof, or a sequence having at least 80%, at least 85%, at least 90%, at least 95%, at least 98%, at least 99% or 100% sequence identity to SEQ ID NO: 4, SEQ ID NO: 181, SEQ ID NO: 349 or SEQ ID NO: 350 or the functional fragment thereof.
A9. The single chain dimeric cytokine polypeptide of Clause A8, wherein the first and/or second cytokine monomer sequences include one or more amino acid substitution selected from the group consisting of: N18I, D28E, S31K, N45S, N92I, K99N and T155M wherein the numbering of mutated positions is based on SEQ ID NO: 181, wherein the Q at the first position of SEQ ID NO: 181 is sequence position Q7.
A10. The single chain dimeric cytokine polypeptide of Clause A9, wherein the first and/or second cytokine monomer sequences include at least the amino acid substitutions: D28E, S31K, N45S and T155M.
A11. The single chain dimeric cytokine polypeptide of Clause A9 or Clause A10, wherein the first cytokine monomer sequence includes all of the amino acid substitutions: N18I, D28E, S31K, N45S, N921, K99N and T155M; and wherein the second cytokine monomer sequence includes all of the amino acid substitutions: D28E, S31K, N45S, N921, K99N and T155M.
A12. The single chain dimeric cytokine polypeptide of any of Clauses A1 to A8, which comprises:
(i) the sequence of SEQ ID NO: 9 (MutSC1) or a sequence having at least 80%, at least 85%, at least 90%, at least 95%, at least 98%, at least 99% or 100% sequence identity thereto; or
(ii) the sequence of SEQ ID NO: 11-(NtCt)-SEQ ID NO: 12 (Foldikine-10) or a sequence having at least 80%, at least 85%, at least 90%, at least 95%, at least 98%, at least 99% or 100% sequence identity thereto.
A13. The single chain dimeric cytokine polypeptide of any of Clauses A1 to A11, which comprises the sequence of SEQ ID NO: 10 (MutSC2) or a sequence having at least 80%, at least 85%, at least 90%, at least 95%, at least 98%, at least 99% or 100% sequence identity thereto.
A14. The single chain dimeric cytokine polypeptide of Clause A12 (ii), wherein the linker peptide NtCt has a sequence of from about 4 to about 8 amino acids or from about 3 to about 6 amino acids.
A15. The single chain dimeric cytokine polypeptide of Clause A14, wherein the linker peptide NtCt comprises a sequence selected from FGGLD (SEQ ID NO: 342), or NGGLD (SEQ ID NO: 318), optionally further comprising one or more amino acid mutation of the linker peptide NtCt flanking residues; and particularly wherein the sequence is SEQ ID NO: 65.
A16. The single chain dimeric cytokine polypeptide of any of Clauses A1 to A6, wherein the first cytokine monomer and the second cytokine monomer are IL-5 monomers; optionally wherein the IL-5 is a human or a mouse sequence.
A17. The single chain dimeric cytokine polypeptide of any of Clauses A1 to A6 or A16, wherein the first and second cytokine monomers comprise the sequence of human IL-5 (SEQ ID NO: 82) or a functional fragment thereof, or a sequence having at least 80%, at least 85%, at least 90%, at least 95%, at least 98%, at least 99% or 100% sequence identity to SEQ ID NO: 82 or the functional fragment thereof.
A18. The single chain dimeric cytokine polypeptide of Clause A16 or Clause A17, which comprises the sequence of SEQ ID NO: 101-(NtCt)-SEQ ID NO:102 (Foldikine-5) or a sequence having at least 80%, at least 85%, at least 90%, at least 95%, at least 98%, at least 99% or 100% sequence identity thereto.
A19. The single chain dimeric cytokine polypeptide of Clause A18, wherein the linker peptide NtCt has a sequence of from about 3 to about 20 amino acids, from about 3 to 16 amino acids, from about 4 to 12 amino acids, from about 4 to 8 amino acids, from about 3 to 8 amino acids or from about 3 to 6 amino acids.
A20. The single chain dimeric cytokine polypeptide of Clause A19, wherein the linker peptide NtCt comprises a sequence selected from FGGLD (SEQ ID NO: 342) or NGGLD (SEQ ID NO: 318).
A21. The single chain dimeric cytokine polypeptide of any of Clauses A1 to A6, wherein the first cytokine monomer and the second cytokine monomer are IFNγ monomers; optionally wherein the IFNγ is a human or a mouse sequence.
A22. The single chain dimeric cytokine polypeptide of any of Clauses A1 to A6 or A21, wherein the first and second cytokine monomers comprise the sequence of human IFNγ (SEQ ID NO: 194) or a functional fragment thereof, or a sequence having at least 80%, at least 85%, at least 90%, at least 95%, at least 98%, at least 99% or 100% sequence identity to SEQ ID NO: 194 or the functional fragment thereof.
A23. The single chain dimeric cytokine polypeptide of Clause A21 or Clause A22, which comprises the sequence of SEQ ID NO: 14-(NtCt)-SEQ ID NO:15 (Foldikine-IFNG) or a sequence having at least 80%, at least 85%, at least 90%, at least 95%, at least 98%, at least 99% or 100% sequence identity thereto.
A24. The single chain dimeric cytokine polypeptide of Clause A23, wherein the linker peptide NtCt has a sequence of from about 3 to about 20 amino acids, from about 3 to 16 amino acids, from about 4 to 12 amino acids, from about 4 to 8 amino acids, from about 3 to 8 amino acids or from about 3 to 6 amino acids.
A25. The single chain dimeric cytokine polypeptide of Clause A24, wherein the linker peptide NtCt comprises a sequence selected from FGGLD (SEQ ID NO: 342), or NGGLD (SEQ ID NO: 318).
A26. An isolated polynucleotide encoding a polypeptide of any of Clauses A1 to A25.
A27. A vector comprising the isolated polynucleotide of Clause A26.
A28. The vector of Clause A27, which is a bacteriophage vector or a viral vector.
A29. A cell expressing a single chain dimeric cytokine polypeptide of any of Clauses A1 to
A25.
A30. The cell of Clause A29, which is a eukaryotic cell; particularly a human cell.
A31. The cell of Clause A29, which is a recombinant bacterial cell; optionally wherein the bacterial cell is selected from a Mycoplasma (e.g. M. pneumoniae), E. coli, L. lactis, S. aureus, or B. subtilis bacterial strain.
A32. The cell of Clause A21, which is a Mycoplasma pneumoniae strain, optionally derived from strain M129-B7 strain (ATTC 29342); Escherichia coli BL21; or Lactococcus lactis strain NZ9000-pepN: nisRK.
A33. A cell comprising a polynucleotide or vector of any of Clauses A26 to A28.
A34. A pharmaceutical composition comprising the single chain dimeric cytokine polypeptide of any of Clauses A1 to A25, the cell of any of Clauses A29 to A33, or the isolated polynucleotide or vector of any of Clauses A26 to A28.
A35. The single chain dimeric cytokine polypeptide of any of Clauses A1 to A25, the cell of any of Clauses A29 to A33, the isolated polynucleotide or vector of any of Clauses A26 to A28, or the pharmaceutical composition of Clause A32, for use in medicine.
A36. The single chain dimeric cytokine polypeptide, isolated polynucleotide or vector, cell or pharmaceutical composition for use according to Clause A35, wherein the use is in a method for the treatment of a disease or disorder selected from inflammatory diseases and disorders; lung diseases; cancer or proliferative diseases or conditions; infectious diseases; allergy, immunological or autoimmune diseases and disorders; tissue injury, degeneration or damage condition; and conditions associated therewith.
A37. The single chain dimeric cytokine polypeptide, isolated polynucleotide or vector, cell or pharmaceutical composition for use according to Clause A36, wherein the cancer or proliferative disease or disorder is a solid tumour, for example, selected from the group consisting of lung cancers (in particular lung adenocarcinomas or lung squamous carcinoma), bladder cancer, cervical cancer, breast cancer, colon cancer, brain glioblastoma, pancreatic cancer, acute monocytic leukemia, kidney cancer, colorectal cancer, skin cancer (e.g. melanoma), stomach cancer, thyroid cancer, bone cancer and liver cancer.
A38. The single chain dimeric cytokine polypeptide, isolated polynucleotide or vector, cell or pharmaceutical composition for use according to Clause A36, wherein the inflammatory disease or disorder is selected from the group consisting of allergy, asthma, autoimmune diseases, coeliac disease, glomerulonephritis, hepatitis, inflammatory bowel disease, preperfusion injury, transplant rejection, diabetes, Alzheimer's disease, osteoarthritis, fibromyalgia, muscular low back pain, arthritis, muscular neck pain, myocarditis, Ankylosing Spondylitis (AS), Antiphospholipid Antibody Syndrome (APS), Myositis, Rheumatoid Arthritis, Scleroderma, Sjogren's Syndrome, fibrosis.
A39. The single chain dimeric cytokine polypeptide, isolated polynucleotide or vector, cell or pharmaceutical composition for use according to Clause A36, wherein the tissue injury, degeneration or damage condition is selected from the group consisting of colitis, wound healing, hair growth, liver regeneration and matrix synthesis.
A40. The single chain dimeric cytokine polypeptide, isolated polynucleotide or vector, cell or pharmaceutical composition for use according to Clause A36, wherein the use is in cancer immunotherapy and/or immune oncology.
B1. A single chain dimeric cytokine polypeptide comprising: (a) a first cytokine monomer domain or a functional portion thereof; and (b) a second cytokine monomer domain or a functional portion thereof, wherein the sequence of the second cytokine monomer domain of functional portion thereof is inserted within the sequence of the first cytokine monomer domain such that a first sequence portion of the first cytokine monomer is arranged at the N-terminus of the second cytokine monomer or functional portion thereof, and a second sequence portion of the first cytokine monomer is arranged at the C-terminus of the second cytokine monomer or functional portion thereof.
B2. The single chain dimeric cytokine polypeptide of Clause B1, wherein the single chain dimeric cytokine polypeptide has a tertiary structure comprising a left 3D cytokine domain and a right 3D cytokine domain, and wherein the left 3D cytokine domain is connected to the right 3D cytokine domain by two bridging linker peptides.
B3. The single chain dimeric cytokine polypeptide of Clause B2, wherein the left 3D cytokine domain is a split domain cytokine formed from first and second sequence portions of the first cytokine monomer domain or a functional portion thereof and the right 3D cytokine domain is a continuous domain cytokine formed from the second cytokine monomer domain or a functional portion thereof.
B4. The single chain dimeric cytokine polypeptide of Clause B2 or Clause B3, wherein the two bridging linker peptides have a sequence of from about 3 to about 20 amino acids, and wherein bridging linker peptide sequences comprises 5 or less and suitably 3 or less adjacent Gly and/or Ser residues; optionally wherein the two bridging linker peptides from structured linkers.
B5. The single chain dimeric cytokine polypeptide of Clause B4, wherein the two bridging linker peptides are the same sequence or are different sequences.
B6. The single chain dimeric cytokine polypeptide of Clause B2 or of any of Clauses B3 to B5 when dependent on Clause B2, wherein a first of the two bridging linker peptides connects the N-terminal portion of the first cytokine monomer domain to the N-terminal portion of the second cytokine monomer domain and the second of the two bridging linker peptides connects the C-terminal portion of the second cytokine monomer domain to the C-terminus of the first cytokine monomer domain.
B7. The single chain dimeric cytokine polypeptide of Clause B2 or of any of Clauses B3 to B6 when dependent on Clause B2, wherein the first cytokine monomer domain is split in a loop region between a pair of adjacent α-helices and wherein the two bridging linker peptides connect the second cytokine monomer domain between the adjacent α-helices of the first cytokine monomer domain; optionally, wherein the first cytokine monomer is split in the loop region between the second and third α-helices or between the third and fourth α-helices of the cytokine monomer domain.
B8. The single chain dimeric cytokine polypeptide of Clause B6 or Clause B7, wherein the second cytokine monomer domain comprises an N-terminal portion and a C-terminal portion, and wherein the N-terminal portion is connected to the C-terminal portion by an engineered peptide linker; optionally, wherein the engineered peptide linker has a sequence of from about 3 to about 20 amino acids, from about 3 to 16 amino acids, from about 4 to 12 amino acids, from about 4 to 8 amino acids, from about 3 to 8 amino acids or from about 3 to 6 amino acids.
B9. The single chain dimeric cytokine polypeptide of Clause B8, wherein the engineered peptide linker comprises 5 or less, 3 or less, or 2 or less adjacent Gly and/or Ser residues.
B10. The single chain dimeric cytokine polypeptide of Clause B8 or Clause B9, wherein:
(i) the second cytokine monomer domain is inverted in comparison to a corresponding wild type cytokine monomer on which it is based, such that the N-terminal portion of the second cytokine monomer domain corresponds to a C-terminal portion of the corresponding wild type cytokine monomer domain sequence and the C-terminal portion of the second cytokine monomer domain corresponds to an N-terminal portion of a corresponding wild-type cytokine monomer domain sequence; optionally, wherein the N-terminal portion of the second cytokine monomer domain comprises 2, 3 or 4 α-helices and the C-terminal portion of the second cytokine monomer domain comprises 2, 3 or 4 α-helices; particularly, wherein the N-terminal portion and the C-terminal portion of the second cytokine monomer domain have the same number of α-helices; and/or
(ii) the N-terminal portion of the first cytokine monomer domain corresponds to an N-terminal portion of the corresponding wild type cytokine monomer domain sequence and the C-terminal portion of the first cytokine monomer domain corresponds to a C-terminal portion of a corresponding wild-type cytokine monomer domain sequence; optionally, wherein the N-terminal portion of the first cytokine monomer domain comprises 2, 3 or 4 α-helices and the C-terminal portion of the first cytokine monomer domain comprises 2, 3 or 4 α-helices; particularly, wherein the N-terminal portion and the C-terminal portion of the second cytokine monomer domain have the same number of α-helices.
B11. The single chain dimeric cytokine polypeptide of any of Clauses B8 to B10, wherein the two bridging linker peptides connect to the second cytokine monomer domain between a pair of adjacent α-helices.
B12. The single chain dimeric cytokine polypeptide of any of Clauses B1 to B11, wherein: (a) the first and second cytokine monomers are of the same type; or (b) wherein the first and second cytokine monomers are of different types.
B13. The single chain dimeric cytokine polypeptide of any of Clauses B1 to B12, wherein the first cytokine monomer is selected from or derived from a class II cytokine of the group consisting of: IL-19, IL-20, IL-22, IL-24, IL-26, IFNλ 2, 3 and 1 (IL-28A, IL-28B and IL-29, respectively), type I interferon IFNα (IFNA1, IFNA2, IFNA4, IFNA5, IFNA6, IFNA7, IFNA8, IFNA10, IFNA13, IFNA14, IFNA16, IFNA17 and IFNA21), IFNω (IFNW1), IFNE (IFNE), IFNκ (IFNK), and IFNβ (IFNB1); and wherein the second cytokine monomer is selected from or derived from a class I cytokine of the group consisting of: IL6, IL11, IL12A, IL23A, IL27A, IL31, CLCF1, CNTF, CTF1, LIF, OSM, CSF3, IL2, IL3, IL4, IL5, IL7, IL9, IL13, IL15, IL21, TSLP, GM-CSF, CSF1, and CSF2; optionally wherein the first and second cytokine monomer sequences are derived from a human or a mouse sequence.
B14. The single chain dimeric cytokine polypeptide of any of Clauses B1 to B13, wherein the first cytokine monomer and/or the second cytokine monomer are IL-22 monomers; optionally wherein the IL-22 is a human or a mouse sequence.
B15. The single chain dimeric cytokine polypeptide of any of Clauses B1 to B14, wherein the first and second cytokine monomers comprise the sequence of IL-22 (SEQ ID NO: 184) or a functional fragment thereof, or a sequence having at least 80%, at least 85%, at least 90%, at least 95%, at least 98%, at least 99% or 100% sequence identity to SEQ ID NO: 184 or the functional fragment thereof.
B16. The single chain dimeric cytokine polypeptide of Clause B14 or Clause B15, wherein the first and/or second cytokine monomer sequences include one or more amino acid substitutions that increase the stability of the hydrophobic core of the IL-22 monomer domain; optionally, wherein the one or more amino acid substitutions are selected from the group consisting of: T56L or T56M, A66M, N68Q, V95I, T99F and S173L, wherein the numbering of mutated positions is based on the IL-22 sequence of SEQ ID NO: 184, wherein the A at the first position of SEQ ID NO: 184 is sequence position A34.
B17. The single chain dimeric cytokine polypeptide of Clause B16, wherein the first and/or second cytokine monomer sequences include a set of amino acid substitutions selected from:
   (i) A66M, V95I and T99F;
   (ii) T56L, A66M, V95I and T99F;
   (iii) T56M, A66M, N68Q, V95I and T99F
   (iii) T56L, A66M, V95I, T99F and S173L; and
   (ii) T56M, A66M, N68Q, V95I, T99F and S173L.
B18. The single chain dimeric cytokine polypeptide of any of Clauses B15 to B17, wherein the first and/or second cytokine monomer sequences include one or more amino acid substitutions that increase the stability and/or binding affinity of the IL-22 monomer domain to its receptor R1 and/or R2; optionally, wherein the one or more amino acid substitutions are selected from the group consisting of: D43E, S45R, K61W, S64M, T70I, E77Y and A132Y, wherein the numbering of mutated positions is based on SEQ ID NO: 184, wherein the A at the first position of SEQ ID NO: 184 is sequence position A34.
B19. The single chain dimeric cytokine polypeptide of Clause B18, wherein the first and/or second cytokine monomer sequences include a set of amino acid substitutions selected from:
   (i) A132Y, D43E, S45R, E77Y, T70I;
   (ii) K61W, S64M; and
   (iii) A132Y, D43E, S45R, E77Y, T70I, K61W, S64M.
B20. The single chain dimeric cytokine polypeptide of Clause B6 or of any of Clauses B7 to B19 when dependent on Clause B6, wherein the first of the two bridging linker peptides comprises or has the sequence TCMPGGSKT (SEQ ID NO: 337) and the second of the two bridging linker peptides comprises or has the sequence RLELLP (SEQ ID NO: 331); or wherein the first of the two bridging linker peptides comprises the sequence NRLKKLMASSD (SEQ ID NO: 332) and the second of the two bridging linker peptides comprises the sequence RLELLP (SEQ ID NO: 331).
B21. The single chain dimeric cytokine polypeptide of Clause B8 or of any of Clauses B9 to B20 when dependent on Clause B8, wherein the N-terminal portion of the second cytokine monomer domain is connected to the C-terminal portion of the second cytokine monomer domain by an engineered peptide linker comprising or having the sequence ANGT (SEQ ID NO: 329).
B22. The single chain dimeric cytokine polypeptide of any of Clauses B1 to B21, which comprises a sequence selected from SEQ ID NOs: 65 (Foldikine-22_1), 66 (Foldikine-22_3), 72 (3dlq. Pdb monomer sequence), 73 (IL-22hydC monomer), 74 (MutantC_IL22 monomer), 75 (MutantD_IL22 monomer), 76 (MutantE_IL22 monomer) and 143 (Foldikine-22), or a sequence having at least 80%, at least 85%, at least 90%, at least 95%, at least 98%, at least 99% or 100% sequence identity thereto.
B23. The single chain dimeric cytokine polypeptide of any of Clauses B1 to B21, which comprises a sequence selected from SEQ ID NOs: 65 (Foldikine-22_1), 66 (Foldikine-22_3) and 143 (Foldikine-22), or a sequence having at least 80%, at least 85%, at least 90%, at least 95%, at least 98%, at least 99% or 100% sequence identity thereto.
B24. The single chain dimeric cytokine polypeptide of any of Clauses B1 to B13, which comprises a sequence selected from the group consisting of SEQ ID NOs: 141, 142, 144, 145, 146, 147, 148, 149, 150, 151, 152 and 153, or a sequence having at least 80%, at least 85%, at least 90%, at least 95%, at least 98%, at least 99% or 100% sequence identity thereto.
B25. The single chain dimeric cytokine polypeptide of any of Clauses B22 to B24, wherein the linker peptide NtCt has a sequence of from about 3 to about 20 amino acids, from about 3 to 16 amino acids, from about 4 to 12 amino acids, from about 4 to 8 amino acids, from about 3 to 8 amino acids or from about 3 to 6 amino acids.
B26. The single chain dimeric cytokine polypeptide of Clause B25, wherein the linker peptide NtCt comprises a sequence selected from FGGLD (SEQ ID NO: 342), NGGLD (SEQ ID NO: 318), NFGGLDY (SEQ ID NO: 316) or ANGT (SEQ NO: 329); particularly ID the sequence of SEQ ID NO: 190.
B27. The single chain dimeric cytokine polypeptide of any of Clauses B22 to B26 when dependent on Clause B6, wherein the bridging linker peptides (Xn) each has a sequence of from about 3 to about 20 amino acids, from about 3 to 16 amino acids, from about 4 to 12 amino acids, from about 4 to 8 amino acids, from about 3 to 8 amino acids or from about 3 to 6 amino acids.
B28. The single chain dimeric cytokine polypeptide of Clause B27, wherein the bridging linker peptides (Xn) have a sequence selected from TCMPGGSKT (SEQ ID NO: 337), RLELLP (SEQ ID NO: 331); and NRLKKLMASSD (SEQ ID NO: 332).
B29. An isolated polynucleotide encoding a polypeptide of any of Clauses B1 to B28.
B30. A vector comprising the isolated polynucleotide of Clause B29.
B31. The vector of Clause B30, which is a bacteriophage vector or a viral vector.
B32. A cell expressing a single chain dimeric cytokine polypeptide of any of Clauses B1 to
B28.
B33. The cell of Clause B32, which is a eukaryotic cell; particularly a human cell.
B34. The cell of Clause B32, which is a recombinant bacterial cell; optionally wherein the bacterial cell is selected from a Mycoplasma (e.g. M. pneumoniae), E. coli, L. lactis, S. aureus, or B. subtilis bacterial strain.
B35. The cell of Clause B34, which is a Mycoplasma pneumoniae strain, optionally derived from strain M129-B7 strain (ATTC 29342); Escherichia coli BL21; or Lactococcus lactis strain NZ9000-pepN::nisRK.
B36. A cell comprising a polynucleotide or vector of any of Clauses B29 to B31.
B37. A pharmaceutical composition comprising the single chain dimeric cytokine polypeptide of any of Clauses B1 to B28, the cell of any of Clauses B32 to B36, or the isolated polynucleotide or vector of any of Clauses B29 to B31.
B38. The single chain dimeric cytokine polypeptide of any of Clauses B1 to B28, the cell of any of Clauses B32 to B36, the isolated polynucleotide or vector of any of Clauses B29 to B31, or the pharmaceutical composition of Clause B35, for use in medicine.
B39. The single chain dimeric cytokine polypeptide, isolated polynucleotide or vector, cell or pharmaceutical composition for use according to Clause B38, wherein the use is in a method for the treatment of a disease or disorder selected from inflammatory diseases and disorders; lung diseases; cancer or proliferative diseases or conditions; infectious diseases; allergy, immunological or autoimmune diseases and disorders; tissue injury, degeneration or damage condition; and conditions associated therewith.
B40. The single chain dimeric cytokine polypeptide, isolated polynucleotide or vector, cell or pharmaceutical composition for use according to Clause B39, wherein the cancer or proliferative disease or disorder is a solid tumour, for example, selected from the group consisting of lung cancers (in particular lung adenocarcinomas or lung squamous carcinoma), bladder cancer, cervical cancer, breast cancer, colon cancer, brain glioblastoma, pancreatic cancer, acute monocytic leukemia, kidney cancer, colorectal cancer, skin cancer (e.g. melanoma), stomach cancer, thyroid cancer, bone cancer and liver cancer.
B41. The single chain dimeric cytokine polypeptide, isolated polynucleotide or vector, cell or pharmaceutical composition for use according to Clause B39, wherein the inflammatory disease or disorder is selected from the group consisting of allergy, asthma, autoimmune diseases, coeliac disease, glomerulonephritis, hepatitis, inflammatory bowel disease, preperfusion injury, transplant rejection, diabetes, Alzheimer's disease, osteoarthritis, fibromyalgia, muscular low back pain, arthritis, muscular neck pain, myocarditis, Ankylosing Spondylitis (AS), Antiphospholipid Antibody Syndrome (APS), Myositis, Rheumatoid Arthritis, Scleroderma, Sjogren's Syndrome, fibrosis.
B42. The single chain dimeric cytokine polypeptide, isolated polynucleotide or vector, cell or pharmaceutical composition for use according to Clause B39, wherein the tissue injury, degeneration or damage condition is selected from the group consisting of colitis, wound healing, hair growth, liver regeneration and matrix synthesis.
B43. The single chain dimeric cytokine polypeptide, isolated polynucleotide or vector, cell or pharmaceutical composition for use according to Clause B39, wherein the use is in cancer immunotherapy and/or immune oncology.
C1. A single chain dimeric cytokine polypeptide comprising: (a) a first cytokine monomer domain or a functional portion thereof; and (b) a second cytokine monomer domain or a functional portion thereof, wherein the sequence of the second cytokine monomer domain or functional portion thereof is inserted within the sequence of the first cytokine monomer domain such that a first sequence portion of the first cytokine monomer is arranged at the N-terminus of the second cytokine monomer or functional portion thereof, and a second sequence portion of the first cytokine monomer is arranged at the C-terminus of the second cytokine monomer or functional portion thereof.
C1A. The single chain dimeric cytokine polypeptide of Clause C1, wherein the first sequence portion of the first cytokine monomer domain corresponds to a C-terminal portion of a natural cytokine monomer, and the second sequence portion of the first cytokine monomer domain corresponds to an N-terminal portion of a natural cytokine monomer.
C2. The single chain dimeric cytokine polypeptide of Clause C1 or Clause C1A, wherein the single chain dimeric cytokine polypeptide has a tertiary structure comprising a left 3D cytokine domain and a right 3D cytokine domain, and wherein the left 3D cytokine domain is connected to the right 3D cytokine domain by two bridging linker peptides.
C3. The single chain dimeric cytokine polypeptide of Clause C2, wherein the left 3D cytokine domain is a split domain cytokine formed from first and second sequence portions of the first cytokine monomer domain or a functional portion thereof and the right 3D cytokine domain is a continuous domain cytokine formed from the second cytokine monomer domain or a functional portion thereof.
C4. The single chain dimeric cytokine polypeptide of Clause C2 or Clause C3, wherein the two bridging linker peptides have a sequence of from about 3 to 20 amino acids, from about 3 to 16 amino acids, from about 4 to 12 amino acids, from about 4 to 8 amino acids, from about 3 to 8 amino acids or from about 3 to 6 amino acids; and wherein the bridging linker peptide sequences comprises 5 or less and suitably 3 or less adjacent Gly and/or Ser residues; optionally wherein the two bridging linker peptides are structured linkers.
C5. The single chain dimeric cytokine polypeptide of Clause C4, wherein the two bridging linker peptides are the same sequence or are different sequences.
C6. The single chain dimeric cytokine polypeptide of Clause C4 or Clause C5, wherein the two bridging linker peptides are structured linkers or are short flexible linkers.
C7. The single chain dimeric cytokine polypeptide of Clause C2 or of any of Clauses C3 to C6 when dependent on Clause C2, wherein a first of the two bridging linker peptides connects the first sequence portion of the first cytokine monomer domain to the N-terminus of the second cytokine monomer domain and the second of the two bridging linker peptides connects the C-terminus of the second cytokine monomer domain to the second sequence portion of the first cytokine monomer domain.
C8. The single chain dimeric cytokine polypeptide of any of Clauses C1 to C7, wherein the first cytokine monomer domain is split in a loop region between a pair of adjacent α-helices to form the N- and C-termini of the single chain dimeric cytokine polypeptide.
C9. The single chain dimeric cytokine polypeptide of any of Clauses C1 to C8, wherein the first cytokine monomer domain is inverted in comparison to a corresponding wild type cytokine monomer on which it is based, such that the N-terminal portion of the first cytokine monomer domain corresponds to a C-terminal portion of the corresponding wild type cytokine monomer domain sequence and the C-terminal portion of the first cytokine monomer domain corresponds to an N-terminal portion of a corresponding wild-type cytokine monomer domain sequence.
C10. The single chain dimeric cytokine polypeptide of Clause C8 or Clause C9, wherein the N-terminal portion of the first cytokine monomer domain comprises 2, 3 or 4 α-helices and the C-terminal portion of the first cytokine monomer domain comprises 2, 3 or 4 α-helices; particularly, wherein the N-terminal portion and the C-terminal portion of the first cytokine monomer domain have the same number of α-helices; optionally, wherein the first cytokine monomer is split in the loop region between the second and third α-helices or between the third and fourth α-helices of the cytokine monomer domain.
C11. The single chain dimeric cytokine polypeptide of any of Clauses C1 to C10, wherein a peptide linker/loop between two adjacent α-helices of the second cytokine monomer domain is replaced with an engineered linker peptide of from about 3 to about 20 amino acids, from about 3 to about 16 amino acids, from about 4 to about 12 amino acids, from about 4 to about 8 amino acids, from about 3 to about 8 amino acids or from about 3 to about 6 amino acids.
C11A. The single chain dimeric cytokine polypeptide of any of Clauses C1 to C11, wherein the engineered peptide linker sequence comprises 5 or less, preferably 3 or less or 2 or less adjacent Gly and/or Ser residues; optionally wherein the engineered peptide linker is a structured linker.
C12. The single chain dimeric cytokine polypeptide of any of Clauses C1 to C11, wherein:
(i) the first and second cytokine monomers are of the same type; or
(ii) the first and second cytokine monomers are of different types.
C13. The single chain dimeric cytokine polypeptide of any of Clauses C1 to C12, wherein
(i) the first cytokine monomer is selected from or derived from a class I cytokine of the group consisting of: IL2, IL3, IL4, IL5, IL7, IL9, IL15, IL21, GMCS-F, IL6, IL11, IL12A, IL23A, IL27A, IL31, CLCF1, CNTF, CTF1, LIF, OSM and CSF3; and wherein the second cytokine monomer is selected from or derived from a class I cytokine of the group consisting of: of IL2, IL3, IL4, IL5, IL7, IL9, IL15, IL21, GMCS-F, IL6, IL11, IL12A, IL23A, IL27A, IL31, CLCF1, CNTF, CTF1, LIF, OSM and CSF3;
(ii) the first cytokine monomer is selected from or derived from a class I cytokine of the group consisting of: IL6, IL11, IL12A, IL23A, IL27A, IL31, CLCF1, CNTF, CTF1, LIF, OSM and CSF3; and wherein the second cytokine monomer is selected from or derived from a class I cytokine of the group consisting of: IL6, IL11, IL12A, IL23A, IL27A, IL31, CLCF1, CNTF, CTF1, LIF, OSM and CSF3; or
(iii) the first cytokine monomer is selected from or derived from a class I cytokine of the group consisting of: IL2, IL3, IL4, IL7, IL9, IL15, IL21, TSL and GMCS-F; and wherein the second cytokine monomer is selected from or derived from a class I cytokine of the group consisting of: IL2, IL3, IL4, IL7, IL9, IL15, IL21, TSL and GMCS-F; optionally wherein the first and second cytokine monomer sequences are derived from a human or a mouse sequence.
C14. The single chain dimeric cytokine polypeptide of any of Clauses C1 to C13, which comprises a sequence selected from the group consisting of SEQ ID NOs: 92, 93, 94, 96, 97, 98, 99, 100 and 101, 114, 115, 116, 117, 118, 119, 120, 121, 122, 123, 124 and 125, or a sequence having at least 80%, at least 85%, at least 90%, at least 95%, at least 98%, at least 99% or 100% sequence identity thereto.
C15. The single chain dimeric cytokine polypeptide of Clause C13 or Clause C14, wherein each of the bridging linker peptides (Xn) has a sequence of from about 3 to 20 amino acids, from about 3 to 16 amino acids, from about 4 to 12 amino acids, from about 4 to 8 amino acids, from about 3 to 8 amino acids or from about 3 to 6 amino acids.
C16. An isolated polynucleotide encoding a polypeptide of any of Clauses C1 to C15.
C17. A vector comprising the isolated polynucleotide of Clause C16.
C18. The vector of Clause C17, which is a bacteriophage vector or viral vector.
C19. A cell expressing a single chain dimeric cytokine polypeptide of any of Clauses C1 to
C15.
C20. The cell of Clause C19, which is a eukaryotic cell; particularly a human cell.
C21. The cell of Clause C19, which is a recombinant bacterial cell; optionally wherein the bacterial cell is selected from a Mycoplasma (e.g. M. pneumoniae), E. coli, L. lactis, S. aureus, or B. subtilis bacterial strain.
C22. The cell of Clause C21, which is a Mycoplasma pneumoniae strain, optionally derived from strain M129-B7 strain (ATTC 29342); Escherichia coli BL21; or Lactococcus lactis strain NZ9000-pepN::nisRK.
C23. A cell comprising the polynucleotide or vector of any of Clauses C16 to C18.
C24. A pharmaceutical composition comprising the single chain dimeric cytokine polypeptide of any of Clauses C1 to C15, the cell of any of Clauses C19 to C23, or the isolated polynucleotide or vector of any of Clauses C16 to C18.
C25. The single chain dimeric cytokine polypeptide of any of Clauses C1 to C15, the cell of any of Clauses C19 to C23, the isolated polynucleotide or vector of any of Clauses C16 to C18, or the pharmaceutical composition of Clause C24, for use in medicine.
C26. The single chain dimeric cytokine polypeptide, isolated polynucleotide or vector, cell or pharmaceutical composition for use according to Clause C25, wherein the use is in a method for the treatment of a disease or disorder selected from inflammatory diseases and disorders; lung diseases; cancer or proliferative diseases or conditions; infectious diseases; allergy, immunological or autoimmune diseases and disorders; tissue injury, degeneration or damage condition; and conditions associated therewith.
C27. The single chain dimeric cytokine polypeptide, isolated polynucleotide or vector, cell or pharmaceutical composition for use according to Clause C26, wherein the cancer or proliferative disease or disorder is a solid tumour, for example, selected from the group consisting of lung cancers (in particular lung adenocarcinomas or lung squamous carcinoma), bladder cancer, cervical cancer, breast cancer, colon cancer, brain glioblastoma, pancreatic cancer, acute monocytic leukemia, kidney cancer, colorectal cancer, skin cancer (e.g. melanoma), stomach cancer, thyroid cancer, bone cancer and liver cancer.
C28. The single chain dimeric cytokine polypeptide, isolated polynucleotide or vector, cell or pharmaceutical composition for use according to Clause C26, wherein the inflammatory disease or disorder is selected from the group consisting of allergy, asthma, autoimmune diseases, coeliac disease, glomerulonephritis, hepatitis, inflammatory bowel disease, preperfusion injury, transplant rejection, diabetes, Alzheimer's disease, osteoarthritis, fibromyalgia, muscular low back pain, arthritis, muscular neck pain, myocarditis, Ankylosing Spondylitis (AS), Antiphospholipid Antibody Syndrome (APS), Myositis, Rheumatoid Arthritis, Scleroderma, Sjogren's Syndrome, fibrosis.
C29. The single chain dimeric cytokine polypeptide, isolated polynucleotide or vector, cell or pharmaceutical composition for use according to Clause C26, wherein the tissue injury, degeneration or damage condition is selected from the group consisting of colitis, wound healing, hair growth, liver regeneration and matrix synthesis.
C30. The single chain dimeric cytokine polypeptide, isolated polynucleotide or vector, cell or pharmaceutical composition for use according to Clause C26, wherein the use is in cancer immunotherapy and/or immune oncology.
D1. A single chain dimeric cytokine polypeptide comprising: (a) a first cytokine monomer domain or a functional portion thereof; and (b) a second cytokine monomer domain or a functional portion thereof, wherein the first cytokine monomer domain sequence is derived from a different type of cytokine to the second cytokine monomer domain sequence; and wherein the left cytokine domain is a split domain cytokine formed from first and second sequence portions of the first cytokine monomer domain or a functional portion thereof and the right 3D cytokine domain is a continuous domain cytokine formed from the second cytokine monomer domain or a functional portion thereof.
D2. The single chain dimeric cytokine polypeptide of Clause D1, wherein the single chain dimeric cytokine polypeptide has a tertiary structure comprising a left 3D cytokine domain formed of the first cytokine monomer domain or a functional portion thereof, and a right 3D cytokine domain formed of the second cytokine monomer domain or a functional portion thereof.
D3. The single chain dimeric cytokine polypeptide of Clause D1 or Clause D2, wherein the first cytokine monomer domain is connected to the second cytokine monomer domain by two bridging linker peptides.
D4. The single chain dimeric cytokine polypeptide of Clause D3, wherein the two bridging linker peptides each has a sequence of from about 3 to about 20 amino acids, from about 3 to about 16 amino acids, from about 4 to about 12 amino acids, from about 4 to about 8 amino acids, from about 3 to about 8 amino acids or from about 3 to about 6 amino acids; preferably wherein the bridging peptide linker sequences comprises 5 or less, 3 or less, or 2 or less adjacent Gly and/or Ser residues.
D5. The single chain dimeric cytokine polypeptide of any of Clauses D1 to D4, wherein the second cytokine monomer domain or functional portion thereof is inserted within the sequence of the first cytokine monomer domain such that a first sequence portion of the first cytokine monomer is arranged at the N-terminus of the second cytokine monomer or functional portion thereof, and a second sequence portion of the first cytokine monomer is arranged at the C-terminus of the second cytokine monomer or functional portion thereof.
D6. The single chain dimeric cytokine polypeptide of Clause D2 or any of Clauses D3 to D5 when dependent on Clause D2, wherein the two bridging linker peptides are the same sequence or are different sequences.
D7. The single chain dimeric cytokine polypeptide of Clause D2 or of any of Clauses D3 to D6 when dependent on Clause D2, wherein a first of the two bridging linker peptides connects the N-terminal portion of the first cytokine monomer domain to the N-terminus of the second cytokine monomer domain and the second of the two bridging linker peptides connects the C-terminus of the second cytokine monomer domain to the N-terminal portion of the first cytokine monomer domain.
D8. The single chain dimeric cytokine polypeptide of any of Clauses D1 to D7, wherein:
(i) the first cytokine monomer domain is derived from a class II cytokine and the second cytokine monomer is derived from a class I cytokine; or
(ii) the first cytokine monomer domain is derived from a class I cytokine and the second cytokine monomer is derived from a class II cytokine.
D9. The single chain dimeric cytokine polypeptide of Clause D8 (i), wherein the first cytokine monomer is split in a loop region between a pair of adjacent α-helices and wherein the two bridging linker peptides connect the second cytokine monomer domain between the adjacent α-helices of the first cytokine monomer domain; optionally, wherein the first cytokine monomer is split in the loop region between the second and third α-helices or between the third and fourth α-helices of the cytokine monomer domain.
D10. The single chain dimeric cytokine polypeptide of Clause D8 (ii), wherein the first cytokine monomer is split in a loop region between a pair of adjacent α-helices to provide N- and C-termini of the single chain dimeric cytokine, and the two bridging linker peptides connect the original C- and N-termini of the first cytokine monomer to the original N- and C-termini, respectively, of the second cytokine monomer domain to form a single chain dimeric cytokine.
D11. The single chain dimeric cytokine polypeptide of Clause D10, wherein the first cytokine monomer is split in the loop region between the second and third α-helices or between the third and fourth α-helices of the cytokine monomer domain.
D12. The single chain dimeric cytokine polypeptide of any of Clauses D8 (ii), D10 or D11, wherein the split portions or the first cytokine monomer domain are inverted in comparison to a corresponding wild type cytokine monomer on which it is based, such that the N-terminal portion of the first cytokine monomer domain corresponds to a C-terminal portion of the corresponding wild type cytokine monomer domain sequence and the C-terminal portion of the first cytokine monomer domain corresponds to an N-terminal portion of a corresponding wild-type cytokine monomer domain sequence; optionally, wherein the N-terminal portion of the first cytokine monomer domain comprises 2, 3 or 4 α-helices and the C-terminal portion of the second cytokine monomer domain comprises 2, 3 or 4 α-helices; particularly, wherein the N-terminal portion and the C-terminal portion of the first cytokine monomer domain have the same number of α-helices.
D13. The single chain dimeric cytokine polypeptide of Clause D8 (ii), wherein the second cytokine monomer domain comprises an N-terminal portion and a C-terminal portion, and wherein the N-terminal portion is connected to the C-terminal portion by an engineered peptide linker; optionally, wherein the engineered linker has a sequence of from about 3 to about 20 amino acids, from about 3 to 16 amino acids, from about 4 to 12 amino acids, from about 4 to 8 amino acids, from about 3 to 8 amino acids or from about 3 to 6 amino acids.
D14. The single chain dimeric cytokine polypeptide of Clause D13, wherein the second cytokine monomer domain is inverted in comparison to a corresponding wild type cytokine monomer on which it is based, such that the N-terminal portion of the second cytokine monomer domain corresponds to a C-terminal portion of the corresponding wild type cytokine monomer domain sequence and the C-terminal portion of the second cytokine monomer domain corresponds to an N-terminal portion of a corresponding wild-type cytokine monomer domain sequence; optionally, wherein the N-terminal portion of the second cytokine monomer domain comprises 2, 3 or 4 α-helices and the C-terminal portion of the second cytokine monomer domain comprises 2, 3 or 4 α-helices; particularly, wherein the N-terminal portion and the C-terminal portion of the second cytokine monomer domain have the same number of α-helices.
D15. The single chain dimeric cytokine polypeptide of any of Clauses D8 (ii), D13 or D14, wherein the first cytokine monomer is split in a loop region between a pair of adjacent α-helices to provide N- and C-termini of the single chain dimeric cytokine; optionally wherein the first cytokine monomer is split in the loop region between the second and third α-helices or between the third and fourth α-helices of the cytokine monomer domain.
D16. The single chain dimeric cytokine polypeptide of any of Clauses D8 (ii), or D13 to D15, wherein the second cytokine monomer is split in a loop region between a pair of adjacent α-helices, and wherein the two bridging linker peptides connect the original C- and N-termini of the first cytokine monomer, respectively, between the adjacent α-helices of the second cytokine monomer domain; optionally, wherein the second cytokine monomer is split in the loop region between the second and third α-helices or between the third and fourth α-helices of the cytokine monomer domain.
D17. The single chain dimeric cytokine polypeptide of any of Clauses D1 to D16, wherein:
(i) the first cytokine monomer is selected from or derived from a class II cytokine of the group consisting of: IL-19, IL-20, IL-22, IL-24, IL-26, IFNλ 2, 3 and 1 (IL-28A, IL-28B and IL-29, respectively), type I interferon IFNα (IFNA1, IFNA2, IFNA4, IFNA5, IFNA6, IFNA7, IFNA8, IFNA10, IFNA13, IFNA14, IFNA16, IFNA17 and IFNA21), IFNω (IFNW1), IFNE (IFNE), IFNκ (IFNK) and IFNβ (IFNB1); and wherein the second cytokine monomer is selected from or derived from a class I cytokine of the group consisting of: IL2, IL3, IL4, IL5, IL7, IL9, IL15, IL21, GMCS-F, IL6, IL11, IL12A, IL23A, IL27A, IL31, CLCF1, CNTF, CTF1, LIF, OSM and CSF3; or
(ii) the first cytokine monomer is selected from or derived from a class I cytokine of the group consisting of: IL2, IL3, IL4, IL5, IL7, IL9, IL15, IL21, GMCS-F, IL6, IL11, IL12A, IL23A, IL27A, IL31, CLCF1, CNTF, CTF1, LIF, OSM and CSF3; and wherein the second cytokine monomer is selected from or derived from a class II cytokine of the group consisting of: IL-19, IL-20, IL-22, IL-24, IL-26, IFNλ 2, 3 and 1 (IL-28A, IL-28B and IL-29, respectively), type I interferon IFNα (IFNA1, IFNA2, IFNA4, IFNA5, IFNA6, IFNA7, IFNA8, IFNA10, IFNA13, IFNA14, IFNA16, IFNA17 and IFNA21), IFNω (IFNW1), IFNE (IFNE), IFNκ (IFNK) and IFNβ (IFNB1).
D18. The single chain dimeric cytokine polypeptide of Clause D9, which comprises the sequence of SEQ ID NO: 267-(Xn)-SEQ ID NO: 268-(Xn)-SEQ ID NO: 269 (Foldikine 22-2_6B) or a sequence having at least 80%, at least 85%, at least 90%, at least 95%, at least 98%, at least 99% or 100% sequence identity thereto.
D19. The single chain dimeric cytokine polypeptide of any of Clauses D13 to D16, which comprises the sequence of SEQ ID NO: 263-(Xn)-SEQ ID NO: 264-(NtCt)-SEQ ID NO: 265-(Xn)-SEQ ID NO: 266 (Foldikine 2-22_6B) or a sequence having at least 80%, at least 85%, at least 90%, at least 95%, at least 98%, at least 99% or 100% sequence identity thereto.
D20. The single chain dimeric cytokine polypeptide of any of Clauses D10 to D12, which comprises the sequence of SEQ ID NO: 260-(Xn)-SEQ ID NO: 261-(Xn)-SEQ ID NO: 262 (Foldikine 2-22_6A) or a sequence having at least 80%, at least 85%, at least 90%, at least 95%, at least 98%, at least 99% or 100% sequence identity thereto.
D21. An isolated polynucleotide encoding a polypeptide of any of Clauses D1 to D20.
D22. A vector comprising the isolated polynucleotide of Clause D21.
D23. The vector of Clause D22, which is a bacteriophage vector or viral vector.
D24. A cell expressing a single chain dimeric cytokine polypeptide of any of Clauses D1 to D20.
D25. The cell of Clause D24, which is a recombinant eukaryotic cell; particularly a human cell.
D26. The cell of Clause D24, which is a recombinant bacterial cell; optionally wherein the bacterial cell is selected from a Mycoplasma (e.g. M. pneumoniae), E. coli, L. lactis, S. aureus, or B. subtilis bacterial strain.
D27. The cell of Clause D26, which is a Mycoplasma pneumoniae strain, optionally derived from strain M129-B7 strain (ATTC 29342); Escherichia coli BL21; or Lactococcus lactis strain NZ9000-pepN: nisRK.
D28. A cell comprising the polynucleotide or vector of any of Clauses D21 to D23.
D29. A pharmaceutical composition comprising the single chain dimeric cytokine polypeptide of any of Clauses D1 to D20, the cell of any of Clauses D24 to D28, or the isolated polynucleotide or vector of any of Clauses D21 to D23.
D30. The single chain dimeric cytokine polypeptide of any of Clauses D1 to D20, the cell of any of Clauses D24 to D28, the isolated polynucleotide or vector of any of Clauses D21 to D23, or the pharmaceutical composition of Clause D29, for use in medicine.
D31. The single chain dimeric cytokine polypeptide, isolated polynucleotide or vector, cell or pharmaceutical composition for use according to Clause D30, wherein the use is in a method for the treatment of a disease or disorder selected from inflammatory diseases and disorders; lung diseases; cancer or proliferative diseases or conditions; infectious diseases; allergy, immunological or autoimmune diseases and disorders; tissue injury, degeneration or damage condition; and conditions associated therewith.
D32. The single chain dimeric cytokine polypeptide, isolated polynucleotide or vector, cell or pharmaceutical composition for use according to Clause D31, wherein the cancer or proliferative disease or disorder is a solid tumour, for example, selected from the group consisting of lung cancers (in particular lung adenocarcinomas or lung squamous carcinoma), bladder cancer, cervical cancer, breast cancer, colon cancer, brain glioblastoma, pancreatic cancer, acute monocytic leukemia, kidney cancer, colorectal cancer, skin cancer (e.g. melanoma), stomach cancer, thyroid cancer, bone cancer and liver cancer.
D33. The single chain dimeric cytokine polypeptide, isolated polynucleotide or vector, cell or pharmaceutical composition for use according to Clause D31, wherein the inflammatory disease or disorder is selected from the group consisting of allergy, asthma, autoimmune diseases, coeliac disease, glomerulonephritis, hepatitis, inflammatory bowel disease, preperfusion injury, transplant rejection, diabetes, Alzheimer's disease, osteoarthritis, fibromyalgia, muscular low back pain, arthritis, muscular neck pain, myocarditis, Ankylosing Spondylitis (AS), Antiphospholipid Antibody Syndrome (APS), Myositis, Rheumatoid Arthritis, Scleroderma, Sjogren's Syndrome, fibrosis.
D34. The single chain dimeric cytokine polypeptide, isolated polynucleotide or vector, cell or pharmaceutical composition for use according to Clause D31, wherein the tissue injury, degeneration or damage condition is selected from the group consisting of colitis, wound healing, hair growth, liver regeneration and matrix synthesis.
D35. The single chain dimeric cytokine polypeptide, isolated polynucleotide or vector, cell or pharmaceutical composition for use according to Clause D31, wherein the use is in cancer immunotherapy and/or immune oncology.
E1. A single chain dimeric cytokine polypeptide comprising: (a) a first cytokine monomer domain or a functional portion thereof; and (b) a second cytokine monomer domain or a functional portion thereof, wherein the first cytokine monomer domain sequence is derived from a different type of cytokine to the second cytokine monomer domain sequence; and wherein the single chain dimeric cytokine polypeptide has a tertiary structure comprising a left 3D cytokine domain formed of the first cytokine monomer domain or a functional portion thereof, and a right 3D cytokine domain formed of the second cytokine monomer domain or a functional portion thereof.
E2. The single chain dimeric cytokine polypeptide of Clause E1, wherein:
(i) the first cytokine monomer domain sequence is derived from an IL-10 or IFNγ cytokine sequence and the second cytokine monomer domain sequence is derived from a class I or a class II cytokine sequence; or
(ii) the first cytokine monomer domain sequence is derived from a class I or a class II cytokine sequence and the second cytokine monomer domain sequence is derived from an IL-10 or IFNγ cytokine sequence.
E3. The single chain dimeric cytokine polypeptide of Clause E1 or Clause E2, wherein the left 3D cytokine domain is connected to the right 3D cytokine domain by two bridging linker peptides.
E4. The single chain dimeric cytokine polypeptide of Clause E3, wherein the two bridging linker peptides each has a sequence of from about 3 to about 20 amino acids, from about 3 to about 16 amino acids, from about 4 to about 12 amino acids, from about 4 to about 8 amino acids, from about 3 to about 8 amino acids or from about 3 to about 6 amino acids; preferably wherein the bridging peptide linker sequences comprises 5 or less, 3 or less, or 2 or less adjacent Gly and/or Ser residues.
E5. The single chain dimeric cytokine polypeptide of any of Clauses E1 to E4, wherein the second cytokine monomer domain or functional portion thereof is inserted within the sequence of the first cytokine monomer domain such that a first sequence portion of the first cytokine monomer is arranged at the N-terminus of the second cytokine monomer or functional portion thereof, and a second sequence portion of the first cytokine monomer is arranged at the C-terminus of the second cytokine monomer or functional portion thereof.
E6. The single chain dimeric cytokine polypeptide of any of Clauses E1 to E5, wherein the left 3D cytokine domain is a split domain cytokine formed from first and second sequence portions of the first cytokine monomer domain or a functional portion thereof and the right 3D cytokine domain is a continuous domain cytokine formed from the second cytokine monomer domain or a functional portion thereof.
E7. The single chain dimeric cytokine polypeptide of Clause E3 or any of Clauses E4 to E6 when dependent on Clause E3, wherein the two bridging linker peptides are the same sequence or are different sequences.
E8. The single chain dimeric cytokine polypeptide of Clause E3 or of any of Clauses E4 to E7 when dependent on Clause E3, wherein a first of the two bridging linker peptides connects the N-terminal portion of the first cytokine monomer domain to the N-terminal portion of the second cytokine monomer domain and the second of the two bridging linker peptides connects the C-terminus of the second cytokine monomer domain to the C-terminal portion of the first cytokine monomer domain.
E8A. The single chain dimeric cytokine polypeptide of Clause E8, wherein the N-terminal portion of the second cytokine monomer domain corresponds to a C-terminal portion of a natural cytokine monomer, and the C-terminal portion of the second cytokine monomer domain corresponds to an N-terminal portion of a natural cytokine monomer,
E9. The single chain dimeric cytokine polypeptide of any of Clauses E1 to E8, wherein:
(i) the first cytokine monomer domain is derived from an IL-10 cytokine and the second cytokine monomer is derived from a class I cytokine;
(ii) the first cytokine monomer domain is derived from an IL-10 cytokine and the second cytokine monomer is derived from a class II cytokine;
(iii) the first cytokine monomer domain is derived from an IFNγ cytokine and the second cytokine monomer is derived from a class I cytokine; or
(iv) the first cytokine monomer domain is derived from an IFNγ cytokine and the second cytokine monomer is derived from a class II cytokine.
E10. The single chain dimeric cytokine polypeptide of Clause E6 or of any of Clauses E7 to E9 when dependent on Clause E6, wherein the first cytokine monomer is split in a loop region between a pair of adjacent α-helices and wherein the two bridging linker peptides connect the second cytokine monomer domain between the adjacent α-helices of the first cytokine monomer domain; optionally, wherein the first cytokine monomer is split in the loop region between the second and third α-helices or between the third and fourth α-helices of the cytokine monomer domain.
E11. The single chain dimeric cytokine polypeptide of Clause E6 or of any of Clauses E7 to E9 when dependent on Clause E6, wherein the first cytokine monomer is split in a loop region between a pair of adjacent α-helices to provide N- and C-termini of the single chain dimeric cytokine, and the two bridging linker peptides connect the original C- and N-termini of the first cytokine monomer to the original N- and C-termini, respectively, of the second cytokine monomer domain to form a single chain dimeric cytokine.
E12. The single chain dimeric cytokine polypeptide of Clause E11, wherein the first cytokine monomer is split in the loop region between the second and third α-helices or between the third and fourth α-helices of the cytokine monomer domain.
E13. The single chain dimeric cytokine polypeptide of Clause E6 or any of Clauses E7 to E12 when dependent on Clause E6, wherein the split portions or the first cytokine monomer domain are inverted in comparison to a corresponding wild type cytokine monomer on which it is based, such that the N-terminal portion of the first cytokine monomer domain corresponds to a C-terminal portion of the corresponding wild type cytokine monomer domain sequence and the C-terminal portion of the first cytokine monomer domain corresponds to an N-terminal portion of a corresponding wild-type cytokine monomer domain sequence; optionally, wherein the N-terminal portion of the first cytokine monomer domain comprises 2, 3 or 4 α-helices and the C-terminal portion of the second cytokine monomer domain comprises 2, 3 or 4 α-helices; particularly, wherein the N-terminal portion and the C-terminal portion of the first cytokine monomer domain have the same number of α-helices.
E14. The single chain dimeric cytokine polypeptide of Clause E6 or any of Clauses E7 to E13 when dependent on Clause E6, wherein the second cytokine monomer domain comprises an N-terminal portion and a C-terminal portion, and wherein the N-terminal portion is connected to the C-terminal portion by an engineered peptide linker; optionally, wherein the engineered linker has a sequence of from about 3 to about 20 amino acids, from about 3 to 16 amino acids, from about 4 to 12 amino acids, from about 4 to 8 amino acids, from about 3 to 8 amino acids or from about 3 to 6 amino acids.
E15. The single chain dimeric cytokine polypeptide of Clause E14, wherein the second cytokine monomer domain is inverted in comparison to a corresponding wild type cytokine monomer on which it is based, such that the N-terminal portion of the second cytokine monomer domain corresponds to a C-terminal portion of the corresponding wild type cytokine monomer domain sequence and the C-terminal portion of the second cytokine monomer domain corresponds to an N-terminal portion of a corresponding wild-type cytokine monomer domain sequence; optionally, wherein the N-terminal portion of the second cytokine monomer domain comprises 2, 3 or 4 α-helices and the C-terminal portion of the second cytokine monomer domain comprises 2, 3 or 4 α-helices; particularly, wherein the N-terminal portion and the C-terminal portion of the second cytokine monomer domain have the same number of α-helices.
E16. The single chain dimeric cytokine polypeptide of Clause E6 or any of Clauses E7 to E15 when dependent on Clause E6, wherein the first cytokine monomer is split in a loop region between a pair of adjacent α-helices to provide N- and C-termini of the single chain dimeric cytokine; optionally wherein the first cytokine monomer is split in the loop region between the second and third α-helices or between the third and fourth α-helices of the cytokine monomer domain.
E17. The single chain dimeric cytokine polypeptide of any of Clauses E14 to E16, wherein the second cytokine monomer is split in a loop region between a pair of adjacent α-helices, and wherein the two bridging linker peptides connect the original C- and N-termini of the first cytokine monomer, respectively, between the adjacent α-helices of the second cytokine monomer domain; optionally, wherein the second cytokine monomer is split in the loop region between the second and third α-helices or between the third and fourth α-helices of the cytokine monomer domain.
E18. The single chain dimeric cytokine polypeptide of any of Clauses E1 to E17, wherein: (i) the class I cytokine is selected and/or derived from a cytokine of the group consisting of: IL2, IL3, IL4, IL5, IL7, IL9, IL15, IL21, GMCS-F, IL6, IL11, IL12A, IL23A, IL27A, IL31, CLCF1, CNTF, CTF1, LIF, OSM and CSF3; or
(ii) the class II cytokine is selected and/or derived from a cytokine of the group consisting of: IL-19, IL-20, IL-22, IL-24, IL-26, IFNλ 2, 3 and 1 (IL-28A, IL-28B and IL-29, respectively), type I interferon IFNα (IFNA1, IFNA2, IFNA4, IFNA5, IFNA6, IFNA7, IFNA8, IFNA10, IFNA13, IFNA14, IFNA16, IFNA17 and IFNA21), IFNω (IFNW1), IFNE (IFNE), IFNκ (IFNK) and IFNβ (IFNB1).
E19. The single chain dimeric cytokine polypeptide of any of Clauses E1 to E18, wherein:
(i) the first cytokine monomer is selected from or derived from a class II cytokine of the group consisting of: IL-19, IL-20, IL-22, IL-24, IL-26, IFNλ 2, 3 and 1 (IL-28A, IL-28B and IL-29, respectively), type I interferon IFNα (IFNA1, IFNA2, IFNA4, IFNA5, IFNA6, IFNA7, IFNA8, IFNA10, IFNA13, IFNA14, IFNA16, IFNA17 and IFNA21), IFNω (IFNW1), IFNε (IFNE), IFNκ (IFNK) and IFNβ (IFNB1); and wherein the second cytokine monomer is selected from or derived from a swapped domain cytokine of the group consisting of: IL-10 and IFNγ; or
(ii) the first cytokine monomer is selected from or derived from a swapped domain cytokine of the group consisting of: IL-10 and IFNγ; and wherein the second cytokine monomer is selected from or derived from a class II cytokine of the group consisting of: IL-19, IL-20, IL-22, IL-24, IL-26, IFNλ 2 2, 3 and 1 (IL-28A, IL-28B and IL-29, respectively), type I interferon IFNα (IFNA1, IFNA2, IFNA4, IFNA5, IFNA6, IFNA7, IFNA8, IFNA10, IFNA13, IFNA14, IFNA16, IFNA17 and IFNA21), IFNω (IFNW1), IFNε (IFNE), IFNκ (IFNK) and IFNβ (IFNB1).
E20. The single chain dimeric cytokine polypeptide of any of Clauses E1 to E18, wherein:
(i) the first cytokine monomer is selected from or derived from a class I cytokine of the group consisting of: IL6, IL11, IL12A, IL23A, IL27A, IL31, CLCF1, CNTF, CTF1, LIF, OSM and CSF3; and wherein the second cytokine monomer is selected from or derived from a swapped domain cytokine of the group consisting of: IL-10 and IFNγ; or
(ii) the first cytokine monomer is selected from or derived from a swapped domain cytokine of the group consisting of: IL-10 and IFNγ; and wherein the second cytokine monomer is selected from or derived from a class I cytokine of the group consisting of: IL6, IL11, IL12A, IL23A, IL27A, IL31, CLCF1, CNTF, CTF1, LIF, OSM and CSF3.
E21. The single chain dimeric cytokine polypeptide of any of Clauses E1 to E18, wherein:
(i) the first cytokine monomer is selected from or derived from a class I cytokine of the group consisting of: IL2, IL3, IL4, IL7, IL9, IL15, IL21 and GMCS-F; and wherein the second cytokine monomer is selected from or derived from a swapped domain cytokine of the group consisting of: IL-10 and IFNγ; or
(ii) the first cytokine monomer is selected from or derived from a swapped domain cytokine of the group consisting of: IL-10 and IFNγ; and wherein the second cytokine monomer is selected from or derived from a class I cytokine of the group consisting of: IL2, IL3, IL4, IL7, IL9, IL15, IL21 and GMCS-F.
E22. The single chain dimeric cytokine polypeptide of any of Clauses E1 to E19, wherein the first cytokine monomer is IL-22 and the second cytokine monomer is IL-10; optionally wherein the IL-22 and IL-10 are derived from a human or a mouse sequence.
E23. The single chain dimeric cytokine polypeptide of any of Clauses E1 to E19, wherein the first cytokine monomer is IL-10 and the second cytokine monomer is IL-22; optionally wherein the IL-22 and IL-10 are derived from a human or a mouse sequence.
E24. The single chain dimeric cytokine polypeptide of any of Clauses E1 to E19 or E23, wherein the first cytokine monomer comprises the sequence of IL-10 ORF (SEQ ID NO: 4), SEQ ID NO: 181, SEQ ID NO: 349 or SEQ ID NO: 350 or a functional fragment thereof, or a sequence having at least 80%, at least 85%, at least 90%, at least 95%, at least 98%, at least 99% or 100% sequence identity to SEQ ID NO: 4 or SEQ ID NO: 181, SEQ ID NO: 349 or SEQ ID NO: 350 or the functional fragment thereof; and wherein the second cytokine monomer comprises the sequence of IL-22 (SEQ ID NO: 184) or a functional fragment thereof, or a sequence having at least 80%, at least 85%, at least 90%, at least 95%, at least 98%, at least 99% or 100% sequence identity to SEQ ID NO: 184 or the functional fragment thereof.
E25. The single chain dimeric cytokine polypeptide of Clause E24, wherein the IL-22 cytokine monomer sequence includes one or more amino acid substitutions that increase the stability of the hydrophobic core of the IL-22 monomer domain; optionally, wherein the one or more amino acid substitutions are selected from the group consisting of: T56L or T56M, A66M, N68Q, V95I, T99F and S317L, wherein the numbering of mutated positions is based on the IL-22 sequence of SEQ ID NO: 184, wherein the A at the first position of SEQ ID NO: 184 is sequence position A34.
E26. The single chain dimeric cytokine polypeptide of Clause E25, wherein the IL-22 cytokine monomer sequence includes a set of amino acid substitutions selected from:
   (i) T56L, A66M, V95I, T99F and S173L; and
   (ii) T56M, A66M, N68Q, V95I, T99F and S173L.
E27. The single chain dimeric cytokine polypeptide of any of Clauses E24 to E26, wherein IL-22 cytokine monomer sequence includes one or more amino acid substitutions that increase the stability and/or binding affinity of the IL-22 monomer domain to its receptor R1 and/or R2; optionally, wherein the one or more amino acid substitutions are selected from the group consisting of: D43E, S45R, K61W, S64M, T70I, E77Y and A132Y, wherein the numbering of mutated positions is based on SEQ ID NO: 129, wherein the A at the first position of SEQ ID NO: 184 is sequence position A34.
E28. The single chain dimeric cytokine polypeptide of Clause E27, wherein the IL-22 cytokine monomer sequence includes a set of amino acid substitutions selected from:
   (i) A132Y, D43E, S45R, E77Y, T70I;
   (ii) K61W, S64M; and
   (iii) A132Y, D43E, S45R, E77Y, T70I, K61W, S64M.
E29. The single chain dimeric cytokine polypeptide of any of Clauses E1 to E19 or E22, wherein the second cytokine monomer comprises the sequence of IL-10 ORF (SEQ ID NO: 4), SEQ ID NO: 181, SEQ ID NO: 349 or SEQ ID NO: 350 or a functional fragment thereof, or a sequence having at least 80%, at least 85%, at least 90%, at least 95%, at least 98%, at least 99% or 100% sequence identity to SEQ ID NO: 4 or SEQ ID NO: 181, SEQ ID NO: 349 or SEQ ID NO: 350 or the functional fragment thereof; and wherein the first cytokine monomer comprises the sequence of IL-22 (SEQ ID NO: 184) or a functional fragment thereof, or a sequence having at least 80%, at least 85%, at least 90%, at least 95%, at least 98%, at least 99% or 100% sequence identity to SEQ ID NO: 184 or the functional fragment thereof.
E30. The single chain dimeric cytokine polypeptide of Clause E29, wherein the IL-22 cytokine monomer sequence includes one or more amino acid substitutions that increase the stability of the IL-22 monomer split 3D domain; optionally, wherein the one or more amino acid substitutions are selected from the group consisting of: T17L, A27M, V56I, T60F and S314L, wherein the numbering of mutated positions is based on the Foldikine1022_3 sequence of SEQ ID NO: 75.
E31. The single chain dimeric cytokine polypeptide of Clause E30, wherein the IL-22 cytokine monomer sequence includes all of the amino acid substitutions: T17L, A27M, V56I, T60F and S314L.
E32. The single chain dimeric cytokine polypeptide of Clause E24 or any of Clauses E25 to E28 when dependent on Clause E24, or the single chain dimeric cytokine polypeptide of Clause E29, or Clause E30 or Clause E31 when dependent on Clause E29, wherein the IL-10 cytokine monomer sequence includes one or more amino acid substitution selected from the group consisting of: N18I, D28E, S31K, N45S, N921, K99N and T155M wherein the numbering of mutated positions is based on SEQ ID NO: 181, wherein the Q at the first position of SEQ ID NO: 181 is sequence position Q7.
E33. The single chain dimeric cytokine polypeptide of Clause E32, wherein the IL-10 cytokine monomer sequence includes at least the amino acid substitutions: D28E, S31K, N45S and T155M.
E34. The single chain dimeric cytokine polypeptide of Clause E32 or Clause E33, wherein the IL-10 cytokine monomer sequence includes all of the amino acid substitutions: D28E, S31K, N45S, N921, K99N and T155M.
E35. The single chain dimeric cytokine polypeptide of Clause E29 or any of Clauses E30 to E34 when dependent on Clause E29, wherein the N-terminal portion of the IL-22 monomer split 3D domain is connected to the N-terminus of the IL-10 monomer continuous 3D domain by a bridging linker peptide comprising or having the sequence DPKAAFKS (SEQ ID NO: 314), and the C-terminus of the IL-10 monomer continuous 3D domain is connected to the C-terminal portion of the IL-22 monomer split 3D domain by a bridging linker peptide comprising or having the sequence DSKVNR (SEQ ID NO: 315).
E36. The single chain dimeric cytokine polypeptide of Clause E29 or any of Clauses E30 to E35 when dependent on Clause E29, wherein the N-terminal portion of the IL-10 monomer continuous 3D domain is connected to the C-terminal portion of the IL-10 monomer continuous 3D domain by an engineered peptide linker comprising or having the sequence NFGGLDY (SEQ ID NO: 316).
E37. The single chain dimeric cytokine polypeptide of any of Clauses E1 to E36, which comprises a sequence selected from the group consisting of SEQ ID NOs: 77 (Foldikine1022), 75 (Foldikine1022_3) and 76 (Foldikine1022_5), or a sequence having at least 80%, at least 85%, at least 90%, at least 95%, at least 98%, at least 99% or 100% sequence identity thereto.
E38. The single chain dimeric cytokine polypeptide of Clause E37, which comprises the sequence of SEQ ID NO: 76 (Foldikine1022_5) or a sequence having at least 80%, at least 85%, at least 90%, at least 95%, at least 98%, at least 99% or 100% sequence identity thereto.
E39. An isolated polynucleotide encoding a polypeptide of any of Clauses E1 to E38.
E40. A vector comprising the isolated polynucleotide of Clause E39.
E41. The vector of Clause E40, which is a bacteriophage vector or a viral vector.
E42. A cell expressing a single chain dimeric cytokine polypeptide of any of Clauses E1 to
E38.
E43. The cell of Clause E42, which is a recombinant eukaryotic cell; particularly a human cell.
E44. The cell of Clause E42, which is a recombinant bacterial cell; optionally wherein the bacterial cell is selected from a Mycoplasma (e.g. M. pneumoniae), E. coli, L. lactis, S. aureus, or B. subtilis bacterial strain.
E45. The cell of Clause E44, which is a Mycoplasma pneumoniae strain, optionally derived from strain M129-B7 strain (ATTC 29342); Escherichia coli BL21; or Lactococcus lactis strain NZ9000-pepN: nisRK.
E46. A cell comprising the polynucleotide or vector of any of Clauses E39 to E41.
E47. A pharmaceutical composition comprising the single chain dimeric cytokine polypeptide of any of Clauses E1 to E38, the cell of any of Clauses E42 to E46, or the isolated polynucleotide or vector of any of Clauses E39 to E41.
E48. The single chain dimeric cytokine polypeptide of any of Clauses E1 to E38, the cell of any of Clauses E42 to E46, the isolated polynucleotide or vector of any of Clauses E39 to E41, or the pharmaceutical composition of Clause E47, for use in medicine.
E49. The single chain dimeric cytokine polypeptide, isolated polynucleotide or vector, cell or pharmaceutical composition for use according to Clause E48, wherein the use is in a method for the treatment of a disease or disorder selected from inflammatory diseases and disorders; lung diseases; cancer or proliferative diseases or conditions; infectious diseases; allergy, immunological or autoimmune diseases and disorders; tissue injury, degeneration or damage condition; and conditions associated therewith.
E50. The single chain dimeric cytokine polypeptide, isolated polynucleotide or vector, cell or pharmaceutical composition for use according to Clause E49, wherein the cancer or proliferative disease or disorder is a solid tumour, for example, selected from the group consisting of lung cancers (in particular lung adenocarcinomas or lung squamous carcinoma), bladder cancer, cervical cancer, breast cancer, colon cancer, brain glioblastoma, pancreatic cancer, acute monocytic leukemia, kidney cancer, colorectal cancer, skin cancer (e.g. melanoma), stomach cancer, thyroid cancer, bone cancer and liver cancer.
E51. The single chain dimeric cytokine polypeptide, isolated polynucleotide or vector, cell or pharmaceutical composition for use according to Clause E49, wherein the inflammatory disease or disorder is selected from the group consisting of allergy, asthma, autoimmune diseases, coeliac disease, glomerulonephritis, hepatitis, inflammatory bowel disease, preperfusion injury, transplant rejection, diabetes, Alzheimer's disease, osteoarthritis, fibromyalgia, muscular low back pain, arthritis, muscular neck pain, myocarditis, Ankylosing Spondylitis (AS), Antiphospholipid Antibody Syndrome (APS), Myositis, Rheumatoid Arthritis, Scleroderma, Sjogren's Syndrome, fibrosis.
E52. The single chain dimeric cytokine polypeptide, isolated polynucleotide or vector, cell or pharmaceutical composition for use according to Clause E51, wherein the tissue injury, degeneration or damage condition is selected from the group consisting of colitis, wound healing, hair growth, liver regeneration and matrix synthesis.

While particular embodiments have been illustrated and described herein, it should be understood that various other changes and modifications may be made without departing from the scope of the claimed subject matter. Moreover, although various aspects of the claimed subject matter have been described herein, such aspects need not be utilised in combination. It is therefore intended that the appended claims cover all such changes and modifications that are within the scope of the claimed subject matter, which is defined by the appended claims.

1. A single chain dimeric cytokine polypeptide comprising: (a) a first cytokine monomer domain or a functional portion thereof; and (b) a second cytokine monomer domain or a functional portion thereof, wherein the sequence of one of the cytokine monomer domains or functional portion is not continuous and is interrupted by the sequence of the other cytokine monomer domain or a functional portion thereof.

2. The single chain dimeric cytokine polypeptide of claim 1 comprising: (a) a first cytokine monomer domain or a functional portion thereof; and (b) a second cytokine monomer domain or a functional portion thereof, wherein the sequence of the second cytokine monomer domain or functional portion thereof is continuous in sequence while the sequence of the first cytokine monomer domain is such that a first sequence portion of the first cytokine monomer domain is arranged at the N-terminus of the second cytokine monomer domain or functional portion thereof, and a second sequence portion of the first cytokine monomer domain is arranged at the C-terminus of the second cytokine monomer domain or functional portion thereof.

3. The single chain dimeric cytokine polypeptide of claim 2, wherein the first sequence portion of the first cytokine monomer domain corresponds to a N-terminal portion of a natural cytokine monomer domain, and the second sequence portion of the first cytokine monomer domain corresponds to an C-terminal portion of the natural cytokine monomer domain.

4. The single chain dimeric cytokine polypeptide of claim 2, which comprises:
i) a split domain that comprises the free N- and C-termini of the single chain dimeric cytokine polypeptide, and the first and second sequence portions of the first cytokine monomer domain in their natural sequence order; and
ii) a continuous domain that comprises the second cytokine monomer domain or a functional portion thereof organized into a first sequence portion and a second sequence portion of the second cytokine monomer domain, the first and second sequence portions being in an order inverted compared to their natural sequence order;
wherein the sequences of the first and second sequence portions of the first cytokine monomer domain, in the split domain, are separated by the sequence of the continuous domain.

5. The single chain dimeric cytokine polypeptide of claim 3, which is a single chain dimeric class I polypeptide that comprises i) a N-terminal portion of a first class I cytokine monomer comprising α-helices A and B of said first class I cytokine monomer, ii) a linker peptide bridging α-helix B of said first class I cytokine monomer with α-helix C of a second class I cytokine monomer, iii) a C-terminal portion of said second class I cytokine monomer comprising α-helices C and D of said second class I cytokine monomer, iv) a linker peptide bridging α-helix D of said second class I cytokine monomer with α-helix A of said second class I cytokine monomer, v) a N-terminal portion of said second class I cytokine monomer comprising α-helices A and B of said second class I cytokine monomer, vi) a linker peptide bridging α-helix B of the second class I cytokine monomer with α-helix C of the first class I cytokine monomer, and vii) a C-terminal portion of said first class I cytokine monomer comprising α-helices C and D of said first class I cytokine monomer.

6. The single chain dimeric cytokine polypeptide of claim 3 or 4 claim 3, which is a single chain dimeric class II polypeptide that comprises i) a N-terminal portion of a first class II cytokine monomer comprising α-helices A to C of said first class II cytokine monomer, ii) a linker peptide bridging α-helix C of said first class II cytokine monomer with α-helix D of a second class II cytokine monomer, iii) a C-terminal portion of said second class II cytokine monomer comprising α-helices D to F of said second class II cytokine monomer, iv) a linker peptide bridging α-helix F of said second class I cytokine monomer with α-helix A of said second class II cytokine monomer, v) a N-terminal portion of said second class II cytokine monomer comprising α-helices A to C of said second class II cytokine monomer, vi) a linker peptide bridging α-helix C of the second class II cytokine monomer with α-helix D of the first class II cytokine monomer, and vii) a C-terminal portion of said first class II cytokine monomer comprising α-helices D to F of said first class II cytokine monomer.

7. The single chain dimeric cytokine polypeptide of claim 3, which is:
i) dimeric IL-22 which comprises sequence SEQ ID NO: 325-(Xn1)-SEQ ID NO: 326-(NtCt)-SEQ ID NO: 327-(Xn2)-SEQ ID NO: 328, or a sequence at least 80% identical thereto that retains at least the same stability, and/or at least the same level of interaction with the respective receptor(s) of said monomeric class II cytokine;
ii) dimeric IL-19 which comprises sequence SEQ ID NO: 195-(Xn1)-SEQ ID NO: 196-(NtCt)-SEQ ID NO: 197-(Xn2)-SEQ ID NO: 198 or a sequence at least 80% identical thereto that retains at least the same stability, and/or at least the same level of interaction with the respective receptor(s) of said monomeric class II cytokine,
iii) dimeric IL-20 which comprises sequence SEQ ID NO: 200-(Xn1)-SEQ ID NO: 201-(NtCt)-SEQ ID NO: 202-(Xn2)-SEQ ID NO: 203 or a sequence at least 80% identical thereto that retains at least the same stability, and/or at least the same level of interaction with the respective receptor(s) of said monomeric class II cytokine,
iv) dimeric IL-26 which comprises sequence SEQ ID NO: 210-(Xn1)-SEQ ID NO: 211-(NtCt)-SEQ ID NO: 212-(Xn2)-SEQ ID NO: 213 or a sequence at least 80% identical thereto that retains at least the same stability, and/or at least the same level of interaction with the respective receptor(s) of said monomeric class II cytokine,
v) dimeric IFN22 which comprises sequence SEQ ID NO: 215-(Xn1)-SEQ ID NO: 216-(NtCt)-SEQ ID NO: 217-(Xn2)-SEQ ID NO: 218 or a sequence at least 80% identical thereto that retains at least the same stability, and/or at least the same level of interaction with the respective receptor(s) of said monomeric class II cytokine,
vi) dimeric IFN23 which comprises sequence SEQ ID NO: 220-(Xn1)-SEQ ID NO: 221-(NtCt)-SEQ ID NO: 222-(Xn2)-SEQ ID NO: 223 or a sequence at least 80% identical thereto that retains at least the same stability, and/or at least the same level of interaction with the respective receptor(s) of said monomeric class II cytokine,
vii) dimeric IFN21 which comprises sequence SEQ ID NO: 225-(Xn1)-SEQ ID NO: 226-(NtCt)-SEQ ID NO: 227-(Xn2)-SEQ ID NO: 228 or a sequence at least 80% identical thereto that retains at least the same stability, and/or at least the same level of interaction with the respective receptor(s) of said monomeric class II cytokine,
viii) dimeric IFNα1/13 which comprises sequence SEQ ID NO: 230-(Xn1)-SEQ ID NO: 231-(NtCt)-SEQ ID NO: 232-(Xn2)-SEQ ID NO: 233 or a sequence at least 80% identical thereto that retains at least the same stability, and/or at least the same level of interaction with the respective receptor(s) of said monomeric class II cytokine, ix) dimeric IFNα2 which comprises sequence SEQ ID NO: 235-(Xn1)-SEQ ID NO: 236-(NtCt)-SEQ ID NO: 237-(Xn2)-SEQ ID NO: 238 or a sequence at least 80% identical thereto that retains at least the same stability, and/or at least the same level of interaction with the respective receptor(s) of said monomeric class II cytokine),
x) dimeric IFNβ which comprises sequence SEQ ID NO: 240-(Xn1)-SEQ ID NO: 241-(NtCt)-SEQ ID NO: 242-(Xn2)-SEQ ID NO: 243 or a sequence at least 80% identical thereto that retains at least the same stability, and/or at least the same level of interaction with the respective receptor(s) of said monomeric class II cytokine,
xi) dimeric IFNΩ1 which comprises sequence SEQ ID NO: 245-(Xn1)-SEQ ID NO: 246-(NtCt)-SEQ ID NO: 247-(Xn2)-SEQ ID NO: 248 or a sequence at least 80% identical thereto that retains at least the same stability, and/or at least the same level of interaction with the respective receptor(s) of said monomeric class II cytokine,
xii) dimeric IFNε which comprises sequence SEQ ID NO: 250-(Xn1)-SEQ ID NO: 251-(NtCt)-SEQ ID NO: 252-(Xn2)-SEQ ID NO: 253 or a sequence at least 80% identical thereto that retains at least the same stability, and/or at least the same level of interaction with the respective receptor(s) of said monomeric class II cytokine, or
xiii) dimeric IFNκ which comprises sequence SEQ ID NO: 255-(Xn1)-SEQ ID NO: 256-(NtCt)-SEQ ID NO: 257-(Xn2)-SEQ ID NO: 258 or a sequence at least 80% identical thereto that retains at least the same stability, and/or at least the same level of interaction with the respective receptor(s) of said monomeric class II cytokine,
wherein (Xn1) and (Xn2) are peptide linkers.

8. The single chain dimeric cytokine polypeptide of claim 3, which is a single chain dimeric IL-22 polypeptide that comprises sequence SEQ ID NO:65, SEQ ID NO:66, SEQ ID NO: 282, SEQ ID NO: 283, SEQ ID NO: 284, SEQ ID NO: 285, SEQ ID NO: 286, SEQ ID NO: 287, or SEQ ID NO: 288, or a sequence at least 80% identical thereto that retains at least the same stability, and/or at least the same level of interaction with IL-22 receptor 1 and/or IL-22 receptor 2.

9. The single chain dimeric cytokine polypeptide of claim 3, which a single chain chimeric IL-10-IL-22 polypeptide is provided that comprises or consists of sequence SEQ ID NO: 76, or a sequence at least 80% identical thereto that retains at least the same stability, and/or at least the same level of interaction with IL-10 receptor and IL-22 receptor.

10. The single chain dimeric cytokine polypeptide of claim 2, wherein the first sequence portion of the first cytokine monomer domain corresponds to a C-terminal portion of a natural cytokine monomer domain, and the second sequence portion of the first cytokine monomer domain corresponds to an N-terminal portion of a natural cytokine monomer domain.

11. The single chain dimeric cytokine polypeptide of claim 10, which comprises:
i) a split domain that comprises the free N- and C-termini of the single chain dimeric cytokine polypeptide, and the first and second sequence portions of the first cytokine monomer domain in an order inverted compared to their natural sequence order; and
ii) a continuous domain that comprises the second cytokine monomer domain or a functional portion thereof in its natural sequence order;
wherein the sequences of the first and second sequence portions of the first cytokine monomer domain, in the split domain, are separated by the sequence of the continuous domain.

12. The single chain dimeric cytokine polypeptide of claim 10, which a single chain dimeric class I polypeptide wherein:
a) a linker peptide bridges the fourth α-helix of a first class I cytokine monomer with the first α-helix of a second class I cytokine monomer;
b) a linker peptide bridges the fourth α-helix of the second class I cytokine monomer with the first α-helix of the first class I cytokine monomer; and
either α-helices A and B of said first class I cytokine monomer are not bridged, or α-helices B and C of said first class I cytokine monomer are not bridged, or α-helices C and D of said first class I cytokine monomer are not bridged.

13. The single chain dimeric cytokine polypeptide of claim 10, which is based on short chain Class I cytokine and is:
i) dimeric IL-2 which comprises SEQ ID NO: 89-(Xn1)-SEQ ID NO:90-(Xn2)-SEQ ID NO: 91,
ii) dimeric IL-4 which comprises SEQ ID NO: 93-(Xn1)-SEQ ID NO:94-(Xn2)-SEQ ID NO: 95,
iii) dimeric IL-3 which comprises SEQ ID NO: 97-(Xn1)-SEQ ID NO:98-(Xn2)-SEQ ID NO: 99,
iv) dimeric IL-7 which comprises SEQ ID NO: 103-(Xn1)-SEQ ID NO:104-(Xn2)-SEQ ID NO: 105,
v) dimeric IL-9 which comprises SEQ ID NO: 107-(Xn1)-SEQ ID NO:108-(Xn2)-SEQ ID NO: 109,
vi) dimeric IL-15 which comprises SEQ ID NO: 110-(Xn1)-SEQ ID NO:111-(Xn22)-SEQ ID NO: 112,
vii) dimeric IL-21 which comprises SEQ ID NO: 113-(Xn1)-SEQ ID NO:114-(Xn)-SEQ ID NO: 115,
viii) dimeric TSLP which comprises SEQ ID NO: 116-(Xn1)-SEQ ID NO:117-(Xn2)-SEQ ID NO: 118, and
ix) dimeric GM-CSF (which may comprise or consist of SEQ ID NO: 120-(Xn1)-SEQ ID NO: 121-(Xn2)-SEQ ID NO:12SEQ ID NO: 101),
or a sequence at least 80% identical thereto that retains at least the same stability, and/or at least the same level of interaction with the respective receptor(s) of said monomeric short chain Class I cytokine;
wherein (Xn1) and (Xn2) are peptide linkers.

14. The single chain dimeric cytokine polypeptide of claim 10, which is based on long chain Class I cytokine and is:
i) dimeric IL-6 which comprises SEQ ID NO: 135-(Xn1)-SEQ ID NO:136-(Xn2)-SEQ ID NO: 137,
ii) dimeric IL-11 which comprises SEQ ID NO: 139-(Xn1)-SEQ ID NO:140-(Xn2)-SEQ ID NO: 141,
iii) dimeric IL-12a which comprises SEQ ID NO: 143-(Xn1)-SEQ ID NO:144-(Xn2)-SEQ ID NO: 145,
iv) dimeric IL-23a which comprises SEQ ID NO: 147-(Xn1)-SEQ ID NO:148-(Xn2)-SEQ ID NO: 149,
v) dimeric IL-27a which comprises SEQ ID NO: 151-(Xn1)-SEQ ID NO:152-(Xn2)-SEQ ID NO: 153,
vi) dimeric IL-31 which comprises SEQ ID NO: 154-(Xn1)-SEQ ID NO:155-(Xn2)-SEQ ID NO: 156,
vii) dimeric CLCF1 which comprises SEQ ID NO: 158-(Xn1)-SEQ ID NO:159-(Xn2)-SEQ ID NO:160,
viii) dimeric ONCM which comprises SEQ ID NO: 161-(Xn1)-SEQ ID NO:162-(Xn2)-SEQ ID NO:163,
ix) dimeric CTF1 which comprises SEQ ID NO: 165-(Xn1)-SEQ ID NO:166-(Xn2)-SEQ ID NO: 167,
x) dimeric CNTF which comprises SEQ ID NO: 169-(Xn1)-SEQ ID NO:170-(Xn2)-SEQ ID NO: 171,
xi) dimeric LIF which comprises SEQ ID NO: 173-(Xn1)-SEQ ID NO:174-(Xn2)-SEQ ID NO: 175, and
xii) dimeric CSF3 which comprises SEQ ID NO: 177-(Xn1)-SEQ ID NO:178-(Xn2)-SEQ ID NO: 179),
or a sequence at least 80% identical thereto that retains at least the same stability, and/or at least the same level of interaction with the respective receptor(s) of said monomeric long chain Class I cytokine;
wherein (Xn1) and (Xn2) are peptide linkers.

15. The single chain dimeric cytokine polypeptide of claim 10, which is dimeric IL-2 and comprises SEQ ID NO: 89-(Xn1)-SEQ ID NO: 90-(Xn2)-SEQ ID NO:91, wherein the peptide linkers Xn1 and Xn2 are identical and comprise, or consist of, SEQ ID NO: 270, SEQ ID NO: 271, SEQ ID NO: 272, SEQ ID NO: 273, SEQ ID NO: 274, or SEQ ID NO: 275.

16. The single chain dimeric cytokine polypeptide of claim 10, which is a single chain dimeric class II polypeptide wherein:
a) a linker sequence bridges the first α-helix of a first class II cytokine monomer with the sixth α-helix of a second class II cytokine monomer;
b) a linker sequence bridges the sixth α-helix of said first class II cytokine monomer with the first α-helix of said second class II cytokine monomer; and
c) the first and second second α-helices, the second and third α-helices, the third and fourth α-helices, the fourth and fifth α-helices, or the fifth and sixth α-helices of said first class II cytokine monomer are not bridged.

17. The single chain dimeric cytokine polypeptide of claim 1, which comprises (a) a first class I cytokine monomer domain or a functional portion thereof; and (b) a second class I cytokine monomer domain or a functional portion thereof, wherein the sequence of the first class I cytokine monomer domain or functional portion thereof is continuous in sequence, while the sequence of the second cytokine monomer domain is such that the first sequence portion of the second class I cytokine monomer which is arranged at the N-terminus of the first class I cytokine monomer or functional portion thereof corresponds to a N-terminal portion of a natural class I cytokine monomer, and the second sequence portion of the second class I cytokine monomer which is arranged at the C-terminus of the second class I cytokine monomer or functional portion thereof corresponds to a C-terminal portion of the natural class I cytokine monomer.

18. The single chain dimeric cytokine polypeptide of claim 17, which comprises:
i) a split domain that comprises the free N- and C-termini of the single chain dimeric class I cytokine polypeptide, and the first and second sequence portions of the second class I cytokine monomer domain; and
ii) a continuous domain that comprises the first class I cytokine monomer domain or a functional portion thereof;
wherein the sequences of the first and second sequence portions of the first class I cytokine monomer domain, in the split domain, are separated by the sequence of the continuous domain.

19. The single chain dimeric cytokine polypeptide of claim 17, wherein:
a) a linker peptide bridges the fourth α-helix of a first class I cytokine monomer with the third α-helix of the second class I cytokine monomer;
b) a linker peptide bridges the first α-helix of the first class I cytokine monomer with the second α-helix of the second class I cytokine monomer;
c) a linker peptide bridges the first α-helix (α-helixA) of the first class I cytokine monomer with the second α-helix of the second class I cytokine monomer; and
d) α-helices B and C of said second class I cytokine monomer are not bridged.

20. The single chain dimeric cytokine polypeptide of claim 17, which is a single chain dimeric IL-2 polypeptide that comprises sequence SEQ ID NO: 296, or a sequence at least 80% identical thereto that retains at least the same stability, and/or at least the same level of interaction with IL-2 receptor.

21. The single chain dimeric cytokine polypeptide of claim 1, which comprises: (a) a first class II cytokine monomer domain or a functional portion thereof; and (b) a second class II cytokine monomer domain or a functional portion thereof, wherein the sequence of the second class II cytokine monomer domain or functional portion thereof is continuous in sequence and in reverse order compared to its natural sequence order, while the sequence of the first class II cytokine monomer domain is such that the first sequence portion of the first class II cytokine monomer domain which is arranged at the N-terminus of the second class II cytokine monomer domain or functional portion thereof corresponds to a N-terminal portion of a natural class II cytokine monomer domain, and the second sequence portion of the first class II cytokine monomer domain which is arranged at the C-terminus of the second class II cytokine monomer domain or functional portion thereof corresponds to a C-terminal portion of the natural class II cytokine monomer domain.

22. The single chain dimeric cytokine polypeptide of claim 21, which is a single chain dimeric class II cytokine polypeptide that comprises:
i) a split domain that comprises the free N- and C-termini of the single chain dimeric class II cytokine polypeptide, and the first and second sequence portions of the first class II cytokine monomer domain in their natural sequence order; and
ii) a continuous domain that comprises the second class II cytokine monomer domain or a functional portion thereof;
wherein the sequences of the first and second sequence portions of the first class II cytokine monomer domain, in the split domain, are separated by the sequence of the continuous domain of the second class II cytokine monomer domain.

23. The single chain dimeric cytokine polypeptide of claim 5, wherein the linker peptides have a sequence of from about 3 to about 20 amino acids, from about 3 to about 16 amino acids, from about 4 to about 12 amino acids, from about 4 to about 8 amino acids, from about 3 to about 8 amino acids or from about 3 to about 6 amino acids; and wherein the linker peptide sequences comprises 5 or less and suitably 3 or less adjacent Gly and/or Ser residues; optionally wherein the linker peptides are structured linkers.

24. The single chain dimeric cytokine polypeptide according to claim 1, which comprises a first and a second cytokine monomer domains that form swapped domain dimers that are Class I cytokines, and said single chain dimeric cytokine polypeptide comprises, from the N-terminus to the C-terminus, a first cytokine monomer domain that comprises helices A to C of the second cytokine monomer, and helix D of the first cytokine monomer, and a second cytokine monomer domain that comprises helix D of the second cytokine monomer and helices A to C of the first cytokine monomer, wherein a linker peptide bridges helix D of the second cytokine monomer with helix A of the first cytokine monomer.

25. The single chain dimeric cytokine polypeptide according to claim 24, which is a dimeric IL-5 polypeptide that comprises sequence SEQ ID NO: 101-(NtCt)-SEQ ID NO: 102, wherein (NtCt) is a peptide linker, or a sequence at least 80% identical thereto and that retain at least the same stability, and/or at least the same level of interaction with IL-5 receptor.

26. The single chain dimeric cytokine polypeptide according to claim 1, which comprises a first and a second cytokine monomer that form swapped domain dimers and that are Class II cytokines, and said single chain dimeric cytokine polypeptide comprises, from the N-terminus to the C-terminus, a first cytokine monomer domain that comprises helices A to C of the second cytokine monomer, and helices D to F of the first cytokine monomer, and a second cytokine monomer domain that comprises helices D to F of the second cytokine monomer and helices A to C of the first cytokine monomer, wherein a linker peptide bridges helix F of the second cytokine monomer with helix A of the first cytokine monomer.

27. The single chain dimeric cytokine polypeptide of claim 26, which is single chain dimeric IL-10 and comprises sequence SEQ ID NO: 11-(NtCt)-SEQ ID NO: 12 wherein (NtCt) is a peptide linker, or a sequence having at least 80% sequence identity thereto and that retain at least the same stability, and/or at least the same level of interaction with IL-10 receptor.

28. The single chain dimeric IL-10 polypeptide according to claim 27 which comprises SEQ ID NO:9 or SEQ ID NO:10, or a sequence at least 80% identical thereto and that retain at least the same stability, and/or at least the same level of interaction with IL-10 receptor.

29. The single chain dimeric cytokine polypeptide of claim 26, which is single chain dimeric IFNγ and comprises sequence SEQ ID NO: 14-(NtCt)-SEQ ID NO:15 wherein (NtCt) is a peptide linker, or a sequence having at least 80% sequence identity thereto and that retain at least the same stability, and/or at least the same level of interaction with IFNγ receptor.

30. The single chain dimeric polypeptide of claim 29, which comprises SEQ ID NO: 18, or a sequence having at least 80% sequence identity thereto and that retain at least the same stability, and/or at least the same level of interaction with IFNγ receptor.

31. A polypeptide comprising the single chain dimeric polypeptide of claim 1.

32. A nucleic acid encoding a polypeptide as defined in claim 1.

33. An expression vector comprising nucleic acid as defined in claim 32.

34. A host cell comprising a nucleic acid as defined in claim 32.

35. A pharmaceutical composition comprising a polypeptide as defined in claim 1.

36. A method of treating a disease, that comprises administering a polypeptide as defined in claim 1 to a subject in need thereof, optionally in a subject that would benefit from a reduction or increase in an inflammatory response or in rate of cell proliferation.

37. (canceled)

38. A polypeptide as defined in claim 1, for the manufacture of a medicament for treating a disease, optionally a disease that would benefit from a reduction or increase in an inflammatory response or in rate of cell proliferation.


SEQUENCE LISTING (354 sequences)

SEQ ID NO: 1 - protein, 6 aa, synthetic construct
FGGLDY

SEQ ID NO: 2 - DNA, 39 bp, synthetic construct
  source 1..39: mol_type = other DNA; note = P3 promoter
tattgcataatattgtataatattgtataattaaaaata

SEQ ID NO: 3 - DNA, 93 bp, synthetic construct
  source 1..93: mol_type = other DNA; note = S142
atgaagtccaagttgaaactcaaacgctatttactctttctccccttgttaccactcggtaccttgagtttagctaacacttacttgttacaa

SEQ ID NO: 4 - protein, 161 aa, Homo sapiens
  source 1..161: note = WT human IL-10
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 5 - protein, 167 aa, synthetic construct
  source 1..167: note = MutM - Engineered monomer IL-10 that has an inserted polyGly loop
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 6 - protein, 161 aa, synthetic construct
  source 1..161: note = IL-10 variant Mut1
MSPGQGTQSENSCTHFPGNLPNMLRDLREAFRRVKTFFQMKDQLDSLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMMMKIRN

SEQ ID NO: 7 - protein, 161 aa, synthetic construct
  source 1..161: note = IL-10 variant Mut2
MSPGQGTQSENSCTHFPGNLPNMLRDLREAFKRVKTFFQMKDQLDSLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMMMKIRN

SEQ ID NO: 8 - protein, 161 aa, synthetic construct
  source 1..161: note = IL-10 variant Mut3
MSPGQGTQSENSCTHFPGILPNMLRDLREAFKRVKTFFQMKDQLDSLLLKESLLEDFKGYLGCQALSEMIQFYLEEVMPQAENQDPDIKAHVISLGENLNTLRLRLRRCHRFLPCENKSKAVEQVKNAFNKLQEKGIYKAMSEFDIFINYIEAYMMMKIRN

SEQ ID NO: 9 - protein, 301 aa, synthetic construct
  source 1..301: note = MutSC1 - Single-chain dimeric IL-10 connecting the N- and C-termini by a designed FGGLDY residue loop mutated to NGGLDY
MTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMNNGGLDYLPNMLRDLRDAFSRVKTFFQMKDQLDNLLLKESLLEDFKGYLGCQALSEMIQFYLEEVMPQAENQDPDIKAHVNSLGENLKTLRLRLRRNHRFLPCENKSKAVEQVKNAFNKLQEKGIYKAMSEFDIFINYIEAYMTMKIRN

SEQ ID NO: 10 - protein, 301 aa, synthetic construct
  source 1..301: note = MutSC2: variant of single chain dimeric IL-10 construct MutSC1
MTQSENSCTHFPGILPNMLRDLREAFKRVKTFFQMKDQLDSLLLKESLLEDFKGYLGCQALSEMIQFYLEEVMPQAENQDPDIKAHVISLGENLNTLRLRLRRCHRFLPCENKSKAVEQVKNAFNKLQEKGIYKAMSEFDIFINYIEAYMMMNNGGLDYLPNMLRDLREAFKRVKTFFQMKDQLDSLLLKESLLEDFKGYLGCQALSEMIQFYLEEVMPQAENQDPDIKAHVISLGENLNTLRLRLRRNHRFLPCENKSKAVEQVKNAFNKLQEKGIYKAMSEFDIFINYIEAYMMMKIRN

SEQ ID NO: 11 - protein, 149 aa, synthetic construct
CTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 12 - protein, 138 aa, synthetic construct
PNMLRDLRDAFSRVKTFFQMKDQLDNLLLKESLLEDFKGYLGCQALSEMIQFYLEEVMPQAENQDPDIKAHVNSLGENLKTLRLRLRRNCHRFLPCENKSKAVEQVKNAFNKLQEKGIYKAMSEFDIFINYIEAYMTM

SEQ ID NO: 13 - protein, 6 aa, synthetic construct
  source 1..6: note = NtCt linker for dimeric SC IL-10
NGGLDY

SEQ ID NO: 14 - protein, 125 aa, synthetic construct
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPMLF

SEQ ID NO: 15 - protein, 144 aa, synthetic construct [sequence omitted]

SEQ ID NO: 16 - protein, 4 aa, synthetic construct
  source 1..4: note = NtCt linker for SC dimeric IFN-gamma
EGPG

SEQ ID NO: 17 - protein, 11 aa, synthetic construct
  source 1..11: note = NtCt linker for SC dimeric IFN-gamma
ASKPHPGQLWY

SEQ ID NO: 18 - protein, 251 aa, synthetic construct
  source 1..251: note = SC dimeric IFN-gamma
GSQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMASKPHPGQLWAYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAA

SEQ ID NO: 19 - protein, 11 aa, synthetic construct
AHVNSLGENLK

SEQ ID NO: 20 - protein, 8 aa, synthetic construct
ESLLEDFK

SEQ ID NO: 21 - protein, 9 aa, synthetic construct
DQLDNLLLK

SEQ ID NO: 22 - protein, 13 aa, synthetic construct
VMPQAENQDPDIK

SEQ ID NO: 23 - protein, 7 aa, synthetic construct
SLLEDFK

SEQ ID NO: 24 - protein, 13 aa, synthetic construct
DFKGYLGCQALSE

SEQ ID NO: 25 - protein, 15 aa, synthetic construct
NSCTHFPGNLPNMLR

SEQ ID NO: 26 - protein, 10 aa, synthetic construct
DQLDNLLLKE

SEQ ID NO: 27 - protein, 16 aa, synthetic construct
LEEVMPQAENQDPDIK

SEQ ID NO: 28 - protein, 17 aa, synthetic construct
YLEEVMPQAENQDPDIK

SEQ ID NO: 29 - protein, 12 aa, synthetic construct
LGCQALSEMIQF

SEQ ID NO: 30 - protein, 14 aa, synthetic construct
AHVISLGENLNTLR

SEQ ID NO: 31 - protein, 16 aa, synthetic construct
AHVISLGENLNTLRLR

SEQ ID NO: 32 - protein, 19 aa, synthetic construct
TQSENSCTHFPGILPNMLR

SEQ ID NO: 33 - DNA, 20 bp, synthetic construct
  source 1..20: mol_type = other DNA
cccactaacatcaaatgggg

SEQ ID NO: 34 - DNA, 20 bp, synthetic construct
  source 1..20: mol_type = other DNA
ccttccacaatgccaaagtt

SEQ ID NO: 35 - DNA, 18 bp, synthetic construct
  source 1..18: mol_type = other DNA
aggcactcccccaaaaga

SEQ ID NO: 36 - DNA, 20 bp, synthetic construct
  source 1..20: mol_type = other DNA
gctcctccacttggtggttt

SEQ ID NO: 37 - DNA, 20 bp, synthetic construct
  source 1..20: mol_type = other DNA
gacagactgctctgatggca

SEQ ID NO: 38 - DNA, 20 bp, synthetic construct
  source 1..20: mol_type = other DNA
tgcacttcttttcgcacaac

SEQ ID NO: 39 - DNA, 21 bp, synthetic construct
  source 1..21: mol_type = other DNA
cccagccaggtgtcattttcc

SEQ ID NO: 40 - DNA, 21 bp, synthetic construct
  source 1..21: mol_type = other DNA
gcattcagttccaggtcagtg

SEQ ID NO: 41 - DNA, 19 bp, synthetic construct
  source 1..19: mol_type = other DNA
cttctgggcctgctgttca

SEQ ID NO: 42 - DNA, 21 bp, synthetic construct
  source 1..21: mol_type = other DNA
ccagcctactcattgggatca

SEQ ID NO: 43 - DNA, 19 bp, synthetic construct
  source 1..19: mol_type = other DNA
ctggcaaaaggatggtgac

SEQ ID NO: 44 - DNA, 20 bp, synthetic construct
  source 1..20: mol_type = other DNA
gctgatggcctgattgtctt

SEQ ID NO: 45 - DNA, 21 bp, synthetic construct
  source 1..21: mol_type = other DNA
ctcctgaagctgttgcgttac

SEQ ID NO: 46 - DNA, 21 bp, synthetic construct
  source 1..21: mol_type = other DNA
gctcccttacaggctgagttc

SEQ ID NO: 47 - DNA, 20 bp, synthetic construct
  source 1..20: mol_type = other DNA
gctgcttccaaacctttgac

SEQ ID NO: 48 - DNA, 20 bp, synthetic construct
  source 1..20: mol_type = other DNA
agcttctccacagccacaat

SEQ ID NO: 49 - DNA, 20 bp, synthetic construct
  source 1..20: mol_type = other DNA
gcaacgaagaacaccacaga

SEQ ID NO: 50 - DNA, 20 bp, synthetic construct
  source 1..20: mol_type = other DNA
gcaacgaagaacaccacaga

SEQ ID NO: 51 - DNA, 20 bp, synthetic construct
  source 1..20: mol_type = other DNA
tgatgcacttgcagaaaaca

SEQ ID NO: 52 - DNA, 21 bp, synthetic construct
  source 1..21: mol_type = other DNA
ggtcttggtccttagccactc

SEQ ID NO: 53 - DNA, 21 bp, synthetic construct
  source 1..21: mol_type = other DNA
cagcaccagcttcttcatcag

SEQ ID NO: 54 - DNA, 22 bp, synthetic construct
  source 1..22: mol_type = other DNA
tcttcaaaggcttcatctgcaa

SEQ ID NO: 55 - DNA, 22 bp, synthetic construct
  source 1..22: mol_type = other DNA
actgtacaaccgcagtaatacg

SEQ ID NO: 56 - DNA, 23 bp, synthetic construct
  source 1..23: mol_type = other DNA
agtgaacattacagatttatccc

SEQ ID NO: 57 - DNA, 21 bp, synthetic construct
  source 1..21: mol_type = other DNA
tgctggattgcagagcagtaa

SEQ ID NO: 58 - DNA, 21 bp, synthetic construct
  source 1..21: mol_type = other DNA
gcatgcagagattccgagaga

SEQ ID NO: 59 - skipped

SEQ ID NO: 60 - DNA, 24 bp, synthetic construct
  source 1..24: mol_type = other DNA
aaaataagggttaattggtaaacc

SEQ ID NO: 61 - DNA, 25 bp, synthetic construct
  source 1..25: mol_type = other DNA
  modified_base 1..4: mod_base = OTHER; note = phosphorotiated base
gtgagcacaatcagcgctcaaactg

SEQ ID NO: 62 - DNA, 23 bp, synthetic construct
  source 1..23: mol_type = other DNA
gtaagttatcgaggtgacttcac

SEQ ID NO: 63 - protein, 299 aa, synthetic construct
  source 1..299: note = MutSC1_polyglycontrol_19
MTQENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMGGGGGYLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 64 - protein, 289 aa, synthetic construct
  source 1..289: note = MutSC1_controlcenter_21
KSKAVEQVKNAFNKLQEKGIYKAMSEFDIFINYIEAYMTMNFGGLDYLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMNFGGLDYLPNMLRDLRDAFSRVKTFFQMKDQLDNLLLKESLLEDFKGYLGCQALSEMIQFYLEEVMPQAENQDPDIKAHVNSLGENLKTLRLRLRRCHFLPCEN

SEQ ID NO: 65 - protein, 280 aa, synthetic construct
  source 1..280: note = SCIL22_1 (foldikine-22_1)
GCRLDKSNFQQPYITNRTFMLAKEASLADNNTDVRLIGEKLFHGVSMSERCYLMKQVLNFTLEEVLFPQSDRFQPYMQEVVPFLARLSNRLSTCMPGGSKTHIQRNVQKLKDTVKKLGESGEIKAIGELDLLFMSLRNACIANGTLDKSNFQQPYITNRTFMLAKEASLADNNTDVRLIGEKLFHGVSMSERCYLMKQVLNFTLEEVLFPQSDRFQPYMQEVVPFLARLSNRLSRLELLPHIQRNVQKLKDTVKKLGESGEIKAIGELDLLFMSLRNACI

SEQ ID NO: 66 - protein, 278 aa, synthetic construct
  source 1..278: note = SCIL22_3 (foldikine-22_3)
GVRLDKSNFQQPYITNRTFMLAKEASLADNNTDVRLIGEKLFHGVSMSERCYLMKQVLNFTLEEVLFPQSDRFQPYMQEVVPFLARLSNRLKKLMASSDHIQRNVQKLKDTVKKLGESGEIKAIGELDLLFMSLRNACIANGTLDKSNFQQPYITNRTFMLAKEASLADNNTDVRLIGEKLFHGVSMSERCYLMKQVLNFTLEEVLFPQSDRFQPYMQEVVPFLARLSNRLSRLELLPHIQRNVQKLKDTVKKLGESGEIKAIGELDLLFMSLRNACI

SEQ ID NO: 67 - protein, 278 aa, synthetic construct
  source 1..278: note = foldikine-22_3_linkerNCpolygly
GVRLDKSNFQQPYITNRTFMLAKEASLADNNTDVRLIGEKLFHGVSMSERCYLMKQVLNFTLEEVLFPQSDRFQPYMQEVVPFLARLSNRLKKLMASSDHIQRNVQKLKDTVKKLGESGEIKAIGELDLLFMSLRNACIGGGGLDKSNFQQPYITNRTFMLAKEASLADNNTDVRLIGEKLFHGVSMSERCYLMKQVLNFTLEEVLFPQSDRFQPYMQEVVPFLARLSNRLSRLELLPHIQRNVQKLKDTVKKLGESGEIKAIGELDLLFMSLRNACI

SEQ ID NO: 68 - protein, 278 aa, synthetic construct
  source 1..278: note = foldikine-22_3_centrallinkers
GVRLDKSNFQQPYITNRTFMLAKEASLADNNTDVRLIGEKLFHGVSMSERCYLMKQVLNFTLEEVLFPQSDRFQPYMQEVVPFLARLSGGSGGSGGSGGHIQRNVQKLKDTVKKLGESGEIKAIGELDLLFMSLRNACIANGTLDKSNFQQPYITNRTFMLAKEASLADNNTDVRLIGEKLFHGVSMSERCYLMKQVLNFTLEEVLFPQSDRFQPYMQEVVPFLARLSNRLSGGSGGSHIQRNVQKLKDTVKKLGESGEIKAIGELDLLFMSLRNACI

SEQ ID NO: 69 - protein, 140 aa, synthetic construct
  source 1..140: note = IL-22 3dlq. Pdb sequence
ARLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 70 - protein, 140 aa, synthetic construct
  source 1..140: note = 3dlq. Pdb sequence - Hydrophobic core stabilised protein (IL-22hydC)
ARLDKSNFQQPYITNRMFMLAKEMSLADNNTDVRLIGEKLFHGVSMSERCYLMKQILNFMLEEVLFPQSDRFQPYMQEVVPFLARLSNRLSTCHIEGDDLHIQRNVQKLKDTVKKLGELGEIKAIGELDLLFMSLRNACI

SEQ ID NO: 71 - protein, 146 aa, synthetic construct
APISSHCRLEKRNFQQPYITNRTFMLAKEASLADNNIDVRLIGYKLFHGVSMSERCYLMKQVLNFTLEEVLFPQSDRFQPYMQEVVPFLYRLSNRLSTCHIEGDDLHIQRNVQKLKDTVKKLGESGEIKAIGELDLLFMSLRNACI

SEQ ID NO: 72 - protein, 146 aa, synthetic construct
APISSHCRLDKSNFQQPYITNRTFMLAWEAMLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 73 - protein, 146 aa, synthetic construct
APISSHCREDRSNFQQPYITNRTFMLAWEAMLADNNIDVRLIGYKLFHGVSMSERCYLMKQVLNFTLEEVLFPQSDRFQPYMQEVVPFLYRLSNRLSTCHIEGDDLHIQRNVQKLKDTVKKLGESGEIKAIGELDLLFMSLRNACI

SEQ ID NO: 74 - protein, 282 aa, synthetic construct
  source 1..282: note = Foldikine10-22
CRLDKSNFQQPYITNRTFMLAKEASLADNNTDVRLIGEKLFHGVSMSERCYLMKQVLNFTLEEVLFPQSDRFQPYMQEVVPFLARLSNRLDPKAAFKSKAVEQVKNAFNKLQEKGIYKAMSEFDIFINYIEAYMTMNFGGLDYLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCDSKVNRRNVQKLKDTVKKLGESGEIKAIGELDLLFMSLRNACI

SEQ ID NO: 75 - protein, 282 aa, synthetic construct
  source 1..282: note = Foldikine10-22_3
ARLDKSNFQQPYITNRTFMLAKEASLADNNTDVRLIGEKLFHGVSMSERCYLMLQVLNFTLEEVLFPQSDRFQPYMQEVVPFLARLSNRLDPKAAFKCKAVEQVKNAFNKLQEKGIYKAMSEFDIFINYIEAYMTMNFGGLDYLPNMLRDLRDAFSRVKTFFQMKDQLDNLLLKESLLEDFKGYLGCLCLSEMIQFYLEEVMPQAENQDPDIKAHVNSLGENLKTLRLRLRRCHRFLPCISKVLRRNVQKLKDTVKKLGESGEIKAIGELDLLFMSLRNACI

SEQ ID NO: 76 - protein, 282 aa, synthetic construct
  source 1..282: note = Foldikine10-22_5
ARLDKSNFQQPYITNRLFMLAKEASLMDNNTDVRLIGEKLFHGVSMSERCYLMLQILNFFLEEVLFPQSDRFQPYMQEVVPFLARLSNRLDPKAAFKCKAVEQVKNAFNKLQEKGIYKAMSEFDIFINYIEAYMTMNFGGLDYLPNMLRDLRDAFSRVKTFFQMKDQLDNLLLKESLLEDFKGYLGCLCLSEMIQFYLEEVMPQAENQDPDIKAHVNSLGENLKTLRLRLRRCHRFLPCISKVLRRNVQKLKDTVKKLGESGEIKAIGELDLLFMLLRNACI

SEQ ID NO: 77 - protein, 317 aa, synthetic construct
  source 1..317: note = heterodimer10-22
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGNGGKSKAVEQVKNAFNKLQEKGIYKAMSEFDIFINYIEAYMTMKIRNGGSGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 78 - protein, 317 aa, synthetic construct
  source 1..317: note = heterodimer22-10
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIGGSGGSMSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGNGGKSKAVEQVKNAFNKLQEKGIYKAMSEFDIFINYIEAYMTMKIRN

SEQ ID NO: 79 - protein, 153 aa, Homo sapiens
  source 1..153: note = Interleukin-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 80 - protein, 138 aa, Homo sapiens
  source 1..138: note = Interleukin-3
RPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 81 - protein, 135 aa, Homo sapiens
  source 1..135: note = Interleukin-4
GNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 82 - protein, 113 aa, Homo sapiens
  source 1..113: note = Interleukin-5
TEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 83 - protein, 157 aa, Homo sapiens
  source 1..157: note = Interleukin-7
PVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 84 - protein, 126 aa, Homo sapiens
  source 1..126: note = Interleukin-9
QGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 85 - protein, 141 aa, Homo sapiens
  source 1..141: note = Interleukin-15
NSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 86 - protein, 137 aa, Homo sapiens
  source 1..137: note = Interleukin-21
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSED

SEQ ID NO: 87 - protein, 131 aa, Homo sapiens
  source 1..131: note = Thymic stromal lymphopoietin
YDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 88 - protein, 117 aa, Homo sapiens
  source 1..117: note = Granulocyte-macrophage colony-stimulating factor
QPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 89 - protein, 52 aa, synthetic construct
PRDLISNINVIVLELKGSETTFMCEYADETATIVEFLNRWITFCQSIISTLT

SEQ ID NO: 90 - protein, 131 aa, synthetic construct
TSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 91 - protein, 71 aa, synthetic construct
TSSSTKKTQLQLEHLLLDLQMILNGINNYKNPKLTRMLTFKFYMPKKATELKHLQCLEEELKPLEEVLNLA

SEQ ID NO: 92 - protein, 8 aa, synthetic construct
QSKNFHLR

SEQ ID NO: 93 - protein, 59 aa, synthetic construct
QQFHRHKQLIRFLKRLDRNLWGLAGLNSCPVKEANQSTLENFLERLKTIMREKYSKCSS

SEQ ID NO: 94 - protein, 135 aa, synthetic construct; identical to SEQ ID NO: 81

SEQ ID NO: 95 - protein, 72 aa, synthetic construct
GNFVHGHKCDITLQEIIKTLNSLTEQKTLCTELTVTDIFAASKNTTEKETFCRAATVLRQFYSHHEKDTRCL

SEQ ID NO: 96 - protein, 4 aa, synthetic construct
GATA

SEQ ID NO: 97 - protein, 52 aa, synthetic construct
SAIESILKNLLPCLPLATAAPTRHPIHIKDGDWNEFRRKLTFYLKTLENAQA

SEQ ID NO: 98 - protein, 113 aa, synthetic construct
SWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQ

SEQ ID NO: 99 - protein, 54 aa, synthetic construct
SWVNCSNMIDEIITHLKQPPLPLLDFNNLNGEDQDILMENNLRRPNLEAFNRAV

SEQ ID NO: 100 - protein, 6 aa, synthetic construct
KSLQNA

SEQ ID NO: 101 - protein, 113 aa, synthetic construct; identical to SEQ ID NO: 82

SEQ ID NO: 102 - protein, 113 aa, synthetic construct; identical to SEQ ID NO: 82

SEQ ID NO: 103 - protein, 79 aa, synthetic construct
DFDLHLLKVSEGTTILLNCTGQVKGRKPAALGEAQPTKSLEENKSLKEQKKLNDLCFLKRLLQEIKTCWNKILMGTKEH

SEQ ID NO: 104 - protein, 157 aa, synthetic construct; identical to SEQ ID NO: 83

SEQ ID NO: 105 - protein, 72 aa, synthetic construct
PVASSDCDIEGKDGKQYESVLMVSIDQLLDSMKEIGSNCLNNEFNFFKRHICDANKEGMFLFRAARKLRQFL

SEQ ID NO: 106 - protein, 6 aa, synthetic construct
KMNSTG

SEQ ID NO: 107 - protein, 58 aa, synthetic construct
LIFSRVKKSVEVLKNNKCPYFSCEQPCNQTTAGNALTFLKSLLEIFQKEKMRGMRGKI

SEQ ID NO: 108 - protein, 126 aa, synthetic construct; identical to SEQ ID NO: 84

SEQ ID NO: 109 - protein, 65 aa, synthetic construct
QGCPTLAGILDINFLINKMQEDPASKCHCSANVTSCLCLGIPSDNCTRPCFSERLSQMTNTTMQT

SEQ ID NO: 110 - protein, 58 aa, synthetic construct
ASIHDTVENLIILANNSLSSNGNVTESGCKECEELEEKNIKEFLQSFVHIVQMFINTS

SEQ ID NO: 111 - protein, 117 aa, synthetic construct
TEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 112 - protein, 61 aa, synthetic construct
NSHFLTEANWVNVISDLKKIEDLIQSMHIDATLYTESDVHPSCKVTAMKCFLLELQVISLE

SEQ ID NO: 113 - protein, 70 aa, synthetic construct
NERIINVSIKKLKRKPPSTNAGRRQKHRLTCPSCDSYEKKPPKEFLERFKSLLQKMIHQHLSSRTHGSED

SEQ ID NO: 114 - protein, 137 aa, synthetic construct; identical to SEQ ID NO: 86

SEQ ID NO: 115 - protein, 63 aa, synthetic construct
HKSSSQGQDRHMIRMRQLIDIVDQLKNYVNDLVPEFLPAPEDVETNCEWSAFSCFQKAQLKSA

SEQ ID NO: 116 - protein, 64 aa, synthetic construct
EMFAMKTKAALAIWCPGYSETQINATQAMKKRRKRKVTTNKCLEQVSQLQGLWRRFNRPLLKQQ

SEQ ID NO: 117 - protein, 121 aa, synthetic construct
NCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNR

SEQ ID NO: 118 - protein, 59 aa, synthetic construct
NCDFEKIKAAYLSTISKDLITYMSGTKSTEFNNTVSCSNRPHCLTEIQSLTFNPTAGCA

SEQ ID NO: 119 - protein, 4 aa, synthetic construct
SLAK

SEQ ID NO: 120 - protein, 53 aa, synthetic construct
LTKLKGPLTMMASHYKQHCPPTPETSCATQIITFESFKENLKDFLLVIPFDCW

SEQ ID NO: 121 - protein, 112 aa, synthetic construct
QPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCW

SEQ ID NO: 122 - protein, 56 aa, synthetic construct
QPWEHVNAIQEARRLLNLSRDTAAEMNETVEVISEMFDLQEPTCLQTRLELYKQGL

SEQ ID NO: 123 - protein, 184 aa, Homo sapiens
  source 1..184: note = Interleukin-6
PVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 124 - protein, 178 aa, Homo sapiens
  source 1..178: note = Interleukin 11
PGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 125 - protein, 197 aa, Homo sapiens
  source 1..197: note = Interleukin-12 subunit alpha
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 126 - protein, 170 aa, Homo sapiens
  source 1..170: note = Interleukin-23 subunit alpha
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 127 - protein, 215 aa, Homo sapiens
  source 1..215: note = Interleukin-27 subunit alpha
FPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 128 - protein, 141 aa, Homo sapiens [sequence omitted]

SEQ ID NO: 129 - protein, 198 aa, Homo sapiens
  source 1..198: note = Cardiotrophin-like cytokine factor 1
LNRTGDPGPGPSIQKTYDLTRYLEHQLRSLAGTYLNYLGPPFNEPDFNPPRLGAETLPRATVDLEVWRSLNDKLRLTQNYEAYSHLLCYLRGLNRQAATAELRRSLAHFCTSLQGLLGSIAGVMAALGYPLPQPLPGTEPTWTPGPAHSDFLQKMDDFWLLKELQTWLWRSAKDFNRLKKKMQPPAAAVTLHLGAHGF

SEQ ID NO: 130 - protein, 228 aa, Homo sapiens [sequence omitted]

SEQ ID NO: 131 - protein, 201 aa, Homo sapiens
  source 1..201: note = Cardiotrophin-1
MSRREGSLEDPQTDSSVSLLPHLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLPGGSA

SEQ ID NO: 132 - protein, 200 aa, Homo sapiens
  source 1..200: note = Ciliary neurotrophic factor
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 133 - protein, 180 aa, Homo sapiens
  source 1..180: note = Leukemia inhibitory factor
SPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 134 - protein, 177 aa, Homo sapiens
  source 1..177: note = Granulocyte colony-stimulating factor
TPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 135 - protein, 77 aa, synthetic construct
SEEQARAVQMSTKVLIQFLQKKAKNLDAITTPDPTTNASLLTKLQAQNQWLQDMTTHLILRSFKEFLQSSLRALRQM

SEQ ID NO: 136 - protein, 166 aa, synthetic construct
LTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 137 - protein, 89 aa, synthetic construct
LTSSERIDKQIRYILDGISALRKETCNKSNMCESSKEALAENNLNLPKMAEKDGCFQSGFNEETCLVKIITGLLEFEVYLEYLQNRFES

SEQ ID NO: 138 - protein, 4 aa, synthetic construct
ESSE

SEQ ID NO: 139 - protein, 82 aa, synthetic construct
SLKTLEPELGTLQARLDRLLRRLQLLMSRLALPQPPPDPPAPPLAPPSSAWGGIRAAHAILGGLHLTLDWAVRGLLLLKTRL

SEQ ID NO: 140 - protein, 168 aa, synthetic construct
SPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 141 - protein, 84 aa, synthetic construct
SPDPRAELDSTVLLTRSLLADTRQLAAQLRDKFPADGDHNLDSLPTLAMSAGALGALQLPGVLTRLRADLLSYLRHVQWLRRAG

SEQ ID NO: 142 - protein, 4 aa, synthetic construct
GSSL

SEQ ID NO: 143 - protein, 69 aa, synthetic construct
RQIFLDQNMLAVIDELMQALNFNSETVPQKSSLEEPDFYKTKIKLCILLHAFRIRAVTIDRVMSYLNAS

SEQ ID NO: 144 - protein, 195 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKIFLDQNMLAVIDELMQALNFNSETVPQKSSLEEPDFYKTKIKLCILLHAFRIRAVTIDRVMSYLNAS

SEQ ID NO: 145 - protein, 124 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLL

SEQ ID NO: 146 - protein, 4 aa, synthetic construct
MDPK

SEQ ID NO: 147 - protein, 74 aa, synthetic construct
LPDSPVGQLHASLLGLSQLLQPEGHHWETQQIPSLSPSQPWQRLLLRFKILRSLQAFVAVAARVFAHGAATLSP

SEQ ID NO: 148 - protein, 162 aa, synthetic construct
SPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLS

SEQ ID NO: 149 - protein, 84 aa, synthetic construct
SPAWTQCQQLSQKLCTLAWSAHPLVGHMDLREEGDEETTNDVPHIQCGDGCDPQGLRDNSQFCLQRIHQGLIFYEKLLGSDIFT

SEQ ID NO: 150 - protein, 7 aa, synthetic construct
TGEPSLL

SEQ ID NO: 151 - protein, 101 aa, synthetic construct
WTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGH

SEQ ID NO: 152 - protein, 187 aa, synthetic construct
LRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGH

SEQ ID NO: 153 - protein, 83 aa, synthetic construct
LRREFTVSLHLARKLLSEVRGQAHRFAESHLPGVNLYLLPLGEQLPDVSLTFQAWRRLSDPERLCFISTTLQPFHALLGGLGT

SEQ ID NO: 154 - protein, 62 aa, synthetic construct
VIDEIIEHLDKLIFQDAPETNISVPTDTHECKRFILTISQQFSECMDLALKSLTSGAQQATT

SEQ ID NO: 155 - protein, 141 aa, synthetic construct [sequence omitted]

SEQ ID NO: 156 - protein, 75 aa, synthetic construct
SHTLPVRLLRPSDDVQKIVEELQSLSKMLLKDVEEEKGVLVSQNYTLPCLSPDAQPPNNIHSPAIRAYLKTIRQL

SEQ ID NO: 157 - protein, 4 aa, synthetic construct
DNKS

SEQ ID NO: 158 - protein, 86 aa, synthetic construct
AELRRSLAHFCTSLQGLLGSIAGVMAALGYPLPQPLPGTEPTWTPGPAHSDFLQKMDDFWLLKELQTWLWRSAKDFNRLKKKMQPP

SEQ ID NO: 159 - protein, 179 aa, synthetic construct
PGPGPSIQKTYDLTRYLEHQLRSLAGTYLNYLGPPFNEPDFNPPRLGAETLPRATVDLEVWRSLNDKLRLTQNYEAYSHLLCYLRGLNRQAATAELRRSLAHFCTSLQGLLGSIAGVMAALGYPLPQPLPGTEPTWTPGPAHSDFLQKMDDFWLLKELQTWLWRSAKDFNRLKKKMQPP

SEQ ID NO: 160 - protein, 89 aa, synthetic construct
PGPGPSIQKTYDLTRYLEHQLRSLAGTYLNYLGPPFNEPDFNPPRLGAETLPRATVDLEVWRSLNDKLRLTQNYEAYSHLLCYLRGLNR

SEQ ID NO: 161 - protein, 86 aa, synthetic construct
NIEDLEKLQMARPNILGLRNNIYCMAQLLDNSDTAEPTKAGRGASQPPTPTPASDAFQRKLEGCRFLHGYHRFMHSVGRVFSKWGE

SEQ ID NO: 162 - protein, 185 aa, synthetic construct
SCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGE

SEQ ID NO: 163 - protein, 90 aa, synthetic construct
SCSKEYRVLLGQLQKQTDLMQDTSRLLDPYIRIQGLDVPKLREHCRERPGAFPSEETLRGLGRRGFLQTLNATLGCVLHRLADLEQRLPK

SEQ ID NO: 164 - protein, 11 aa, synthetic construct
KAQDLERSGLN

SEQ ID NO: 165 - protein, 82 aa, synthetic construct
PRLLRRLEDAARQARALGAAVEALLAALGAANRGPRAEPPAATASAASATGVFPAKVLGLRVCGLYREWLSRTEGDLGQLLP

SEQ ID NO: 166 - protein, 176 aa, synthetic construct
HLEAKIRQTHSLAHLLTKYAEQLLQEYVQLQGDPFGLPSFSPPRLPVAGLSAPAPSHAGLPVHERLRLDAAALAALPPLLDAVCRRQAELNPRAPRLLRRLEDAARQARALGAAVEALLAALGAANRGPRAEPPAATASAASATGVFPAKVLGLRVCGLYREWLSRTEGDLGQLLP

SEQ ID NO: 167 - protein, 90 aa, synthetic construct
HLEAKIRQTHSLAHLLTKYAEQLLQEYVQLQGDPFGLPSFSPPRLPVAGLSAPAPSHAGLPVHERLRLDAAALAALPPLLDAVCRRQAEL

SEQ ID NO: 168 - protein, 4 aa, synthetic construct
NPRA

SEQ ID NO: 169 - protein, 96 aa, synthetic construct
FHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 170 - protein, 173 aa, synthetic construct
PHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQ

SEQ ID NO: 171 - protein, 88 aa, synthetic construct
PHRRDLCSRSIWLARKIRSDLTALTESYVKHQGLNKNINLDSADGMPVASTDQWSELTEAERLQENLQAYRTFHVLLARLLEDQQVHF

SEQ ID NO: 172 - protein, 6 aa, synthetic construct
TPTEGD

SEQ ID NO: 173 - protein, 72 aa, synthetic construct
LSLHSKLNATADILRGLLSNVLCRLCSKYHVGHVDVTYGPDTSGKDVFQKKKLGCQLLGKYKQIIAVLAQAF

SEQ ID NO: 174 - protein, 165 aa, synthetic construct
HPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 175 - protein, 89 aa, synthetic construct
HPCHNNLMNQIRSQLAQLNGSANALFILYYTAQGEPFPNNLDKLCGPNVTDFPPFHANGTEKAKLVELYRIVVYLGTSLGNITRDQKIL

SEQ ID NO: 176 - protein, 4 aa, synthetic construct
NPSA

SEQ ID NO: 177 - protein, 77 aa, synthetic construct
ELGPTLDTLQLDVADFATTIWQQMEELGMAPALQPTQGAMPAFASAFQRRAGGVLVASHLQSFLEVSYRVLRHLAQP

SEQ ID NO: 178 - protein, 171 aa, synthetic construct
SSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 179 - protein, 89 aa, synthetic construct
SSLPQSFLLKCLEQVRKIQGDGAALQEKLVSECATYKLCHPEELVLLGHSLGIPWAPLSSCPSQALQLAGCLSQLHSGLFLYQGLLQAL

SEQ ID NO: 180 - protein, 4 aa, synthetic construct
EGIS

SEQ ID NO: 181 - protein, 154 aa, Homo sapiens
QSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 182 - protein, 158 aa, Homo sapiens
VDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 183 - protein, 151 aa, Homo sapiens
KTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 184 - protein, 146 aa, Homo sapiens
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 185 - protein, 149 aa, Homo sapiens
HKQSSFTKSCYPRGTLSQAVDALYIKAAWLKATIPEDRIKNIRLLKKKTKKQFMKNCQFQEQLLSFFMEDVFGQLQLQGCKKIRFVEDFHSLRQKLSHCISCASSAREMKSITRMKRIFYRIGNKGIYKAISELDILLSWIKKLLESSQ

SEQ ID NO: 186 - protein, 162 aa, Homo sapiens
RGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 187 - protein, 175 aa, Homo sapiens
VPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 188 - protein, 181 aa, Homo sapiens
GPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 189 - protein, 165 aa, Homo sapiens
DLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 190 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 191 - protein, 174 aa, Homo sapiens
  source 1..174: note = Interferon omega-1
LGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 192 - protein, 187 aa, Homo sapiens
  source 1..187: note = Interferon epsilon
LDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 193 - protein, 180 aa, Homo sapiens
  source 1..180: note = Interferon kappa
LDCNLLNVHLRRVTWQNLRHLSSMSNSFPVECLRENIAFELPQEFLQYTQPMKRDIKKAFYEMSLQAFNIFSQHTFKYWKERHLKQIQIGLDQQAEYLNQCLEEDKNENEDMKEMKENEMKPSEARVPQLSSLELRRYFHRIDNFLKEKKYSDCAWEIVRVEIRRCLYYFYKFTALFRRK

SEQ ID NO: 194 - protein, 143 aa, Homo sapiens
  source 1..143: note = Interferon gamma
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 195 - protein, 101 aa, synthetic construct
VDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQ

SEQ ID NO: 196 - protein, 47 aa, synthetic construct
QEATNATRVIHDNYDQLEVHAAAIKSLGELDVFLAWINKNHEVMFSA

SEQ ID NO: 197 - protein, 101 aa, synthetic construct; identical to SEQ ID NO: 195

SEQ ID NO: 198 - protein, 47 aa, synthetic construct; identical to SEQ ID NO: 196

SEQ ID NO: 199 - protein, 5 aa, synthetic construct
CQQCR

SEQ ID NO: 200 - protein, 104 aa, synthetic construct
KTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAH

SEQ ID NO: 201 - protein, 42 aa, synthetic construct
GEEAMKKYSQILSHFEKLEPQAAVVKALGELDILLQWMEETE

SEQ ID NO: 202 - protein, 104 aa, synthetic construct; identical to SEQ ID NO: 200

SEQ ID NO: 203 - protein, 42 aa, synthetic construct; identical to SEQ ID NO: 201

SEQ ID NO: 204 - protein, 5 aa, synthetic construct
MTCHC

SEQ ID NO: 205 - protein, 90 aa, synthetic construct
CRLDKSNFQQPYITNRTFMLAKEASLADNNTDVRLIGEKLFHGVSMSERCYLMKQVLNFTLEEVLFPQSDRFQPYMQEVVPFLARLSNRL

SEQ ID NO: 206 - protein, 40 aa, synthetic construct
HIQRNVQKLKDTVKKLGESGEIKAIGELDLLFMSLRNACI

SEQ ID NO: 207 - protein, 96 aa, synthetic construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRL

SEQ ID NO: 208 - protein, 40 aa, synthetic construct; identical to SEQ ID NO: 206

SEQ ID NO: 209 - protein, 10 aa, synthetic construct
STCHIEGDDL

SEQ ID NO: 210 - protein, 100 aa, synthetic construct
HKQSSFTKSCYPRGTLSQAVDALYIKAAWLKATIPEDRIKNIRLLKKKTKKQFMKNCQFQEQLLSFFMEDVFGQLQLQGCKKIRFVEDFHSLRQKLSHCI

SEQ ID NO: 211 - protein, 41 aa, synthetic construct
MKSITRMKRIFYRIGNKGIYKAISELDILLSWIKKLLESSQ

SEQ ID NO: 212 - protein, 100 aa, synthetic construct; identical to SEQ ID NO: 210

SEQ ID NO: 213 - protein, 41 aa, synthetic construct; identical to SEQ ID NO: 211

SEQ ID NO: 214 - protein, 8 aa, synthetic construct
SCASSARE

SEQ ID NO: 215 - protein, 102 aa, synthetic construct
RGCHIAQFKSLSPQELQAFKRAKDALEESLLLKDCRCHSRLFPRTWDLRQLQVRERPMALEAELALTLKVLEATADTDPALVDVLDQPLHTLHHILSQFRAC

SEQ ID NO: 216 - protein, 47 aa, synthetic construct
RLHHWLYRLQEAPKKESPGCLEASVTFNLFRLLTRDLNCVASGDLCV

SEQ ID NO: 217 - protein, 102 aa, synthetic construct; identical to SEQ ID NO: 215

SEQ ID NO: 218 - protein, 47 aa, synthetic construct; identical to SEQ ID NO: 216

SEQ ID NO: 219 - protein, 13 aa, synthetic construct
IQPQPTAGPRTRG

SEQ ID NO: 220 - protein, 102 aa, synthetic construct
RGCHIAQFKSLSPQELQAFKRAKDALEESLLLKDCKCRSRLFPRTWDLRQLQVRERPVALEAELALTLKVLEATADTDPALGDVLDQPLHTLHHILSQLRAC

SEQ ID NO: 221 - protein, 47 aa, synthetic construct
RLHHWLHRLQEAPKKESPGCLEASVTFNLFRLLTRDLNCVASGDLCV

SEQ ID NO: 222 - protein, 102 aa, synthetic construct; identical to SEQ ID NO: 220

SEQ ID NO: 223 - protein, 47 aa, synthetic construct; identical to SEQ ID NO: 221

SEQ ID NO: 224 - protein, 14 aa, synthetic construct
IQPQPTAGPRTRGR

SEQ ID NO: 225 - protein, 102 aa, synthetic construct
TGKGCHIGRFKSLSPQELASFKKARDALEESLKLKNWSCSSPVFPGNWDLRLLQVRERPVALEAELALTLKVLEAAAGPALEDVLDQPLHTLHHILSQLQAC

SEQ ID NO: 226 - protein, 55 aa, synthetic construct
LHHWLHRLQEAPKKESAGCLEASVTFNLFRLLTRDLKYVADGNLCLRTSTHPEST

SEQ ID NO: 227 - protein, 102 aa, synthetic construct; identical to SEQ ID NO: 225

SEQ ID NO: 228 - protein, 55 aa, synthetic construct; identical to SEQ ID NO: 226

SEQ ID NO: 229 - protein, 14 aa, synthetic construct
IQPQPTAGPRTRGR

SEQ ID NO: 230 - protein, 93 aa, synthetic construct
SLDNRRTLMLLAQMSRISPSSCLMDRHDFGFPQEEFDGNQFQKAPAISVLHELIQQIFNLFTTKDSSAAWDEDLLDKFCTELYQQLNDLEACV

SEQ ID NO: 231 - protein, 53 aa, synthetic construct
ADSILAVKKYFRRITLYLTEKKYSPCAWEVVRAEIMRSLSLSTNLQERLRRKE

SEQ ID NO: 232 - protein, 93 aa, synthetic construct; identical to SEQ ID NO: 230

SEQ ID NO: 233 - protein, 53 aa, synthetic construct; identical to SEQ ID NO: 231

SEQ ID NO: 234 - protein, 13 aa, synthetic construct
MQEERVGETPLMN

SEQ ID NO: 235 - protein, 92 aa, synthetic construct
SLGSRRTLMLLAQMRKISLFSCLKDRHDFGFPQEEFGNQFQKAETIPVLHEMIQQIFNLFSTKDSSAAWDETLLDKFYTELYQQLNDLEACV

SEQ ID NO: 236 - protein, 53 aa, synthetic construct
EDSILAVRKYFQRITLYLKEKKYSPCAWEVVRAEIMRSFSLSTNLQESLRSKE

SEQ ID NO: 237 - protein, 92 aa, synthetic construct; identical to SEQ ID NO: 235

SEQ ID NO: 238 - protein, 44 aa, synthetic construct
EDSILAVRKYFQRITLYLKEKKYSPCAWEVVRAEIMRSFSLSTN

SEQ ID NO: 239 - protein, 13 aa, synthetic construct
IQGVGVTETPLMK

SEQ ID NO: 240 - protein, 107 aa, synthetic construct
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLE

SEQ ID NO: 241 - protein, 51 aa, synthetic construct
LMSSLHLKRYYGRILHYLKAKEYSHCAWTIVRVEILRNFYFINRLTGYLRN

SEQ ID NO: 242 - protein, 107 aa, synthetic construct; identical to SEQ ID NO: 240

SEQ ID NO: 243 - protein, 51 aa, synthetic construct; identical to SEQ ID NO: 241

SEQ ID NO: 244 - protein, 8 aa, synthetic construct
KEDFTRGK

SEQ ID NO: 245 - protein, 101 aa, synthetic construct
LGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETC

SEQ ID NO: 246 - protein, 45 aa, synthetic construct
SPALTLRRYFQGIRVYLKEKKYSDCAWEVVRMEIMKSLFLSTNMQ

SEQ ID NO: 247 - protein, 99 aa, synthetic construct
CDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETC

SEQ ID NO: 248 - protein, 45 aa, synthetic construct; identical to SEQ ID NO: 246

SEQ ID NO: 249 - protein, 15 aa, synthetic construct
LLQVVGEGESAGAIS

SEQ ID NO: 250 - protein, 103 aa, synthetic construct
LDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALM

SEQ ID NO: 251 - protein, 72 aa, synthetic construct
SDNLRLQVKMYFRRIHDYLENQDYSTCAWAIVQVEISRCLFFVFSLTEKLSKQGRPLNDMKQELTTEFRSPR

SEQ ID NO: 252 - protein, 103 aa, synthetic construct; identical to SEQ ID NO: 250

SEQ ID NO: 253 - protein, 72 aa, synthetic construct; identical to SEQ ID NO: 251

SEQ ID NO: 254 - protein, 12 aa, synthetic construct
GLEAEKLSGTLG

SEQ ID NO: 255 - protein, 102 aa, synthetic construct
LDCNLLNVHLRRVTWQNLRHLSSMSNSFPVECLRENIAFELPQEFLQYTQPMKRDIKKAFYEMSLQAFNIFSQHTFKYWKERHLKQIQIGLDQQAEYLNQCL

SEQ ID NO: 256 - protein, 56 aa, synthetic construct
NIFSQHTFKYWKERHLKQIQIGLDQQAEYLNQCLXNVEIRRCLYYFYKFTALFRRK

SEQ ID NO: 257 - protein, 102 aa, synthetic construct; identical to SEQ ID NO: 255

SEQ ID NO: 258 - protein, 54 aa, synthetic construct
VPQLSSLELRRYFHRIDNFLKEKKYSDCAWEIVRVEIRRCLYYFYKFTALFRRK

SEQ ID NO: 259 - protein, 24 aa, synthetic construct
EEDKNENEDMKEMKENEMKPSEAR

SEQ ID NO: 260 - protein, 56 aa, synthetic construct
FHLRPRDLISNINVIVLELKGSETTFMCEYADETATIVEFLNRWITFCQSIISTLT

SEQ ID NO: 261 - protein, 140 aa, synthetic construct
CRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 262 - protein, 75 aa, synthetic construct
TSSSTKKTQLQLEHLLLDLQMILNGINNYKNPKLTRMLTFKFYMPKKATELKHLQCLEEELKPLEEVLNLAQSKN

SEQ ID NO: 263 - protein, 56 aa, synthetic construct; identical to SEQ ID NO: 260

SEQ ID NO: 264 - protein, 41 aa, synthetic construct
LHIQRNVQKLKDTVKKLGESGEIKAIGELDLLFMSLRNACI

SEQ ID NO: 265 - protein, 90 aa, synthetic construct; identical to SEQ ID NO: 205

SEQ ID NO: 266 - protein, 75 aa, synthetic construct; identical to SEQ ID NO: 262

SEQ ID NO: 267 - protein, 90 aa, synthetic construct; identical to SEQ ID NO: 205

SEQ ID NO: 268 - protein, 131 aa, synthetic construct; identical to SEQ ID NO: 90

SEQ ID NO: 269 - protein, 41 aa, synthetic construct; identical to SEQ ID NO: 264

SEQ ID NO: 270 - protein, 6 aa, synthetic construct
GPPPPG

SEQ ID NO: 271 - protein, 7 aa, synthetic construct
GPPPPPG

SEQ ID NO: 272 - protein, 8 aa, synthetic construct
GPPPPPPG

SEQ ID NO: 273 - protein, 6 aa, synthetic construct
GGGSGG

SEQ ID NO: 274 - protein, 7 aa, synthetic construct
GGGSGGG

SEQ ID NO: 275 - protein, 8 aa, synthetic construct
GGGSGGGG

SEQ ID NO: 276 - protein, 284 aa, synthetic construct
MYRMQLLSCIALSLALVTNSLRPRDLISNINVIVLELKGSETTFMCEYADETATIVEFLNRWITFCQSIISTLTGPPPPPGTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGPPPPPGTKKTQLQLEHLLLDLQMILNGINNYKNPKLTRMLTFKFYMPKKATELKHLQCLEEELKPLEEVLNLAQS

SEQ ID NO: 277 - protein, 286 aa, synthetic construct
MYRMQLLSCIALSLALVTNSLRPRDLISNINVIVLELKGSETTFMCEYADETATIVEFLNRWITFCQSIISTLTGPPPPPPGTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGPPPPPPGTKKTQLQLEHLLLDLQMILNGINNYKNPKLTRMLTFKFYMPKKATELKHLQCLEEELKPLEEVLNLAQS

SEQ ID NO: 278 - protein, 282 aa, synthetic construct
MYRMQLLSCIALSLALVTNSLRPRDLISNINVIVLELKGSETTFMCEYADETATIVEFLNRWITFCQSIISTLTGGGSGGTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGTKKTQLQLEHLLLDLQMILNGINNYKNPKLTRMLTFKFYMPKKATELKHLQCLEEELKPLEEVLNLAQS

SEQ ID NO: 279 - protein, 284 aa, synthetic construct
MYRMQLLSCIALSLALVTNSLRPRDLISNINVIVLELKGSETTFMCEYADETATIVEFLNRWITFCQSIISTLTGGGSGGGTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGTKKTQLQLEHLLLDLQMILNGINNYKNPKLTRMLTFKFYMPKKATELKHLQCLEEELKPLEEVLNLAQS

SEQ ID NO: 280 - protein, 286 aa, synthetic construct
MYRMQLLSCIALSLALVTNSLRPRDLISNINVIVLELKGSETTFMCEYADETATIVEFLNRWITFCQSIISTLTGGGSGGGGTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGSGGGGTKKTQLQLEHLLLDLQMILNGINNYKNPKLTRMLTFKFYMPKKATELKHLQCLEEELKPLEEVLNLAQS

SEQ ID NO: 281 - protein, 262 aa, synthetic construct
ARLDKSNFQQPYITNRTFMLAKEASLADNNTDVRLIGEKLFHGVSMSERCYLMKQVLNFTLEEVLFPQSDRFQPYMQEVVPFLARLSGHIQRNVQKLKDTVKKLGESGEIKAIGELDLLFMSLRNACIANGVLDKSNFQQPYITNRTFMLAKEASLADNNTDVRLIGEKLFHGVSMSERCYLMKQVLNFTLEEVLFPQSDRFQPYMQEVVPFLARLSNRLSGHIQRNVQKLKDTVKKLGESGEIKAIGELDLLFMSLRNACI

SEQ ID NO: 282 - protein, 266 aa, synthetic construct
ARLDKSNFQQPYITNRTFMLAKEASLADNNTDVRLIGEKLFHGVSMSERCYLMKQVLNFTLEEVLFPQSDRFQPYMQEVVPFLARLSGGGHIQRNVQKLKDTVKKLGESGEIKAIGELDLLFMSLRNACIANGVLDKSNFQQPYITNRTFMLAKEASLADNNTDVRLIGEKLFHGVSMSERCYLMKQVLNFTLEEVLFPQSDRFQPYMQEVVPFLARLSNRLSGGGHIQRNVQKLKDTVKKLGESGEIKAIGELDLLFMSLRNACI

SEQ ID NO: 283 - protein, 272 aa, synthetic construct
ARLDKSNFQQPYITNRTFMLAKEASLADNNTDVRLIGEKLFHGVSMSERCYLMKQVLNFTLEEVLFPQSDRFQPYMQEVVPFLARLSGGGSGGHIQRNVQKLKDTVKKLGESGEIKAIGELDLLFMSLRNACIANGVLDKSNFQQPYITNRTFMLAKEASLADNNTDVRLIGEKLFHGVSMSERCYLMKQVLNFTLEEVLFPQSDRFQPYMQEVVPFLARLSNRLSGGGSGGHIQRNVQKLKDTVKKLGESGEIKAIGELDLLFMSLRNACI

SEQ ID NO: 284 - protein, 278 aa, synthetic construct
ARLDKSNFQQPYITNRTFMLAKEASLADNNTDVRLIGEKLFHGVSMSERCYLMKQVLNFTLEEVLFPQSDRFQPYMQEVVPFLARLSGGGSGGSGGHIQRNVQKLKDTVKKLGESGEIKAIGELDLLFMSLRNACIANGVLDKSNFQQPYITNRTFMLAKEASLADNNTDVRLIGEKLFHGVSMSERCYLMKQVLNFTLEEVLFPQSDRFQPYMQEVVPFLARLSNRLSGGGSGGSGGHIQRNVQKLKDTVKKLGESGEIKAIGELDLLFMSLRNACI

SEQ ID NO: 285 - protein, 284 aa, synthetic construct
ARLDKSNFQQPYITNRTFMLAKEASLADNNTDVRLIGEKLFHGVSMSERCYLMKQVLNFTLEEVLFPQSDRFQPYMQEVVPFLARLSGGGSGGSGGSGGHIQRNVQKLKDTVKKLGESGEIKAIGELDLLFMSLRNACIANGVLDKSNFQQPYITNRTFMLAKEASLADNNTDVRLIGEKLFHGVSMSERCYLMKQVLNFTLEEVLFPQSDRFQPYMQEVVPFLARLSNRLSGGGSGGSGGSGGHIQRNVQKLKDTVKKLGESGEIKAIGELDLLFMSLRNACI

SEQ ID NO: 286 - protein, 266 aa, synthetic construct
ARLDKSNFQQPYITNRTFMLAKEASLADNNTDVRLIGEKLFHGVSMSERCYLMKQVLNFTLEEVLFPQSDRFQPYMQEVVPFLARLSGPGHIQRNVQKLKDTVKKLGESGEIKAIGELDLLFMSLRNACIANGVLDKSNFQQPYITNRTFMLAKEASLADNNTDVRLIGEKLFHGVSMSERCYLMKQVLNFTLEEVLFPQSDRFQPYMQEVVPFLARLSNRLSGPGHIQRNVQKLKDTVKKLGESGEIKAIGELDLLFMSLRNACI

SEQ ID NO: 287 - protein, 278 aa, synthetic construct
ARLDKSNFQQPYITNRTFMLAKEASLADNNTDVRLIGEKLFHGVSMSERCYLMKQVLNFTLEEVLFPQSDRFQPYMQEVVPFLARLSGPPPPPPPGHIQRNVQKLKDTVKKLGESGEIKAIGELDLLFMSLRNACIANGVLDKSNFQQPYITNRTFMLAKEASLADNNTDVRLIGEKLFHGVSMSERCYLMKQVLNFTLEEVLFPQSDRFQPYMQEVVPFLARLSNRLSGPPPPPPPGHIQRNVQKLKDTVKKLGESGEIKAIGELDLLFMSLRNACI

SEQ ID NO: 288 - protein, 282 aa, synthetic construct
ARLDKSNFQQPYITNRTFMLAKEASLADNNTDVRLIGEKLFHGVSMSERCYLMKQVLNFTLEEVLFPQSDRFQPYMQEVVPFLARLSGPPPPPPPPPGHIQRNVQKLKDTVKKLGESGEIKAIGELDLLFMSLRNACIANGVLDKSNFQQPYITNRTFMLAKEASLADNNTDVRLIGEKLFHGVSMSERCYLMKQVLNFTLEEVLFPQSDRFQPYMQEVVPFLARLSNRLSGPPPPPPPPPGHIQRNVQKLKDTVKKLGESGEIKAIGELDLLFMSLRNACI

SEQ ID NO: 289 - protein, 10 aa, synthetic construct
GGPPPPPPGG

SEQ ID NO: 290 - protein, 11 aa, synthetic construct
GGGGGGGGGGA

SEQ ID NO: 291 - protein, 179 aa, Homo sapiens
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 292 - protein, 349 aa, synthetic construct
MAALQKSVSSFLMGTLATSCLLLLALLVQGGAAAPISESGEIKAIGELDLLFMSLRNACIGGPPPPPPGGAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIGGPPPPPPGGAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLG

SEQ ID NO: 293 - protein, 349 aa, synthetic construct
MAALQKSVSSFLMGTLATSCLLLLALLVQGGAAAPISESGEIKAIGELDLLFMSLRNACIGGGGGGGGGGAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIGGGGGGGGGGAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLG

SEQ ID NO: 294 - protein, 153 aa, synthetic construct [sequence omitted]

SEQ ID NO: 295 - protein, 286 aa, synthetic construct
MYRMQLLSCIALSLALVTNSLRPRDLISNINVIVLELKGSETTFMCEYADETATIVEFLNRWITFCQSIISTLTGPPPPPGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSGPPPPPGTKKTQLQLEHLLLDLQMILNGINNYKNPKLTRMLTFKFYMPKKATELKHLQCLEEELKPLEEVLNLAQS

SEQ ID NO: 296 - protein, 292 aa, synthetic construct
MYRMQLLSCIALSLALVTNSNFLRPRDLISNINVIVLELKGSETTFMCEYADETATIVEFLNRWITFCQSIISTLTGGSGGNFLRPRDLISNINVIVLELKGSETTFMCEYADETATIVEFLNRWITFCQSIISTLTGGTSSSTKKTQLQLEHLLLDLQMILNGINNYKNPKLTRMLTFKFYMPKKATELKHLQCLEEELKPLEEVLNLAQSKGGSGGTSSSTKKTQLQLEHLLLDLQMILNGINNYKNPKLTRMLTFKFYMPKKATELKHLQCLEEELKPLEEVLNLAQSK

SEQ ID NO: 297 - protein, 180 aa, synthetic construct
MAALQKSVSSFLMGTLATSCLLLLALLVQGGAANNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACTDYDSQTNCRLDKSNFQQPYITNRTFMLAKEASLAD

SEQ ID NO: 298 - protein, 180 aa, synthetic construct
MAALQKSVSSFLMGTLATSCLLLLALLVQGGAAEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACTDYDSQTNCRLDKSNFQQPYITNRTFMLAKEASLADNNTDVRLIG

SEQ ID NO: 299 - protein, 180 aa, synthetic construct
MAALQKSVSSFLMGTLATSCLLLLALLVQGGAASERCYLMKQVLNFTLEEVLFPQSDRFQPYMQEVVPFLARLSNRLSTCHIEGDDLHIQRNVQKLKDTVKKLGESGEIKAIGELDLLFMSLRNACTDYDSQTNCRLDKSNFQQPYITNRTFMLAKEASLADNNTDVRLIGEKLFHGVSM

SEQ ID NO: 300 - protein, 180 aa, synthetic construct
MAALQKSVSSFLMGTLATSCLLLLALLVQGGAADRFQPYMQEVVPFLARLSNRLSTCHIEGDDLHIQRNVQKLKDTVKKLGESGEIKAIGELDLLFMSLRNACTDYDSQTNCRLDKSNFQQPYITNRTFMLAKEASLADNNTDVRLIGEKLFHGVSMSERCYLMKQVLNFTLEEVLFPQS

SEQ ID NO: 301 - protein, 180 aa, synthetic construct
MAALQKSVSSFLMGTLATSCLLLLALLVQGGAAEGDDLHIQRNVQKLKDTVKKLGESGEIKAIGELDLLFMSLRNACTDYDSQTNCRLDKSNFQQPYITNRTFMLAKEASLADNNTDVRLIGEKLFHGVSMSERCYLMKQVLNFTLEEVLFPQSDRFQPYMQEVVPFLARLSNRLSTCHI

SEQ ID NO: 302 - protein, 180 aa, synthetic construct
MAALQKSVSSFLMGTLATSCLLLLALLVQGGAAGESGEIKAIGELDLLFMSLRNACTDYDSQTNCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKL

SEQ ID NO: 303 - protein, 8 aa, synthetic construct
TDYDSQTN

SEQ ID NO: 304 - protein, 4 aa, synthetic construct
EGPG

SEQ ID NO: 305 - protein, 21 aa, synthetic construct
MTNKCLLQIALLLCFSTTALS

SEQ ID NO: 306 - protein, 183 aa, synthetic construct
MTNKCLLQIALLLCFSTTALSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYL

SEQ ID NO: 307 - protein, 187 aa, synthetic construct
MTNKCLLQIALLLCFSTTALSGPGYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLE

SEQ ID NO: 308 - protein, 187 aa, synthetic construct
MTNKCLLQIALLLCFSTTALSGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLEGPGYNLLGFLQRSSNFQCQKLLWQLN

SEQ ID NO: 309 - protein, 187 aa, synthetic construct
MTNKCLLQIALLLCFSTTALSFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLEGPGYNLLGFLQRSSNFQCQKLLWQLNGRLEYCLKDRMNFDIPEEIKQLQQ

SEQ ID NO: 310 - protein, 187 aa, synthetic construct
MTNKCLLQIALLLCFSTTALSSTGWNETIVENLLANVYHQINHLKTVLEEKLEKEDFTRGKLMSSLHLKRYYGRILHYLKAKEYSHCAWTIVRVEILRNFYFINRLTGYLEGPGYNLLGFLQRSSNFQCQKLLWQLNGRLEYCLKDRMNFDIPEEIKQLQQFQKEDAALTIYEMLQNIFAIFRQDSS

SEQ ID NO: 311 - protein, 187 aa, synthetic construct
MTNKCLLQIALLLCFSTTALSSSLHLKRYYGRILHYLKAKEYSHCAWTIVRVEILRNFYFINRLTGYLEGPGYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLM

SEQ ID NO: 312 - protein, 125 aa, Homo sapiens
SQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAA

SEQ ID NO: 313 - protein, 278 aa, synthetic construct
MYRMQLLSCIALSLALVTNSLRPRDLISNINVIVLELKGSETTFMCEYADETATIVEFLNRWITFCQSIISTLTGPPPPGTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGPPPPGTKKTQLQLEHLLLDLQMILNGINNYKNPKLTRMLTFKFYMPKKATELKHLQCLEEELKPLEEVLN

SEQ ID NO: 314 - protein, 8 aa, synthetic construct
DPKAAFKS

SEQ ID NO: 315 - protein, 6 aa, synthetic construct
DSKVNR

SEQ ID NO: 316 - protein, 7 aa, synthetic construct
NFGGLDY

SEQ ID NO: 317 - protein, 146 aa, Homo sapiens; identical to SEQ ID NO: 184

SEQ ID NO: 318 - protein, 5 aa, synthetic construct
NGGLD

SEQ ID NO: 319 - protein, 6 aa, synthetic construct
LPKLGM

SEQ ID NO: 320 - protein, 20 aa, synthetic construct
MYRMQLLSCIALSLALVTNS

SEQ ID NO: 321 - protein, 151 aa, synthetic construct
MYRMQLLSCIALSLALVTNSNPKLTRMLTFKFYMPKKATELKHLQCLEEELKPLEEVLNLAQSKNFHLRPRDLISNINVIVLELKGSETTFMCEYADETATIVEFLNRWITFSQSIISLLPKLGMNKKTQLQLEHLLLDLQMILNGINNYK

SEQ ID NO: 322 - protein, 151 aa, synthetic construct
MYRMQLLSCIALSLALVTNSKATELKHLQCLEEELKPLEEVLNLAQSKNFHLRPRDLISNINVIVLELKGSETTFMCEYADETATIVEFLNRWITFSQSIISLLPKLGMNKKTQLQLEHLLLDLQMILNGINNYKNPKLTRMLTFKFYMPK

SEQ ID NO: 323 - protein, 151 aa, synthetic construct
MYRMQLLSCIALSLALVTNSNFHLRPRDLISNINVIVLELKGSETTFMCEYADETATIVEFLNRWITFSQSIISLLPKLGMNKKTQLQLEHLLLDLQMILNGINNYKNPKLTRMLTFKFYMPKKATELKHLQCLEEELKPLEEVLNLAQSK

SEQ ID NO: 324 - protein, 151 aa, synthetic construct
MYRMQLLSCIALSLALVTNSTTFMCEYADETATIVEFLNRWITFSQSIISLLPKLGMNKKTQLQLEHLLLDLQMILNGINNYKNPKLTRMLTFKFYMPKKATELKHLQCLEEELKPLEEVLNLAQSKNFHLRPRDLISNINVIVLELKGSE

SEQ ID NO: 325 - protein, 90 aa, synthetic construct
RLDKSNFQQPYITNRTFMLAKEASLADNNTDVRLIGEKLFHGVSMSERCYLMKQVLNFTLEEVLFPQSDRFQPYMQEVVPFLARLSNRLS

SEQ ID NO: 326 - protein, 40 aa, synthetic construct; identical to SEQ ID NO: 206

SEQ ID NO: 327 - protein, 89 aa, synthetic construct
LDKSNFQQPYITNRTFMLAKEASLADNNTDVRLIGEKLFHGVSMSERCYLMKQVLNFTLEEVLFPQSDRFQPYMQEVVPFLARLSNRLS

SEQ ID NO: 328 - protein, 40 aa, synthetic construct; identical to SEQ ID NO: 206

SEQ ID NO: 329 - protein, 4 aa, synthetic construct
ANGT

SEQ ID NO: 330 - protein, 4 aa, synthetic construct
ANGV

SEQ ID NO: 331 - protein, 6 aa, synthetic construct
RLELLP

SEQ ID NO: 332 - protein, 11 aa, synthetic construct
NRLKKLMASSD

SEQ ID NO: 333 - protein, 9 aa, synthetic construct
GGGSGGSGG

SEQ ID NO: 334 - protein, 12 aa, synthetic construct
GGGSGGSGGSGG

SEQ ID NO: 335 - protein, 9 aa, synthetic construct
GPPPPPPPG

SEQ ID NO: 336 - protein, 11 aa, synthetic construct
GPPPPPPPPPG

SEQ ID NO: 337 - protein, 9 aa, synthetic construct
TCMPGGSKT

SEQ ID NO: 338 - protein, 270 aa, synthetic construct
SSSTKKTQLQLEHLLLDLQMILNGINNYKNPKLTRMLTFKFYMPKKATELKHLQCLEEELKPLEEVLNLAQSKGGSGGSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGSGGNFLRPRDLISNINVIVLELKGSETTFMCEYADETATIVEFLNRWITFCQSIISTLTG

SEQ ID NO: 339 - DNA, 63 bp, synthetic construct
  source 1..63: mol_type = other DNA
gttaacgattaagatcaaaaagtgcctggtatcgtaaataatattgtataattaaaaaagaat

SEQ ID NO: 340 - protein, 17 aa, unidentified
LDALFAGVKEAFGGLDY

SEQ ID NO: 341 - protein, 11 aa, synthetic construct
LDALFAGVKEA

SEQ ID NO: 342 - protein, 5 aa, synthetic construct
FGGLD

SEQ ID NO: 343 - protein, 140 aa, synthetic construct
ARLDKSNFQQPYITNRMFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 344 - protein, 140 aa, synthetic construct
ARLDKSNFQQPYITNRTFMLAKEMSLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 345 - protein, 140 aa, synthetic construct
ARLDKSNFQQPYITNRTFMLAKEASLADNNTDVRLIGEKLFHGVSMSERCYLMKQILNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 346 - protein, 140 aa, synthetic construct
ARLDKSNFQQPYITNRTFMLAKEASLADNNTDVRLIGEKLFHGVSMSERCYLMKQVLNFMLEEVLFPQSDRFQPYMQEVVPFLARLSNRLSTCHIEGDDLHIQRNVQKLKDTVKKLGESGEIKAIGELDLLFMSLRNACI

SEQ ID NO: 347 - protein, 140 aa, synthetic construct
ARLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGEMGEIKAIGELDLLFMSLRNACI

SEQ ID NO: 348 - protein, 33 aa, synthetic construct
MAALQKSVSSFLMGTLATSCLLLLALLVQGGAA

SEQ ID NO: 349 - protein, 160 aa, Homo sapiens
  source 1..160: note = IL-10
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 350 - protein, 160 aa, Mus musculus
  source 1..160: note = IL-10
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 351 - protein, 160 aa, Macaca mulatta
SPGQGTQSENSCTRFPGNLPHMLRDLRDAFSRVKTFFQMKDQLDNILLKESLLEDFKGYLGCQALSEMIQFYLEEVMPQAENHDPDIKEHVNSLGENLKTLRLRLRRCHRFLPCENKSKAVEQVKNAFSKLQEKGVYKAMSEFDIFINYIEAYMTMKIQN

SEQ ID NO: 352 - protein, 154 aa, Gallus gallus
LEPTCLHFSELLPARLRELRVKFEEIKDYFQSRDDELNIQLLSSELLDEFKGTFGCQSVSEMLRFYTDEVLPRAMQTSTSHQQSMGDLGNMLLGLKATMRRCHRFFTCEKRSKAIKQIKETFEKMDENGIYKAMGEFDIFINYIEEYLLMRRRK

SEQ ID NO: 353 - protein, 160 aa, Cavia porcellus
SQGTNTQSEDSCAHFPAGLPHMLRELRAAFGRVKTFFQTQDQLDNVLLNKSLLEDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 354 - protein, 158 aa, unidentified
RRVECKTDCCSFVEGFPLRLRELRSAYKEIQKFYESNDDLEPLLNEDIKHNINSPYGCHVMNEILHFYLETILPTALQKNPLKHSTTPIDSIGNIFQELKRDMVKCKRYFSCQNPFEVNSLKNSYEKMKEKGVYKAMGELDLLFRYIEQYLASKRVKH